(19) United States
(12) Patent Application Publication
(10) Pub. No.: US 2009/0062282 A1
(43) Pub. Date: Mar. 5, 2009

(54) Substituted Amino-Pyrimidones and Uses Thereof

(75) Inventors: Jeffrey Scott Albert, Wilmington, DE (US); Don Andisik, Wilmington, DE (US); James Arnold, Wilmington, DE (US); Dean Brown, Wilmington, DE (US); Owen Callaghan, Den Bosch (NL); James Campbell, Wilmington, DE (US); Robin Arthur Ellis Carr, Bassingbourn (GB); Gianni Chessari, Cambridge (GB); Miles Stuart Congreve, Cambridge (GB); Phil Edwards, Wilmington, DE (US); James R. Empfield, Wilmington, DE (US); Martyn Frederickson, Cambridge (GB); Gerard M. Koether, Wilmington, DE (US); Jennifer Krumrine, Wilmington, DE (US); Russ Mauger, Wilmington, DE (US); Christopher William Murray, Cambridge (GB); Sahil Patel, Cambridge (GB); Mark Sylvester, Wilmington, DE (US); Scott Throner, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 11/577,154

(22) PCT Filed: Oct. 14, 2005
(86) PCT No.: PCT/SE05/01534

§ 371 Date: Jan. 9, 2008

Related U.S. Application Data
(60) Provisional application No. 60/619,514, filed on Oct. 15, 2004.

(51) Int. Cl. A61K 31/5377; C07D 239/22; A61K 31/513; C12N 9/99; A61P 25/28; C07D 413/10

(52) U.S. Cl. 514/235.8; 544/321; 514/272; 544/123; 435/184

(57) ABSTRACT

This invention relates to novel compounds having the structural formula Ia or formula Ib below: (Ia, Ib), and their pharmaceutically acceptable salts, tautomers or in vivo hydrolysable precursors, compositions and methods of use thereof. These novel compounds provide a treatment or prophylaxis of Aβ related pathologies such as cognitive impairment, Alzheimer Disease, neurodegeneration and dementia.

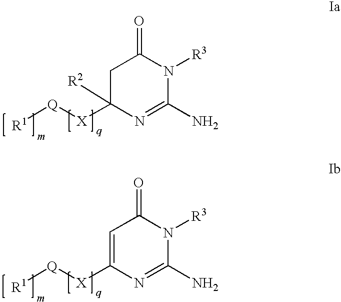

### FIELD OF THE INVENTION

The present invention relates to novel substituted amino-pyrimidones, their pharmaceutical compositions, methods of use and processes to make such compounds. In addition, the present invention relates to therapeutic methods for the treatment and/or prevention of amyloid-β-protein-related pathologies (“Aβ-related pathologies”) such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

### BACKGROUND OF THE INVENTION

Several groups have identified and isolated aspartate proteinases that have β-secretase activity (Hussain et al., 1999; Lin et. al, 2000; Yan et. al, 1999; Sinha et. al., 1999 and Vassar et. al., 1999). β-secretase is also known in the literature as Asp2 (Yan et. al, 1999), Beta site APP (amyloid precursor protein) Cleaving Enzyme (BACE) (Vassar et. al., 1999) or memapsin-2 (Lin et al., 2000). BACE was identified using a number of experimental approaches such as EST database analysis (Hussain et al. 1999); expression cloning (Vassar et al. 1999); identification of human homologs from public databases of predicted C. elegans proteins (Yan et al. 1999) and finally utilizing an inhibitor to purify the protein from human brain (Sinha et al. 1999). Thus, five groups employing three different experimental approaches led to the identification of the same enzyme, making a strong case that BACE is a β-secretase. Mention is also made of the patent literature: WO96/40885, EP871720, U.S. Pat. Nos. 5,942,400 and 5,744,346, EP855444, U.S. Pat. No. 6,319,689, WO99/64587, WO99/31236, EP1037977, WO00/17369, WO01/23533, WO0047618, WO00/58479, WO00/69262, WO01/00663, WO01/00665, U.S. Pat. No. 6,313,268.

BACE was found to be a pepsin-like aspartic proteinase, the mature enzyme consisting of the N-terminal catalytic domain, a transmembrane domain, and a small cytoplasmic domain. BACE has an optimum activity at pH 4.0-5.0 (Vassar et al, 1999)) and is inhibited weakly by standard pepsin inhibitors such as pepstatin. It has been shown that the catalytic domain minus the transmembrane and cytoplasmic domain has activity against substrate peptides (Lin et al, 2000). BACE is a membrane bound type 1 protein that is synthesized as a partially active proenzyme, and is abundantly expressed in brain tissue. It is thought to represent the major β-secretase activity, and is considered to be the rate-limiting step in the production of amyloid-β-protein (Aβ). It is thus of special interest in the pathology of Alzheimer's disease, and in the development of drugs as a treatment for Alzheimer's disease.

Aβ or amyloid-β-protein is the major constituent of the brain plaques which are characteristic of Alzheimer's disease (De Strooper et al, 1999). Aβ is a 3942 residue peptide formed by the specific cleavage of a class I transmembrane protein called APP, or amyloid precursor protein. Aβ-secretase activity cleaves this protein between residues Met671 and Asp672 (numbering of 770aa isoform of APP) to form the N-terminus of Aβ. A second cleavage of the peptide is associated with γ-secretase to form the C-terminus of the Aβ peptide.

Alzheimer's disease (AD) is estimated to afflict more than 20 million people worldwide and is believed to be the most common form of dementia. Alzheimer's disease is a progressive dementia in which massive deposits of aggregated protein breakdown products—amyloid plaques and neurofibrillary tangles accumulate in the brain. The amyloid plaques are thought to be responsible for the mental decline seen in Alzheimer's patients.

The likelihood of developing Alzheimer's disease increases with age, and as the aging population of the developed world increases, this disease becomes a greater and greater problem. In addition to this, there is a familial link to Alzheimer's disease and consequently any individuals possessing the double mutation of APP known as the Swedish mutation (in which the mutated APP forms a considerably improved substrate for BACE) have a much greater chance of developing AD, and also of developing it at an early age (see also U.S. Pat. No. 6,245,964 and U.S. Pat. No. 5,877,399 pertaining to transgenic rodents comprising APP-Swedish). Consequently, there is also a strong need for developing a compound that can be used in a prophylactic fashion for these individuals.

The gene encoding APP is found on chromosome 21, which is also the chromosome found as an extra copy in Down's syndrome. Down's syndrome patients tend to acquire Alzheimer's disease at an early age, with almost all those over 40 years of age showing Alzheimer's-type pathology (Oyama et al., 1994). This is thought to be due to the extra copy of the APP gene found in these patients, which leads to overexpression of APP and therefore to increased levels of APPβ causing the high prevalence of Alzheimer's disease seen in this population. Thus, inhibitors of BACE could be useful in reducing Alzheimer's-type pathology in Down's syndrome patients.

Drugs that reduce or block BACE activity should therefore reduce Aβ levels and levels of fragments of Aβ in the brain, or elsewhere where Aβ or fragments thereof deposit, and thus slow the formation of amyloid plaques and the progression of Aβ or other maladies involving deposition of Aβ or fragments thereof (Yankner, 1996; De Strooper and Konig, 1999). BACE is therefore an important candidate for the development of drugs as a treatment and/or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

It would therefore be useful to inhibit the deposition of Aβ and portions thereof by inhibiting BACE through inhibitors such as the compounds provided herein.

The therapeutic potential of inhibiting the deposition of Aβ has motivated many groups to isolate and characterize secretase enzymes and to identify their potential inhibitors (see, e.g., WO01/23533 A2, EP0855444, WO00/17369, WO00/58479, WO00/47618, WO00/77030, WO01/00665, WO01/00663, WO01/29563, WO02/25276, U.S. Pat. No. 5,942,400, U.S. Pat. No. 6,245,884, U.S. Pat. No. 6,221,667, U.S. Pat. No. 6,211,235, WO02/02505, WO02/02506, WO02/02512, WO02/02518, WO02/02520, WO02/14264).

### SUMMARY OF THE INVENTION

Provided herein are novel compounds of structural formula Ia or formula Ib:

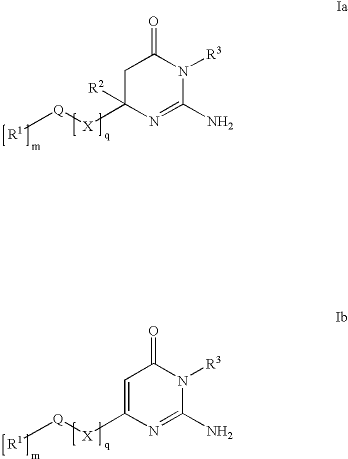

or pharmaceutically acceptable salts, tautomers or in vivo-hydrolysable precursors thereof, wherein:

Q is C₆₋₁₀aryl or C₅₋₁₀heterocyclyl;

X is C₁₋₃alkyl;

q is 0 or 1;

m is 0, 1 or 2;

each R¹ is, independently, selected from H, halogen, OH, C₁₋₆alkyl, C₂₋₆alkenyl, OC₁₋₄alkyl, OC₁₋₃alkylOC₁₋₃alkyl, OC₁₋₃alkylOC₁₋₃alkylOC₁₋₃alkyl, -L-R⁴, C₆₋₁₀aryl, OC₆₋₁₀aryl or C₅₋₁₅heterocyclyl, wherein said C₆₋₁₀aryl, OC₆₋₁₀aryl or C₅₋₁₅heterocyclyl is optionally substituted by 1, 2, or 3 substituents, independently, selected from: halogen, OH, NH₂, CN, C(═O)NH₂, C₁₋₆alkyl, C₁₋₆haloalkyl, OC₁₋₆alkyl, OC₁₋₆haloalkyl, C₁₋₄alkylOH, C₁₋₄alkylCN, C₁₋₄alkylN(C₁₋₆alkyl)₂, Si(C₁₋₄alkyl)₃, C₁₋₄alkylOC₁₋₃alkyl, CH₂OH, SO₂H, SO₂NHC(CH₃)₃, SO₂C₁₋₆alkyl, SO₂NHC₁₋₆-alkyl, OC₁₋₃alkylOC₁₋₃alkyl, OC₁₋₃alkylOH, OC₁₋₃alkylOC(═O)C₁₋₃alkyl, C(═O)C₁₋₆alkyl, C(═O)OC₁₋₆alkyl, C(═O)NH₂, C₆₋₁₀aryl, SO₂C₆₋₁₀aryl, C₅₋₆heterocyclyl optionally substituted by OC₁₋₆alkyl, OC₅₋₆aryl, OC₁₋₆alkyl-C₆₋₁₀aryl, C(═O)H, COOH, C(═O)NHC₁₋₆alkyl, C(═O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(═O)H, NHC(═O)C₅₋₆aryl, N(C₁₋₆alkyl)C(═O)C₁₋₆alkyl, NHC(═O)OC₅₋₆alkyl-C₆₋₁₀aryl, C₃₋₉cycloalkyl, C₅₋₆aryl, C(═O)C₅₋₆aryl, C(═O)OC₅₋₆aryl, C(═O)OC₁₋₆alkyl-C₆₋₁₀aryl, C(═O)NHC₅₋₆aryl, C(═O)N(C₅₋₆aryl)₂, SO₂C₅₋₆aryl SO₂NHC₅₋₆aryl, SO₂N(C₅₋₆aryl)₂, NH(C₅₋₆aryl), N(C₅₋₆aryl)₂, NHC(═O)C₅₋₆aryl, C₁₋₆alkyl-C₅₋₆heterocyclyl, OC₅₋₆heterocyclyl, C(═O)C₅₋₆heterocyclyl, C(═O)OC₅₋₆heterocyclyl, C(═O)NHC₅₋₆heterocyclyl, C(═O)N(C₅₋₆heterocyclyl)₂, SO₂C₅₋₆heterocyclyl, SO₂NHC₅₋₆heterocyclyl, SO₂N(C₅₋₆heterocyclyl)₂, NHSO₂C₁₋₆alkyl, NH(C₅₋₆heterocyclyl), N(C₅₋₆heterocyclyl)₂, or NHC(═O)C₅₋₆heterocyclyl;

R² is H, C₁₋₆alkyl, C₁₋₆haloalkyl, C₆₋₁₀aryl, or C₁₋₆alkyl-C₆₋₁₀aryl;

R³ is selected from H, C₁₋₆alkyl, C₁₋₆alkylR^{c}, C₁₋₆alkyl, C₁₋₆-alkylOR^{c}, C₁₋₆alkylNHC₁₋₆alkylC₆₋₁₀aryl, C₁₋₆alkylNHC₆₋₁₀arylR^{d}, C₁₋₆alkylNHC(O)C₆₋₁₀arylR^{d}, C₁₋₆alkylOC₁₋₆alkylC₅₋₆aryl, C₁₋₆alkylC₆₋₁₀arylR^{d}, C₁₋₆alkylC₅₋₉heterocyclylR^{d}, C₁₋₆alkylC₃₋₉cycloalkylR^{d}, C₁₋₆alkylC₁₋₆alkylC₅₋₉heterocyclylR^{d}, C₁₋₆alkylNHC₅₋₉heterocyclyl(R^{d})ₙ, C₁₋₆alkylNHC(O)C₅₋₉heterocyclyl, C₁₋₆alkylOC₁₋₆alkylC₅₋₉heterocyclylR^{d}, C₁₋₆alkylNHC₁₋₆alkylC₃₋₉cycloalkylR^{d}, or C₁₋₆alkylOC₁₋₆alkylC₃₋₉cycloalkyl;

n is 0, 1, 2, 3, 4 or 5;

each L is, independently, absent, O, O—C₁₋₄alkylenyl, C₁₋₄alkylenyl, or C₂₋₄alkenylenyl;

each R⁴ is, independently, C₆₋₁₀aryl or C₅₋₁₅heterocyclyl, wherein each said aryl and heterocyclyl is optionally substituted by 1, 2, or 3 substituents, independently, selected from: halogen, OH, NH₂, CN, C(═O)NH₂, C₁₋₆alkyl, C₁₋₆haloalkyl, OC₁₋₆alkyl, OC₁₋₆haloalkyl, C₁₋₄alkylOH, C₁₋₄alkylCN, C₁₋₄alkylN(C₁₋₆alkyl)₂, Si(C₁₋₄alkyl)₃, C₁₋₄alkylOC₁₋₃alkyl, CH₂OH, SO₂H, SO₂NHC(CH₃)₃, SO₂C₁₋₆alkyl, SO₂NHC₁₋₆alkyl, OC₁₋₃alkylOC₁₋₃alkyl, OC₁₋₃alkylOH, OC₁₋₃alkylOC(═O)C₁₋₃alkyl, C(═O)C₁₋₆alkyl, C(═O)OC₁₋₆alkyl, C(═O)NH₂, C₆₋₁₀aryl, SO₂C₆₋₁₀aryl, C₅₋₆heterocyclyl optionally substituted by OC₁₋₆alkyl, OC₅₋₆aryl, OC₁₋₆alkyl-C₆₋₁₀aryl, C(═O)H, COOH, C(═O)NHC₁₋₆alkyl, C(═O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(═O)H, NHC(═O)C₁₋₆alkyl, N(C₁₋₆alkyl)C(═O)C₁₋₆alkyl, NHC(═O)OC₁₋₆alkyl-C₆₋₁₀aryl, C₃₋₉cycloalkyl, C₅₋₆aryl, C(═O)C₅₋₆aryl, C(═O)OC₅₋₆aryl, C(═O)OC₁₋₆alkyl-C₆₋₁₀aryl, C(═O)NHC₅₋₆aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl), N(C₅₋₆aryl)₂, NHC(═O)C₅₋₆aryl, C₁₋₆alkyl-C₅₋₆heterocyclyl, OC₅₋₆heterocyclyl, C(═O)C₅₋₆heterocyclyl, C(═O)OC₅ heterocyclyl, C(═O)NHC₅₋₆heterocyclyl, C(═O)N(C₅₋₆heterocyclyl)₂, SO₂C₅₋₆heterocyclyl, SO₂NHC₅₋₆heterocyclyl, SO₂N(C₅₋₆heterocyclyl)₂, NHSO₂C₁₋₆alkyl, NH(C₅₋₆heterocyclyl), N(C₅₋₆heterocyclyl)₂, or NHC(═O)C₅₋₆heterocyclyl;

each R^{c} is, independently, selected from H, C₁₋₄alkyl, C(═O)C₁₋₄alkyl, C(═O)C₁₋₄alkylOC₁₋₄alkyl, C(═O)C₁₋₄alkylC(═O)OC₁₋₄alkyl, C(═O)C₁₋₄alkylC(═O)OH, C(═O)C₁₋₄alkylOC(═O)C₁₋₄alkyl, C₅₋₆arylR^{d}, C₅₋₉heterocyclylR^{d}, C₃₋₉cycloalkylR^{d}, C(═O)C₅₋₆arylR^{d}, C(═O)C₅₋₉heterocyclylR^{d}, C(═O)C₃₋₉cycloalkylR^{d}, C₁₋₄alkyl-C₅₋₆arylR^{d}, C₁₋₄alkyl-C₅₋₉heterocyclylR^{d}, or C₁₋₄alkyl-C₃₋₉cycloalkylR^{d}; and

R^{d} is selected from H, C₁₋₃alkyl, C₅₋₉heterocyclyl, NH₂, NO₂, OH, CN, COOH, C(═O)C₁₋₄alkyl, OC₁₋₃alkyl, or OC₁₋₃alkylOH.

The present invention further provides compositions comprising a compound of formula Ia or formula Ib, or a pharmaceutically acceptable salt, tautomer or in vivo-hydrolysable precursor thereof, and at least one pharmaceutically acceptable carrier, diluent or excipient.

The present invention further provides methods of modulating activity of BACE comprising contacting the BACE with a compound of formula Ia or formula Ib, or a pharmaceutically acceptable salt, tautomer or in vivo-hydrolysable precursor thereof.

The present invention further provides methods of treating or preventing an Aβ-related pathology in a patient comprising administering to the patient a therapeutically effective amount of a compound of formula Ia or formula Ib, or a pharmaceutically acceptable salt, tautomer or in vivo-hydrolysable precursor thereof.

The present invention further provides a compound of formula Ia or formula Ib, or a pharmaceutically acceptable salt, tautomer or in vivo-hydrolysable precursor thereof, described herein for use as a medicament.

The present invention further provides a compound of formula Ia or formula Ib, or a pharmaceutically acceptable salt, tautomer or in vivo-hydrolysable precursor thereof, described herein for the manufacture of a medicament.

### DETAILED DESCRIPTION OF THE INVENTION

Provided herein are novel compounds of structural formula Ia or formula Ib:

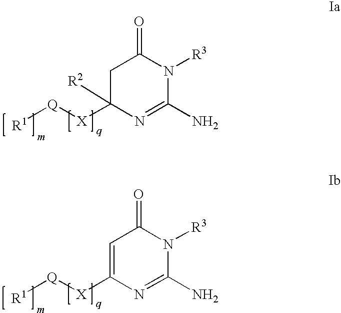

or pharmaceutically acceptable salts, tautomers or in vivo-hydrolysable precursors thereof, wherein:

Q is C₆₋₁₀aryl or C₅₋₁₀heterocyclyl;

X is C₁₋₃alkyl;

q is 0 or 1;

m is 0, 1 or 2;

each R¹ is, independently, selected from H, halogen, OH, C₁₋₆alkyl, C₂₋₆alkenyl, OC₁₋₄alkyl, OC₁₋₃alkylOC₁₋₃alkyl, OC₁₋₃alkylOC₁₋₃alkylOC₁₋₃alkyl, -L-R⁴, C₆₋₁₀aryl, OC₆₋₁₀aryl or C₅₋₁₅heterocyclyl, wherein said C₆₋₁₀aryl, OC₆₋₁₀aryl or C₅₋₁₅heterocyclyl is optionally substituted by 1, 2, or 3 substituents, independently, selected from: halogen, OH, NH₂, CN, C(═O)NH₂, C₁₋₆alkyl, C₁₋₆haloalkyl, OC₁₋₆alkyl, OC₁₋₆haloalkyl, C₁₋₄alkylOH, C₁₋₄alkylCN, C₁₋₄alkylN(C₁₋₆alkyl)₂, Si(C₁₋₄alkyl)₃, C₁₋₄alkylOC₁₋₃alkyl, CH₂OH, SO₂H, SO₂NHC(CH₃)₃, SO₂C₁₋₆alkyl, SO₂NHC₁₋₆alkyl, OC₁₋₃alkylOC₁₋₃alkyl, OC₁₋₃alkylOH, OC₁₋₃alkylOC(═O)C₁₋₃alkyl, C(═O)C₁₋₆alkyl, C(═O)OC₁₋₆alkyl, C(═O)NH₂, C₆₋₁₀aryl, SO₂C₆₋₁₀aryl, C₅₋₆heterocyclyl optionally substituted by OC₁₋₆alkyl, OC₅₋₆aryl, OC₁₋₆alkyl-C₆₋₁₀aryl, C(═O)H, COOH, C(═O)NHC₁₋₆alkyl, C(═O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(═O)H, NHC(═O)C₁₋₆alkyl, N(C₁₋₆alkyl)C(═O)C₁₋₆alkyl, NHC(═O)OC₁₋₆alkyl-C₆₋₁₀aryl, C₃₋₉cycloalkyl, C₅₋₆aryl, C(═O)C₅₋₆aryl, C(═O)OC₅₋₆aryl, C(═O)OC₁₋₆alkyl-C₆₋₁₀aryl, C(═O)NHC₅₋₆aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl), N(C₅₋₆aryl)₂, NHC(═O)C₅₋₆aryl, C₁₋₆alkyl-C₅₋₆heterocyclyl, OC₅₋₆heterocyclyl, C(═O)C₅₋₆heterocyclyl, C(═O)OC₅₋₆heterocyclyl, C(═O)NHC₅₋₆heterocyclyl, C(═O)N(C₅₋₆heterocyclyl)₂, SO₂C₅₋₆heterocyclyl, SO₂NHC₅₋₆heterocyclyl, SO₂N(C₅₋₆heterocyclyl)₂, NHSO₂C₁₋₆alkyl, NH(C₅₋₆heterocyclyl), N(C₅₋₆heterocyclyl)₂, or NHC(═O)C₅₋₆heterocyclyl;

R² is H, C₁₋₆alkyl, C₁₋₆haloalkyl, C₆₋₁₀aryl, or C₁₋₆alkyl-C₆₋₁₀aryl;

R³ is selected from H, C₁₋₆alkyl, C₁₋₆alkylR^{c}, C₁₋₆alkylNR^{c}R^{c}, C₁₋₆alkylOR^{c}, C₁₋₆alkylNHC₁₋₆alkylC₆₋₁₀arylR^{d}, C₁₋₆alkylNHC₆₋₁₀arylR^{d}, C₁₋₆alkylNHC(O)C₆₋₁₀arylR^{d}, C₁₋₆alkylOC₁₋₆alkylC₅₋₆arylR^{d}, C₁₋₆alkylC₆₋₁₀arylR^{d}, C₁₋₆alkylC₅₋₉heterocyclylR^{d}, C₁₋₆alkylC₃₋₉cycloalkylR^{d}, C₁₋₆alkylNHC₁₋₆alkylC₅₋₉heterocyclylR^{d}, C₁₋₆alkylNHC₅₋₉heterocyclyl(R^{d})ₙ, C₁₋₆alkylNHC(O)C₅₋₉heterocyclylR^{d}, C₁₋₆alkylOC₁₋₆alkylC₅₋₉heterocyclylR^{d}, C₁₋₆alkylNHC₁₋₆alkylC₃₋₉cycloalkylR^{d}, or C₁₋₆alkylOC₁₋₆alkylC₃₋₉cycloalkylR^{d};

n is 0, 1, 2, 3, 4 or 5;

each L is, independently, absent, O, O—C₁₋₄alkylenyl, C₁₋₄alkylenyl, or C₂₋₄alkenylenyl;

each R⁴ is, independently, C₆₋₁₀aryl or C₅₋₁₅heterocyclyl, wherein each said aryl and heterocyclyl is optionally substituted by 1, 2, or 3 substituents, independently, selected from: halogen, OH, NH₂, CN, C(═O)NH₂, C₁₋₆alkyl, C₁₋₆haloalkyl, OC₁₋₆alkyl, OC₁₋₆haloalkyl, C₁₋₄alkylOH, C₁₋₄alkylCN, C₁₋₄alkylN(C₁₋₆alkyl)₂, Si(C₁₋₄alkyl)₃, C₁₋₄alkylOC₁₋₃alkyl, CH₂OH, SO₂H, SO₂NHC(CH₃)₃, SO₂C₁₋₆alkyl, SO₂NHC₁₋₆alkyl, OC₁₋₃alkylOC₁₋₃alkyl, OC₁₋₃alkylOH, OC₁₋₃alkylOC(═O)C₁₋₃alkyl, C(═O)C₁₋₆alkyl, C(═O)OC₁₋₆alkyl, C(═O)NH₂, C₆₋₁₀aryl, SO₂C₆₋₁₀aryl, C₅₋₆heterocyclyl optionally substituted by OC₁₋₆alkyl, OC₅₋₆aryl, OC₁₋₆alkyl-C₆₋₁₀aryl, C(═O)H, COOH, C(═O)NHC₁₋₆alkyl, C(═O)N(C₁₋₆alkyl)₂, SO₂C₁₋₆alkyl, SO₂NHC₁₋₆alkyl, SO₂N(C₁₋₆alkyl)₂, NH(C₁₋₆-alkyl), N(C₁₋₆alkyl)₂, NHC(═O)H, NHC(═O)C₁₋₆alkyl, N(C₁₋₆alkyl)C(═O)C₁₋₆alkyl, NHC(═O)OC₁₋₆alkyl-C₆₋₁₀aryl, C₃₋₉cycloalkyl, C₅₋₆aryl, C(═O)C₅₋₆aryl, C(═O)OC₅₋₆aryl, C(═O)OC₁₋₆alkyl-C₆₋₁₀aryl, C(═O)NHC₅₋₆-aryl, C(═O)N(C₅₋₆aryl)₂, SO₂C₅₋₆aryl, SO₂NHC₅₋₆aryl, SO₂N(C₅₋₄aryl)₂, NH(C₅₋₆aryl), N(C₅₋₆aryl)₂, NHC(═O)C₅₋₆aryl, C₁₋₄alkyl-C₅₋₆heterocyclyl, OC₅₋₆heterocyclyl, C(═O)C₅₋₆heterocyclyl, C(═O)OC₅₋₆heterocyclyl, C(═O)NHC₅₋₆heterocyclyl, C(═O)N(C₅₋₆heterocyclyl)₂, SO₂C₅₋₆heterocyclyl, SO₂NHC₅₋₆heterocyclyl, SO₂N(C₅₋₆heterocyclyl)₂, NHSO₂C₁₋₆alkyl, NH(C₅₋₆heterocyclyl), N(C₅₋₆heterocyclyl)₂, or NHC(═O)C₅₋₆heterocyclyl;

each R^{c} is, independently, selected from H, C₁₋₄alkyl, C(═O)C₁₋₄alkyl, C(═O)C₁₋₄alkylOC₁₋₄alkyl, C(═O)C₁₋₄alkylC(═O)OC₁₋₄alkyl, C(═O)C₁₋₄-alkylC(═O)OH, C(═O)C₁₋₄alkylOC(═O)C₁₋₄alkyl, C₅₋₆arylR^{d}, C₅₋₉heterocyclylR^{d}, C₃₋₉cycloalkylR^{d}, C(═O)C₅₋₆arylR^{d}, C(═O)C₅₋₉heterocyclylR^{d}, C(═O)C₃₋₉cycloalkylR^{d}, C₁₋₄alkyl-C₅₋₆aryl, C₁₋₄alkyl-C₅₋₉heterocyclylR^{d}, or C₁₋₄alkyl-C₃₋₉cycloalkylR^{d}; and

R^{d} is selected from H, C₁₋₃alkyl, C₅₋₉heterocyclyl, NH₂, NO₂, OH, CN, COOH, C(═O)C₁₋₄alkyl, OC₁₋₃alkyl, or OC₁₋₃alkylOH.

In some embodiments, when the compound has formula Ia, R² is, C₁₋₄alkyl, R³ is H, C₁₋₄alkyl, C₁₋₄alkylNR^{c}R^{c} or C₁₋₄alkylOR^{c}, and q is 0, then [R¹—]ₘ-Q is other than phenyl.

In some embodiments, when the compound has formula Ia, R² is C₁₋₄alkyl, q is 0, Q is phenyl, R³ is H, C₁₋₄alkyl, C₁₋₄alkylNR^{c}R^{c} or C₁₋₄alkylOR^{c}, and m is 1, then R¹ is other than bromo, pyridyl, or methoxyphenyl.

In some embodiments, when the compound has formula Ib, X is CH₂, R³ is H, C₁₋₄alkyl, C₁₋₄alkylNR^{c}R^{c} or C₁₋₄alkylOR^{c}, and q is 1, then [R¹—]ₘ-Q is other than phenyl.

In some embodiments, when the compound has formula Ib, X is CH₂ or CH(CH₃), q is 1, Q is phenyl, R³ is H, C₁₋₄alkyl, C₁₋₄alkylNR^{c}R^{c} or C₁₋₄alkylOR^{c}, and m is 2, then at least one of R¹ is other than fluoro.

In some embodiments, compounds of the invention have the structure of said formula Ia.

In some embodiments, compounds of the invention have the structure of said formula Ib.

In some embodiments, R³ is selected from H, C₁₋₆alkyl, C₁₋₆alkylNR^{c}R^{c}, C₁₋₆alkylOR^{c}, C₁₋₆alkylNHC₁₋₆alkylC₆₋₁₀arylR^{d}, C₁₋₆alkylNHC(O)C₆₋₁₀arylR^{d}, C₁₋₆alkylOC₁₋₆alkylC₅₋₆arylR^{d}, C₁₋₆alkylC₆₋₁₀arylR^{d}, C₁₋₆alkylC₅₋₉heterocyclylR^{d}, and C₁₋₆alkylC₃₋₉cycloalkylR^{d}.

In some embodiments, R³ is selected from H, C₁₋₆alkyl, C₁₋₆alkylNR^{c}R^{c}, or C₁₋₆alkyl-C₅₋₉heterocyclylR^{d}.

In some embodiments, R³ is C₁₋₃alkyl.

In some embodiments, Q is C₆₋₁₀aryl.

In some embodiments, Q is C₆aryl.

In some embodiments, X is methylenyl, ethylenyl or propylenyl, and q is 1; wherein the ethylenyl and propylenyl can be attached from the same or different carbon atoms.

In some embodiments, X is methylenyl or ethylenyl, and q is 1.

In some embodiments, X is CH₂CH₂, and q is 1.

In some embodiments, q is 0.

In some embodiments, m is 1 or 2, and each R¹ is, independently, selected from halogen, and -L-R⁴.

In some embodiments, m is 1 and R¹ is -L-R⁴.

In some embodiments, m is 1, R¹ is -L-R⁴, and L is OCH₂:

In some embodiments, m is 1, and R¹ is R⁴.

In some embodiments, m is 1, R¹ is R⁴, and R⁴ is C₆₋₁₀aryl or C₅₋₁₅heterocyclyl wherein each said aryl and heterocyclyl is optionally substituted by 1, or 2 substituents independently selected from: halogen, CN, C₁₋₄alkyl, C₁₋₄haloalkyl, OC₁₋₄alkyl, OC₁₋₄haloalkyl, —C(O)H, COOH, OC₁₋₄alkyl-C₆₋₁₀aryl, OH, and NHC(═O)C₁₋₄alkyl.

In some embodiments, m is 2, and each of R¹ is independently phenyl or heteroaryl, each said phenyl or heteroaryl is optionally substituted by 1, 2 or 3 substituents independently selected from: methoxy, halogen, OCF₃, methyl, NH₂ and NHC(═O)CH₃. In some further embodiments wherein Q is phenyl, one R¹ is at the meta-position of Q and the other R¹ is at the para-position of Q.

In some embodiments, the compounds of the invention have the structure of formula Ib, and R² is H or C₁₋₃alkyl.

In some embodiments, compounds of the invention have the structure of formula Ib and

R² is C₁₋₃alkyl.

In some embodiments:

compounds of the invention have the structure of formula Ib;

each R¹ is, independently, selected from H, halogen, C₂₋₆alkenyl, -L-R⁴, C₁₋₁₀aryl, OC₆₋₁₀aryl or C₅₋₁₀heterocyclyl, wherein said C₆₋₁₀aryl, OC₆₋₁₀aryl or C₅₋₁₀heterocyclyl is optionally substituted by 1, 2, or 3 substituents, independently, selected from: halogen, OH, NH₂, CN, C(═O)NH₂, C₁₋₆alkyl, OC₁₋₆alkyl, C₁₋₄alkylOH, C₁₋₄alkylOC₁₋₃alkyl, CH₂OH, SO₂H, SO₂NHC(CH₃)₃, SO₂C₁₋₆alkyl, SO₂NHC₁₋₆alkyl, OC₁₋₃alkylOC₁₋₃alkyl, OC₁₋₃alkylOH, OC₁₋₃alkylOC(═O)C₁₋₃alkyl, C(═O)C₁₋₆alkyl, C(═O)OC₁₋₆alkyl, C(═O)NH₂, C₅₋₆heterocyclyl, OC₅₋₆aryl, or OC₁₋₆alkyl-C₅₋₆aryl;

R² is H or C₁₋₆alkyl; and

R³ is H or C₁₋₃alkyl.

In some embodiments:

the compounds of the invention have the structure of the formula Ib;

Q is C₆aryl or C₉heterocyclyl;

R¹ is, independently, selected from H, halogen, -L-R⁴, C₆aryl, or C₅₋₆heterocyclyl wherein said C₆aryl, or C₅₋₆heterocyclyl is optionally substituted by 1, 2, or 3 substituents, independently, selected from: halogen, OH, NH₂, CN, C(═O)NH₂, C₁₋₆alkyl, OC₁₋₆alkyl, C₁₋₄alkylOH, C₁₋₄alkylOC₁₋₃alkyl, CH₂OH, SO₂H, SO₂NHC(CH₃)₃, SO₂C₁₋₆alkyl, SO₂NHC₁₋₆alkyl, OC₁₋₃alkylOC₁₋₃alkyl, OC₁₋₃alkylOH, OC₁₋₃alkylOC(═O)C₁₋₃alkyl, C(═O)C₁₋₆alkyl, C(═O)OC₁₋₆alkyl, C(═O)NH₂, C₅₋₆heterocyclyl, OC₅₋₆aryl, or OC₁₋₆alkyl-C₅₋₆aryl; and

R² is C₁₋₃alkyl.

In some embodiments, compounds of the invention have the structure of formula Ia; R² is methyl; q is 0; Q is phenyl; m is 1; R¹ is phenyl or heteroaryl, each optionally substituted by 1, 2 or 3 substituents independently selected from: methoxy, halogen, OCF₃, methyl, NH₂ and NHC(═O)CH₃, and R³ is methyl. In further embodiments, R¹ is at the meta- or para-position of Q. In some further embodiments, R¹ is at the meta-position of Q. In further embodiments, R¹ is optionally substituted phenyl, optionally substituted pyridyl or optionally substituted furanyl. In further embodiments, the optional substituents on R¹ are at the meta and/or para position. In some further embodiments where there is 1 optional substituent on R¹ it is at the meta position or where there are 2 optional substituents on R¹ they are at the meta and para positions.

In some embodiments, compounds of the invention have the structure of formula Ia; R² is methyl; q is 1; X is C₂ alkyl; Q is phenyl; m is 1; R¹ is phenyl or heteroaryl, each optionally substituted by 1, 2 or 3 substituents independently selected from: methoxy, halogen, OCF₃, methyl, NH₂ and NHC(═O)CH₃; and R³ is methyl. In further embodiments, R¹ is at the meta- or para-position of Q. In further embodiments, R¹ is at the meta-position of Q. In further embodiments, R¹ is optionally substituted phenyl, optionally substituted pyridyl or optionally substituted furanyl. In further embodiments, X is CH₂CH₂. In further embodiments, the optional substituents on R¹ are at the meta and/or para position. In some further embodiments where there is 1 optional substituent on R¹ it is at the meta position or where there are 2 optional substituents on R¹ they are at the meta and para positions.

In some embodiments, compounds of the invention have the structure of formula Ib; q is 1; X is C₂ alkyl; Q is phenyl; m is 1; and R¹ is phenyl or heteroaryl, each optionally substituted by 1, 2 or 3 substituents independently selected from: methoxy, halogen, OCF₃, methyl, NH₂ and NHC(═O)CH₃. In further embodiments, R¹ is at the meta- or para-position of Q. In further embodiments, R¹ is at the meta-position of Q. In further embodiments, R¹ is optionally substituted phenyl, optionally substituted pyridyl or optionally substituted furanyl. In further embodiments, X is CH₂CH₂. In further embodiments, the optional substituents on R¹ are at the meta and/or para position. In some further embodiments where there is 1 optional substituent on R¹ it is at the meta position or where there are 2 optional substituents on R¹ they are at the meta and para positions.

In some embodiments, the present invention provides compounds of formula Ia or formula Ib selected from the following:
2-amino-3,6-dimethyl-6-naphthalen-2-yl-5,6-dihydro-3H-pyrimidin-4-one;
2-Amino-6-[3-(5-chloro-thiophen-2-yl)-phenyl]-3,6-dimethyl-5,6-dihydro-3H-pyrimidinone;
2-amino-6-(3,4-dichlorophenyl)-6-ethyl-5,6-dihydropyrimidin-4(3H)-one;
2-amino-6-(3,4-dichlorophenyl)-6-isobutyl-5,6-dihydropyrimidin-4(3H)-one;
2-amino-6-(3,4-dichlorophenyl)-3-methyl-6-(2-phenylethyl)-5,6-dihydropyrimidin-4(3H)-one;
2-amino-6-(3-bromo-4-chlorophenyl)-3,6-dimethyl-5,6-dihydropyrimidin-4(3H)-one;
2-amino-3,6-dimethyl-6-(2-phenylethyl)-5,6-dihydropyrimidin-4(3H)-one;
2-Amino-6-[2-(3-bromo-phenyl)-ethyl]-3,6-dimethyl-5,6-dihydro-3H-pyrimidin-4-one;
2-amino-6-[3-(benzyloxy)phenyl]-3,6-dimethyl-5,6-dihydropyrimidin-4(3H)-one;
2-amino-6-methyl-6-(3-phenoxyphenyl)-5,6-dihydropyrimidin-4(3H)-one;
2-amino-6-(3-bromo-4-chlorophenyl)-6-methyl-5,6-dihydropyrimidin-4(3H)-one;
2-amino-6-(3′-methoxy-1,1′-biphenyl-3-yl)-6-methyl-5,6-dihydropyrimidin-4(3H)-one;
2-amino-6-methyl-6-[3-(5-methylthien-2-yl)phenyl]-5,6-dihydropyrimidin-4(3H)-one;
2-amino-6-[3-(2-furyl)phenyl]-6-methyl-5,6-dihydropyrimidin-4(3H)-one;
2-amino-6-(3′-butoxy-1,1′-biphenyl-3-yl)-6-methyl-5,6-dihydropyrimidin-4(3H)-one;
2-amino-6-(6-chloro-3′-methoxy-1,1′-biphenyl-3-yl)-6-methyl-5,6-dihydropyrimidin-4(3H)-one;
2-amino-3,6-dimethyl-6-[3-(5-methylthien-2-yl)phenyl]-5,6-dihydropyrimidin 4(3H)-one;
2-amino-6-(3′-isopropoxy-1,1′-biphenyl-3-yl)-3,6-dimethyl-5,6-dihydropyrimidin-4(3H)-one;
2-amino-6-(6-chloro-3′-methoxy-1,1′-biphenyl-3-yl)-3,6-dimethyl-5,6-dihydropyrimidin-4(3H)-one;
2-amino-6-(4-chloro-3-quinolin-5-yl-phenyl)-3,6-dimethyl-5,6-hydro-3H-pyrimidin-4-one;
2-Amino-6-(2-(3′-methoxy-biphenyl-3-yl)-ethyl]-3,6-dimethyl-5,6-dihydro-3H-pyrimidin-4-one;
2-Amino-6-methyl-6-quinolin-6-yl-5,6-dihydro-3H-pyrimidin-4-one;
2-Amino-6-(3,4-dichloro-phenyl)-3,6-dimethyl-5,6-dihydro-3H-pyrimidin-4-one;
2-Amino-6-(3,4-dichloro-phenyl)-6-methyl-5,6-dihydro-3H-pyrimidin-4-one;
2-Amino-6-[2-(3-bromophenyl)ethyl]-3-methylpyrimidin-4(3H)-one;
2-Amino-6-(2-(2-bromophenyl)ethyl]-3-methylpyrimidin-4(3H)-one;
2-Amino-6-[2-(4-bromophenyl)ethyl]-3-methylpyrimidin-4(3H)-one;
2-Amino-6-[2-(1H-indol-6-yl)ethyl]-3-methylpyrimidin-4(3H)-one;
2-Amino-6-[2-(1H-indol-6-yl)ethyl]pyrimidin-4(3H)-one;
2-Amino-3-methyl-6-[2-(2-naphthyl)ethyl]pyrimidin-4(3H)-one;
2-amino-3-(1,3-dioxolan-2-ylmethyl)-6-[2-(1H-indol-6-yl)ethyl]pyrimidin-4(3H)-one;
2-Amino-3-methyl-6-{2-[3-(2-thienyl)phenyl]ethyl}pyrimidin-4(3H)-one;
2-amino-3-methyl-6-{2-[4′-(methylsulfonyl)biphenyl-3-yl]ethyl}pyrimidin-4(3H)-one;
2-amino-6-{2-[3-(1H-indol-5-yl)phenyl]ethyl}-3-methylpyrimidin-4(3H)-one;
2-amino-6-{2-[3-(2,3-dihydro-1,4-benzodioxin-6-yl)phenyl]ethyl}-3-methylpyrimidin-4(3H)-one;
2-amino-3-methyl-6-{2-[3′-(1H-pyrazol-1-yl)biphenyl-3-yl]ethyl}pyrimidin-4(3H)-one;
2-amino-6-{2-[3-(2,3-dihydro-1-benzofuran-5-yl)phenyl]ethyl}-3-methylpyrimidin-4(3H)-one;
2-amino-3-methyl-6-{2-[3-(1H-pyrrol-2-yl)phenyl]ethyl}pyrimidin-4(3H)-one;
methyl 3′-[2-(2-amino-1-methyl-6-oxo-1,6-dihydropyrimidin-4-yl)ethyl]biphenyl-4-carboxylate;
2-amino-6-{2-[3′-(hydroxymethyl)biphenyl-3-yl]ethyl}-3-methylpyrimidin-4(3H)-one;
2-amino-6-[2-(3′-hydroxybiphenyl-3-yl)ethyl]-3-methylpyrimidin-4(3H)-one;
6-[2-(3′-acetylbiphenyl-3-yl)ethyl]-2-amino-3-methylpyrimidin-4(3H)-one;
2-amino-3-methyl-6-[2-(3-pyridin-4-ylphenyl)ethyl]pyrimidin-4(3H)-one;
2-amino-6-{2-[3-(2-furyl)phenyl]ethyl}-3-methylpyrimidin-4(3H)-one;
2-amino-3-methyl-6-{2-[3-(5-methyl-2-thienyl)phenyl]ethyl}pyrimidin-4(3H)-one;
2-amino-6-[2-(3′-methoxybiphenyl-3-yl)ethyl]-3-methylpyrimidin-4(3H)-one;
2-amino-3-methyl-6-{2-[3-(4-methyl-2-thienyl)phenyl]ethyl}pyrimidin-4(3H)-one;
2-amino-6-(2-biphenyl-3-ylethyl)-3-methylpyrimidin-4(3H)-one;
2-amino-6-[2-(4′-chlorobiphenyl-3-yl)ethyl]-3-methylpyrimidin-4(3H)-one;
2-amino-6-[2-(4′-hydroxybiphenyl-3-yl)ethyl]-3-methylpyrimidin-4(3H)-one;
{3′-[2-(2-amino-1-methyl-6-oxo-1,6-dihydropyrimidin-4-yl)ethyl]biphenyl-4-yl}acetonitrile;
2-amino-3-methyl-6-{2-[3-(3-thienyl)phenyl]ethyl}pyrimidin-4(3H)-one;
2-amino-6-[2-(4′-methoxybiphenyl-3-yl)ethyl]-3-methylpyrimidin-4(3H)-one;
2-amino-6-(2-{3-[5-(hydroxymethyl)-2-thienyl]phenyl}ethyl)-3-methylpyrimidin-4(3H)-one;
2-amino-3-methyl-6-[2-(3-pyridin-3-ylphenyl)ethyl]pyrimidin-4(3H)-one;
2-amino-6-[2-(3′-ethoxybiphenyl-3-yl)ethyl]-3-methylpyrimidin-4(3H)-one;
2-amino-6-{2-[3-(3-furyl)phenyl]ethyl}-3-methylpyrimidin-4(3H)-one;
2-amino-6-[2-(2′-methoxybiphenyl-3-yl)ethyl]-3-methylpyrimidin-4(3H)-one;
3′-[2-(2-amino-1-methyl-6-oxo-1,6-dihydropyrimidin-4-yl)ethyl]-N-(tert-butyl) biphenyl-2-sulfonamide;
methyl 3′-[2-(2-amino-1-methyl-6-oxo-1,6-dihydropyrimidin-4-yl)ethyl]biphenyl-2-carboxylate;
2-amino-3-methyl-6-{2-[2-(2-thienyl)phenyl]ethyl}pyrimidin-4(3H)-one;
2-amino-6-{2-[3′-(hydroxymethyl)biphenyl-4-yl]ethyl}-3-methylpyrimidin-4(3H)-one;
2-amino-6-[2-(3′,4′-dimethoxybiphenyl-4-yl)ethyl]-3-methylpyrimidin-4(3H)-one;
2-amino-6-[2-(3′-methoxybiphenyl-4-yl)ethyl]-3-methylpyrimidin-4(3H)-one;
2-amino-3-methyl-6-{2-[4-(2-thienyl)phenyl]ethyl}pyrimidin-4(3H)-one;
2-amino-6-[2-(1,1′-biphenyl-3-yl)ethyl]pyrimidin-4(3H)-one;
N-{2-[2-Amino-4-{2-[3-(2-furyl)phenyl]ethyl}-6-oxopyrimidin-1(6H)-yl]ethyl}acetamide;
N-{2-[2-amino-4-{2-[3-(2-furyl)phenyl]ethyl}-6-oxopyrimidin-1(6H)-yl]ethyl}butanamide;
1-{2-[2-amino-4-{2-[3-(2-furyl)phenyl]ethyl}-6-oxopyrimidin-1(6H)-yl]ethyl}pyrrolidine-2,5-dione;
methyl 3-({2-[2-amino-4-{2-[3-(2-furyl)phenyl]ethyl}-6-oxopyrimidin-1(6H)-yl]ethyl}amino)-3-oxopropanoate;
methyl 5-({2-[2-amino-4-{2-[3-(2-furyl)phenyl]ethyl}-6-oxopyrimidin-1(6H)-yl]ethyl}amino)-5-oxopentanoate;
methyl 6-({2-[2-amino-4-{2-[3-(2-furyl)phenyl]ethyl}-6-oxopyrimidin-1(6H)-yl]ethyl}amino)-6-oxohexanoate;
6-({2-[2-amino-4-{2-[3-(2-furyl)phenyl]ethyl}-6-oxopyrimidin-1(6H)-yl]ethyl}amino)-6-oxohexanoic acid;
N-{2-[2-amino-4-{2-[3-(2-furyl)phenyl]ethyl}-6-oxopyrimidin-1(6H)-yl]ethyl}-2-methoxyacetamide;
2-Amino-3-[2-(benzylamino)ethyl]-6-{2-[3-(2-furyl)phenyl]ethyl}pyrimidin-4(3H)-one;
2-amino-6-{2-[3-(2-furyl)phenyl]ethyl}-3-{2-[(3-methoxybenzyl)amino]ethyl}pyrimidin-4(3H)-one;
2-({3′-[2-(2-Amino-1-methyl-6-oxo-1,6-dihydropyrimidin-4-yl)ethyl]-1,1′-biphenyl-3-yl}oxy)ethyl acetate;
2-Amino-6-{2-[3′-(2-hydroxyethoxy)-1,1′-biphenyl-3-yl]ethyl}-3-methylpyrimidin-4(3H)-one;
2-Amino-6-{2-[3′-(2-methoxyethoxy)-1,1′-biphenyl-3-yl]ethyl}-3-methylpyrimidin-4(3H)-one;
2-({3′-[2-(2-Amino-1-methyl-6-oxo-1,6-dihydropyrimidin-4-yl)ethyl]-1,1′-biphenyl-4-yl}oxy)ethyl acetate;
2-Amino-6-{2-[4′-(2-methoxyethoxy)-1,1′-biphenyl-3-yl]ethyl}-3-methylpyrimidin-4(3H)-one;
2-Amino-3-[2-(benzyloxy)ethyl]-6-{2-[3-(2-furyl)phenyl]ethyl}pyrimidin-4(3H)-one;
2-Amino-3-methyl-6-(3′-methyl-biphenyl-3-ylmethyl)-3H-pyrimidin-4-one;
2-Amino-6-(3′-methoxy-biphenyl-3-ylmethyl)-3-methyl-3H-pyrimidin-4-one;
2-Amino-6-(3′-ethoxy-biphenyl-3-ylmethyl)-3-methyl-3H-pyrimidin-4-one;
3′-(2-Amino-1-methyl-6-oxo-1,6-dihydro-pyrimidin-4-ylmethyl)-biphenyl-3-carbonitrile;
2-Amino-3-methyl-6-(3′-vinyl-biphenyl-3-ylmethyl)-3H-pyrimidin-4-one;
2-Amino-6-(3′-isopropyl-biphenyl-3-ylmethyl)-3-methyl-3H-pyrimidin-4-one;
2-Amino-6-(4′-methoxy-biphenyl-3-ylmethyl)-3-methyl-3H-pyrimidin-4-one;
2-amino-6-[2-[3-(3-methoxyphenyl)phenyl]ethyl]-3-methyl-6-phenyl-5,6-dihydro-3H-pyrimidin-4-one;
2-amino-6-[3-(3-methoxyphenyl)phenyl]-3-methyl-6-(trifluoromethyl)-5,6-dihydro-3H-pyrimidin-4-one;
(R)-2-Amino-6-[2-(3′-methoxy-biphenyl-3-yl)-ethyl]-3,6-dimethyl-5,6-dihydro-3H-pyrimidin-4-one;
2-Amino-6-[2-(2′-fluoro-3′-methoxy-biphenyl-3-yl)-ethyl]-3,6-dimethyl-5,6-dihydro-3H-pyrimidin-4-one;
2-Amino-6-{2-[3-(5-chloro-thiophen-2-yl)-phenyl]-ethyl}-3,6-dimethyl-5,6-dihydro-3H-pyrimidin-4-one;
2-Amino-6-[2-(3-furan-2-yl-phenyl)-ethyl]-3,6-dimethyl-5,6-dihydro-3H-pyrimidin-4-one;
2-Amino-6-[2-(3′,5′-dimethoxy-biphenyl-3-yl)-ethyl]-3,6-dimethyl-5,6-dihydro-3H-pyrimidinone;
2-Amino-6-[2-(4′-methoxy-biphenyl-3-yl)-ethyl]-3,6-dimethyl-5,6-dihydro-3H-pyrimidin-4-one;
2-Amino-6-(2-biphenyl-3-yl-ethyl)-3,6-dimethyl-5,6-dihydro-3H-pyrimidin-4-one;
2-Amino-3-benzyl-6-[2-(3′-methoxy-biphenyl-3-yl)-ethyl]-6-methyl-5,6-dihydro-3H-pyrimidin-4-one;
N-{3′-[2-(2-Amino-1,4-dimethyl-6-oxo-1,4,5,6-tetrahydro-pyrimidin-4-yl)-ethyl]-biphenyl-3-yl}-acetamide;
2-Amino-6-[2-(3′-hydroxymethyl-biphenyl-3-yl)-ethyl]-3,6-dimethyl-5,6-dihydro-3H-pyrimidin-4-one;
2-Amino-6-methyl-6-naphthalen-2-yl-5,6-dihydro-3H-pyrimidin-4-one;
2-Amino-3-benzyl-6-[2-(3-bromo-phenyl)-ethyl]-6-methyl-5,6-dihydro-3H-pyrimidin-4-one;
2-Amino-6-[2-(4′-methanesulfonyl-biphenyl-3-yl)-ethyl]-3,6-dimethyl-5,6-dihydro-3H-pyrimidin-4-one;
2-Amino-6-[2-(3′,4′-dimethoxy-biphenyl-3-yl)-ethyl]-3,6-dimethyl-5,6-dihydro-3H-pyrimidin-4-one;
3′-[2-(2-Amino-1,4-dimethyl-6-oxo-1,4,5,6-tetrahydro-pyrimidin-4-yl)-ethyl]-biphenyl-4-sulfonic acid dimethylamide;
2-Amino-6-(2′-fluoro-3′-methoxy-biphenyl-3-yl)-3,6-methyl-5,6-dihydro-3H-pyrimidin 4-one;
2-Amino-6-(3′,5′-dimethoxy-biphenyl-3-yl)-3,6-dimethyl-5,6-dihydro-3H-pyrimidin 4-one;
2-Amino-6-(4′-ethylbiphenyl-3-yl)-3,6-dimethyl-5,6-dihydropyrimidin-4(3H)-one;
2-amino-6-(2′-ethylbiphenyl-3-yl)-3,6-dimethyl-5,6-dihydropyrimidin-4(3H)-one;
2-amino-6-(3-isoquinolin-5-ylphenyl)-3,6-diethyl-5,6-dihydropyrimidin-4(3H)-one;
2-amino-3,6-dimethyl-6-[3′-(1H-pyrazol-1-yl)biphenyl-3-yl]-5,6-dihydropyrimidin-4(3H)-one;
2-amino-6-(4′-isopropoxybiphenyl-3-yl)-3,6-dimethyl-5,6-dihydropyrimidin-4(3H)-one;
2-amino-6-(3′-ethoxybiphenyl-3-yl)-3,6-dimethyl-5,6-dihydropyrimidin-4(3H)-one;
2-amino-3,6-dimethyl-6-[2′-(trifluoromethyl)biphenyl-3-yl]-5,6-dihydropyrimidin-4(3H)-one;
2-amino-6-(2′-chlorobiphenyl-3-yl)-3,6-dimethyl-5,6-dihydropyrimidin-4(3H)-one;
2-amino-6-(2′-fluorobiphenyl-3-yl)-3,6-dimethyl-5,6-dihydropyrimidin-4(3H)-one;
2-amino-6-(2′-methoxybiphenyl-3-yl)-3,6-dimethyl-5,6-dihydropyrimidin-4(3H)-one;
2-amino-6-(2′-ethoxybiphenyl-3-yl)-3,6-dimethyl-5,6-dihydropyrimidin-4(3H)-one;
2-amino-6-(4′-ethoxybiphenyl-3-yl)-3,6-dimethyl-5,6-dihydropyrimidin-4(3H)-one;
2-amino-6-(2′,6′-dimethylbiphenyl-3-yl)-3,6-dimethyl-5,6-dihydropyrimidin-4(3H)-one;
2-amino-3,6-dimethyl-6-[3′-(trifluoromethyl)biphenyl-3-yl]-5,6-dihydropyrimidin-4(3H)-one;
2-amino-3,6dimethyl-6-[4′-(trifluoromethyl)biphenyl-3-yl]-5,6-dihydropyrimidin-4(3H)-one;
2-amino-6-[3′,5′-bis(trifluoromethyl)biphenyl-3-yl]-3,6-dimethyl-5,6-dihydropyrimidin-4(3M)-one;
2-amino-6-(4′-tert-butylbiphenyl-3-yl)-3,6-dimethyl-5,6-dihydropyrimidin-4(3H)-one;
2-amino-6-(3′,4′-dimethoxybiphenyl-3-yl)-3,6-dimethyl-5,6-dihydropyrimidin-4(3H)-one;
2-amino-3,6-dimethyl-6-(4′-phenoxybiphenyl-3-yl)-5,6-dihydropyrimidin-4(3H)-one;
2-amino-6-(2′,4′-dimethoxybiphenyl-3-yl)-3,6-dimethyl-5,6-dihydropyrimidin-4(311)-one;
2-amino-6-[3-(1-benzofuran-2-yl)phenyl]-3,6-dimethyl-5,6-dihydropyrimidin-4(3H)-one;
2-amino-6-[3-(1,3-benzodioxol-5-yl)phenyl]-3,6-dimethyl-5,6-dihydropyrimidin-4(31)-one;
2-amino-3,6-dimethyl-6-[3′-(trifluoromethoxy)biphenyl-3-yl]-5,6-dihydropyrimidin-4(3H)-one;
2-amino-6-[3′-(hydroxymethyl)biphenyl-3-yl]-3,6-dimethyl-5,6-dihydropyrimidin-4(3H)-one;
2-amino-3,6-dimethyl-6-[3-(1-naphthyl)phenyl]-5,6-dihydropyrimidin-4(3H)-one;
2-amino-3,6-dimethyl-6-(1,1′:3′,1″-terphenyl-3-yl)-5,6-dihydropyrimidin-4(3H)-one;
2-amino-6-[4′-(benzyloxy)biphenyl-3-yl]-3,6-dimethyl-5,6-dihydropyrimidin-4(3M-one;
2-amino-6-[3′-(benzyloxy)biphenyl-3-yl]-3,6-dimethyl-5,6-dihydropyrimidin-4(3H)-one;
2-amino-6-(4′-butylbiphenyl-3-yl)-3,6-dimethyl-5,6-dihydropyrimidin-4(3H)-one;
2-amino-6-[3-(2,3-dihydro-1,4-benzodioxin-6-yl)phenyl]-3,6-dimethyl-5,6-dihydropyrimidin-4(3H)-one;
2-amino-6-[3-(2,3-dihydro-1-benzofuran-5-yl)phenyl]-3,6-dimethyl-5,6-dihydropyrimidin-4(3H)-one;
2-amino-3,6-dimethyl-6-(4′-propylbiphenyl-3-yl)-5,6-dihydropyrimidin-4(3H)-one;
2-amino-6-(2′,3′-dimethoxybiphenyl-3-yl)-3,6-dimethyl-5,6-dihydropyrimidin-4(3H)-one;
3′-(2-amino-1,4-dimethyl-6-oxo-1,4,5,6-tetrahydropyrimidin-4-yl)biphenyl-3-carboxylic acid;
2-amino-3,6-dimethyl-6-(3-thianthren-1-ylphenyl)-5,6-dihydropyrimidin-4(3H)-one;
2-amino-3,6-dimethyl-6-(1,1′:2′,1″-terphenyl-3-yl)-5,6-dihydropyrimidin-4(3H)-one;
2-amino-3,6-dimethyl-6-[3-(2-thienyl)phenyl]-5,6-dihydropyrimidin-4(3H)-one;
2-amino-3,6-dimethyl-6-[3-(3-thienyl)phenyl]-5,6-dihydropyrimidin-4(3H)-one;
2-amino-6-(3′,5′-dimethylbiphenyl-3-yl)-3,6-dimethyl-5,6-dihydropyrimidin-4(3H)-one;
2-amino-3,6-dimethyl-6-[3-(2-naphthyl)phenyl]-5,6-dihydropyrimidin-4(3H)-one;
2-amino-6-[4′-(hydroxymethyl)biphenyl-3-yl]-3,6-dimethyl-5,6-dihydropyrimidin-4(3H)-one;
2-amino-6-[3-(2-furyl)phenyl]-3,6-dimethyl-5,6-dihydropyrimidin-4(3H)-one;
2-amino-6-(4′-hydroxybiphenyl-3-yl)-3,6-dimethyl-5,6-dihydropyrimidin-4(3H)-one;
6-(4′-acetylbiphenyl-3-yl)-2-amino-3,6-dimethyl-5,6-dihydropyrimidin-4(3H)-one;
6-(3′-acetylbiphenyl-3-yl)-2-amino-3,6-dimethyl-5,6-dihydropyrimidin-4(3H)-one;
2-amino-6-[3-(1-benzothien-2-yl)phenyl]-3,6-dimethyl-5,6-dihydropyrimidin-4(3H)-one;
2-amino-3,6-dimethyl-6-(3′,4′,5′-trimethoxybiphenyl-3-yl)-5,6-dihydropyridin-4(3H)-one;
6-[3-(5-acetyl-2-thienyl)phenyl]-2-amino-3,6-dimethyl-5,6-dihydropyrimidin-4(3H)-one;
2-amino-6-(3′-fluoro-1,1′:4′, 1″-terphenyl-3-yl)-3,6-dimethyl-5,6-dihydropyrimidin-4(3H)-one;
2-amino-3,6-dimethyl-6-(3-pyridin-3-ylphenyl)-5,6-dihydropyrimidin-4(3H)-one;
2-amino-6-[3-(1-benzothien-3-yl)phenyl]-3,6-dimethyl-5,6-dihydropyrimidin-4(3H)-one;
3′-(2-amino-1,4-dimethyl-6-oxo-1,4,5,6-tetrahydropyrimidin-4-yl)biphenyl-3-carbonitrile; and
3′-(2-amino-1,4-dimethyl-6-oxo-1,4,5,6-tetrahydropyrimidin-4-yl)biphenyl 4-carbonitrile;
6-(2′-acetylbiphenyl-3-yl)-2-amino-3,6-dimethyl-5,6-dihydropyrimidin-4(3H)-one;
2-amino-3,6-dimethyl-6-[4′-(methylsulfonyl)biphenyl-3-yl]-5,6-dihydropyrimidin-4(3H)-one;
2-amino-6-(4′-cyclohexylbiphenyl-3-yl)-3,6-dimethyl-5,6-dihydropyrimidin-4(3H)-one;
2-amino-3,6-dimethyl-6-(3′,4′,5′-trifluorobiphenyl-3-yl)-5,6-dihydropyrimidin-4(3H)-one;
2-amino-6-[4′-(ethylsulfonyl)biphenyl-3-yl]-3,6-dimethyl-5,6-dihydropyrimidin-4(3H)-one;
2-amino-6-(4′-hydroxy-3′-methoxybiphenyl-3-yl)-3,6-dimethyl-5,6-dihydropyrimidin-4(3H)-one;
2-amino-6-(4′-hydroxy-3′,5′-dimethylbiphenyl-3-yl)-3,6-dimethyl-5,6-dihydropyrimidin-4(3H)-one;
2-amino-6-(2′-hydroxybiphenyl-3-yl)-3,6-dimethyl-5,6-dihydropyrimidin-4(3H)-one;
2-amino-6-(3′-hydroxybiphenyl-3-yl)-3,6-dimethyl-5,6-dihydropyrimidin-4(3H)-one;
5-[3-(2-amino-1,4-dimethyl-6-oxo-1,4,5,6-tetrahydropyrimidin-4-yl)phenyl]thiophene-2-carbonitrile;
N-[3′-(2-amino-1,4-dimethyl-6-oxo-1,4,5,6-tetrahydropyrimidin-4-yl)biphenyl-4-yl]acetamide;
2-amino-3,6-dimethyl-6-{3-[1-(phenylsulfonyl)-1H-indol-3-yl]phenyl}-5,6-dihydropyrimidin-4(3H)-one;
2-amino-6-[3-(3,4-dihydro-2H-1,5-benzodioxepin-7-yl)phenyl]-3,6-dimethyl-5,6-dihydropyrimidin-4(3H)-one;
2-amino-6-[3-(6-ethoxy-2-naphthyl)phenyl]-3,6-dimethyl-5,6-dihydropyrimidin-4(3H)-one;
2-amino-3,6-dimethyl-6-(3′-propoxybiphenyl-3-yl)-5,6-dihydropyrimidin-4(3H)-one;
methyl 3′-(2-amino-1,4-dimethyl-6-oxo-1,4,5,6-tetrahydropyrimidinyl) biphenyl-4-carboxylate;
2-amino-3,6-dimethyl-6-(2′-phenoxybiphenyl-3-yl)-5,6-dihydropyrimidin-4(3H)-one;
2-amino-6-(4′-isobutylbiphenyl-3-yl)-3,6-dimethyl-5,6-dihydropyrimidin-4(3H)-one;
2-amino-6-(2′-(benzyloxy)biphenyl-3-yl)-3,6-dimethyl-5,6-dihydropyrimidin-4(3H)-one;
methyl 3′-(2-amino-1,4-dimethyl-6-oxo-1,4,5,6-tetrahydropyrimidin-4-yl) biphenyl-3-carboxylate;
benzyl 3′-(2-amino-1,4-dimethyl-6-oxo-1,4,5,6-tetrahydropyrimidin-4-yl) biphenyl-3-carboxylate;
2-amino-6-(2′-methoxy-5′-methylbiphenyl-3-yl)-3,6-dimethyl-5,6-dihydropyrimidin 4(3H)-one;
2-amino-6-(4′-methoxy-3′,5′-dimethylbiphenyl-3-yl)-3,6-dimethyl-5,6-dihydropyrimidin 4(3H)-one;
benzyl [3′-(2-amino-1,4-dimethyl-6-oxo-1,4,5,6-tetrahydropyrimidin-4-yl) biphenyl-4-yl]carbamate;
ethyl 3′-(2-amino-1,4-dimethyl-6-oxo-1,4,5,6-tetrahydropyrimidin-4-yl) biphenyl-4-carboxylate;
benzyl 3′-(2-amino-1,4-diethyl-6-oxo-1,4,5,6-tetrahydropyrimidin-4-yl) biphenyl-4-carboxylate;
N-[3′-(2-amino-1,4-dimethyl-6-oxo-1,4,5,6-tetrahydropyrimidin-4-yl)biphenyl-2-yl]methanesulfonamide;
3′-(2-amino-1,4-dimethyl-6-oxo-1,4,5,6-tetrahydropyrimidin-4-yl)-N-(tert-butyl)biphenyl-2-sulfonamide;
2-amino-6-[3-(3,5-dimethylisoxazol-4-yl)phenyl]-3,6-dimethyl-5,6-dihydropyrimidin-4(3H)-one;
N-[3′-(2-amino-1,4-dimethyl-6-oxo-1,4,5,6-tetrahydropyrimidin-4-yl)biphenyl-3-yl]acetamide;
2-amino-6-(2′,6′-dichlorobiphenyl-3-yl)-3,6-dimethyl-5,6-dihydropyrimidin-4(3H)-one;
2-amino-6-(3′-chloro-2′-fluorobiphenyl-3-yl)-3,6-dimethyl-5,6-dihydropyrimidin-4(3H)-one;
2-amino-6-(4′-butoxy-3′-chlorobiphenyl-3-yl)-3,6-dimethyl-5,6-dihydropyrimidin-4(3H)-one;
[3′-(2-amino-1,4-dimethyl-6-oxo-1,4,5,6-tetrahydropyrimidin-4-yl)biphenyl-3-yl]formamide;
2-amino-6-[3-(2-fluoropyridin-3-yl)phenyl]-3,6-dimethyl-5,6-dihydropyrimidin-4(3H)-one;
2-amino-6-(3′-bromo-2′-ethoxy-5′-methylbiphenyl-3-yl)-3,6-dimethyl-5,6-dihydropyrimidin-4(3H)-one;
2-amino-6-[3-(2-ethoxy-1-naphthyl)phenyl]-3,6-dimethyl-5,6-dihydropyrimidin 4(3H)-one;
2-amino-6-[4′-(benzyloxy)-2′-fluorobiphenyl-3-yl]-3,6-dimethyl-5,6-dihydropyrimidin 4(3H)-one;
2-amino-3,6-dimethyl-6-[2′-(morpholin-4-ylmethyl)biphenyl-3-yl]-5,6-dihydropyrimidin 4(3H)-one;
3′-(2-amino-1,4-dimethyl-6-oxo-1,4,5,6-tetrahydropyrimidin-4-yl)-3-fluorobiphenyl-4-carbaldehyde;
2-amino-3,6-dimethyl-6-[4′-(trimethylsilyl)biphenyl-3-yl]-5,6-dihydropyrimidin-4(3H)-one;
2-amino-6-(3′-butoxybiphenyl-3-yl)-3,6-dimethyl-5,6-dihydropyrimidin-4(3H)-one;
2-amino-3,6-dimethyl-6-(2′,4′,6′-trimethylbiphenyl-3-yl)-5,6-dihydropyrimidin-4(3H)-one;
2-amino-6-[3-(2-chloro-3-fluoropyridin-4-yl)phenyl]-3,6-dimethyl-5,6-dihydropyrimidin-4(3H)-one;
2-amino-6-(5′-chloro-2′-methylbiphenyl-3-yl)-3,6-dimethyl-5,6-dihydropyrimidin-4(3H)-one;
2-amino-6-(2′,5′-difluorobiphenyl-3-yl)-3,6-dimethyl-5,6-dihydropyrimidin-4(3H)-one;
2-amino-3,6-dimethyl-6-(1,1′:4′,1″-terphenyl-3-yl)-5,6-dihydropyrimidin-4(3H)-one;
2-amino-6-(3′-chloro-4′-fluorobiphenyl-3-yl)-3,6-dimethyl-5,6-dihydropyrimidin-4(3H)-one;
2-amino-6-(4′-fluorobiphenyl-3-yl)-3,6-dimethyl-5,6-dihydropyrimidin-4(3M)-one;
2-amino-6-(3-dibenzo[b,d]thien-4-ylphenyl)-3,6-dimethyl-5,6-dihydropyrimidin-4(3H)-one;
2-amino-6-biphenyl-3-yl-3,6-dimethyl-5,6-dihydropyrimidin-4(3H)-one;
2-amino-6-(2′,3′-dimethylbiphenyl-3-yl)-3,6-dimethyl-5,6-dihydropyrimidin-4(3H)-one;
2-amino-6-{3-[(E)-2-biphenyl-4-ylvinyl]phenyl}-3,6-dimethyl-5,6-dihydropyrimidin-4(3H)-one;
2-amino-6-[4′-(benzyloxy)-3′-chlorobiphenyl-3-yl]-3,6-dimethyl-5,6-dihydropyrimidin-4(3H)-one;
2-amino-6-(4′-butoxy-2′-methylbiphenyl-3-yl)-3,6-dimethyl-5,6-dihydropyrimidin-4(3H)-one;
3′-(2-amino-1,4-dimethyl-6-oxo-1,4,5,6-tetrahydropyrimidin-4-yl)biphenyl-2-carbaldehyde;
2-amino-6-(3′-bromo-2′-isopropoxy-5′-methylbiphenyl-3-yl)-3,6-dimethyl-5,6-dihydropyrimidin-4(3H)-one;
3′-(2-amino-1,4-dimethyl-6-oxo-1,4,5,6-tetrahydropyrimidin-4-yl)biphenyl-4-carboxamide;
2-amino-6-(3′,5′-difluorobiphenyl-3-yl)-3,6-dimethyl-5,6-dihydropyrimidin-4(3H)-one;
2-amino-3,6-dimethyl-6-[3-(1H-pyrazol-3-yl)phenyl]-5,6-dihydropyrimidin-4(3H)-one;
2-amino-3,6-dimethyl-6-(3-quinolin-5-ylphenyl)-5,6-dihydropyrimidin-4(3H)-one;
2-amino-6-(2′,5′-dimethoxybiphenyl-3-yl)-3,6-dimethyl-5,6-dihydropyrimidin-4(3H)-one;
2-amino-6-(3′-chlorobiphenyl-3-yl)-3,6-dimethyl-5,6-dihydropyrimidin-4(3H)-one;
2-amino-6-(4′-chlorobiphenyl-3-yl)-3,6-dimethyl-5,6-dihydropyrimidin-4(31)-one;
2-amino-6-(3′-bromo-2′-butoxy-5′-methylbiphenyl-3-yl)-3,6-dimethyl-5,6-dihydropyrimidin-4(3H)-one;
2-amino-6-[2-[3-(2-aminophenyl)phenyl]ethyl]-3-methyl-5H-pyrimidin-4-one;
2-amino-3-methyl-6-[2-[3-(p-tolyl)phenyl]ethyl]-3H-pyrimidin-4-one;
2-amino-3-methyl-6-[2-[3-(m-tolyl)phenyl]ethyl]-3H-pyrimidin-4-one;
2-amino-3-methyl-6-[2-[3-(o-tolyl)phenyl]ethyl]-3H-pyrimidin-4-one;
2-amino-6-[2-[3-[4-(hydroxymethyl)phenyl]phenyl]ethyl]-3-methyl-3H-pyrimidin-4-one;
2-amino-6-[2-[3-(4-ethylsulfonylphenyl)phenyl]ethyl]-3-methyl-3H-pyrimidin-4-one;
2-amino-6-[2-[3-[2-(hydroxymethyl)phenyl]phenyl]ethyl]-3-methyl-3H-pyrimidin-4-one;
2-amino-6-[2-[3-(4-hydroxy-2-methyl-phenyl)phenyl]ethyl]-3-methyl-3H-pyrimidin-4-one;
2-amino-6-[2-[3-(4-methoxy-2-methyl-phenyl)phenyl]ethyl]-3-methyl-3H-pyrimidin-4-one;
2-amino-6-[2-[3-(5-chloro-2-thienyl)phenyl]ethyl]-3-methyl-3H-pyrimidin-4-one;
6-[2-[3-(5-acetyl-2-thienyl)phenyl]ethyl]-2-amino-3-methyl-3H-pyrimidinone;
2-amino-6-[2-[3-[4-(methoxymethyl)phenyl]phenyl]ethyl]-3-methyl-3H-pyrimidin-4-one;
4-[3-[2-(2-amino-1-methyl-6-oxo-1H-pyrimidin-4-yl)ethyl]phenyl]benzamide;
2-amino-3-methyl-6-[2-[3-(2-methylsulfonylphenyl)phenyl]ethyl]-3H-pyrimidinone;
2-amino-3-methyl-6-[2-[3-(5-quinolyl)phenyl]ethyl]-3H-pyrimidin-4-one;
2-Amino-3-(cyclohexylmethyl)-6-[2-[3-(2-furyl)phenyl]ethyl]-3H-pyrimidin-4-one;
2-Amino-6-[2-[3-(2-furyl)phenyl]ethyl]-3-(tetrahydrofuran-2-ylmethyl)-3H-pyrimidin-4-one;
2-Amino-6-[2-[3-(2-furyl)phenyl]ethyl]-3-(3-hydroxypropyl)-3H-pyrimidin-4-one;
2-Amino-6-[2-[3-(2-furyl)phenyl]ethyl]-3-[(3-methoxyphenyl)methyl]-3H-pyrimidin-4-one;
2-amino-6-[2-[3-(2-furyl)phenyl]ethyl]-3-[[3-(1H-tetrazol-5-yl)phenyl]methyl]-3H-pyrimidin-4-one;
3-[[2-amino-4-[2-(3-furan-2-yl-phenyl)-ethyl]-6-oxo-6H-pyrimidin-1-ylmethyl]-benzonitrile;
3-[[2-Amino-4-[2-[3-(2-furyl)phenyl]ethyl]-6-oxo-1H-pyrimidin-1-yl]methyl]benzoic acid;
3-[[2-Amino-4-[2-[3-(2-furyl)phenyl]ethyl]-6-oxo-6H-pyrimidin-1-yl]methyl]benzoic acid methyl ester;
3-[2-[2-Amino-4-[2-[3-(2-furyl)phenyl]ethyl]-6-oxo-1H-pyrimidin-1-yl]ethylamino]-3-oxo-propanoic acid;
2-[2-[2-Amino-4-[2-[3-(2-furyl)phenyl]ethyl]-6-oxo-1H-pyrimidin-1-yl]ethylcarbamoyl]benzoic acid;
2-Amino-6-3′-ethyl-biphenyl-3-ylmethyl)-3-methyl-3H-pyrimidin-4-one;
2-Amino-6-(3-[(E)-2-(3-methoxy-phenyl)-vinyl]-benzyl)-3-methyl-3H-pyrimidin-4-one;
2-Amino-6-{3-[2-(3-methoxy-phenyl)-ethyl]-benzyl}-3-methyl-3H-pyrimidin-4-one;
2-Amino-3-methyl-6-[3-((E)-styryl)-benzyl]-3H-pyrimidin-4-one;
2-Amino-6-{3-[(E)-2-(4-methoxy-phenyl)-vinyl]-benzyl}-3-methyl-3H-pyrimidin-4-one;
2-Amino-3-methyl-6-[3-((E)-3-methyl-pent-1-enyl)-benzyl]-3H-pyrimidin-4-one;
2-Amino-3-methyl-6-[3-((E)-4-methyl-pent-1-enyl)-benzyl]-3H-pyrimidin-4-one;
2-Amino-3-methyl-6-(3-phenethyl-benzyl)-3H-pyrimidin-4-one;
2-Amino-3-methyl-6-[3-(2-pyridin-4-yl-ethyl)-benzyl]-3H-pyrimidin-4-one;
2-Amino-3-methyl-6-[3-(3-methyl-pentyl)-benzyl]-3H-pyrimidin-4-one;
2-Amino-3-methyl-6-[3-(4-methyl-pentyl)-benzyl]-3H-pyrimidin-4-one;
2-amino-3,6-dimethyl-6-naphthalen-1-yl-5,6-dihydro-3H-pyrimidin-4-one;
2-amino-6-(4-chloro-3-naphthalen1-yl-phenyl)-3,6-dimethyl-5,6-dihydro-3H-pyrimidin-4-one;
2-amino-6-(6,4′-dichloro-biphenyl-3-yl)-3,6-dimethyl-5,6-dihydro-3H-pyrimidin-4-one;
2-amino-6-(4-chloro-3-isoquinolin-5-yl-phenyl)-3,6-dimethyl-5,6-dihydro-3H-pyrimidin-4-one;
2-amino-6-(6-chloro-4′-hydroxy-biphenyl-3-yl-phenyl)-3,6-dimethyl-5,6-dihydro-3H-pyrimidin-4-one;
2-amino-6-(4-chloro-3-dibenzofuran-4-yl-phenyl)-3,6-dimethyl-5,6-dihydro-3H-pyrimidin-4-one;
2-amino-6-[4-chloro-3-(2,3-dihydrobenzo[1,4]dioxin-6-yl-phenyl]-3,6-dimethyl-5,6-dihydro-3H-pyrimidin-4-one;
2-amino-3-benzyl-6-(3-bromo-phenyl)-6-methyl-5,6-dihydro-3H-pyrimidin-4-one;
2-amino-6-[4-chloro-3-(1H-indol-6-yl)-phenyl]-3,6-dimethyl-5,6-dihydro-3H-pyrimidin-4-one;
2-amino-3-benzyl-6-(3′-methoxy-biphenyl-3-yl)-6-methyl-5,6-dihydro-3H-pyrimidin-4-one;
2-amino-3-benzyl-6-methyl-6-phenyl-5,6-dihydro-3H-pyrimidin-4-one;
2-amino-6-(6-chloro-biphenyl-3-yl)-3,6-dimethyl-5,6-dihydro-3H-pyrimidin-4-one;
2-amino-6-(6-chloro-4′-methoxy-biphenyl-3-yl)-3,6-dimethyl-5,6-dihydro-3H-pyrimidin-4-one;
2-amino-6-(3-methoxy-phenyl)-3,6-dimethyl-5,6-dihydro-3H-pyrimidin-4-one;
2-amino-3-methyl-6-[2-[3-(2-thienyl)phenyl]ethyl]-3H-pyrimidin-4-one;
2-amino-6-[2-[3-(3-methoxyphenyl)phenyl]ethyl]-3,6-dimethyl-5,6-dihydro-3H-pyrimidin-4-one;
2-amino-3-methyl-6-phenyl-5,6-dihydro-3H-pyrimidin-4-one;
3-[2-[2-amino-4-[2-[3-(2-furyl)phenyl]ethyl]-6-oxo-1H-pyrimidin-1-yl]ethylcarbamoyl]benzoic acid;
2-amino-6-[2-[3-(2-furyl)phenyl]ethyl]-3-[2-(1H-imidazol-1-yl)ethyl]-3H-pyrimidin-4-one;
2-amino-3-[2-[bis(3-furylmethyl)amino]ethyl]-6-[2-[3-(2-furyl)phenyl]ethyl]-3H-pyrimidin-4-one;
N-[2-[2-amino-4-[2-[3-(2-furyl)phenyl]ethyl]-6-oxo-1H-pyrimidin-1-yl]ethyl]-2-(1H-tetrazol-5-yl)acetamide;
5-[2-[2-amino-4-[2-[3-(2-furyl)phenyl]ethyl]-6-oxo-1H-pyrimidin-1-yl]ethylamino]-5-oxo-pentanoic acid;

acetic acid 2-[2-amino-4-[2-[3-(2-furyl)phenyl]ethyl]-6-oxo-1H-pyrimidin-1-yl]ethylcarbamoylmethyl ester;
N-[2-[2-amino-4-[2-[3-(2-furyl)phenyl]ethyl]-6-oxo-1H-pyrimidin-1-yl]ethyl]-3-hydroxy-benzamide;
N-[2-[2-amino-4-[2-[3-(2-furyl)phenyl]ethyl]-6-oxo-1H-pyrimidin-1-yl]ethyl]-4-hydroxy-benzamide;
4-amino-N-[2-[2-amino-4-[2-[3-(2-furyl)phenyl]ethyl]-6-oxo-1H-pyrimidin-1-yl]ethyl]benzamide;
N-[2-[2-amino-4-[2-[3-(2-furyl)phenyl]ethyl]-6-oxo-1H-pyrimidin-1-yl]ethyl]piperidine-4-carboxamide;
2-amino-6-[2-[3-(2-furyl)phenyl]ethyl]-3-(2-phenethylaminoethyl)-3H-pyrimidin-4-one;
4-[2-[2-amino-4-[2-[3-(2-furyl)phenyl]ethyl]-6-oxo-1H-pyrimidin-1-yl]ethylaminomethyl]benzoic acid;
2-amino-6-[2-[3-(2-furyl)phenyl]ethyl]-3-[2-[(3-hydroxyphenyl)methylamino]ethyl]-3H-pyrimidin-4-one;
2-amino-6-[2-[3-(2-furyl)phenyl]ethyl]-3-[2-[[4-(2-hydroxyethoxy)phenyl]methylamino]ethyl]-3H-pyrimidin-4-one;
2-amino-6-[2-[3-(2-furyl)phenyl]ethyl]-3-(2-isobutylaminoethyl)-3H-pyrimidin-4-one;
2-amino-6-[2-[3-(2-furyl)phenyl]ethyl]-3-[2-(1H-indol-5-ylmethylamino)ethyl]-3H-pyrimidin-4-one;
2-amino-6-[2-[3-(2-furyl)phenyl]ethyl]-3-[2-[(4-hydroxyphenyl)methylamino]ethyl]-3H-pyrimidin-4-one;
2-amino-6-[2-[3-(2-furyl)phenyl]ethyl]-3-[2-(3-hydroxyphenyl)methylamino]ethyl]-3H-pyrimidin-4-one;
2-amino-3-benzyl-6-[2-[3-(2-furyl)phenyl]ethyl]-3H-pyrimidin-4-one;
2-amino-6-[2-[3-(2-furyl)phenyl]ethyl]-3-(2-hydroxyethyl)-3H-pyrimidin-4-one;
2-amino-6-[2-[3-(2-furyl)phenyl]ethyl]-3-isopentyl-3H-pyrimidin-4-one;
2-amino-6-[2-[3-(2-furyl)phenyl]ethyl]-3-[2-[(1-hydroxy-2,2,6,6-tetramethyl-4-piperidyl)amino]ethyl]-3H-pyrimidin-4-one;
2-amino-6-[2-[3-(2-fi)phenyl]ethyl]-3-[2-[(1-hydroxy-2,2,6,6-tetramethyl-4-piperidyl)amino]ethyl]-3H-pyrimidin-4-one;
2-amino-3-[2-(1,3-dioxan-2-yl)ethyl]-6-[2-[3-(2-furyl)phenyl]ethyl]-3H-pyrimidin-4-one;
4-[[2-amino-4-[2-[3-(2-furyl)phenyl]ethyl]-6-oxo-1H-pyrimidin-1-yl]methyl]benzonitrile;
2-amino-6-[2-[3-(2-furyl)phenyl]ethyl]-3-[[4-(1H-tetrazol-5-yl)phenyl]methyl]-3H-pyrimidin-4-one;
2-amino-6-[2-[3-(2-furyl)phenyl]ethyl]-3-(2-morpholinoethyl)₃H-pyrimidin-4-one;
2-[[2-amino-4-[2-[3-(2-furyl)phenyl]ethyl]-6-oxo-1H-pyrimidin-1-yl]methyl]benzonitrile;
2-amino-6-[2-(3-(2-furyl)phenyl]ethyl]-3-[2-(1-piperidyl)ethyl]-3H-pyrimidin-4-one;

or a pharmaceutically acceptable salt thereof.

In another aspect, provided herein are novel compounds of structural formula IIa or formula Ib, or a pharmaceutically acceptable salt, tautomer or in vivo-hydrolysable precursors thereof:

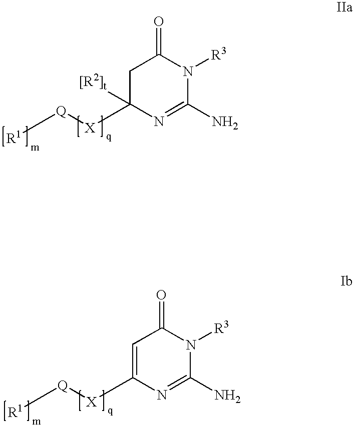

wherein

Q is C₆₋₁₀aryl or C₅₋₁₀heterocyclyl;

X is C₁₋₃alkyl;

q is 0 or 1;

m is 0 or 1 or 2;

R¹ is independently selected from H, halogen, optionally substituted C₅₋₁₀aryl, optionally substituted OC₅₋₁₀aryl or optionally substituted C₅₋₁₀heterocyclyl wherein such substituent(s) are independently selected from: halogen, OH, NH₂, CN, C(═O)NH₂, C₁₋₆alkyl, OC₁₋₆alkyl, C₁₋₄alkylOH, C₁₋₄alkylOC₁₋₃alkyl, CH₂OH, SO₂H, SO₂NHC(CH₃)₃, SO₂C₁₋₆alkyl, SO₂NHC₁₋₆alkyl, OC₁₋₃alkylOC₁₋₃alkyl, OC₁₋₃alkylOH, OC₁₋₃alkylOC(═O)C₁₋₃alkyl, C(═O)C₁₋₆alkyl, C(═O)OC₁₋₆alkyl, C(═O)NH₂, C₅₋₆heterocyclyl, OC₅₋₆aryl, OC₁₋₆alkyl-C₅₋₆aryl, COOH, C(═O)NHC₁₋₆alkyl, C(═O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(═O)C₁₋₆alkyl, NC(═O)(C₁₋₆alkyl)₂, C₅₋₆aryl, C(═O)C₅₋₆aryl, C(═O)OC₅₋₆aryl, C(═O)NHC₅₋₄aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl), N(C₅₋₆aryl)₂, NC(═O)C₅₋₄aryl, C₅₋₆heterocyclyl, OC₅heterocyclyl, C(═O)C₅₋₆heterocyclyl, C(═O)OC₅₋₆heterocyclyl, C(═O)NHC₅₋₆heterocyclyl,

One embodiment of the present invention provides compounds of formula IIa or formula Ib, or a pharmaceutically acceptable salt, tautomer or in vivo-hydrolysable precursors wherein Q is C₆aryl and R¹, X, m, q, t, R², R³, R^{c} and R^{d} have any of the meanings defined hereinabove.

One embodiment of the present invention provides compounds of formula IIa or a pharmaceutically acceptable salt, tautomer or in vivo-hydrolysable precursors wherein Q is C₆aryl and R¹, X, m, q, t, R², R³, R^{c} and R^{d} have any of the meanings defined hereinabove.

One embodiment of the present invention provides compounds of formula IIa or formula Ib, or a pharmaceutically acceptable salt, tautomer or in vivo-hydrolysable precursors wherein Q is C₉heterocyclyl and R¹, X, m, q, t, R², R³, R^{c} and R^{d} have any of the meanings defined hereinabove.

One embodiment of the present invention provides compounds of formula IIa, or a pharmaceutically acceptable salt, tautomer or in vivo-hydrolysable precursors wherein R² is C₁₋₃alkyl and Q, R¹, X, m, q, t, R³, R^{c} and R^{d} have any of the meanings defined hereinabove.

One embodiment of the present invention provides compounds of formula IIa or formula Ib, or a pharmaceutically acceptable salt, tautomer or in vivo-hydrolysable precursors wherein R³ is C₁₋₃alkyl and Q, R¹, X, m, q, t, R², R^{c} and R^{d} have any of the meanings defined hereinabove.

One embodiment of the present invention provides compounds of formula IIa or formula Ib, or a pharmaceutically acceptable salt, tautomer or in vivo-hydrolysable precursors wherein:

Q is C₆aryl or C₉heterocyclyl;

X is C₁₋₃alkyl;

q is 0 or 1;

m is 0 or 1 or 2;

R¹ is independently selected from H, halogen, optionally substituted C₆aryl, or optionally substituted C₅₋₆heterocyclyl wherein such substituent(s) are independently selected from: halogen, OH, NH₂, CN, C(═O)NH₂, C₁₋₆alkyl, OC₁₋₆alkyl, C₁₋₄alkylOH, C₁₋₄alylOC₁₋₃alkyl, CH₂OH, SO₂H, SO₂NHC(CH₃)₃, SO₂C₁₋₆alkyl, SO₂NHC₁₋₆alkyl, OC₁₋₃alkylOC₁₋₃alkyl, OC₁₋₃alkylOH, OC₁₋₃alkylOC(═O)C₁₋₃alkyl, C(═O)C₁₋₆alkyl, C(═O)OC₁₋₆alkyl, C(═O)NH₂, C₅₋₆heterocyclyl, OC₅₋₆aryl, OC₁₋₆alkyl-C₅₋₄aryl;

R² is C₁₋₃alkyl;

t is 0 or 1;

and R³, R^{c} and R^{d} have any of the meanings defined hereinabove.

One embodiment of the present invention provides compounds of formula IIa or formula Ib, or a pharmaceutically acceptable salt, tautomer or in vivo-hydrolysable precursors wherein:

Q is C₆aryl or C₉heterocyclyl;

X is C₁₋₃alkyl;

q is 0 or 1;

m is 0 or 1 or 2;

R¹ is independently selected from H, halogen, optionally substituted C₆aryl, or optionally substituted C₄₋₆heterocyclyl wherein such substituent(s) are independently selected from: halogen, OH, NH₂, CN, C(═O)NH₂, C₁₋₆alkyl, OC₁₋₆alkyl, C₁₋₄alkylOH, C₁₋₄alkylOC₁₋₃alkyl, CH₂OH, SO₂H, SO₂NHC(CH₃)₃, SO₂C₁₋₆alkyl, SO₂NHC₁₋₆alkyl, OC₁₋₃alkylOC₁₋₃alkyl, OC₁₋₃alkylOH, OC₁₋₃alkylOC(═O)C₁₋₃alkyl, C(═O)C₁₋₆alkyl, C(═O)OC₁₋₆alkyl, C(═O)NH₂, C₅₋₆heterocyclyl, OC₅₋₆aryl, OC₁₋₆alkyl-C₅₋₆aryl;

R² is C₁₋₃alkyl;

t is 0 or 1;

R³ is H, C₁₋₆alkyl, C₁₋₆alkylNR^{c}R^{c}, C₁₋₆alkyl-C₅₋₉heterocyclyl; and R^{c} and R^{d} have any of the meanings defined hereinabove

One embodiment of the present invention provides compounds of formula IIa or formula Ib, or a pharmaceutically acceptable salt, tautomer or in vivo-hydrolysable precursors wherein:

Q is C₆aryl;

X is C₁₋₃alkyl;

q is 0 or 1;

m is 0 or 1 or 2;

R¹ is independently selected from H, halogen, optionally substituted C₆aryl, or optionally substituted C₅₋₆heterocyclyl wherein such substituent(s) are independently selected from: halogen, OH, NH₂, CN, C(═O)NH₂, C₁₋₆alkyl, OC₁₋₆alkyl, C₁₋₄alkylOH, C₁₋₄alkylOC₁₋₃alkyl, CH₂OH, SO₂H, SO₂NHC(CH₃)₃, SO₂C₁₋₆alkyl, SO₂NHC₁₋₆alkyl, OC₁₋₃alkylOC₁₋₃alkyl, OC₁₋₃alkylOH, OC₁₋₃alkylOC(═O)C₁₋₃alkyl, C(═O)C₁₋₆alkyl, C(═O)OC₁₋₆alkyl, C(═O)NH₂, C₅₋₆heterocyclyl, OC₅₋₆aryl, OC₁₋₆alkyl-C₅₋₆aryl;

R² is C₁₋₃alkyl;

t is 1;

R³ is C₁₋₃alkyl

u is 1.

One embodiment of the present invention provides compounds of formula IIa or formula Ib, or a pharmaceutically acceptable salt, tautomer or in vivo-hydrolysable precursors thereof wherein:

Q is C₆₋₁₀aryl or C₅₋₁₀heteroaryl;

X is C₁₋₃alkyl;

q is 0 or 1;

m is 0 or 1 or 2;

R¹ is independently selected from H, halogen, optionally substituted C₅₋₁₀aryl, optionally substituted OC₅₋₁₀aryl or optionally substituted C₅₋₁₀heterocyclyl wherein such substituent(s) are independently selected from: halogen, OH, NH₂, CN, C(═O)NH₂, C₁₋₆alkyl, OC₁₋₆alkyl, C₁₋₄alkylOH, C₁₋₄alkylOC₁₋₃alkyl, CH₂OH, SO₂H, SO₂NHC(CH₃)₃, SO₂C₁₋₆alkyl, SO₂NHC₁₋₆alkyl, OC₁₋₃alkylOC₁₋₃alkyl, OC₁₋₃alkylOH, OC₁₋₃alkylOC(═O)C₁₋₃alkyl, C(═O)C₁₋₆alkyl, C(═O)OC₁₋₆alkyl, C(═O)NH₂, C₅₋₆heterocyclyl, OC₅₋₆aryl, OC₁₋₆alkyl-C₅₋₆aryl,

R² is H, C₁₋₆alkyl,

t is 0 or 1;

R³ is independently selected from H, C₁₋₃alkyl.

One embodiment of the present invention provides compounds of formula IIa or formula Ib, or a pharmaceutically acceptable salt, tautomer or in vivo-hydrolysable precursors thereof wherein:

Q is C₆aryl or C₉heterocyclyl;

X is C₁₋₃alkyl;

q is 0 or 1;

m is 0 or 1 or 2;

R¹ is independently selected from H, halogen, optionally substituted C₆aryl, or optionally substituted C₄₋₆heterocyclyl wherein such substituent(s) are independently selected from: halogen, OH, NH₂, CN, C(═O)NH₂, C₁₋₆alkyl, OC₁₋₆alkyl, C₁₋₄alkylOH, C₁₋₄alkylOC₁₋₃alkyl, CH₂OH, SO₂H, SO₂NHC(CH₃)₃, SO₂C₁₋₆alkyl, SO₂NHC₁₋₆alkyl, OC₁₋₃alylOC₁₋₃alkyl, OC₁₋₃alkylOH, OC₁₋₃alkylOC(═O)C₁₋₃alkyl, C(═O)C₁₋₆alkyl, C(═O)OC₁₋₆alkyl, C(═O)NH₂, C₅₋₆heterocyclyl, OC₅₋₆aryl, OC₁₋₆alkyl-C₅₋₆aryl;

R² is C₁₋₃alkyl;

t is 0 or 1;

R³ is C₁₋₃alkyl

u is 1.

One embodiment of the present invention provides compounds of formula IIa or formula Ib, selected from the following:
2-amino-6-(3-bromo-phenyl)-6-methyl-5,6-dihydro-3H-pyrimidin-4-one;
2-amino-6-(3′-methoxy-biphenyl-3-yl)-3,6-dimethyl-5,6-dihydro-3H-pyrimidin-4-one;
6-(3′-methoxy-1,1′-biphenyl-3-yl)-6-methyl-2-(methylamino)-5,6-dihydropyrimidin-4(3H)-one;
2-amino-6-(3-bromo-phenyl)-3,6-dimethyl-5,6-dihydro-3H-pyrimidin-4-one;
2-amino-6-(3-(5-chloro-thiophen-2-yl)-phenyl]-3,6-dimethyl-5,6-dihydro-3H-pyrimidin-4-one;
2-amino-6-(3,4-dichlorophenyl)-6-ethyl-5,6-dihydropyrimidin-4(3H)-one;
2-amino-6-(3,4-dichlorophenyl)-6-isobutyl-5,6-dihydropyrimidin-4(3H)-one;
2-amino-6-(3,4-dichlorophenyl)-3-methyl-6-(2-phenylethyl)-5,6-dihydropyrimidin-4(3H)-one;
2-amino-6-(3-bromochlorophenyl)-3,6-dimethyl-5,6-dihydropyrimidin-4(3H)-one;
2-amino-3,6-dimethyl-6-(2-phenylethyl)-5,6-dihydropyrimidin-4(3H)-one;
2-amino-6-[2-(3-bromophenyl)-ethyl]-3,6-dimethyl-5,6-dihydro-3H-pyrimidin-4-one;
2-amino-6-[3-(benzyloxy)phenyl]-3,6-dimethyl-5,6-dihydropyrimidin-4(3H)-one;
2-amino-6-methyl-6-(3-phenoxyphenyl)-5,6-dihydropyrimidin-4(3H)-one;
2-amino-6-(3-bromo-4-chlorophenyl)-6-methyl-5,6-dihydropyrimidin-4(3H)-one;
2-amino-6-(3′-methoxy-1,1′-biphenyl-3-yl)-6-methyl-5,6-dihydropyrimidin-4(3H)-one;
2-amino-6-methyl-6-[3-(5-methylthien-2-yl)phenyl]-5,6-dihydropyrimidin-4(3H)-one;
2-amino-6-[3-(2-furyl)phenyl]-6-methyl-5,6-dihydropyrimidin-4(3H)-one;
2-amino-6-(3′-butoxy-1,1′-biphenyl-3-yl)-6-methyl-5,6-dihydropyrimidin-4(3H)-one;
2-amino-6-(6-chloro-3′-methoxy-1,1′-biphenyl-3-yl)-6-methyl-5,6-dihydropyrimidin-4(3H)-one;
6-(3′-methoxy-1,1′-biphenyl-3-yl)-6-methyl-2-(methylamino)-5,6-dihydropyrimidin-4(3H)-one;
2-amino-3,6-dimethyl-6-[3-(5-methylthien-2-yl)phenyl]-5,6-dihydropyrimidin-4(3H)-one;
2-amino-6-(3′-isopropoxy-1,1′-biphenyl-3-yl)-3,6-dimethyl-5,6-dihydropyrimidin-4(3H)-one;
2-amino-6-(6-chloro-3′-methoxy-1,1′-biphenyl-3-yl)-3,6-dimethyl-5,6-dihydropyrimidin-4(3H)-one;
2-amino-6-(4-chloro-3-quinolin-5-yl-phenyl)-3,6-dimethyl-5,6-dihydro-3H-pyrimidin-4-one;
2-amino-3,6-dimethyl-6-naphthalen-2-yl-5,6-dihydro-3H-pyrimidin-4-one;
2-Amino-6-[2-(3′-methoxy-biphenyl-3-yl)-ethyl]-3,6-dimethyl-5,6-dihydro-3H-pyrimidin-4-one;
2-Amino-6-methyl-6-quinolin-6-yl-5,6-dihydro-3H-pyrimidin-4-one
2-Amino-6-(3,4-dichloro-phenyl)-3,6-dimethyl-5,6-dihydro-3H-pyrimidin-4-one;
2-Amino-6-(3,4-dichloro-phenyl)-6-methyl-5,6-dihydro-3H-pyrimidin-4-one;
2-Amino-6-[2-(3-bromophenyl)ethyl]-3-methylpyrimidin-4(3H)-one;
2-Amino-6-[2-(2-bromophenyl)ethyl]-3-methylpyrimidin-4(3H)-one;
2-Amino-6-[2-(4-bromophenyl)ethyl]-3-methylpyrimidin-4(3H)-one;
2-Amino-6-[2-(1H-indol-6-yl)ethyl]-3-methylpyrimidin-4(3H)-one;
2-Amino-6-[2-(1H-indol-6-yl)ethyl]pyrimidin-4(3H)-one;
2-Amino-3-methyl-6-[2-(2-naphthyl)ethyl]pyrimidin-4(3H)-one;
2-amino-3-(1,3-dioxolan-2-ylmethyl)-6-[2-(1H-indol-6-yl)ethyl]pyrimidin-4(3H)-one;
2-Amino-3-methyl-6-{2-[3-(2-thienyl)phenyl]ethyl}pyrimidin-4(31)-one;
2-amino-3-methyl-6-{2-[4′-(methylsulfonyl)biphenyl-3-yl]ethyl}pyrimidin-4(3H)-one;
2-amino-6-{2-[3-(1H-indol-5-yl)phenyl]ethyl}-3-methylpyrimidin-4(3H)-one;
2-amino-6-{2-[3-(2,3-dihydro-1,4-benzodioxin-6-yl)phenyl]ethyl}-3-methylpyrimidin-4(3H)-one;
2-amino-3-methyl-6-{2-[3′-(1H-pyrazol-1-yl)biphenyl-3-yl]ethyl}pyrimidin-4(3H)-one;
2-amino-6-{2-[3-(2,3-dihydro-1-benzofuran-5-yl)phenyl]ethyl}-3-methylpyrimidin-4(3H)-one;
2-amino-3-methyl-6-{2-[3-(1H-pyrrol-2-yl)phenyl]ethyl}pyrimidin-4(3H)-one;
methyl 3′-[2-(2-amino-1-methyl-6-oxo-1,6-dihydropyrimidin-4-yl)ethyl]biphenyl-4-carboxylate;
2-amino-6-{2-[3′-(hydroxymethyl)biphenyl-3-yl]ethyl}-3-methylpyrimidin-4(3H)-one;
2-amino-6-[2-(3′-hydroxybiphenyl-3-yl)ethyl]-3-methylpyrimidin-4(3H)-one;
6-[2-(3′-acetylbiphenyl-3-yl)ethyl]-2-amino-3-methylpyrimidin-4(3H)-one;
2-amino-3-methyl-6-[2-(3-pyridin-4-ylphenyl)ethyl]pyrimidin-4(3H)-one;
2-amino-6-{2-[3-(2-furyl)phenyl]ethyl}-3-methylpyrimidin-4(3H)-one;
2-amino-3-methyl-6-{2-[3-(5-methyl-2-thienyl)phenyl]ethyl}pyrimidin-4(3H)-one;
2-amino-6-[2-(3′-methoxybiphenyl-3-yl)ethyl]-3-methylpyrimidin-4(3H)-one;
2-amino-3-methyl-6-{2-[3-(4-methyl-2-thienyl)phenyl]ethyl}pyrimidin-4(3H)-one;
2-amino-6-(2-biphenyl-3-ylethyl)-3-methylpyrimidin-4(3H)-one;
2-amino-6-[2-(4′-chlorobiphenyl-3-yl)ethyl]-3-methylpyrimidin-4(3H)-one;
2-amino-6-[2-(4′-hydroxybiphenyl-3-yl)ethyl]-3-methylpyrimidin-4(3H)-one;
{3′-[2-(2-amino-1-methyl-6-oxo-1,6-dihydropyrimidin-4-yl)ethyl]biphenyl-4-yl}acetonitrile;
2-amino-3-methyl-6-{2-[3-(3-thienyl)phenyl]ethyl}pyrimidin-4(3H)-one;
2-amino-6-[2-(4′-methoxybiphenyl-3-yl)ethyl]-3-methylpyrimidin-4(3H)-one;
2-amino-6-(2-{3-[5-(hydroxymethyl)-2-thienyl]phenyl}ethyl)-3-methylpyrimidin-4(3H)-one;
2-amino-3-methyl-6-[2-(3-pyridin-3-ylphenyl)ethyl]pyrimidin-4(3H)-one;
2-amino-6-[2-(3′-ethoxybiphenyl-3-yl)ethyl]-3-methylpyrimidin-4(3H)-one;
2-amino-6-{2-[3-(3-furyl)phenyl]ethyl}-3-methylpyrimidin-4(3H)-one;
2-amino-6-[2-(2′-methoxybiphenyl-3-yl)ethyl]-3-methylpyrimidin-4(3H)-one;
3′-[2-(2-amino-1-methyl-6-oxo-1,6-dihydropyrimidin-4-yl)ethyl]-N-(tert-butyl)biphenyl-2-sulfonamide;
methyl 3′-[2-(2-amino-1-methyl-6-oxo-1,6-dihydropyrimidin-4-yl)ethyl]biphenyl-2-carboxylate;
2-amino-3-methyl-6-{2-[2-(2-thienyl)phenyl]ethyl}pyrimidin-4(3H)-one;
2-amino-6-{2-[3′-(hydroxymethyl)biphenyl-4-yl]ethyl}-3-methylpyrimidin-4(3H)-one;
2-amino-6-[2-(3′,4′-dimethoxybiphenyl-4-yl)ethyl]-3-methylpyrimidin-4(3H)-one;
2-amino-6-[2-(3′-methoxybiphenyl-4-yl)ethyl]-3-methylpyrimidin-4(3H)-one;
2-amino-3-methyl-6-{2-[4-(2-thienyl)phenyl]ethyl}pyrimidin-4(3H)-one;
2-amino-6-[2-(1,1′-biphenyl-3-yl)ethyl]pyrimidin-4(3H)-one;
N-{2-[2-Amino-4-{2-[3-(2-furyl)phenyl]ethyl}-6-oxopyrimidin-1(6H)-yl]ethyl}acetamide;
N-{2-[2-amino-4-{2-[3-(2-furyl)phenyl]ethyl}-6-oxopyrimidin-1(6H)-yl]ethyl}butanamide;
1-{2-[2-amino {2-[3-(2-furyl)phenyl]ethyl}-6-oxopyrimidin-1(6H)-yl]ethyl}pyrrolidine-2,5-dione;
methyl 3-({2-[2-amino-4-{2-[3-(2-furyl)phenyl]ethyl}-6-oxopyrimidin-1(6H)-yl]ethyl}amino)-3-oxopropanoate;
methyl 5-({2-[2-amino-4-{2-[3-(2-furyl)phenyl]ethyl}-6-oxopyrimidin-1(6H)-yl]ethyl}amino)-5-oxopentanoate;
methyl 6-({2-[2-amino-4-{2-[3-(2-furyl)phenyl]ethyl}-6-oxopyrimidin-1(6H)-yl]ethyl}amino)-6-oxohexanoate;
6-({2-[2-amino-4-{2-[3-(2-furyl)phenyl]ethyl}-6-oxopyrimidin-1(6H)-yl]ethyl}amino)-6-oxohexanoic acid;
N-{2-[2-amino-4-{2-[3-(2-furyl)phenyl]ethyl}-6-oxopyrimidin-1(6H)-yl]ethyl}-2-methoxyacetamide;
2-Amino-3-[2-(benzylamino)ethyl]-6-{2-[3-(2-furyl)phenyl]ethyl}pyrimidin-4(3H)-one;
2-amino-6-{2-[3-(2-furyl)phenyl]ethyl}-3-{2-[(3-methoxybenzyl)amino]ethyl}pyrimidin 4(3H)-one;
2-({3′-[2-(2-Amino-1-methyl-6-oxo-1,6-dihydropyrimidin-4-yl)ethyl]-1,1′-biphenyl-3-yl}oxy)ethyl acetate;
2-Amino-6-{2-[3′-(2-hydroxyethoxy)-1,1′-biphenyl-3-yl]ethyl}-3-methylpyrimidin-4(3H)-one;
2-Amino-6-{2-[3′-(2-methoxyethoxy)-1,1′-biphenyl-3-yl]ethyl}-3-methylpyrimidin-4(3H)-one;
2-({3′-[2-(2-Amino-1-methyl-6-oxo-1,6-dihydropyrimidin-4-yl)ethyl]-1,1′-biphenyl-4-yl}oxy)ethyl acetate;
2-Amino-6-{2-[4′-(2-methoxyethoxy)-1,1′-biphenyl-3-yl]ethyl}-3-methylpyrimidin-4(3H)-one;
2-Amino-3-[2-(benzyloxy)ethyl]-6-{2-[3-(2-furyl)phenyl]ethyl}pyrimidin-4(3H)-one;
2-Amino-3-methyl-6-(3′-methyl-biphenyl-3-ylmethyl)-3H-pyrimidin-4-one;
2-Amino-6-(3′-methoxy-biphenyl-3-ylmethyl)-3-methyl-3H-pyrimidin-4-one;
2-Amino-6-(3′-ethoxy-biphenyl-3-ylmethyl)-3-methyl-3H-pyrimidin-4-one;
3′-(2-Amino-1-methyl-6-oxo-1,6-dihydro-pyrimidin-4-ylmethyl)biphenyl-3-carbonitrile;
2-Amino-3-methyl-6-(3′-vinyl-biphenyl-3-ylmethyl)₃H-pyrimidin-4-one;
2-Amino-6-(3′-isopropyl-biphenyl-3-ylmethyl)-3-methyl-3H-pyrimidin-4-one;
2-Amino-6-(4′-methoxy-biphenyl-3-ylmethyl)-3-methyl-3H-pyrimidin-4-one.

Compounds of the present invention also include pharmaceutically acceptable salts, tautomers and in vivo-hydrolysable precursors of the compounds of formula Ia and/or formula Ib. Compounds of the invention further include hydrates and solvates.

Compounds of the invention can be used as medicaments. In some embodiments, the present invention provides compounds of formula Ia or formula Ib, or pharmaceutically acceptable salts, tautomers or in vivo-hydrolysable precursors thereof, for use as medicaments. In some embodiments, the present invention provides compounds described herein for use as medicaments for treating or preventing an Aβ-related pathology. In some further embodiments, the Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer. Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

In some embodiments, the present invention provides compounds of formula Ia or formula Ib, or pharmaceutically acceptable salts, tautomers or in vivo-hydrolysable precursors thereof, in the manufacture of a medicament for the treatment or prophylaxis of Aβ-related pathologies. In some further embodiments, the Aβ-related pathologies include such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

In some embodiments, the present invention provides a method of inhibiting activity of BACE comprising contacting the BACE with a compound of the present invention. BACE is thought to represent the major β-secretase activity, and is considered to be the rate-limiting step in the production of amyloid-β-protein (Aβ). Thus, inhibiting BACE through inhibitors such as the compounds provided herein would be useful to inhibit the deposition of Aβ and portions thereof. Because the deposition of Aβ and portions thereof is linked to diseases such as Alzheimer Disease, BACE is an important candidate for the development of drugs as a treatment and/or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

In some embodiments, the present invention provides a method for the treatment of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, comprising administering to a mammal (including human) a therapeutically effective amount of a compound of formula Ia or formula Ib, or a pharmaceutically acceptable salt, tautomer or in vivo-hydrolysable precursor thereof.

In some embodiments, the present invention provides a method for the prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration comprising administering to a mammal (including human) a therapeutically effective amount of a compound of formula Ia or formula Ib or a pharmaceutically acceptable salt, tautomer or in vivo-hydrolysable precursors.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration by administering to a mammal (including human) a compound of formula Ia or formula Ib or a pharmaceutically acceptable salt, tautomer or in vivo-hydrolysable precursors and a cognitive and/or memory enhancing agent.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration by administering to a mammal (including human) a compound of formula Ia or formula Ib or a pharmaceutically acceptable salt, tautomer or in vivo-hydrolysable precursors thereof wherein constituent members are provided herein, and a choline esterase inhibitor or anti-inflammatory agent.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, or any other disease, disorder, or condition described herein, by administering to a mammal (including human) a compound of the present invention, and an atypical antipsychotic agent Atypical antipsychotic agents includes, but not limited to, Olanzapine (marketed as Zyprexa), Aripiprazole (marketed as Abilify), Risperidone (marketed as Risperdal), Quetiapine (marketed as Seroquel), Clozapine (marketed as Clozaril), Ziprasidone (marketed as Geodon) and Olanzapine/Fluoxetine (marketed as Symbyax).

In some embodiments, the mammal or human being treated with a compound of the present invention, has been diagnosed with a particular disease or disorder, such as those described herein. In these cases, the mammal or human being treated is in need of such treatment. Diagnosis, however, need not be previously performed.

The present invention also includes pharmaceutical compositions which contain, as the active ingredient, one or more of the compounds of the invention herein together with at least one pharmaceutically acceptable carrier, diluent or excipent.

When used for pharmaceutical compositions, medicaments, manufacture of a medicament, inhibiting activity of BACE, or treating or preventing Aβ-related pathologies, compounds of the present invention include the compounds of formula Ia and/or formula Ib, and pharmaceutically acceptable salts, tautomers and in vivo-hydrolysable precursors thereof. Compounds of the present invention further include hydrates and solvates.

The definitions set forth in this application are intended to clarify terms used throughout this application. The term “herein” means the entire application.

As used in this application, the term “optionally substituted,” as used herein, means that substitution is optional and therefore it is possible for the designated atom or moiety to be unsubstituted. In the event a substitution is desired then such substitution means that any number of hydrogens on the designated atom or moiety is replaced with a selection from the indicated group, provided that the normal valency of the designated atom or moiety is not exceeded, and that the substitution results in a stable compound. For example, if a methyl group (i.e., CH₃) is optionally substituted, then 3 hydrogens on the carbon atom can be replaced. Examples of suitable substituents include, but are not limited to: halogen, CN, NH₂, OH, SO, SO₂, COOH, OC-alkyl, CH₂OH, SO₂H, C₁₋₆alkyl, OC₁₋₆alkyl, C(═O)C₁₋₆alkyl, C(═O)OC₁₋₆alkyl, C(═O)NH₂, C(═O)NHC₁₋₆alkyl, C(═O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(═O)C₁₋₆alkyl, NC(═O)(C₁₋₆alkyl)₂, C₅₋₆aryl, OC₅₋₆aryl, C(═O)C₅₋₆aryl, C(═O)OC₅₋₆aryl, C(═O)NHC₅₋₆aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl), N(C₅₋₆aryl)₂, NC(═O)C₅₋₆aryl, NC(═O)(C₅₋₆aryl)₂, C₅₋₆heterocyclyl, OC₅₋₆heterocyclyl, C(═O)C₅₋₆heterocyclyl, C(═O)OC₅₋₆heterocyclyl, C(═O)NHC₅₋₆heterocyclyl, C(═O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NC(═O)C₅₋₆heterocyclyl, NC(═O)(C₅₋₆heterocyclyl)₂.

A variety of compounds in the present invention may exist in particular geometric or stereoisomeric forms. The present invention takes into account all such compounds, including cis- and trans isomers, R- and S-enantiomers, diastereomers, (D)-isomers, (L)-isomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. When required, separation of the racemic material can be achieved by methods known in the art. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

As used herein, “alkyl”, “alkylenyl” or “alkylene” used alone or as a suffix or prefix, is intended to include both branched and straight-chain saturated aliphatic hydrocarbon groups having from 1 to 12 carbon atoms or if a specified number of carbon atoms is provided then that specific number would be intended. For example “C₁₋₆alkyl” denotes alkyl having 1, 2, 3, 4, 5 or 6 carbon atoms. Examples of alkyl include, but are not limited to, methyl, ethyl, n-propyl, i-propyl, n-butyl, i-butyl, sec-butyl, t-butyl, pentyl, and hexyl. As used herein, “C₁₋₃alkyl”, whether a terminal substituent or an alkylene (or alkylenyl) group linking two substituents, is understood to specifically include both branched and straight-chain methyl, ethyl, and propyl.

As used herein, “alkenyl” refers to an alkyl group having one or more double carbon-carbon bonds. Example alkenyl groups include ethenyl, propenyl, cyclohexenyl, and the like. The term “alkenylenyl” refers to a divalent linking alkenyl group.

As used herein, “alkynyl” refers to an alkyl group having one or more triple carbon-carbon bonds. Example alkynyl groups include ethynyl, propynyl, and the like. The term “alkynylenyl” refers to a divalent linking alkynyl group.

As used herein, “aromatic” refers to hydrocarbyl groups having one or more polyunsaturated carbon rings having aromatic characters, (e.g., 4n+2 delocalized electrons) and comprising up to about 14 carbon atoms.

As used herein, the term “aryl” refers to an aromatic ring structure made up of from 5 to 14 carbon atoms. Ring structures containing 5, 6, 7 and 8 carbon atoms would be single-ring aromatic groups, for example, phenyl. Ring structures containing 8, 9, 10, 11, 12, 13, or 14 would be a polycyclic moiety in which at least one carbon is common to any two adjoining rings therein (for example, the rings are “fused rings”), for example naphthyl. The aromatic ring can be substituted at one or more ring positions with such substituents as described above. The term “aryl” also includes polycyclic ring systems having two or more cyclic rings in which two or more carbons are common to two adjoining rings (the rings are “fused rings”) wherein at least one of the rings is aromatic, for example, the other cyclic rings can be cycloalkyls, cycloalkenyls or cycloalkynyls. The terms ortho, meta and para apply to 1,2-, 1,3- and 1,4-disubstituted benzenes, respectively. For example, the names 1,2-dimethylbenzene and ortho-dimethylbenzene are synonymous.

As used herein, “cycloalkyl” refers to non-aromatic cyclic hydrocarbons including cyclized alkyl, alkenyl, and alkynyl groups, having the specified number of carbon atoms. Cycloalkyl groups can include mono- or polycyclic (e.g., having 2, 3 or 4 fused or bridged rings) groups. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (i.e., having a bond in common with) to the cycloalkyl ring, for example, benzo derivatives of cyclopentane (i.e., indanyl), cyclopentene, cyclohexane, and the like. The term “cycloalkyl” further includes saturated ring groups, having the specified number of carbon atoms. These may include fused or bridged polycyclic systems. Preferred cycloalkyls have from 3 to 10 carbon atoms in their ring structure, and more preferably have 3, 4, 5, and 6 carbons in the ring structure. For example, “C₃₋₆ cycloalkyl” denotes such groups as cyclopropyl, cyclobutyl, cyclopentyl, or cyclohexyl.

As used herein, “cycloalkenyl” refers to ring-containing hydrocarbyl groups having at least one carbon-carbon double bond in the ring, and having from 3 to 12 carbons atoms.

As used herein, “halo” or “halogen” refers to fluoro, chloro, bromo, and iodo.

“Counterion” is used to represent a small, negatively or positively charged species such as chloride (Cl⁻), bromide (Br⁻), hydroxide (OH⁻), acetate (CH₃COO⁻), sulfate (SO4²⁻), tosylate (CH₃-phenyl-SO₃—), benezensulfonate (phenyl-SO₃⁻), sodium ion (Na⁺), potassium (K⁺), ammonium (NH₄⁺), and the like.

As used herein, the term “heterocyclyl” or “heterocyclic” or “heterocycle” refers to a ring-containing monovalent and divalent structures having one or more heteroatoms, independently selected from N, O and S, as part of the ring structure and comprising from 3 to 20 atoms in the rings, more preferably 3- to 7-membered rings. The number of ring-forming atoms in heterocyclyl are given in ranges herein. For example, C₅₋₁₀ heterocyclyl refers to a ring structure comprising from 5 to 10 ring-forming atoms wherein at least one of the ring-forming atoms is N, O or S. Heterocyclic groups may be saturated or partially saturated or unsaturated, containing one or more double bonds, and heterocyclic groups may contain more than one ring as in the case of polycyclic systems. The heterocyclic rings described herein may be substituted on carbon or on a heteroatom atom if the resulting compound is stable. If specifically noted, nitrogen in the heterocyclyl may optionally be quaternized. It is understood that when the total number of S and O atoms in the heterocyclyl exceeds 1, then these heteroatoms are not adjacent to one another.

Examples of heterocyclyls include, but are not limited to, 1H-indazole, 2-pyrrolidonyl, 2H, 6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azabicyclo, azetidine, azepane, aziridine, azocinyl, benzimidazolyl, benzodioxol, benzofuranyl, benzothiofuranyl, benzothiophenyl, benzoxazolyl, benzthiazolyl, benzotriazolyl, benzotetrazolyl, benzisoxazolyl, benzisothiazolyl, benzimidazalonyl, carbazolyl, 4aH-carbazolyl, b-carbolinyl, chromanyl, chromenyl, cinnolinyl, diazepane, decahydroquinolinyl, 2H,6H-1,5,2-dithiazinyl, dioxolane, furyl, 2,3-dihydrofuran, 2,5-dihydrofuran, dihydrofuro[2,3-b]tetrahydrofuran, furanyl, furazanyl, homopiperidinyl, imidazolidine, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxirane, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, purinyl, pyranyl, pyrrolidinyl, pyrroline, pyrrolidine, pyrazinyl, pyrazolidinyl, pyrazolinyl, pyrazolyl, pyridazinyl, pyridooxazole, pyridoimidazole, pyridothiazole, pyridinyl, N-oxide-pyridinyl, pyridyl, pyrimidinyl, pyrrolidinyl, pyrrolidinyl dione, pyrrolinyl, pyrrolyl, pyridine, quinazolinyl, quinolinyl, 4H-quinolizinyl, quinoxalinyl, quinuclidinyl, carbolinyl, tetrahydrofuranyl, tetramethylpiperidinyl, tetrahydroquinoline, tetrahydroisoquinolinyl, thiophane, thiotetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, thiirane, triazinyl, 1,2,3-triazolyl, 1,2,4-triazolyl, 1,2,5-triazolyl, 1,3,4-triazolyl, xanthenyl.

As used herein, “heteroaryl” refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 4 to about 14, 3 to about 7, or 5 to 6 ring-forming atoms. In some embodiments, the heteroaryl group has 1 to about 4, 1 to about 3, or 1 to 2 heteroatoms. In some embodiments, the heteroaryl group has 1 heteroatom.

As used herein, “alkoxy” or “alkyloxy” represents an alkyl group as defined above with the indicated number of carbon atoms attached through an oxygen bridge. Examples of alkoxy include, but are not limited to, methoxy, ethoxy, n-propoxy, isopropoxy, n-butoxy, isobutoxy, t-butoxy, n-pentoxy, isopentoxy, cyclopropylmethoxy, allyloxy and propargyloxy. Similarly, “alkylthio” or “thioalkoxy” represent an alkyl group as defined above with the indicated number of carbon atoms attached through a sulphur bridge.

As used herein, the term “carbonyl” is art recognized and includes such moieties as can be represented by the general formula:

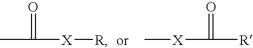

wherein X is a bond or represents an oxygen or sulfur, and R represents a hydrogen, an alkyl, an alkenyl, —(CH₂)ₘ—R″ or a pharmaceutically acceptable salt, R′ represents a hydrogen, an alkyl, an alkenyl or —(CH₂)ₘ—R″, where m is an integer less than or equal to ten, and R″ is alkyl, cycloalkyl, alkenyl, aryl, or heteroaryl. Where X is an oxygen and R and R′ is not hydrogen, the formula represents an “ester”. Where X is an oxygen, and R is as defined above, the moiety is referred to herein as a carboxyl group, and particularly when R′ is a hydrogen, the formula represents a “carboxylic acid.” Where X is oxygen, and R′ is a hydrogen, the formula represents a “formate.” In general, where the oxygen atom of the above formula is replaced by sulfur, the formula represents a “thiolcarbonyl” group. Where X is a sulfur and R and R′ is not hydrogen, the formula represents a “thiolester.” Where X is sulfur and R is hydrogen, the formula represents a “thiolcarboxylic acid.” Where X is sulfur and R′ is hydrogen, the formula represents a “thiolformate.” On the other hand, where X is a bond, and R is not a hydrogen, the above formula represents a “ketone” group. Where X is a bond, and R is hydrogen, the above formula is represents an “aldehyde” group.

As used herein, the term “sulfonyl” refers to a moiety that can be represented by the general formula:

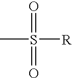

wherein R is represented by but not limited to hydrogen, alkyl, cycloalkyl, alkenyl, aryl, heteroaryl, aralkyl or heteroaralkyl.

As used herein, some substituents are described in a combination of two or more groups. For example, the expression of “C(═O)C₃₋₉cycloalkylR^{d}” is meant to refer to a structure:

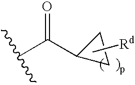

wherein p is 1, 2, 3, 4, 5, 6 or 7 (i.e., C₃₋₉cycloalkyl); the C₃₋₉cycloalkyl is substituted by R^{d}; and the point of attachment of the “C(═O)C₃₋₉cycloalkylR^{d}” is through the carbon atom of the carbonyl group, which is on the left of the expression.

As used herein, the phrase “protecting group” means temporary substituents which protect a potentially reactive functional group from undesired chemical transformations. Examples of such protecting groups include esters of carboxylic acids, silyl ethers of alcohols, and acetals and ketals of aldehydes and ketones respectively. The field of protecting group chemistry has been reviewed (Greene, T. W.; Wuts, P.G.M. Protective Groups in Organic Synthesis, 3^{rd} ed.; Wiley: New York, 1999).

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof (i.e., also include counterions). Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, phosphoric, and the like; and the salts prepared from organic acids such as lactic, maleic, citric, benzoic, methanesulfonic, and the like.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; nonaqueous media like ether, ethyl acetate, ethanol, isopropanol, or acetonitrile can be used.

As used herein, “in vivo hydrolysable precursors” means an in vivo hydroysable (or cleavable) ester of a compound of formula Ia or formula Ib that contains a carboxy or a hydroxy group. For example amino acid esters, C₁₋₆ alkoxymethyl esters like methoxymethyl; C₁₋₆alkanoyloxymethyl esters like pivaloyloxymethyl; C₃₋₉cycloalkoxycarbonyloxy C₁₋₆alkyl esters like 1-cyclohexylcarbonyloxyethyl, acetoxymethoxy, or phosphoramidic cyclic esters.

As used herein, “tautomer” means other structural isomers that exist in equilibrium resulting from the migration of a hydrogen atom. For example, keto-enol tautomerism where the resulting compound has the properties of both a ketone and an unsaturated alcohol.

As used herein “stable compound” and “stable structure” are meant to indicate a compound that is sufficiently robust to survive isolation to a useful degree of purity from a reaction mixture, and formulation into an efficacious therapeutic agent.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled compound” is a compound that has incorporated at least one radionuclide. In some embodiments the radionuclide is selected from the group consisting of ³H, ¹⁴C, ¹²⁵I, ³⁵S and ⁸²Br.

The antidementia treatment defined herein may be applied as a sole therapy or may involve, in addition to the compound of the invention, conventional chemotherapy. Such chemotherapy may include one or more of the following categories of agents:

Such conjoint treatment may be achieved by way of the simultaneous, sequential or separate dosing of the individual components of the treatment. Such combination products employ the compounds of this invention.

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of dementia is an amount sufficient to symptomatically relieve in a warm-blooded animal, particularly a human the symptoms of dementia, to slow the progression of dementia, or to reduce in patients with symptoms of dementia the risk of getting worse.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Some of the compounds of the present invention are capable of forming salts with various inorganic and organic acids and bases and such salts are also within the scope of this invention. For example, such conventional non-toxic salts include those derived from inorganic acids such as hydrochloric, phosphoric, and the like; and the salts prepared from organic acids such as lactic, maleic, citric, benzoic, methanesulfonic, trifluoroacetate and the like.

In some embodiments, the present invention provides a compound of formula Ia or formula Ib or a pharmaceutically acceptable salt thereof for the therapeutic treatment (including prophylactic treatment) of mammals including humans, it is normally formulated in accordance with standard pharmaceutical practice as a pharmaceutical composition.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition, 1975.

The compounds of the invention may be derivatised in various ways. As used herein “derivatives” of the compounds includes salts (e.g. pharmaceutically acceptable salts), any complexes (e.g. inclusion complexes or clathrates with compounds such as cyclodextrins, or coordination complexes with metal ions such as Mn²⁺ and Zn²⁺), esters such as in vivo hydrolysable esters, free acids or bases, polymorphic forms of the compounds, solvates (e.g. hydrates), prodrugs or lipids, coupling partners and protecting groups. By “prodrugs” is meant for example any compound that is converted in vivo into a biologically active compound.

Salts of the compounds of the invention are preferably physiologically well tolerated and non toxic. Many examples of salts are known to those skilled in the art. All such salts are within the scope of this invention, and references to compounds include the salt forms of the compounds.

Compounds having acidic groups, such as carboxylate, phosphates or sulfates, can form salts with alkaline or alkaline earth metals such as Na, K, Mg and Ca, and with organic amines such as triethylamine and Tris (2-hydroxyethyl)amine. Salts can be formed between compounds with basic groups, e.g. amines, with inorganic acids such as hydrochloric acid, phosphoric acid or sulfuric acid, or organic acids such as acetic acid, citric acid, benzoic acid, fumaric acid, or tartaric acid. Compounds having both acidic and basic groups can form internal salts.

Acid addition salts may be formed with a wide variety of acids, both inorganic and organic. Examples of acid addition salts include salts formed with hydrochloric, hydriodic, phosphoric, nitric, sulphuric, citric, lactic, succinic, maleic, malic, isethionic, fumaric, benzenesulphonic, toluenesulphonic, methanesulphonic, ethanesulphonic, naphthalenesulphonic, valeric, acetic, propanoic, butanoic, malonic, glucuronic and lactobionic acids.

If the compound is anionic, or has a functional group which may be anionic (e.g., COOH may be COO), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺) Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds contain an amine function, these may form quaternary ammonium salts, for example by reaction with an alkylating agent according to methods well known to the skilled person. Such quaternary ammonium compounds are within the scope of the invention. Compounds containing an amine function may also form N-oxides. A reference herein to a compound that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March; 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Esters can be formed between hydroxyl or carboxylic acid groups present in the compound and an appropriate carboxylic acid or alcohol reaction partner, using techniques well known in the art. Examples of esters are compounds containing the group C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, C(═O)OCH₃, C(═O)OCH₂CH₃, C(═O)OC(CH₃)₃, and —C(═O)OPh. Examples of acyloxy (reverse ester) groups are represented by OC(═O)R, wherein R is an acyloxy substituent, for example, a C₁₋₇ alkyl group, a C₃₋₂₀ heterocyclyl group, or a C₅₂₀ aryl group, preferably a C₁₋₇ alkyl group. Particular examples of acyloxy groups include, but are not limited to, OC(═O)CH₃ (acetoxy), OC(═O)CH₂CH₃, OC(═O)C(CH₃)₃, OC(═O)Ph, and OC(═O)CH₂Ph.

Derivatives which are prodrugs of the compounds are convertible in vivo or in vitro into one of the parent compounds. Typically, at least one of the biological activities of compound will be reduced in the prodrug form of the compound, and can be activated by conversion of the prodrug to release the compound or a metabolite of it. Some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is: C₁₇alkyl (e.g., Me, Et, -nPr, -iPr, -nBu, -sBu, -iBu, tBu); C₁₇aminoalkyl (e.g., aminoethyl; 2-(N,N-diethylamino)ethyl; 2(4morpholino)ethyl); and acyloxy-C₁₇alkyl (e.g., acyloxymethyl; acyloxyethyl; pivaloyloxymethyl; acetoxymethyl; 1acetoxyethyl; 1-(1-methoxy-1-methyl)ethyl-carbonyloxyethyl; 1-(benzoyloxy)ethyl; isopropoxy-carbonyloxymethyl; isopropoxy-carbonyloxyethyl; cyclohexyl-carbonyloxymethyl; 1cyclohexyl-carbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetrahydropyranyloxy)carbonyloxyethyl; (4-tetrahydropyranyl)carbonyloxymethyl; and 1(4-tetrahydropyranyl)carbonyloxyethyl).

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

Other derivatives include coupling partners of the compounds in which the compounds is linked to a coupling partner, e.g. by being chemically coupled to the compound or physically associated with it. Examples of coupling partners include a label or reporter molecule, a supporting substrate, a carrier or transport molecule, an effector, a drug, an antibody or an inhibitor. Coupling partners can be covalently linked to compounds of the invention via an appropriate functional group on the compound such as a hydroxyl group, a carboxyl group or an amino group. Other derivatives include formulating the compounds with liposomes.

Where the compounds contain chiral centres, all individual optical forms such as enantiomers, epimers and diastereoisomers, as well as racemic mixtures of the compounds are within the scope of the invention.

Compounds may exist in a number of different geometric isomeric, and tautomeric forms and references to compounds include all such forms. For the avoidance of doubt, where a compound can exist in one of several geometric isomeric or tautomeric forms and only one is specifically described or shown, all others are nevertheless embraced by the scope of this invention.

The quantity of the compound to be administered will vary for the patient being treated and will vary from about 100 ng/kg of body weight to 100 mg/kg of body weight per day and preferably will be from 10 pg/kg to 10 mg/kg per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention.

Compounds of the present invention have been shown to inhibit beta secretase (including BACE) activity in vitro. Inhibitors of beta secretase have been shown to be useful in blocking formation or aggregation of Aβ peptide and therefore have a beneficial effects in treatment of Alzheimer's Disease and other neurodegenerative diseases associated with elevated levels and/or deposition of Aβ peptide. Therefore it is believed that the compounds of the present invention may be used for the treatment of Alzheimer disease and disease associated with dementia Hence compounds of the present invention and their salts are expected to be active against age-related diseases such as Alzheimer, as well as other Aβ related pathologies such as Downs syndrome and b-amyloid angiopathy. It is expected that the compounds of the present invention would most likely be used in combination with a broad range of cognition deficit enhancement agents but could also be used as a single agent.

Generally, the compounds of the present invention have been identified in one or both assays described below as having an IC₅₀ value of 100 micromolar or less. For example the compound of example number 34 has an IC₅₀ value of 36 nM.

### IGEN Assay

Enzyme is diluted 1:30 in 40 mM MES pH 5.0. Stock substrate is diluted to 12 μM in 40 mM MES pH 5.0. PALMEB solution is added to the substrate solution (1:100 dilution). DMSO stock solutions of compounds or DMSO alone are diluted to the desired concentration in 40 mM MES pH 5.0. The assay is done in a 96 well PCR plate from Nunc. Compound in DMSO (3 μL) is added to the plate then enzyme is added (27 μL) and pre-incubated with compound for 5 minutes. Then the reaction is started with substrate (30 μL). The final dilution of enzyme is 1:60; the final concentration of substrate is 6 μM (Km is 150 μM). After a 20 minute reaction at room temperature, the reaction is stopped by removing 101 of the reaction mix and diluting it 1:25 in 0.20M Tris pH 8.0. The compounds are added to the plate by hand then all the rest of the liquid handling is done on the CyBi-well instrument.

All antibodies and the streptavidin coated beads are diluted into PBS containing 0.5% BSA and 0.5% Tween20. The product is quantified by adding 50 μL of a 1:5000 dilution of the neoepitope antibody to 50 μL of the 1:25 dilution of the reaction mix. Then, 100 μL of PBS (0.5% BSA, 0.5% Tween20) containing 0.2 mg/ml IGEN beads and a 1:5000 dilution of ruthinylated goat anti-rabbit (Ru-Gar) antibody is added. The final dilution of neoepitope antibody is 1:20,000, the final dilution of Ru-GAR is 1:10,000 and the final concentration of beads is 0.1 mg/ml. The mixture is read on the IGEN instrument with the CindyAB40 program after a 2-hour incubation at room temperature. Addition of DMSO alone is used to define the 100% activity. 20 μM control inhibitor is used to define 0% of control activity and 100 nM inhibitor defines 50% control of control activity in single-poke assays. Control inhibitor is also used in dose response assays with an IC50 of 100 nM.

### Fluorescent Assay

Enzyme is diluted 1:30 in 40 mM MES pH 5.0. Stock substrate is diluted to 30 μM in 40 mM MES pH 5.0. PALMEB solution is added to the substrate solution (1:100 dilution). Enzyme and substrate stock solutions are kept on ice until the placed in the stock plates. The Platemate-plus instrument is used to do all liquid handling. Enzyme (9 μL) is added to the plate then 1 μL of compound in DMSO is added and pre-incubated for 5 minutes. When a dose response curve is being tested for a compound, the dilutions are done in neat DMSO and the DMSO stocks are added as described above. Substrate (10 mL) is added and the reaction proceeds in the dark for 1 hour at room temperature. The assay is done in a Corning 384 well round bottom, low volume, non-binding surface (Corning #3676). The final dilution of enzyme is 1:60; the final concentration of substrate is 15 μM (Km of 25 μM). The fluorescence of the product is measured on a Victor II plate reader with an excitation wavelength of 360 nm and an emission wavelength of 485 nm using the protocol labeled Edans peptide. The DMSO control defines the 100% activity level and 0% activity is defined by using 50 μM of the control inhibitor, which completely blocks enzyme function. The control inhibitor is also used in dose response assays and has an IC50 of 95 nM.

### Beta-Secretase Whole Cell Assay

Generation of HEK-Fc33-1:

The cDNA encoding full length BACE was fused in frame with a three amino acid linker (Ala-Val-Thr) to the Fc portion of the human IgG1 starting at amino acid 104. The BACE-Fc construct was then cloned into a GFP/pGEN-IRES-neoK vector (a proprietary vector of AstraZeneca) for protein expression in mammalian cells. The expression vector was stably transfected into HEK-293 cells using a calcium phosphate method. Colonies were selected with 250 μg/mL of G418. Limited dilution cloning was performed to generate homogeneous cell lines. Clones were characterized by levels of APP expression and Aβ secreted in the conditioned media using an ELISA assay developed in-house. Aβ secretion of BACE/Fc clone Fc33-1 was moderate.

Cell Culture:

HEK293 cells stably expressing human BACE (HEK-Fc33) were grown at 37° C. in DMEM containing 10% heat-inhibited FBS, 0.5 mg/mL antibiotic-antimycotic solution, and 0.05 mg/mL of the selection antibiotic G-418.

Aβ40 Release Assay:

Cells were harvested when between 80 to 90% confluent. 100 μL of cells at a cell density of 1.5 million/mL were added to a white 96-well cell culture plate with clear flat bottom (Costar 3610), or a clear, flat bottom 96-well cell culture plate (Costar 3595), containing 100 μL of inhibitor in cell culture medium with DMSO at a final concentration of 1%. After the plate was incubated at 37° C. for 24 h, 100 μL cell medium was transferred to a round bottom 96-well plate (Costar 3365) to quantify A040 levels. The cell culture plates were saved for ATP assay as described in ATP assay below. To each well of the round bottom plate, 50 μL of detection solution containing 0.2 μg/m of the RαAβ40 antibody and 0.25 μg/mL of a biotinylated 4G8 antibody (prepared in DPBS with 0.5% BSA and 0.5% Tween-20) was added and incubated at 4° C. for at least 7 h. Then a 50 μL solution (prepared in the same buffer as above) containing 0.062 μg/mL of a ruthenylated goat anti-rabbit antibody and 0.125 mg/mL of streptavidin coated Dynabeads was added per well. The plate was shaken at 22° C. on a plate shaker for 1 h, and then the plates were then measured for ECL counts in an IGEN M8 Analyzer. Aβ standard curves were obtained with 2-fold serial dilution of an Aβ stock solution of known concentration in the same cell culture medium used in cell-based assays.

ATP Assay:

As indicated above, after transferring 100 μL medium from cell culture plates for Aβ40 detection, the plates, which still contained cells, were saved for cytotoxicity assays by using the assay kit (ViaLight™ Plus) from Cambrex BioScience that measures total cellular ATP. Briefly, to each well of the plates, 50 μL cell lysis reagent was added. The plates were incubated at room temperature for 10 min. Two min following addition of 100 μL reconstituted ViaLight™ Plus reagent for ATP measurement, the luminescence of each well was measured in an LJL plate reader or Wallac Topcount.

### BACE Biacore Protocol

### Sensor Chip Preparation:

BACE was assayed on a Biacore3000 instrument by attaching either a peptidic transition state isostere (TSI) or a scrambled version of the peptidic TSI to the surface of a Biacore CM5 sensor chip. The surface of a CM5 sensor chip has 4 distinct channels that can be used to couple the peptides. The scrambled peptide KFES-statine-ETIAEVENV was coupled to channel 1 and the TSI inhibitor KTEEISEVN-statine-VAEF was couple to channel 2 of the same chip. The two peptides were dissolved at 0.2 mg/ml in 20 mM Na Acetate pH 4.5, and then the solutions were centrifuged at 14K rpm to remove any particulates. Carboxyl groups on the dextran layer were activated by injecting a one to one mixture of O.5M N-ethyl-N′ (3-dimethylaminopropyl)-carbodiimide (EDC) and 0.5M N-hydroxysuccinimide (NHS) at 5 μL/minute for 7 minutes. Then the stock solution of the control peptide was injected in channel 1 for 7 minutes at 5 μL/min., and then the remaining activated carboxyl groups were blocked by injecting 1M ethanolamine for 7 minutes at 5 μL/minute.

### Assay Protocol:

The BACE Biacore assay was done by diluting BACE to 0.5 μM in Na Acetate buffer at pH 4.5 (running buffer minus DMSO). The diluted BACE was mixed with DMSO or compound diluted in DMSO at a final concentration of 5% DMSO. The BACE/inhibitor mixture was incubated for 1 hour at 4° C. then injected over channel 1 and 2 of the CM5 Biacore chip at a rate of 20 μL/minute. As BACE bound to the chip the signal was measured in response units (RU). BACE binding to the TSI inhibitor on channel 2 gave a certain signal. The presence of a BACE inhibitor reduced the signal by binding to BACE and inhibiting the interaction with the peptidic TSI on the chip. Any binding to channel 1 was non-specific and was subtracted from the channel 2 responses. The DMSO control was defined as 100% and the effect of the compound was reported as percent inhibition of the DMSO control. 2-Amino-3-methyl-6-(2-naphthalen-2-yl-ethyl)-3H-pyrimidin-4-one, AZ12066871, inhibited BACE binding in the BACE Biacore assay by 69% when tested at a concentration of 1 mM.

The compounds of the present invention can be prepared in a number of ways well known to one skilled in the art of organic synthesis. The compounds of the present invention can be synthesized using the methods described below, together with synthetic methods known in the art of synthetic organic chemistry, or variations thereon as appreciated by those skilled in the art. Such methods include, but are not limited to, those described below. All references cited herein are hereby incorporated in their entirety by reference.

The novel compounds of this invention may be prepared using the reactions and techniques described herein. The reactions are performed in solvents appropriate to the reagents and materials employed and are suitable for the transformations being effected. Also, in the description of the synthetic methods described below, it is to be understood that all proposed reaction conditions, including choice of solvent, reaction atmosphere, reaction temperature, duration of the experiment and workup procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents, which are not compatible with the reaction conditions, will be readily apparent to one skilled in the art and alternate methods must then be used.

The starting materials for the examples contained herein are either commercially available or are readily prepared by standard methods from known materials. For example the following reactions are illustrations but not limitations of the preparation of some of the starting materials and examples used herein.

General Procedures for Making the Compounds of the Invention is as Follows:

The invention will now be illustrated by the following nonlimiting examples, in which, unless stated otherwise:
I. temperatures are given in degrees Celsius (° C.); unless otherwise stated, operations were carried out at room or ambient temperature, that is, at a temperature in the range of 1825° C.;
II. organic solutions were dried over anhydrous magnesium sulfate; evaporation of solvent was carried out using a rotary evaporator under reduced pressure (6004000 Pascals; 4.530 mm Hg) with a bath temperature of up to 60° C.;
II. chromatography means flash chromatography on silica gel; thin layer chromatography (TLC) was carried out on silica gel plates;
IV. in general, the course of reactions was followed by TLC or HPLC and reaction times are given for illustration only;
V. melting points are uncorrected and (dec) indicates decomposition;
VI. final products had satisfactory proton nuclear magnetic resonance (NMR) spectra;
VII. when given, NMR data is in the form of delta values for major diagnostic protons, given in parts per million (ppm) relative to tetramethylsilane (TMS) as an internal standard, determined at 300 MHz using deuterated chloroform (CDCl₃), dimethylsulphoxide (d₆-DMSO) or dimethylsulphoxide/TFA (d₆-DMSO/TFA) as solvent; conventional abbreviations for signal shape are used; for AB spectra the directly observed shifts are reported; coupling constants (J) are given in Hz; Ar designates an aromatic proton when such an assignment is made;
VIII. reduced pressures are given as absolute pressures in pascals (Pa); elevated pressures are given as gauge pressures in bars;
IX. non-aqueous reactions were run under a nitrogen atmosphere
X. solvent ratios are given in volume:volume (v/v) terms; and
XI. Mass spectra (MS) were run using an automated system with atmospheric pressure chemical (APCI) or electrospry (+ES) ionization. Generally, only spectra where parent masses are observed are reported. The lowest mass major ion is reported for molecules where isotope splitting results in multiple mass spectral peaks (for example when chlorine is present).
XII. Commercial reagents were used without further purification.
XIII. The ketone starting materials for compounds prepared according to schemes 1 or 2, unless otherwise noted, were either commercially available or prepared according to the procedures in the following references: Example 14, Chemical Abstracts, CAN 123:115721, AN 2000:718846; Example 10, Broxton et al, J. Chem. Soc. Perkin Trans. 1, 1974, 1769-1771; Example 12 Boatman et al., J. Org. Chem, 1965, 30, 3321-3324.
XIV. Phosphonoacetate used to prepare olefins such as that in (Scheme 1, A), may be either trirethylphosphonoacetate, ethyl dimethylphosphonoacetate, tert-butyl dimethylphosphonoacetate, triethylphosphonoacetate, methyl diethylphosphonoacetate, or tert-butyl diethylphosphonoacetate.
XV. Mass spectra were recorded using either a Hewlett Packard 5988A or a MicroMass Quattro-1 Mass Spectrometer and are reported as m/z for the parent molecular ion.
XVI. Room temperature refers to 20-25° C.
XVII. LC-MS HPLC conditions: Column: Agilent Zorbax SB-C8 2 mm ID×50 mm Flow: 1.4 mL/minGradient: 95% A to 90% B over 3 min. hold 1 minute ramp down to 95% A over 1 minute and hold 1 minute. Where A=2% acetonitrile in water with 0.1% formic acid and B=2% water in acetonitrile with 0.1% formic acid. UV-DAD 210400 nm
XVIII. Preparative reverse phase HPLC conditions (A): Compounds were purified on a Phenomenex Luna C18 reverse phase column (250×21 mm, 10 micron particle size) using an Agilent system. The crude compounds were solubilized in acetonitrile:water:TFA (75:25:0.1). An elution gradient (0-50% acetonitrile over 12 mins, hold at 50% acetonitrile for 3 mins, 50-100% acetonitrile over 7 mins, flow rate at 40 ml/min, 220 nm) yielded the purified title compounds. Retention time (t_{R})=mins. This method was used for Examples 1-28.
XIX. Preparative reverse phase HPLC conditions (B): Compounds were purified on a Phenomenex Luna C18(2) reverse phase column (60×21.2 mm, 10 micron particle size) using a Gilson system. Gradient elution performed with aqueous 0.1% trifluoroacetic acid in water and acetonitrile (typically 25-75% acetonitrile over 15 min.) with flow rate of 50 mL/min, UV collection at 220 nm. This method was used for Examples 29-87.
XX. Preparative reverse phase HPLC conditions (C): Gilson instrumentation (215 Injector, 333 Pumps and 155 UV/Vis Detector): Varian C8 reverse phase column (60 Angstrom irregular load in 8 mm particle size, 21 mm ID×25 cm). The crude compounds were solubilized in dimethyl sulfoxide:methanol (˜1:1). Gradient elution performed with aqueous 0.1% trifluoroacetic acid/acetonitrile (typically 25-75% acetonitrile over 30 min., 95% acetonitrile over 7 min.) flow rate at 22 mL/min, UV collection at 254 nm. Retention time (t_{R})=mins. This method was used for Examples 88-94.
XXI. Normal phase chromatography conditions: Flash chromatography employed as a method for purification for selected intermediates. Isco CombiFlash Sq 16× instrument: pre-packaged disposable RediSep SiO₂ stationary phase columns (4, 12, 40, 120 gram sizes) with gradient elution at 5-125 mL/min of selected bi-solvent mixture, UV detection (190-760 nm range) or timed collection, 0.1 mm flow cell path length.
XXI. Microwave heating instrumentation: A Personal Chemistry Smith Synthesizer unit (monomodal, 2.45 GHz, 300W max) was utilized for microwave heating of reactions.
XXIII. Terms and abbreviations: Solvent mixture compositions are given as volume percentages or volume ratios. In cases where the NMR spectra are complex; only diagnostic signals are reported. atm: atmospheric pressure; Boc: t-butoxycarbonyl; Cbz: benzyloxycarbonyl; DCM: methylene chloride; DIPEA: diisopropylethylamine; DMF: N;N-dimethyl formamide; DMSO: dimethyl sulfoxide; Et₂O: diethyl ether; EtOAc: ethyl acetate; h: hour(s); HPLC: high pressure liquid chromatography; minute(s): min.; NMR: nuclear magnetic resonance; psi: pounds per square inch; TFA: trifluoroacetic acid; THF: tetrahydrofuran; ACN: acetonitrile.

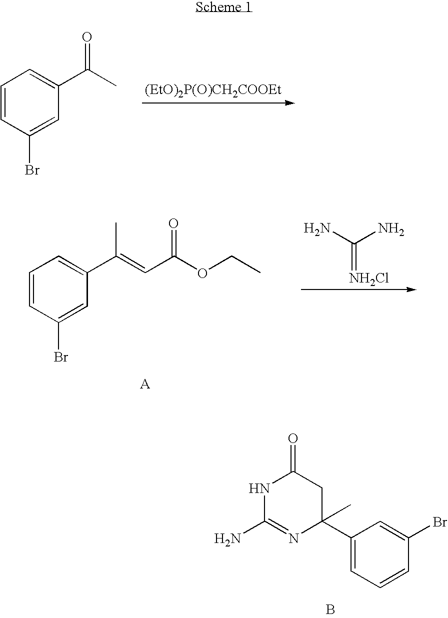

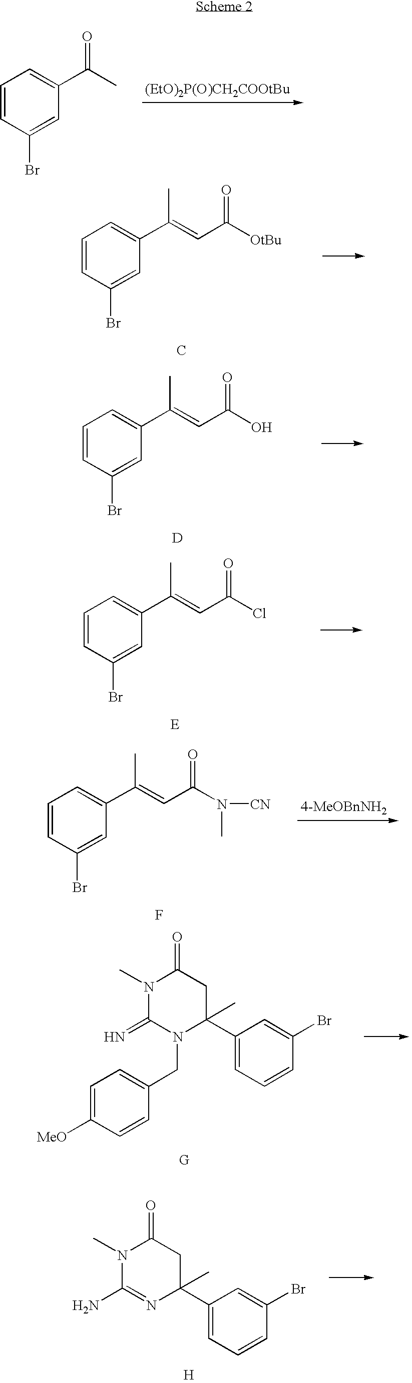

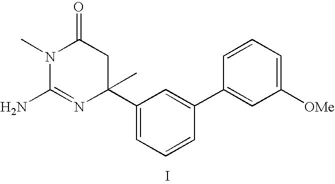

### EXAMPLE 1

### 2-Amino-6-(3-bromo-phenyl)-6-methyl-5,6-dihydro-3H-pyrimidin-4-one (Scheme 1, B)

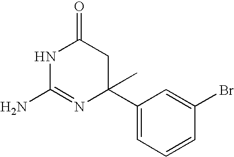

To a solution of guanidine HCl salt (0.35 g, 3.72 mmol) and sodium methoxide (0.16 g, 4.09 mmol) in NMP (2 mL) was added (E)-3-(3-bromo-phenyl)-but-2-enoic acid ethyl ester (Scheme 1, A) (0.5 g, 1.86 mmol) and the reaction was subjected to microwaves at 200° C. for 15 min. The NMP was removed under reduced pressure to yield a dark amber syrup. To this was added acetonitrile:water:TFA (75:25:0.1, 10 ml) and the resulting precipitate was removed. The filtrate was purified using RP-HPLC (t_{R}=8.33). The combined purified fractions were lyophilized to give the title compound as a light tan powder (0.21 g, 40%). ¹H NMR (300 MHz, DMSO-d₆): δ 1.64 (s, 3H); 3.14 (d, 1H, J=16.5 Hz); 3.34 (d, 1H, J=16.5 Hz); 7.44 (m, 2H); 7.55 (m, 1H); 7.64 (s, 1H). m/z (ES) 282 M⁺.

To one skilled in the art, it is appreciated that the olefin used in the cyclization may be one of a diverse set of esters, for example methyl, ethyl, isopropyl or t-butyl. However, t-butyl esters are sometimes less efficient in the cyclization reaction. In these cases, the t-butyl ester can be converted to a methyl ester via Fisher ester synthesis, i.e. by treatment with concentrated sulfuric acid in methanol (1:10 V:V).

### (E)-3-(S-bromo-phenyl)-but-2-enoic acid ethyl ester (Scheme 1, A) was Prepared as Follows

### (E)-3-(3-bromo-phenyl)-but-2-enoic acid ethyl ester (Scheme 1, A)

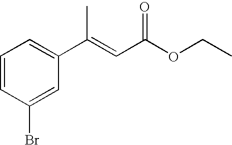

To a −78° C. stirred solution of triethyl phosphonoacetate (6.19 g, 27.63 mmol) in THF (70 mL) was added n-BuLi in hexanes (1.6 N, 18.06 mL, 28.89 mmol) and the reaction was stirred at −78° C. for 30 min. To this mixture was added 3′-bromoacetophenone (3.34 mL, 25.12 mmole) and the reaction stirred at −78° C. for 30 min. The mixture was warmed to room temperature and stirred for 18 hours. The THF was removed under reduced pressure to yield a cloudy yellow oil. To this was added hexanes (250 mL) and the reaction stirred for 10 min. The resulting precipitate was removed and the filtrate collected and concentrated under reduced pressure. The crude compound was purified using flash chromatography (silica gel, 5:95 ethyl acetate:hexanes) to give the title compound as a pale, clear yellow oil (5.63 g, 83%). ¹H NMR (300 MHz, DMSO-d₆): δ 1.26 (t, 3H, J=7.2 Hz); 2.44 (s, 3H); 4.10 (q, 2H, J=7.2 Hz); 6.17 (s, 1H); 7.35 (t, 1H, J=7.8 Hz); 7.56 (m, 2H); 7.68 (s, 1H). m/z (ES) 269 M⁺.

### EXAMPLE 2

### 2-Amino-6-(3′-methoxy-biphenyl-3-yl)-3,6-dimethyl-5,6-dihydro-3H-pyrimidin-4-one (Scheme 2, I)

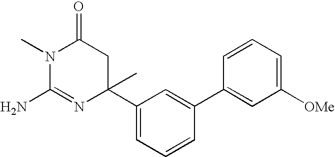

General Suzuki Conditions Method A: To a solution of 2-amino-6-(3-bromo-phenyl)-3,6-dimethyl-5,6-dihydro-3H-pyrimidin-4-one (Scheme 2, H) (47 mg, 0.132 mmol) in 1.5 mL 7:3:2 1,2-dimethoxyethane:water:ethanol was added was added cesium carbonate (129 mg, 0.396 mmol), 3-methoxyphenylboronic acid (26 mg, 0.172 mmol), and dichlorobis(triphenylphosphine)palladium(II) (4.6 mg, 0.0065 mmol). The reaction was subjected to microwaves for 15 min. at 150° C. after which the solvents were removed under a stream of nitrogen. To this brown gum was added ACN:water:TFA (75:25:0.1, 2.0 ml) and the resulting precipitate removed. The filtrate was purified using RP-HPLC (Ret. time: 14.2 mins). The combined purified fractions were lyophilized to give the title compound as a white powder (25 mg g, 43%). ¹H NMR (300 MHz, DMSO-dr/TFA-d): δ 1.71 (s, 3H); 3.13 (s, 3H); 3.21 (d, 1H, J=16.5 Hz); 3.59 (d, 1H, J=16.2 Hz); 3.85 (s, 3H); 6.98 (d, 1H, J=3.9 Hz); 7.23 (m, 2H); 7.41 (m, 2H); 7.51 (t, 1H, J=7.8 Hz); 7.64 (d, 1H, J=7.5 Hz); 7.71 (s, 1H); m/z (APCI+) M+1 (324.17); LCMS t_{R} 1.97 min.

### EXAMPLE 3

### 6-(3′-methoxy-1,1′-biphenyl-3-yl)-6-methyl-2-(methylamino)-5,6-dihydropyrimidin-4(3H)-one

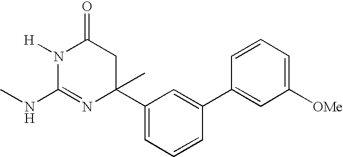

The HPLC purification of Example 2 resulted in isolation of the title compound as a white powder (4.7 mg, 10%). ¹H NMR (300.132 MHz, DMSO) d 1.77 (s, 3H), 3.04 (s, 3H), 3.13 (d, J=16.6 Hz, 1H), 3.48 (d, J=16.6 Hz, 1H), 3.85 (s, 3H), 6.99 (dd, J=10.0 Hz, J=2.4 Hz, 1H) 7.23 (m, 2H), 7.42 (m, 2H), 7.52 (t, J=7.7 Hz, 1H), 7.65 (d, J=7.5 Hz, 1H), 7.73 (s, 1H); m/z (ES+) M+1=324; LCMS t_{R}=1.7 min.

### 2-Amino-6-(3-bromo-phenyl)-3,6-dimethyl-5,6-dihydro-3H-pyrimidin-4-one (Example 4, Scheme 2, H) was Prepared as Follows

### (E)-3-(3-Bromo-phenyl)-but-2-enoic acid tert-butyl ester (Scheme 2, C)

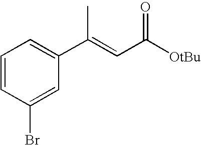

To a −78° C. stirred solution of tert-butyldimethylphosphonoacetate (21.9 mL, 0.111 mol) in THF (150 mL) was added n-BuLi in hexanes (1.6 N, 72.0 mL, 0.116 mol) and the reaction stirred at −78° C. for 10 min. To this mixture was added 3′-bromoacetophenone (13.4 mL, 0.100 mole) and the reaction allowed to warm to room temperature and was stirred for 18 hours. The THF was removed under reduced pressure to yield a yellow solid. To this was added hexanes (300 mL) and the solids triturated for one hour. The mixture was filtered through Celite and the filtrate concentrated under reduced pressure to give the title compound as a crude oil (28.9 g). This was carried directly into the next reaction. ¹H NMR (300 MHz, DMSO-d₆): δ 1.47 (s, 9H); 2.44 (s, 3H); 6.05 (s, 1H); 7.36 (t, 1H, J=7.8 Hz); 7.53 (m, 2H); 7.71 (s, 1H).

### (E)-3-(3-Bromo-phenyl)-but-2-enoic acid (Scheme 2, D)

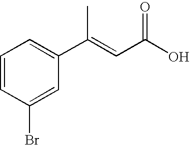

A solution of crude (E)-3-(3-Bromo-phenyl)-but-2-enoic acid tert-butyl ester C (28.9 g) in trifluororacetic acid:methylene chloride (1:1, 300 mL) was stirred at room temperature for 15 min. and the solvents removed under reduced pressure. The crude yellow solid was triturated in hexanes (400 mL), filtered, and dried under vacuum to give the title compound as a white solid (8.87 g, 38%). ¹H NMR (300 MHz, DMSO-d₆): δ 2.46 (s, 3H); 6.11 (s, 1H); 7.37 (t, 1H, J=7.8 Hz); 7.53 (m, 2H); 7.72 (t, 1H, J=1.5 Hz).

### (E)-3-(3-Bromo-phenyl)-but-2-enoyl chloride (Scheme 2, E)

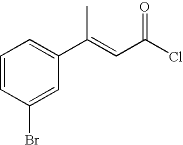

To a suspension of (E)-3-(3-Bromo-phenyl)-but-2-enoic acid (Scheme 2, D) (1.00 g, 4.148 mmol) in 10 mL methylene chloride was added oxalyl chloride (434 μL, 4.98 mmol) followed by DMF (15 μL, 0.207 mmol) and the reaction stirred at room temperature. After two hours the solvent was removed under reduced pressure to give the title compound as a yellow oil that solidified to an off white solid. ¹H NMR (300 MHz, DMSO-d₆): δ 2.51 (s, 3H); 6.44 (s, 1H); 7.29 (t, 1H, J=7.8 Hz); 7.43 (d, 1H, J=7.8 Hz); 7.57 (d, 1H, J=8.7 Hz); 7.63 (t, 1H, J=1.8 Hz).

### (E)-3-(3-bromo-phenyl)-N-cyano-N-methyl-but-2-enamide (Scheme 2, F)

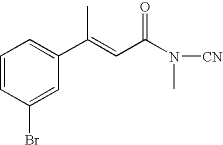

To a −60° C. stirred solution of cyanogen bromide (4.24 g, 40.00 mmol) in 100 mL THF was added sodium carbonate (6.36 g, 60.00 mmol) followed by drop wise addition of a solution of 2.0 M methyl amine solution in THF (20.0 mL 40.00 mmol). The bath temperature was kept below −20° C. for two hours. The reaction was filtered cold under a blanket of nitrogen through Celite and a solution (E)-3-(3-Bromo-phenyl)-but-2-enoyl chloride (Scheme 2, E) (5.19 g, 20.00 mmol) in 100 mL THF was added to the filtrate. To this mixture was added N,N-diisopropylethylamine (4.2 mL, 24.00 mmol) and the reaction stirred at room temperature for two hours. The solvent was removed under reduced pressure and the resulting oil put under high vacuum over night. The crude compound was purified using flash chromatography on silica gel eluting with DCM to give the title compound as an off white solid (4.29 g, 75%). ¹H NMR (300 MHz, DMSO-d₆): δ 2.44 (s, 3H); 3.22 (s, 3M); 6.65 (s, 1H); 7.42 (t, 1H, J=7.8 Hz); 7.58 (d, 1H, J=8.4 Hz); 7.65 (d, 1H, J=7.8 Hz); 7.76 (t, 1H, J=1.8 Hz).

### 6-(3-Bromo-phenyl)-1-(4-methoxy-benzylamino)-3,6-dimethyl-5,6-dihydro-3H-pyrimidin-4-one (Scheme 2, G)

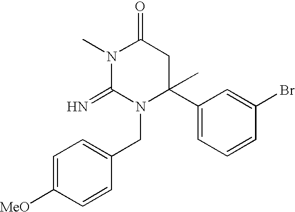

To a solution of (E)-3-(3-bromo-phenyl)-N-cyano-N-methyl-but-2-enamide (Scheme 2, F) (12.77 g, 45.75 mmol) in 50 mL DMF was added 4-methoxybenzyl amine (14.9 mL, 114.38 mmol). After four hours the solvent was removed under reduced pressure and the resulting viscous oil put under high vacuum over night. The crude compound was purified using sequential flash chromatography. The first purification was on silica gel eluting with DCM, 2.5:97.5 MeOH:DCM, 5:95 MeOH:DCM to give 18.96 g crude product. The second purification was done on silica gel eluting Et₂O, EtOAc, 5:95 MeOH:EtOAc, 10:90 MeOH: EtOAc to give clean title compound as an off white solid (15.48 g, 81%). ¹H NMR (300 MHz, DMSO-d₆/TFA-d): δ 1.65 (s, 3H); 3.20 (s, 3H); 3.30 (d, 1H, J=16.5 Hz); 3.58 (d, 1H, J=16.8 Hz); 3.78 (s, 3H); 4.97 (dd, 2H, J=4.8 Hz); 6.96 (d, 21, J=8.7 Hz); 7.34 (m, 4H); 7.57 (m, 2H); m/z (APCI+) M+1 (416.08); LCMS t_{R} 1.80 min.

### EXAMPLE 4

### 2-Amino-6-(3-bromo-phenyl)-3,6-dimethyl-5,6-dihydro-3H-pyrimidin-4-one (Scheme 2, H)

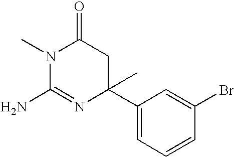

To a solution of 6-(3-Bromo-phenyl)-(4-methoxy-benzylamino)-3,6-dimethyl-5,6-dihydro-3H-pyrimidin-4-one (Scheme 2, G) (15.48 g, 37.18 mmol) in 150 mL ACN was added 50 mL water followed by ammonium cerium-nitrate (61.15 g, 111.55 mmol) and the reaction stirred for 18 hours. Celite (32 g) was added followed by sodium bicarbonate (31.23 g, 371.8 mmol) and reaction stirred for two hours. Additional Celite (15 g) was added at the halfway point. The reaction was filtered through Celite and filtrate concentrated under reduced pressure. The resulting orange solid was put under high vacuum. A crude purification was done using silica gel eluting with 15:85:0.1 MeOH:DCM: acetic acid. The resulting orange solid was triturated with methanol to give the first batch of the title compound. The solvents were removed from the filtrate under reduced pressure and the resulting orange solid was triturated with ethanol to give a second batch of the title compound. The batches were combined to give the title compound (8.75 g, 79%) as an off white solid H NMR (300 MHz, DMSO-d₆/TFA-d): δ 1.64 (s, 3H); 3.14 (s, 3H); 3.19 (d, 1H, J=16.5 Hz); 3.49 (d, 1H, J=16.2 Hz); 7.39 (m, 2H); 7.55 (m, 1H); 7.67 (s, 1H); m/z (APCI+) M+1 (296.0); LCMS t_{R} 1.30 min.

In some examples, the 1-(4-methyloxy-benzylamino) group can be replaced with a 1-benzylamino group. In this case cleavage of the benzyl group can be accomplished by catalytic transfer hydrogenation 10% Pd/C in 5% formic acid/methanol. (e.g. Example 5, Table 1).

### EXAMPLE 6

### 2-Amino-6-[3-(5-chloro-thiophen-2-yl)-phenyl]-3,6-dimethyl-5,6-dihydro-3H-pyrimidinone

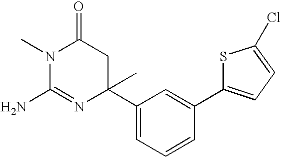

General Suzuki Conditions Method B: To the solid product from Example 4 (47 mg, 0.13 mmol) in a vial was added tripotassium phosphate (83 mg, 0.39 mmol), 5-chlorothiophene-2-boronic acid (55 mg, 0.33 mmol), dichlorobis(triphenylphosphine)palladium(II) (12 mg, 0.016 mmol), and 2.0 mL-7:3:2 1,2-dimethoxyethane:water:ethanol. The reaction was sealed and placed in a 100° C. bath for 15 min. after which the solvents were removed under vacuum. To this brown gum was added acetonitrile:water:TFA (75:25:0.1, 2.0 ml) and the resulting precipitate removed. The filtrate was purified using RP-HPLC (Ret. time: 15.0 mins). The combined purified fractions were lyophilized to give the title compound as a white powder (46 mg, 61%). ¹H NMR (300 MHz, DMSO-d₆/TFA-d): δ 1.68 (s, 3H); 3.12 (s, 3H); 3.21 (d, 1H, J=16.5 Hz); 3.54 (d, 1H, J=16.2 Hz); 7.16 (d, 1H, J=3.9 Hz); 7.43 (m, 3H); 7.57 (d, 1H, J=7.8 Hz); 7.66 (s, 1H); m/z (APCI+) M+1 (334.0); LCMS t_{R} 1.91 min.

The following compounds were synthesized using methods analogous to those previously described for Examples 1 or 4 employing the appropriate commercially available boronic acid. The column “Method” contains three rows: the first is the scheme used; the second is the Suzuki method described in Example 2 (A) or Example 6 (B); and the third is the arylbromide used in the Suzuki. NA denotes that the Suzuki coupling with an arylbromide was not used.

TABLE 1

| | | | | | m/z M + 1 | LCMS |
|---|---|---|---|---|---|---|
| | | | | | (Ioniza- | t_{R} |
| Ex. | Compound | Structure | Method | NMR | tion) | (min) |
| 5 | 2-amino-3,6-dimethyl-6-naphthalen-2-yl-5,6-dihydro-3H-pyrimidin-4-one | 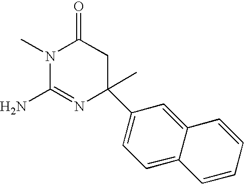 | Scheme-2NA | ¹H NMR (300.132 MHz,DMSO-d6/TFA-d) δ 1.72(s, 3H), 3.07 (s, 3H), 3.21(d, J = 16.5 Hz, 1H), 3.52(d, J = 16.5 Hz, 1H), 7.57(m, 3H), 7.94 (m, 4H) | 268(APCI+) | 1.67 |
| 7 | 2-amino-6-(3,4-dichlorophenyl)-6-ethyl-5,6-dihydropyrimidin-4(3H)-one | 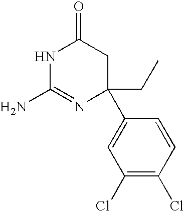 | Scheme-1NA | ¹H NMR (300 MHz,DMSO-d₆/TFA-d): δ 0.80(t, 3H, J = 7.4 Hz); 1.94(m, 2H); 3.18 (d, 1H, J =16.5 Hz); 3.34 (d, 1H, J =16.5 Hz); 6.37 (dd, 1H, J =8.6 Hz); 7.69 (m, 2H) | 288 (ES+) | 1.45 |
| 8 | 2-amino-6-(3,4-dichlorophenyl)-6-isobutyl-5,6-dihydropyimidin-4(3H)-one | 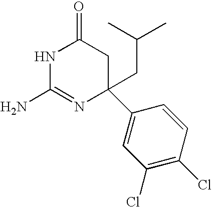 | Scheme-1NA | ¹H NMR (300.132 MHz,DMSO-d6/TFA-d) δ 0.7(d, J = 6.6 Hz, 3H), 0.82(d, J = 6.6 Hz, 3H), 1.65(m, 1H), 1.70 (m, 2H),3.20 (d, J = 16.5 Hz, 1H),3.37 (d, J = 16.5 Hz, 1H),7.40 (dd, J = 8.4, 2.3 Hz,1H), 7.68 (d, J = 2.3 Hz,1H), 7.76 (s, 1H) | 314 (ES+) | 1.99 |
| 9 | 2-amino-6-(3,4-dichlorophenyl)-3-methyl-6-(2-phenylethyl)-5,6-dihydropyrimidin-4(3H)-one | 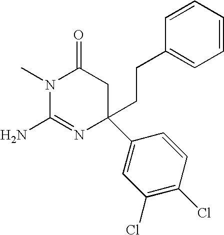 | Scheme-2NA | ¹H NMR (300.132 MHz,DMSO-d6/TFA-d) δ 2.26(m, 2H, 2.48 (m, 1H),2.60 (m, 1H), 3.35 (d, J =16.5 Hz, 1H), 3.53 (d, J =16.5 Hz, 1H), 7.18 (m,3H), 7.26 (m, 2H), 7.40(dd, J = 8.4, 2.3 Hz, 1H),7.68 (d, J = 2.3 Hz, 1H),7.73 (s, 1H) | 376 (ES+) | 1.93 |
| 10 | 2-amino-6-(3-bromo-4-chlorophenyl)-3,6-dimethyl-5,6-dihydropyrimidin-4(3H)-one | 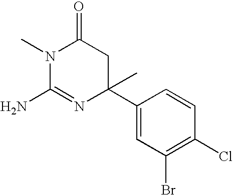 | Scheme-2NA | ¹H NMR (300.132 MHz,DMSO-d6/TFA-d) δ 1.63(s, 3H), 3.11 (s, 3H), 3.20(d, J = 16.5 Hz, 1H), 3.50(d, J = 16.5 Hz, 1H), 7.40(dd, J = 8.4, 2.3 Hz, 1H),7.66 (d, J = 8.4 Hz, 1H),7.85 (d, J = 2.4 Hz, 1H) | 330 (ES+) | 1.76 |
| 11 | 2-amino-3,6-dimethyl-6-(2-phenylethyl)-5,6-dihydropyrimidin-4(3H)-one | 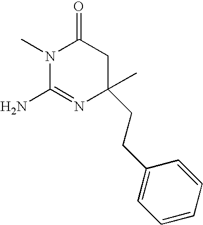 | Scheme-2NA | ¹H NMR (300.132 MHz,DMSO-d6/TFA-d) δ 1.36(s, 3H), 1.87 (m, 2H), 2.59(m, 2H), 2.73 (d, J = 16.5Hz, 1H), 2.94 (d, J = 16.5Hz, 1H), 3.43 (s, 3H),7.16-7.32 (br m, 5H) | 246 (ES+) | 1.33 |
| 12 | 2-Amino-6-[2-(3-bromo-phenyl)-ethyl]-3,6-dimethyl-5,6-dihydro-3H-pyrimidin-4-one | 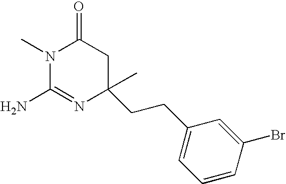 | Scheme-2NA | ¹H NMR (300 MHz,DMSO-d6/TFA-d) δ 1.33(s, 3H), 1.86 (m, 2H),2.64 (t, J = 8.5 Hz, 2H),2.79 (d, J = 16.4 Hz, 1H),2.94 (d, J = 16.4 Hz, 1H),3.20 (s, 3H), 7.26 (d, J =5.0 Hz, 2H), 7.40 (td, J =4.5, 1.9 Hz, 1H), 7.49 (s,1H) | 324(APCI+) | 1.83 |
| 13 | 2-amino-6-[3-(benzyloxy)phenyl]-3,6-dimethyl-5,6-dihydropyrimidin-4(3H)-one | 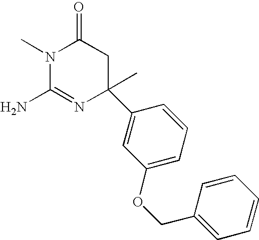 | Scheme-2NA | ¹H NMR (300.132 MHz,DMSO-d6/TFA-d) δ 1.61(s, 3H), 3.06 (s, 3H), 3.15(d, J = 16.5 Hz, 1H), 3.44(d, J = 16.5 Hz, 1H), 5.10(s, 2H), 6.94-7.05 (br m,3H), 7.31-7.47 (br m, 6H) | 324 (ES+) | 1.65 |
| 14 | 2-amino-6-methyl-6-(3-phenoxyphenyl)-5,6-dihydropyrimidin-4(3H)-one | 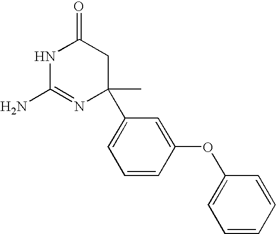 | Scheme-1NA | ¹H NMR (300 MHz,DMSO-d₆/TFA-d): δ 1.65(s, 3H); 3.14 (d, 1H, J =16.5 Hz); 3.31 (d, 1H, J =16.5 Hz); 6.93 (dd, 1H, J =8.0 Hz); 7.02 (d, 2H, J =7.8 Hz); 7.17 (m, 3H);7.42 (m, 3H) | 296 (ES+) | 1.46 |
| 15 | 2-amino-6-(3-bromo-4-chlorophenyl)-6-methyl-5,6-dihydropyrimidin-4(3H)-one | 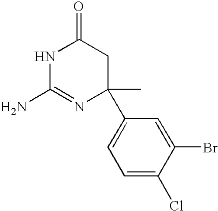 | Scheme-1NA | ¹H NMR (300.132 MHz,DMSO-d6/TFA-d) δ 1.65(s, 3H), 3.15 (d, J = 16.6Hz, 1H), 3.36 (d, J = 16.6Hz, 1H), 7.45 (dd, J = 8.5,2.4 Hz, 1H), 7.70 (d, J =8.5 Hz, 1H), 7.85 (d, J =2.3 Hz, 1H) | 316 (ES+) | 1.37 |
| 16 | 2-amino-6-(3′-methoxy-1,1′-biphenyl-3-yl)-6-methyl-5,6-dihydropyrimidin-4(3H)-one | 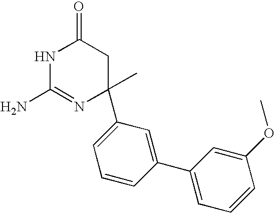 | Scheme-1Suzuki-A 1 | ¹H NMR (300 MHz,DMSO-d₆/TFA-d): δ 1.71(s, 3H); 3.17 (d, 1H, J =16.5 Hz); 3.45 (d, 1H, J =16.5 Hz); 3.85 (s, 3H);6.98 (d, 1H, J = 8.1 Hz);7.23 (m, 2H); 7.41 (m,2H); 7.52 (m, 1H); 7.68(m, 2H) | 310(APCI+) | 1.89 |
| 17 | 2-amino-6-methyl-6-[3-(5-methylthien-2-yl)phenyl]-5,6-dihydropyrimidin-4(3H)-one | 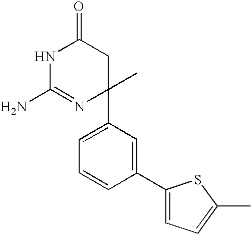 | Scheme-1Suzuki-A 1 | | 300 (ES+) | 1.65 |
| 18 | 2-amino-6-[3-(2-furyl)phenyl]-6-methyl-5,6-dihydropyrimidin-4(3H)-one | 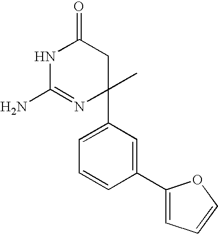 | Scheme-1Suzuki-A 1 | | 270(APCI+) | 1.65 |
| 19 | 2-amino-6-(3′-butoxy-1,1′-biphenyl-3-yl)-6-methyl-5,6-dihydropyrimidin-4(3H)-one | 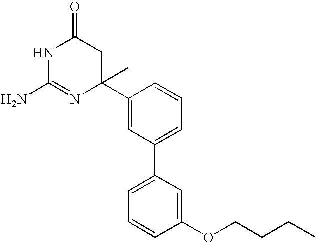 | Scheme-1Suzuki-A 1 | ¹H NMR (300 MHz,DMSO-d₆/TFA-d): δ 0.95(m, 3H); 1.47 (m, 2H);1.71 (m, 5H); 3.17 (d, 1H,J = 16.5 Hz); 3.46 (d, 1H,J = 16.5 Hz); 4.06 (t, 2H,J = 6.3 Hz); 6.95 (m, 1H);7.24 (m, 2H); 7.38 (m,2H); 7.52 (t, 1H, J = 7.8);7.66 (m, 2H) | 352(APCI+) | 2.22 |
| 20 | 2-amino-6-(6-chloro-3′-methoxy-1,1′-biphenyl-3-yl)-6-methyl-5,6-dihydropyrimidin-4(3H)-one | 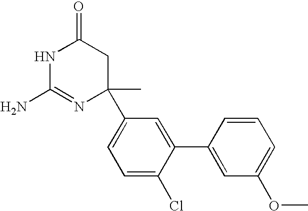 | Scheme-1Suzuki A 15 | ¹H NMR (300.132 MHz,DMSO-d6/TFA-d) δ 1.68(s, 3H), 3.15 (d, J = 16.6Hz, 1H), 3.40 (d, J = 16.5Hz, 1H), 3.82 (s, 3H), 6.99-7.03 (m, 3H), 7.38-7.53(m, 3H), 7.60-7.64 (m,1H) | 344 (ES+) | 1.65 |
| 21 | 2-amino-3,6-dimethyl-6-[3-(5-methylthien-2-yl)phenyl]-5,6-dihydropyrimidin-4(3H)-one | 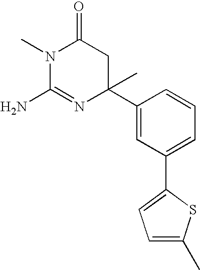 | Scheme-2Suzuki B 4 | 1H NMR (300.132 MHz,DMSO) δ 1.68 (s, 3H),2.49 (s, 3H), 3.12 (s,3H), 3.20 (d, J = 16.4 Hz,1H), 3.52 (d, J = 16.4 Hz,1H), 6.84 (d, J = 2.5 Hz,1H), 7.31 (m, 2H), 7.43 (t,J = 7.7 Hz, 1H), 7.54 (d,J = 8.0 Hz, 1H), 7.62 (s,1H) | 314(APCI+) | 1.97 |
| 22 | 2-amino-6-(3′-isopropoxy-1,1′-biphenyl-3-yl)-3,6-dimethyl-5,6-dihydropyrimidin-4(3H)-one | 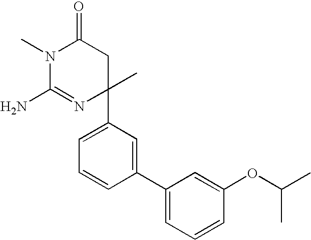 | Scheme-2Suzuki B 4 | 1H NMR (300.132 MHz,DMSO) δ 1.32 (d, J = 6.0Hz, 6H), 1.70 (s, 3H),3.12 (s, 3H), 3.21 (d, J =16.4 Hz, 1H), 3.60 (d, J =16.4 Hz, 1H), 4.72 (septet,J = 6.0 Hz, 1H), 6.96 (dd,J = 8.2, 1.9 Hz, 1H), 7.21(m, 2H), 7.38 (t, J = 8.0Hz, 2H), 7.50 (t, J = 7.7Hz, 1H), 7.63 (d, J = 7.8Hz, 1H), 7.71 (s, 1H) | 352(ES+) | 1.82 |
| 23 | 2-amino-6-(6-chloro-3′-methoxy-1,1′-biphenyl-3-yl)-3,6-dimethyl-5,6-dihydropyrimidin-4(3H)-one | 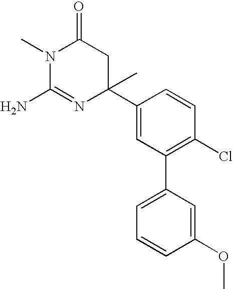 | Scheme-1Suzuki A 15 | ¹H NMR (300.132 MHz,DMSO-d6/TFA-d) δ 1.66(s, 3H), 3.12 (s, 3H), 3.19(d, J = 16.5 Hz, 1H), 3.53(d, J = 16.5 Hz, 1H), 3.82(s, 3H), 7.00 (m, 3H),7.38-7.48 (br m, 3H), 7.60(d, J = 8.4 Hz, 1H) | 358(ES+) | 2.03 |
| 24 | 2-amino-6-(4-chloro-3-quinolin-5-yl-phenyl)-3,6-dimethyl-5,6-dihydro-3H-pyrimidin-4-one | 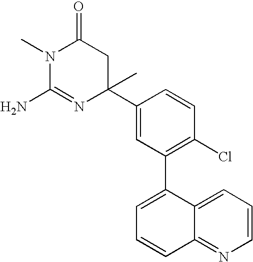 | Scheme-1Suzuki A 15 | ¹H NMR (300.132 MHz,DMSO-d6/TFA-d) δ 1.68(s, 3H), 3.12 (s, 3H), 3.23(d, J = 16.5 Hz, 1H), 3.52(d, J = 16.5 Hz, 1H), 7.61(m, 2H), 7.78 (m, 2H),7.83 (m, 1H), 8.19 (t, J =8.4 Hz, 1H), 8.31 (m, 2H),9.31 (d, J = 4.8 Hz, 1H) | 379(APCI+) | 1.50 |
| 25 | 2-Amino-6-[2-(3′-methoxy-biphenyl-3-yl)-ethyl]-3,6-dimethyl-5,6-dihydro-3H-pyrimidin-4-one | 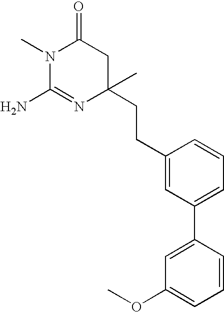 | Scheme-2Suzuki-B 12 | 1H NMR (300 MHz,DMSO-d6/TFA-d) δ 1.37(s, 3H), 1.94 (m, 2H),2.71 (m, 2H), 2.82 (d, J =16.4 Hz, 1H), 2.98 (d, J =16.4 Hz, 1H), 3.21 (s,3H), 3.84 (s, 3H), 6.95(dd, J = 8.0, 2.2 Hz, 1H),7.19 (d, J = 2.2 Hz, 1H),7.23 (t, J = 6.9 Hz, 2H),7.38 (t, J = 7.9 Hz, 2H),7.51 (m, 2H) | 352 (ES+) | 1.83 |
| 26 | 2-Amino-6-methyl-6-quinolin-6-yl-5,6-dihydro-3H-pyrimidin-4-one | 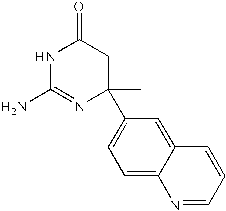 | Scheme-1NA | 1H NMR (300 MHz,DMSO-d6/TFA-d) δ 1.79(s, 3H), 3.33 (d, J = 16.6Hz, 1H), 3.48 (d, J = 16.6Hz, 1H), 8.18 (dd, J = 8.4,5.3 Hz, 1H), 8.29 (dd, J =9.2, 1.8 Hz, 1H), 8.39 (t, J =4.4 Hz, 2H), 9.27 (d, J =8.3 Hz, 1H), 9.40 (d, J =5.3 Hz, 1H) | 255(APCI+) | 0.36 |

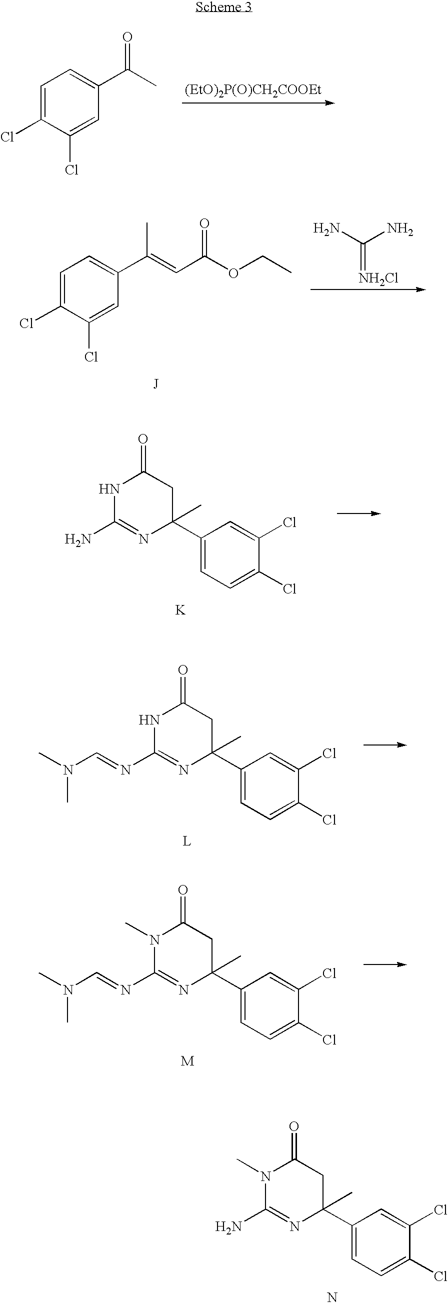

### EXAMPLE 27

### 2-Amino-6-(3,4-dichloro-phenyl)-3,6-dimethyl-5,6-dihydro-3H-pyrimidin-4-one (Scheme 3, N)

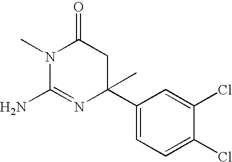

To a solution of N′-[4-(3,4-Dichloro-phenyl)-1,4-dimethyl-6-oxo-1,4,5,6-tetrahydro-pyrimidin-2-yl]-N,N-dimethyl-formamidine (Scheme 3, M) (0.16 g, 0.47 mmol) in MeOH (15 mL) was added 7 N methanolic ammonia (3 mL, 21.0 mmol) and the reaction was heated to 60° C. for 3 hrs. The MeOH was removed under reduced pressure to yield an amber syrup. To this was added acetonitrile:water:TFA (75:25:0.1, 4 ml) and the resulting precipitate was removed. The filtrate was purified using RP-HPLC (Ret. time: 13.03 mins). The combined purified fractions were lyophilized to give the title compound as a white powder (0.03 g, 22%). ¹H NMR (300 MHz, DMSO-d₆): δ 1.65 (s, 3H); 3.12 (s, 3H); 3.19 (d, 1H, J=16.5 Hz); 3.50 (d, 1H, J=16.5 Hz); 7.41 (d, 1H, J=8.4 Hz); 7.67 (d, 1H, J=8.4 Hz); 7.72 (s, 1H). m/z MS (ES) 286 M⁺.

### N′-[4-(3,4-Dichloro-phenyl)-1,4-dimethyl-6-oxo-1,4,5,6-tetrahydro-pyrimidin-2-yl]-N,N-dimethyl-formamidine (Scheme 3, M) was Prepared as Follows

### (E)-3-(3,4-Dichloro-phenyl)-but-2-enoic acid ethyl ester (Scheme 3, J)

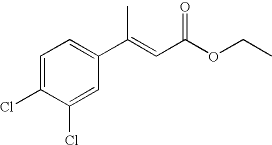

To a −78° C. stirred solution of triethylphosphonoacetate (11.5 mL, 58.2 mmol) in THF (100 mL) was added n-BuLi in hexanes (1.6 N, 38 mL, 61 mmol) and the reaction stirred at −78° C. for 10 min. To this mixture was added a solution of 3,4-dichloroacetophenone (10.0 g, 52.9 mmol) in THF (10 mL) and the reaction allowed to warm to room temperature and stirred for 18 hours. The solvent was removed under reduced pressure to yield a yellow solid. To this was added 400 mL 1:3 Et₂O: hexanes and the solids triturated for one hour. The resulting precipitate was removed by filtering through Celite and the filtrate collected, concentrated under reduced pressure, and put under high vacuum to give the title compound as a crude oil (12.14 g). This was carried directly into the next reaction. ¹H NMR (300 MHz, DMSO-d₆): δ 1.24 (t, 3H, J=6.9); 2.48 (s, 3H); 6.22 (s, 1H); 7.50 (d, 1H, J=2.4 Hz); 7.84 (d, 2H, J=2.1 Hz).

### EXAMPLE 28

### 2-Amino-6-(3,4-dichloro-phenyl)-6-methyl-5,6-dihydro-3H-pyrimidin-4-one (Scheme 3, K)

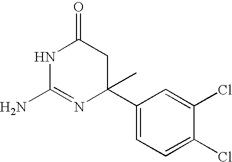

To a solution of (E)-3-(3,4-Dichloro-phenyl)-but-2-enoic acid ethyl ester (Scheme 3, J) (100 mg, 0.386 mmol) in 2.0 mL NMP was added guanidine hydrochloride (147 mg, 1.54 mmol), sodium methoxide (62 mg, 1.62 mmol), and the reaction was subjected to microwaves at 200° C. for 10 min. The solids were filtered from the reaction and the filtrate used directly for purification using RP-HPLC (Ret. time: 12.6 mins). The combined purified fractions were lyophilized to give the title compound as white solid (49 mg, 33%). ¹H NMR (300 MHz, DMSO-d₆/TFA-d): δ 1.65 (s, 3H); 3.17 (m, 1H); 3.36 (d, 1H, J=16.5 Hz); 7.42 (d, 1H, J=8.4 Hz); 7.70 (m, 2H); m/z (+ES) M+1 (271.98); LCMS t_{R} 1.35 min.

### N′-[4-(3,4-Dichloro-phenyl)-4-methyl-6-oxo-1,4,5,6-tetrahydro-pyrimidin-2-yl]-N,N-dimethyl-formamidine (Scheme 3, L)

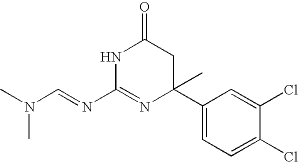

To an ambient stirred solution of Example 28 (0.25 g, 0.94 mmol) in DMF (5 mL) was added dimethylformamide dimethylacetal (0.16 mL, 1.17 mmol) and the reaction was stirred for 2 hrs. The DMF was removed under reduced pressure to yield a pale yellow oil. The oil was purified by ether tritruation (2×20 mL) to give the title compound as a clear, colorless oil (0.30 g, 99%). ¹H NMR (300 MHz, DMSO-d₆): δ 1.67 (s, 3H); 3.11-3.17 (br s, 4H); 3.20-3.29 (br s, 4H); 7.47 (d, 1H, J=8.4 Hz); 7.69 (d, 1H, J=8.4 Hz); 7.76 (s, 1H); 8.56 (s, 1H). m/z (ES) 327 M⁺.

### N′-[4-(3,4-Dichloro-phenyl)-1,4-dimethyl-6-oxo-1,4,5,6-tetrahydro-pyrimidin-2-yl]-N,N-dimethyl-formamidine (Scheme 3, M)

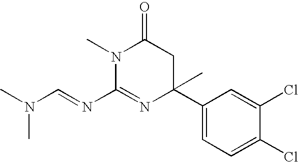

To an ambient stirred solution of N′-[4-(3,4-Dichloro-phenyl)-4-methyl-6-oxo-1,4,5,6-tetrahydro-pyrimidin-2-yl]-N,N-dimethyl-formamidine (Scheme 3, L) (0.31 g, 0.94 mmol) and potassium carbonate (0.14 g, 1.03 mmol) in DMF (70 mL) was added iodomethane (0.06 mL, 1.03 mmol) and the reaction was stirred for 18 hrs. To this mixture was added additional potassium carbonate (0.14 g, 1.03 mmole) and iodomethane (0.06 mL, 1.03 mmole) and the reaction stirred an additional 18 hrs at ambient temperature. The THF was removed under reduced pressure to yield a cloudy yellow oil. To this was added hexanes (250 mL) and the reaction stirred for 10 min. The resulting precipitate was removed and the filtrate collected and concentrated under reduced pressure. The crude compound was purified using flash chromatography (silica gel, 5.95 ethyl acetate:hexanes) to give the title compound as a pale, clear yellow oil (5.63 g, 83%). ¹H NMR (300 MHz, DMSO-d₆): δ 1.67 (s, 3H); 3.11 (s, 3H); 3.19 (s, 3H); 3.22 (d, 1H, J=16.5 Hz); 3.36 (s, 3H); 3.57 (d, 1H, J=16.5 Hz); 7.49 (d, 1H, J=8.3 Hz); 7.65 (d, 1H, J=8.4 Hz); 7.82 (s, 1H); 8.65 (s, 1H). m/z (ES) 341 M⁺.

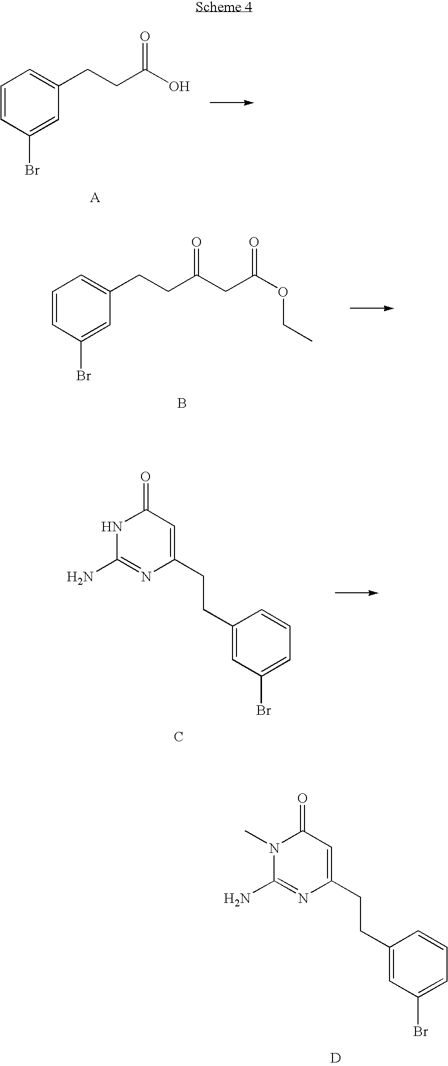

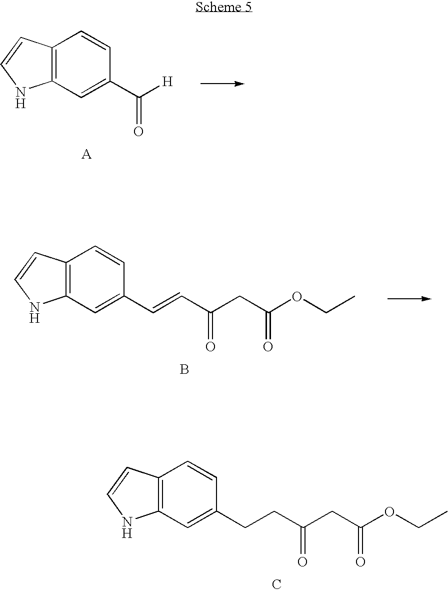

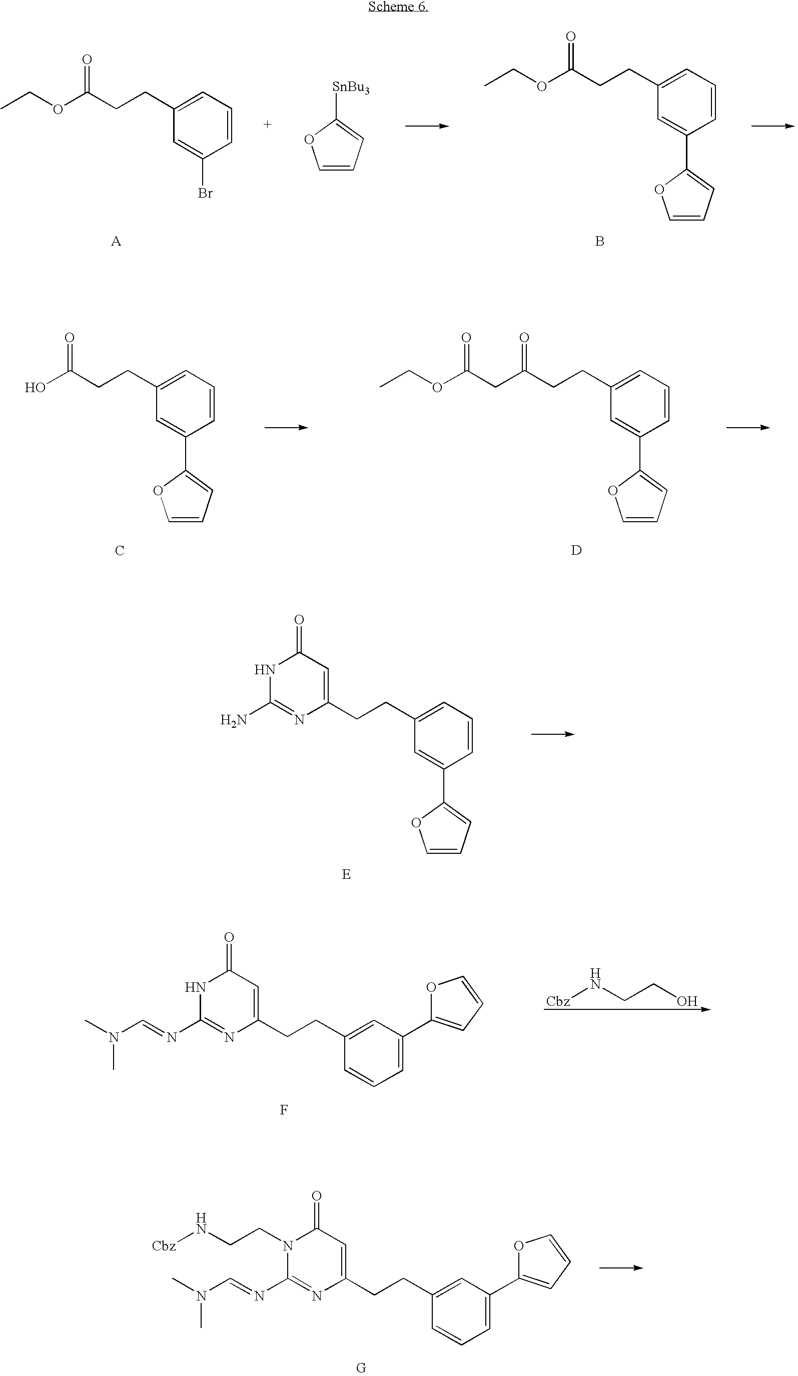

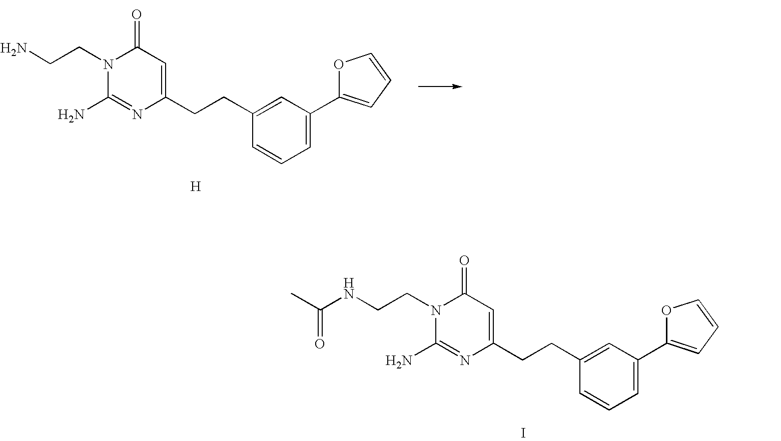

### EXAMPLE 29

### 2-Amino-6-[2-(3-bromophenyl)ethyl]-3-methylpyrimidin-4(3H)-one (Scheme 4, D)

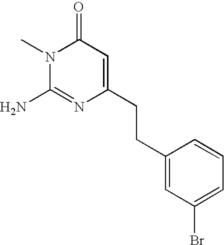

This material was prepared according to Scheme 4. To a stirred solution of 2-amino-6-[2-(3-bromophenyl)ethyl]pyrimidin-4(3H)-one (6.7 g, 23 mmol) (Scheme 4, C) in DMF (410 mL) was added potassium carbonate (2.8 g, 20 mmol) and iodomethane (1.3 mL, 20 mmol). The reaction was allowed to stir 3 days, then another portion of potassium carbonate (0.94 g, 7 mmol) and iodomethane (0.43 mL, 7 mmol) was added. The reaction was allowed to stir overnight, then another portion of potassium carbonate (0.94 g, 7 mmol) and iodomethane (0.43 mL, 7 mmol) was added. The reaction was again allowed to stir overnight then added to a large volume of water (approximately 8 L). The material was extracted into diethyl ether (6×200 mL) and the resulting solution was concentrated under reduced pressure. A portion of the resulting solid (3.0 g) was stirred in methylene chloride (260 mL) then filtered to give the desired product as a white solid (2.2 g, 92%). ¹H NMR (300 MHz, DMSO-d₆): δ 7.43 (s, 1H), 7.37 (mult, 1H), 7.24 (mult, 2H), 7.07 (s, 2H), 5.50 (s, 1H), 3.22 (s, 3H), 2.87 (t, 2H, J=7.7 Hz), 2.54 (t, 2H, J=8.5 Hz); m/z (APCI) 308 (MH⁺), HRMS (ES) M⁺, found 308.0348; C₁₃H₁₄BrN₃O requires 308.0398.

### 2-Amino-6-[2-(3-bromophenyl)ethyl]pyrimidin-4(3H)-one (Scheme 4, C) was prepared as follows.

### Ethyl 5-(3-bromophenyl)-3-oxopentanoate (Scheme 4, B)

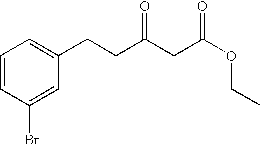

To a round bottom flask was added magnesium chloride (10.4 g, 109 mmol), acetonitrile (580 mL), potassium malonate (15.6 g, 92 mmol), and triethlamine (19.5 mL, 140 mmol). Separately, 3-(3-bromophenyl)propionic acid (10 g, 44 mmol) (Scheme 4, A) was dissolved in acetonitrile (200 mL) and to this was added 1,1′-carbonyldiimidazole (CDI) (7.8 g, 48 mmol). Both were allowed to stir for approximately 2.5 hours then the 3-(3-bromophenyl)propionic acid/CDI solution was added dropwise to the mixture of MgCl₂, potassium ethyl malonate and Et₃N. The reaction was stirred overnight, then was heated at 90° C. for 3 h. It was then allowed to cool to room temperature, filtered and rinsed with acetonitrile (3×100 mL). The combined filtrates were concentrated under reduced pressure then partitioned between methylene chloride and water. The product was extracted into the methylene chloride layer which was then washed 10% aqueous citric acid solution, dried over sodium sulfate and concentrated under reduced pressure to give the desired product (9.72 g, 75%). This material was used without purification.

### 2-Amino-6-[2-(3-bromophenyl)ethyl]pyrimidin-4(3H)-one (Scheme 4, C)

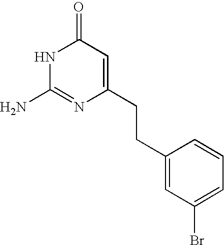

To a solution of ethyl 5-(3-bromophenyl)-3-oxopentanoate (9.72 g, 32 mmol) in ethanol (120 mL) was added guanidine carbonate (2.9 g, 16 mmol) and the reaction was heated under reflux overnight. The reaction was allowed to cool then the resulting solid collected by filtration and rinsed with ethanol (20 mL). The solid was dried under high-vacuum to give the desired product as a white solid (6.8 g, 71%). ¹H NMR (300 MHz, DMSO-d₆) δ 10.58 (s, 1H), 7.42 (s, 1H), 7.37 (mult, 1H), 7.23 (mult, 2H), 6.46 (s, 2H), 5.39 (s, 1H), 2.86 (t, J=7.8 Hz, 2H), 2.53 (t, J=8.1 Hz, 2H); m/z (APCI) 294 (MH⁺).

### EXAMPLE 30

### 2-Amino-6-[2-(2-bromophenyl)ethyl]-3-methylpyrimidin-4(3H)-one

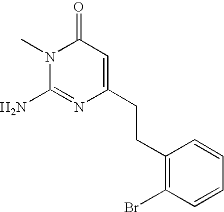

This compound was prepared according to the method described for 2-amino-6-[2-(3-bromophenyl)ethyl]-3-methylpyrimidin-4(3H)-one except 3-(2-bromophenyl)propionic acid was used in place of 3-(3-bromophenyl)propionic acid. ¹H NMR (300 MHz, DMSO-d₆) δ 7.58 (d, J=7.7 Hz, 1H), 7.31 (mult, 2H), 7.15 (mult, 1H), 7.07 (s, 2H), 5.50 (s, 1H), 3.22 (s, 3H), 2.97 (t, J=8.0 Hz, 2H), 2.54 (t, J=8.4 Hz, 2H); m/z (APCI) 308.2 (MH⁺), HRMS (ES) M, found 308.037; C₁₃H₁₄BrN₃O requires 308.0398.

### EXAMPLE 31

### 2-Amino-6-[2-(4-bromophenyl)ethyl]-3-methylpyrimidin-4(3H)-one

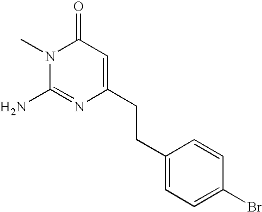

This compound was prepared according to the method described for 2-amino-6-[2-(3-bromophenyl)ethyl]-3-methylpyrimidin-4(3H)-one except 3-(4-bromophenyl)propionic acid was used in place of 3-(3-bromophenyl)propionic acid. ¹H NMR (300 MHz, DMSO-d₆) δ 7.45 (d, J=8.3 Hz, 2H), 7.17 (d, J=8.3 Hz, 2H), 7.05 (s, 2H), 5.48 (s, 1H), 3.21 (s, 3H), 2.84 (t, J=7.8 Hz, 2H), 2.53 (t, J=8.4 Hz, 2H); m/z (APCI) 294 (MH⁺), HRMS (ES) M⁺, found 308.0388; C₁₃H₁₄BrN₃O requires 308.0398.

### EXAMPLE 32

### 2-Amino-6-[2-(1H-indol-6-yl)ethyl]-3-methylpyrimidin-4(3H)-one

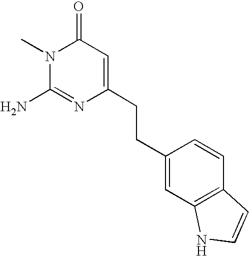

This compound was prepared according to the method described for 2-amino-6-[2-(3-bromophenyl)ethyl]-3-methylpyrimidin-4(3H)-one except 2-amino-6-[2-(1H-indol-6-yl)ethyl]pyrimidin-4(3H)-one was used in place of 2-amino-6-[2-(3-bromophenyl)ethyl]pyrimidin-4(3H)-one. ¹H NMR (300 MHz, DMSO-d₆) δ 10.97 (s, 1H), 8.40 (bs, 1H), 7.46 (d, J=8.5 Hz, 1H), 7.26 (mult, 2H), 6.90 (d, J=8.5 Hz, 1H), 6.37 (s, 1H), 5.82 (s, 1H), 3.95 (bs, 1H), 3.27 (s, 3H), 2.96 (t, J=9.0 Hz, 2H), 2.75 (t, J=9.0 Hz, 2H);); m/z (APCI) 269 (MH⁺).

### EXAMPLE 33

### 2-Amino-6-[2-(1H-indol-6-yl)ethyl]pyrimidin-4(3H)-one

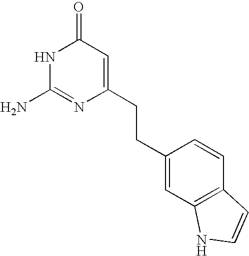

Ethyl 5-(1H-indol-6-yl)-3-oxopentanoate (approximately 65% pure with over-reduced product as the major contaminant), (20 g, 77 mmol) was dissolved ethanol (160 mL) under argon and to this was added guanidine carbonate (9.0 g, 50 mmol). The reaction was heated under reflux overnight then concentrated until approximately 50 mL of ethanol remained. To this was added water-(50 mL) and the mixture was stirred for 3 h. The resulting solid was collected by filtration and rinsed with water (approximately 50 mL) then dried under high vacuum at 60° C. overnight to give the desired product as a yellow solid (7.9 g, 62%). Another batch (2.4 g, 19%) slowly crystallized from the filtrate. ¹H NMR (300.132 MHz, DMSO-d₆) δ 10.90 (s, 1H), 7.41 (d, J=8.1 Hz, 1H), 7.23 (d, J=3.1 Hz, 1H), 7.18 (s, 1H), 6.85 (d, J=8.0 Hz, 1H), 6.62 (s, 2H), 6.34 (d, J=2.9 Hz, 1H), 5.36 (s, 1H), 2.93 (t, J=7.9 Hz, 2H), 2.56 (t, J=7.9 Hz, 2H); m/z (APCI) 255 (MH⁺).

### Ethyl 5-(1H-indol-6-yl)-3-oxopentanoate (Scheme 5, C) was Prepared as Follows

### Ethyl (4E)-5-(1H-indol-6-yl)-3-oxopent-4-enoate (Scheme 5, B)

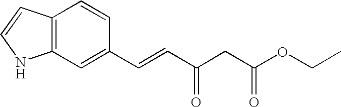

6-Formylindole (15 g, 103 mmol) (Scheme 5, A) was dissolved in dry THF (410 mL) under argon and to this was added [3-(ethoxycarbonyl)-2-oxopropyl]triphenylphosphonium chloride (66 g, 155 mmol) and the reaction cooled to 5° C. Sodium hydride (60%, 6.5 g, 412 mmol) was then added in portions over 10 min., the cooling bath removed, and the reaction was allowed to stir overnight. Another portion of sodium hydride (60%, 4.1 g, 102 mmol) was added, the reaction allowed to stir for 2 hours, then another portion of the [3-(ethoxycarbonyl)-2-oxopropyl]triphenylphosphonium chloride (22 g, 51 mmol) was added. The reaction was again allowed to stir overnight, cooled to 5° C., and to this was added saturated aqueous ammonium chloride (200 mL) and water (100 mL). Ethyl acetate (100 mL) was added and the product extracted into the organic phase. It was then dried over sodium sulfate, filtered, concentrated under reduced pressure and purified by silica gel chromatography (30% ethyl acetate/hexanes) to give the desired product as an oil which later solidified (20.6 g, 78%). ¹H NMR (300 MHz, DMSO-d₆) δ 11.38 (s, 1H), 7.79 (d, J=16.1 Hz, 1H), 7.72 (s, 1H), 7.59 (d, J=8.3 Hz, 1H), 7.49 (mult, 1H), 7.39 (d, J=8.3 Hz, 1H), 6.83 (d, J=16.1 Hz, 1H), 6.49 (s, 1H), 4.12 (q, J=7.1 Hz, 2H), 3.85 (s, 2H), 1.20 (t, J=7.1 Hz, 3H); m/z (ES) 256 (M-H)⁻.

### Ethyl 5-(1H-indol-6-yl)-3-oxopentanoate (Scheme 5, C)

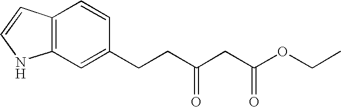

Ethyl (4E)-5-(1H-indol-6-yl)-3-oxopent-4-enoate (20.6 g, 80 mmol) (Scheme 5, B) was dissolved in ethanol (160 mL) under argon. The solvent was degassed with argon then 10% Pd/C (4.25 g, 4.0 mmol) was added. The mixture was placed under 1 atmosphere of hydrogen and stirred vigorously for 2 hours. It was then filtered through celite rinsing with ethanol, then concentrated under reduced pressure to give desired product (20 g, 65% pure with the remainder being the over-reduced material); m/z (ES) 258 (M-H)⁻.

### EXAMPLE 34

### 2-Amino-3-methyl-6-[2-(2-naphthyl)ethyl]pyrimidin-4(3H)-one

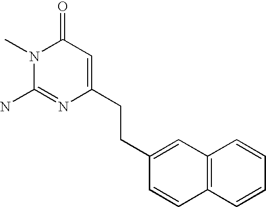

This compound was prepared according to the method described for 2-amino-6-[2-(3-bromophenyl)ethyl]-3-methylpyrimidin-4(3H)-one except ethyl 5-(2-naphthyl)-3-oxopentanoate was used in place of ethyl 5-(3-bromophenyl)-3-oxopentanoate. ¹H NMR (300 MHz, DMSO-d₆) δ 8.46 (s, 2H), 7.86 (mult, 3H), 7.76 (s, 1H), 7.48 (mult, 3H), 5.82 (s, 1H), 3.26 (s, 3H), 3.07 (t, J=7.8 Hz, 2H), 2.81 (t, J=7.7 Hz, 2H); m/z (APCI) 280 (MH⁺), HRMS (ES) M⁺, found 280.1417; C₁₇H₁₇N₃O requires 280.145.

### Ethyl 5-(2-naphthyl)-3-oxopentanoate was Prepared as Follows

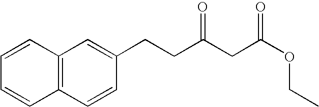

Diisopropylamine (8.7 mL, 62 mmol) was dissolved in THF (100 mL) under nitrogen, cooled to 0° C., and to this was added N-butlyllithim (1.6 M, 3.8 mL, 65 mmol). The resulting solution was then added to ethyl acetoacetate (3.8 mL, 30 mmol) and allowed to stir at 0° C. for approximately 25 min. Next, a solution of 2-(bromomethyl)naphthalene (6.6 g, 30 mmol) in THF (90 mL) was added over approximately 45 min. The reaction was allowed to stir for 3 h at 0° C. then quenched with a mixture of concentrated HCl (5.2 mL), water (14 mL), and diethyl ether (40 mL). The mixture was stirred 20 min. then partitioned between diethyl ether (300 mL) and water (150 mL). The layers were separated and the aqueous again extracted with diethyl ether (150 mL). The combined organics were washed approximately 10 times with water (10×100 mL), dried over sodium sulfate, and concentrated under reduced pressure. The resulting material was purified by silica gel chromatography using methylene chloride/hexanes as the eluant to afford the desired product as a light yellow liquid (2.83 g, 36%). ¹H NMR (300 MHz, CDCl₃) δ 7.78 (mult, 3H), 7.62 (s, 1H), 7.44 (mult, 2H), 7.31 (mult, 1H), 4.17 (q, J=7.1 Hz, 2H), 3.43 (s, 2H), 3.08 (mult, 2H), 2.97 (mult, 2H), 1.24 (t, J=7.1 Hz, 3H); m/z (APCI) 293 (MNa⁺).

### EXAMPLE 35

### 2-amino-3-(1,3-dioxolan-2-ylmethyl)-6-[2-(1H-indol-6-yl)ethyl]pyrimidin-4(3H)-one

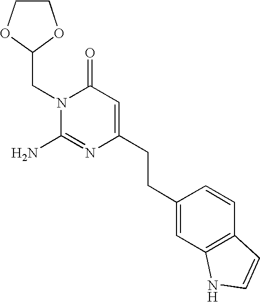

To a stirred solution of 2-amino-6-[2-(1H-indol-6-yl)ethyl]pyrimidin-4(3H)-one (1.0 g, 3.9 mmol) in DM (20 mL) was added potassium carbonate (0.82 g, 5.91 mmol) and 2-bromomethyl-1,3-dioxolane (0.57 mL, 5.51 mmol). The reaction was heated at 90° C. for 2 h, sodium iodide was added (0.03 g, 0.2 mmol), then the mixture was heated at 100° C. overnight. The temperature was increased to 110° C. for 2 h, then to 120° C. for another 2 h. The temperature was then lowered to 100° C. and additional portions of 2-bromomethyl-1,3-dioxolane (0.08 mL, 0.8 mmol) and potassium carbonate (0.11 g, 0.8 mmol) were added. The reaction was stirred 8 h, cooled, then concentrated under reduced pressure. It was diluted with ethyl acetate, washed with water, and again concentrated under reduced pressure. The resulting material was purified by silica gel chromatography (60-100% ethyl acetate/hexanes, 1% methanol/ethyl acetate) to afford the desired product as a solid (370 mg, 28%). ¹H NMR (300 MHz, MeOH-d₄) δ 7.42 (d, J=8.1 Hz, 1H), 7.19 (s, 1H), 7.13 (d, J=3.1 Hz, 1H), 6.87 (mult, 1H), 6.35 (mult, 1H), 5.66 (s, 1H), 5.09 (t, J=4.1 Hz, 1H), 4.18 (d, J=4.0 Hz, 2H), 3.93 (mult, 4H), 3.00 (t, J=7.8 Hz, 2H), 2.69 (t, J=7.8 Hz, 2H); m/z (APCI) 341 (MH⁺), HRMS (ES) M⁺, found 341.1595; C₁₈H₂₀N₄O₃ requires 341.1613.

### EXAMPLE 36

### 2-Amino-3-methyl-6-{2-[3-(2-thienyl)phenyl]ethyl}pyrimidin-4(3H)-one

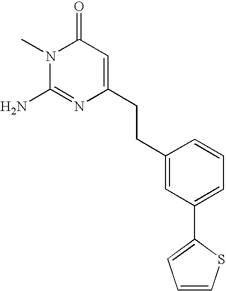

2-Amino-6-[2-(3-bromophenyl)ethyl]-3-methylpyrimidin-4(3H)-one (0.15 g, 0.49 mmol) was combined with a solution of dimethoxyethane/water/ethanol (7:3:2 ratio, 4 mL). Next, thiophene-2-boronic acid (0.081 g, 0.63 mmol), cesium carbonate (0.32 g, 0.97 mmol), and dichlrorbis(triphenylphosohine)palladium(II) (0.017 g, 0.024 mmol) were added and the mixture was heated by microwave at 150° C. for 15 min. Insoluble materials were then removed by filtration and the solution purified by reverse phase HPLC (CH₃CN/H₂O/0.1% TFA) to afford the desired product as a white solid (70 mg, 45%). ¹H NMR (300 MHz, DMSO) δ 8.30 (s, 2H), 7.52 (mult, 4H), 7.34 (t, J=7.6 Hz, 1H), 7.20 (d, J=7.5 Hz, 1H), 7.14 (mult, 1H), 5.81 (s, 1H), 3.26 (s, 3H), 2.94 (t, J=7.8 Hz, 2H), 2.74 (t, J=7.8 Hz, 2H); m/z (APCI) 312 (MH⁺).

The following compounds were synthesized using methods analogous to those previously described for Example 36 employing the appropriate boronic acid with the precursor aryl bromide as indicated.

TABLE 2

| | | | | | Precursor |
|---|---|---|---|---|---|
| | | | | | aryl |
| Ex. | Compound | Structure | ¹H NMR | m/z | bromide |
| 37 | 2-amino-3-methyl-6-{2-[4′-(methylsulfonyl)biphenyl-3-yl]ethyl}pyrimidin-4(3H)-one | 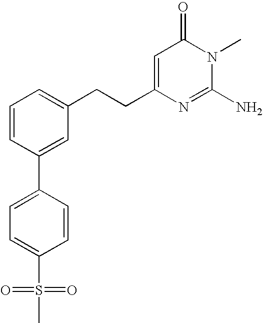 | ¹H NMR (300 MHz,DMSO) δ 7.99 (d, J = 8.4 Hz, 2 H), 7.91 (d,J = 8.4 Hz,2 H), 7.57 (mult, 2 H), 7.42(t, J = 7.6 Hz, 1 H), 7.30 (d,J = 7.5 Hz, 1 H), 7.07 (s,2 H), 5.54 (s, 1 H), 3.25 (s,3 H), 3.22 (s, 3 H), 2.97 (t,J = 7.8 Hz, 2 H), 2.62 (t,J = 7.8 Hz, 2 H) | 384 | 29 |
| 38 | 2-amino-6-{2-[3-(1 H-indol-5-yl)phenyl]ethyl}-3-methyl-pyrimidin-4(3H)-one | 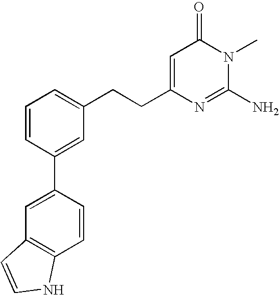 | ¹H NMR (300 MHz,DMSO) δ 11.12 (s, 1 H),8.30 (s, 1 H), 7.79 (s, 1 H),7.56 (s, 1 H), 7.48 (mult, 3 H),7.36 (mult, 3 H), 7.17(d, J = 7.4 Hz, 1 H), 6.48(s, 1 H), 5.83 (s, 1 H),3.27 (s, 3 H), 2.97 (t,J = 7.7 Hz, 2 H),2.77 (t, J = 7.7 Hz,2 H) | 345 | 29 |
| 39 | 2-amino-6-{2-[3-(2,3-dihydro-1,4-benzo-dioxin-6-yl)phenyl]ethyl}-3-methylpyrimidin-4(3H)-one | 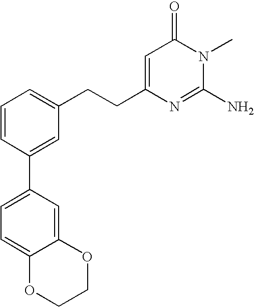 | ¹H NMR (300 DMSO) δ7.39 (mult, 2 H), 7.30 (t,J = 7.5 Hz, 1 H), 7.11 (mult,5 H), 6.91 (d, J = 8.9 Hz,1 H), 5.52 (s, 1 H), 4.27 (s,4 H), 3.22 (s, 3 H), 2.91 (t,J = 7.8 Hz, 2 H), 2.59 (t,J = 7.9 Hz, 2 H) | 364 | 29 |
| 40 | 2-amino-3-methyl-6-{2-[3′-(1H-pyrazol-1-yl)biphenyl-3-yl]ethyl}pyrimidin-4(3H)-one | 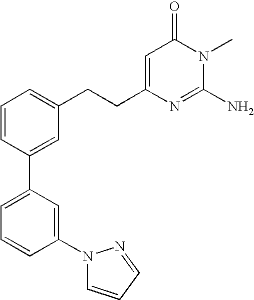 | ¹H NMR (300 MHz,DMSO) δ 8.63 (d,J = 2.4 Hz, 1 H), 8.07 (s, 1 H), 7.84(mult, 1 H), 7.77 (d,J = 1.4 Hz, 1 H), 7.58 (mult, 4 H),7.40 (t, J = 7.6 Hz, 1 H),7.26 (d, J = 7.5 Hz, 1 H),7.06 (s, 2 H), 6.57 (t,J = 2.1 Hz, 1 H), 5.55 (s, 1 H), 3.22(s, 3 H), 2.97 (t, J = 7.9 Hz,2 H), 2.63 (t, J = 7.9 Hz, 2 H) | 372 | 29 |
| 41 | 2-amino-6-{2-[3-(2,3-dihydro-1-benzofuran-5-yl)phenyl]ethyl}-3-methylpyrimidin-4(3H)-one | 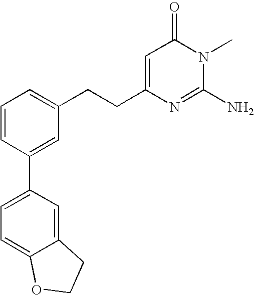 | ¹H NMR (300 MHz,DMSO) δ 7.49 (s, 1 H)7.34 (mult, 4 H), 7.12 (d,J = 7.4 Hz, 1 H), 7.05 (s, 2 H),6.82 (d, J = 8.2 Hz, 1 H),5.53 (s, 1 H), 4.56 (t,J = 8.7 Hz, 2 H), 3.22 (s, 3 H), 2.91(t, J = 7.8 Hz, 2 H), 2.59 (t,J = 7.7 Hz, 2 H) | 348 | 29 |
| 42 | 2-amino-3-methyl-6-{2-[3-(1H-pyrrol-2-yl)phenyl]ethyl}pyrimidin-4(3H)-one | 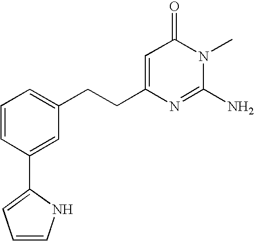 | ¹H NMR (300 MHz,DMSO) δ 11.25 (s, 1 H)8.41 (s, 2 H), 7.52 (mult,2 H), 7.26 (t, J = 7.6 Hz,1 H), 7.03 (d, J = 7.5 Hz,1 H), 6.84 (s, 1 H), 6.48 (s,1 H), 6.10 (mult, 1 H), 5.81(s, 1 H), 3.25 (s, 3 H), 2.90(mult, 2 H), 2.75 (t,J = 7.5 Hz, 2 H) | 295 | 29 |
| 43 | methyl3′-[2-(2-amino-1-methyl-6-oxo-1,6-dihydro-pyrimidin-4-yl)ethyl]biphenyl-4-carboxylate | 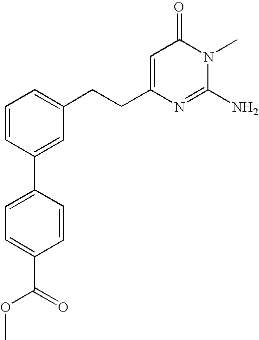 | ¹H NMR (300 MHz,DMSO) δ 8.03 (d,J = 8.3 Hz, 2 H), 7.80 (d, J = 8.4 Hz,2 H), 7.56 (mult, 2 H), 7.40(t, J = 7.6 Hz, 1 H), 7.27 (d,J = 7.4 Hz, 1 H), 7.07 (s,2 H), 5.53 (s, 1 H), 3.88 (s,3 H), 3.22 (s, 3 H), 2.96 (t,J = 7.8 Hz, 2 H), 2.61 (t,J = 7.8 Hz, 2 H) | 364 | 29 |
| 44 | 2-amino-6-{2-[3′-(hydroxy-methyl)biphenyl-3-yl]ethyl}-3-methylpyrimidin-4(3H)-one | 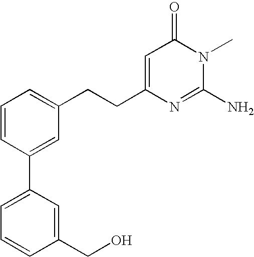 | ¹H NMR (300 MHz,DMSO) δ 7.57 (s, 1 H),7.40 (mult, 6 H), 7.20 (d,J = 7.4 Hz, 1 H), 7.06 (s, 2 H),5.53 (s, 1 H), 5.20 (s, 1 H),4.56 (s, 2 H), 3.22 (s, 3 H),2.94 (t, J = 7.8 Hz, 2 H),2.60 (t, J = 7.8 Hz, 2 H) | 336 | 29 |
| 45 | 2-amino-6-[2-(3′-hydroxy-biphenyl-3-yl)-ethyl]-3-methyl-pyrimidin-4(3H)-one | 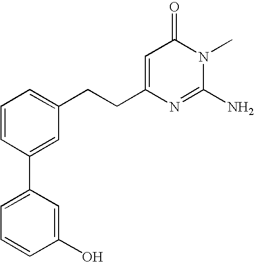 | ¹H NMR (300 MHz,DMSO) δ 9.51 (s, 1 H), 7.20(mult, 9 H), 6.75 (mult, 1 H),5.54 (s, 1 H), 3.22 (s, 3 H),2.93 (t, J = 7.9 Hz, 2 H),2.59 (t, J = 7.9 Hz, 2 H) | 322 | 29 |
| 46 | 6-[2-(3′-acetylbiphenyl-3-yl)ethyl]-2-amino-3-methyl-pyrimidin-4(3H)-one | 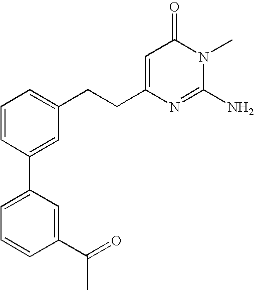 | ¹H NMR (300 MHz,DMSO) δ 8.16 (s, 1 H),7.92 (mult, 2 H), 7.58 (mult, 3 H),7.40 (t, J = 7.6 Hz, 1 H),7.26 (d, J = 7.5 Hz, 1 H),7.06 (s, 2 H), 5.54 (s, 1 H),3.22 (s, 3 H), 2.97 (t,J = 7.8 Hz, 2 H), 2.63 (mult, 5 H) | 348 | 29 |
| 47 | 2-amino-3-methyl-6-[2-(3-pyridin-4-ylphenyl)ethyl]pyrimidin-4(3H)-one | 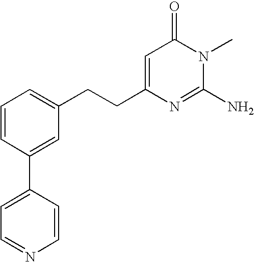 | ¹H NMR (300 MHz,DMSO) δ 8.81 (d,J = 6.3 Hz, 2 H), 8.55 (s, 2 H), 8.06(d, J = 6.3 Hz, 2 H), 7.83 (s,1 H), 7.77 (d, J = 7.5 Hz,1 H), 7.49 (mult, 2 H), 5.82 (s,1 H), 3.01 (t, J = 7.8 Hz,2 H), 2.79 (t, J = 7.8 Hz,2 H) | 307 | 29 |
| 48 | 2-amino-6-{2-[3-(2-furyl)phenyl]ethyl}-3-methylpyrimidin-4(3H)-one | 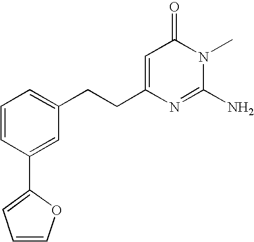 | ¹H NMR (300 MHz,DMSO) δ 7.73 (d,J = 0.9 Hz, 1 H), 7.52 (mult, 2 H),7.32 (t, J = 7.6 Hz, 1 H),7.13 (d, J = 7.6 Hz, 1 H),7.06 (s, 2 H), 6.91 (d,J = 3.3 Hz, 1 H), 6.58 (mult, 1 H),5.53 (s, 1 H), 3.22 (s, 3 H),2.91 (t, J = 7.9 Hz, 2 H),2.58 (t, J = 7.9 Hz, 2 H). | 296 | 29 |
| 49 | 2-amino-3-methyl-6-{2-[3-(5-methyl-2-thienyl)phenyl]ethyl}pyrimidin-4(3H)-one | 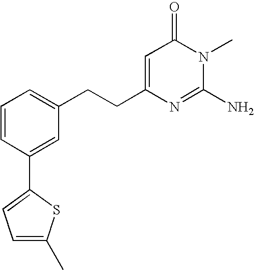 | ¹H NMR (300.132 MHz,DMSO) δ 8.23 (s, 2 H), 7.42(mult, 2 H), 7.30 (mult, 2 H),7.15 (d, J = 6.5 Hz, 1 H),6.81 (s, 1 H), 5.79 (s, 1 H),3.26 (s, 3 H), 2.92 (mult,2 H), 2.72 (mult, 2 H), 2.46(s, 3 H) | 326 | 29 |
| 50 | 2-amino-6-[2-(3′-methoxy-biphenyl-3-yl)ethyl]-3-methyl-pyrimidin-4(3H)-one | 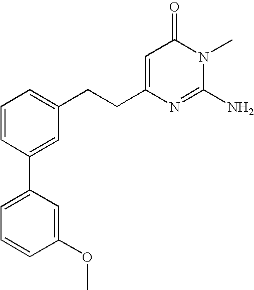 | ¹H NMR (300 MHz,DMSO) δ 7.47 (mult, 2 H),7.36 (mult, 2 H), 7.18 (mult,3 H), 7.06 (s, 2 H), 6.93(mult, 1 H), 5.53 (s, 1 H),3.82 (s, 3 H), 3.22 (s, 3 H), 2.94(t, J = 7.7 Hz, 2 H),2.61 (t, J = 7.7 Hz, 2 H) | 336 | 29 |
| 51 | 2-amino-3-methyl-6-{2-[3-(4-methyl-2-thienyl)phenyl]ethyl}pyrimidin-4(3H)-one | 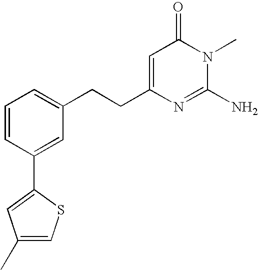 | ¹H NMR (300.132 MHz,DMSO) δ 8.29 (s, 2 H), 7.47(mult, 2 H), 7.32 (mult,2 H),7.18 (d, J = 7.5 Hz, 1 H),7.11 (s, 1 H), 5.80 (s, 1 H),3.26 (s, 3 H), 2.92 (mult,2 H), 2.73 (t, J = 7.7 Hz, 2 H), 2.23 (s, 3 H) | 326 | 29 |
| 52 | 2-amino-6-(2-biphenyl-3-ylethyl)-3-methylpyrimidin-4(3H)-one | 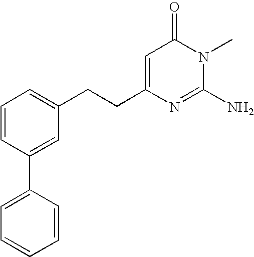 | ¹H NMR (300 MHz,DMSO) δ 7.63 (d,J = 7.3 Hz, 2 H), 7.46 (mult, 4 H),7.36 (mult, 2 H), 7.21 (d,J = 7.1 Hz, 1 H), 7.07 (s, 2 H),5.54 (s, 1 H), 3.22 (s, 3 H),2.94 (t, J = 7.7 Hz, 2 H),2.61 (t, J = 7.7 Hz, 2 H) | 306 | 29 |
| 53 | 2-amino-6-[2-(4′-chloro-biphenyl-3-yl)ethyl]-3-methyl-pyrimidin-4(3H)-one | 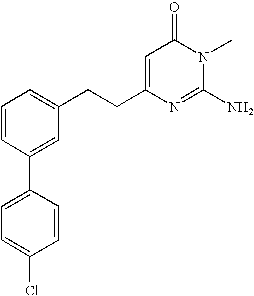 | ¹H NMR (300 MHz,DMSO) δ 7.67 (d,J = 8.5 Hz, 2 H), 7.49 (mult, 4 H),7.37 (t, J = 7.6 Hz, 1 H),7.22 (d, J = 7.5 Hz, 1 H),7.05 (s, 2 H), 5.53 (s, 1 H),3.22 (s, 3 H), 2.94 (t,J = 7.8 Hz, 2 H), 2.60 (mult, 2 H) | 340 | 29 |
| 54 | 2-amino-6-[2-(4′-hydroxy-biphenyl-3-yl)ethyl]-3-methyl-pyrimidin-4(3H)-one | 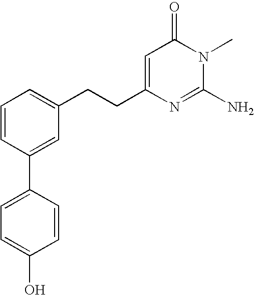 | ¹H NMR (300 MHz,DMSO) δ 9.48 (s, 1 H), 7.45(d, J = 8.6 Hz, 2 H), 7.33(mult, 3 H), 7.09 (mult, 3 H),6.83 (d, J = 8.5 Hz, 2 H),5.53 (s, 1 H), 3.22 (s, 3 H),2.91 (t, J = 7.9 Hz, 2 H),2.59 (t, J = 7.8 Hz, 2 H) | 322 | 29 |
| 55 | {3′-[2-(2-amino-1-methyl-6-oxo-1,6-dihydro-pyrimidin-4-yl)ethyl]biphenyl-4-yl}aceto-nitrile | 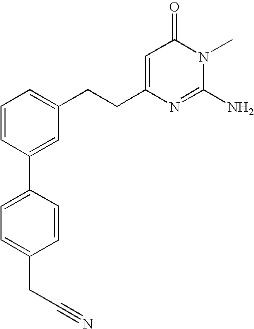 | ¹H NMR (300 MHz,DMSO) δ 8.29 (s, 2 H), 7.68(d, J = 8.2 Hz, 2 H), 7.56 (s,1 H), 7.51 (d, J = 7.8 Hz,1 H), 7.41 (mult, 3 H), 7.26(d, J = 7.4 Hz, 1 H), 5.81 (s,1 H), 4.08 (s, 2 H), 3.26 (s,3 H), 2.97 (t, J = 7.8 Hz,2 H), 2.76 (t, J = 7.8 Hz, 2 H) | 345 | 29 |
| 56 | 2-amino-3-methyl-6-{2-[3-(3-thienyl)phenyl]ethyl}pyrimidin-4(3H)-one | 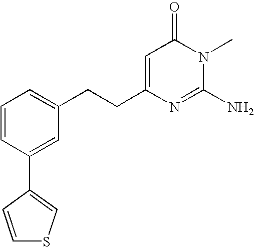 | ¹H NMR (300 MHz,DMSO) δ 7.82 (mult, 1 H),7.62 (mult, 1 H), 7.53 (mult,3 H), 7.30 (t, J = 7.6 Hz,1 H), 7.13 (d, J = 7.5 Hz,1 H), 7.06 (s, 2 H), 5.54 (s,1 H), 3.22 (s, 3 H), 2.91(mult, 2 H), 2.60 (mult, 2 H) | 312 | 29 |
| 57 | 2-amino-6-[2-(4′-methoxy-biphenyl-3-yl)ethyl]-3-methyl-pyrimidin-4(3H)-one | 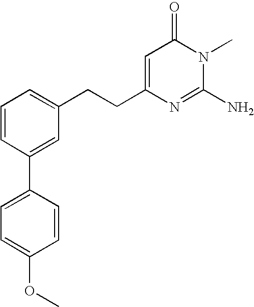 | ¹H NMR (300 MHz,DMSO) δ 8.32 (s, 2 H), 7.58(d, J = 8.7 Hz, 2 H), 7.50 (s,1 H), 7.45 (d, J = 7.8 Hz,1 H), 7.35 (t, J = 7.6 Hz,1 H), 7.19 (d, J = 7.4 Hz,1 H), 7.02 (d, J = 8.7 Hz,2 H), 5.81 (s, 1 H), 3.80 (s,3 H), 3.26 (s, 3 H), 2.95(mult, 2 H), 2.75 (t,J = 7.7 Hz, 2 H) | 336 | 29 |
| 58 | 2-amino-6-(2-{3-[5-(hydroxy-methyl)-2-thienyl]phenyl}ethyl)-3-methyl-pyrimidin-4(3H)-one | 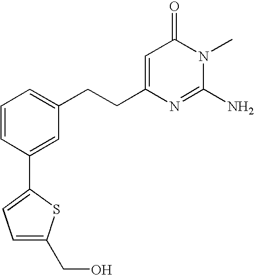 | ¹H NMR (300 MHz,DMSO) δ 7.98 (s, 2 H), 7.46(mult, 2 H), 7.32 (mult, 2 H)7.16 (d, J = 7.4 Hz, 1 H),6.95 (d, J = 3.6 Hz, 1 H),5.76 (s, 1 H), 4.63 (s, 2 H),3.25 (s, 3 H), 2.92 (mult,2 H), 2.71 (t, J = 7.3 Hz, 2 H) | 342 | 29 |
| 59 | 2-amino-3-methyl-6-[2-(3-pyridin-3-ylphenyl)ethyl]pyrimidin-4(3H)-one | 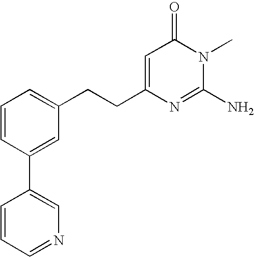 | ¹H NMR (300 MHz,DMSO) δ 8.99 (s, 1 H), 8.68(d, J = 4.9 Hz, 3 H), 8.30 (d,J = 8.0 Hz, 1 H), 7.65 (mult,3 H), 7.47 (t, J = 7.6 Hz,1 H), 7.36 (d, J = 7.6 Hz,1 H), 5.86 (s, 1 H), 3.26 (s,3 H), 2.99 (t, J = 7.7 Hz,2 H), 2.80 (t, J = 7.7 Hz, 2 H) | 307 | 29 |
| 60 | 2-amino-6-[2-(3′-ethoxy-biphenyl-3-yl)ethyl]-3-methyl-pyrimidin-4(3H)-one | 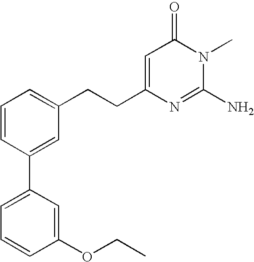 | ¹H NMR (300. MHz,DMSO) δ 7.46 (mult, 2 H),7.35 (mult, 2 H), 7.19 (mult,2 H), 7.12 (s, 1 H), 7.06 (s,2 H), 6.91 (mult, 1 H), 5.53(s, 1 H), 4.10 (q, J = 6.9 Hz,2 H), 3.22 (s, 3 H), 2.94 (t,J = 7.8 Hz, 2 H), 2.60 (t,J = 7.9 Hz, 2 H), 1.35 (t,J = 6.9 Hz, 3 H) | 350 | 29 |
| 61 | 2-amino-6-{2-[3-(3-furyl)phenyl]ethyl}-3-methyl-pyrimidin-4(3H)-one | 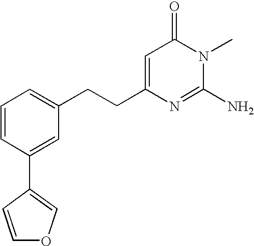 | ¹H NMR (300 MHz,DMSO) δ 7.29 (mult, 5 H),7.08 (mult, 5 H), 5.53 (s,1 H), 3.75 (s, 3 H), 3.22 (s,3 H), 2.90 (t, J = 7.8 Hz,2 H), 2.58 (t, J = 7.8 Hz, 2 H) | 296 | 29 |
| 62 | 2-amino-6-[2-(2′-methoxy-biphenyl-3-yl)ethyl]-3-methyl-pyrimidin-4(3H)-one | 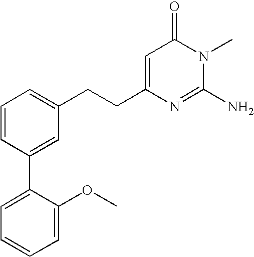 | ¹H NMR (300 MHz,DMSO) δ 7.29 (mult, 5 H),7.08 (mult, 5 H), 5.53 (s,1 H), 3.75 (s, 3 H), 3.22 (s,3 H), 2.90 (t, J = 7.8 Hz,2 H), 2.58 (t, J = 7.8 Hz, 2 H) | 336 | 29 |
| 63 | 3′-[2-(2-amino-1-methyl-6-oxo-1,6-dihydro-pyrimidin-4-yl)ethyl]-N-(tert-butyl)biphenyl-2-sulfonamide | 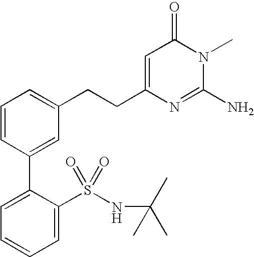 | ¹H NMR (300 MHz,DMSO) δ 8.04 (d,J = 6.8 Hz, 1 H), 7.58 (mult, 2 H),7.28 (mult, 5 H), 7.04 (s,2 H), 6.18 (s, 1 H), 5.51 (s, 1 H),3.21 (s, 3 H), 2.91 (t,J = 7.8 Hz, 2 H), 2.58 (t,J = 7.8 Hz, 2 H), 0.98 (s, 9 H) | 441 | 29 |
| 64 | methyl3′-[2-(2-amino-1-methyl-6-oxo-1,6-dihydro-pyrimidin-4-yl)ethyl]biphenyl-2-carboxylate | 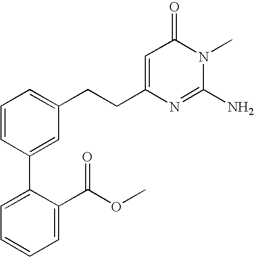 | ¹H NMR (300 MHz,DMSO) δ 8.26 (s, 2 H),7.73 (d, J = 7.2 Hz, 1 H),7.62 (t, J = 8.5 Hz, 1 H),7.49 (t, J = 7.2 Hz, 1 H),7.37 (mult, 2 H), 7.25 (d,J = 6.9 Hz, 1 H), 7.19 (s, 1 H),7.13 (d, J = 6.8 Hz, 1 H),5.79 (s, 1 H), 3.26 (s, 3 H),2.93 (t, J = 7.4 Hz, 2 H),2.72 (t, J = 7.4 Hz, 2 H). | 364 | 29 |
| 65 | 2-amino-3-methyl-6-{2-[2-(2-thienyl)phenyl]ethyl}pyrimidin-4(3H)-one | 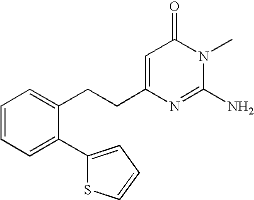 | ¹H NMR (300. MHz,DMSO) δ 7.60 (mult, 1 H),7.29 (mult, 4 H), 7.16 (mult,2 H), 7.03 (mult, 2 H), 5.37(s, 1 H), 3.20 (s, 3 H), 2.97 (t,J = 8.1 Hz, 2 H), 2.46 (t,J = 8.1 Hz, 2 H) | 312 | 30 |
| 66 | 2-amino-6-{2-[3′-(hydroxy-methyl)biphenyl-4-yl]ethyl}-3-methylpyrimidin-4(3H)-one | 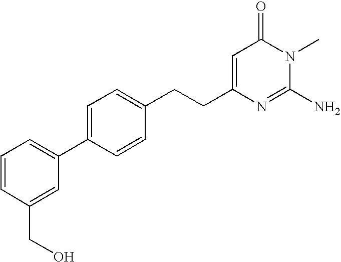 | ¹H NMR (300. MHz,DMSO) δ 7.53 (mult, 4 H),7.39 (mult, 2 H), 7.29 (mult,2 H), 7.06 (s, 2 H), 5.53 (s,1 H), 5.18 (t, J = 5.6 Hz,1 H), 4.56 (d, J = 4.5 Hz,2 H), 3.22 (s, 3 H), 2.91 (t,J = 7.8 Hz, 2 H), 2.58 (t,J = 7.9 Hz, 2 H) | 336 | 31 |
| 67 | 2-amino-6-[2-(3′,4′-dimethoxy-biphenyl-4-yl)ethyl]-3-methyl-pyrimidin-4(3H)-one | 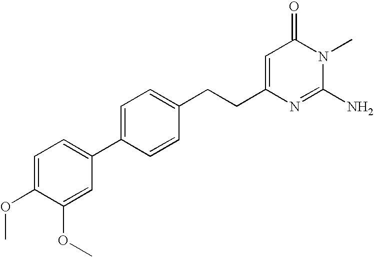 | ¹H NMR (300. MHz,DMSO) δ 7.60 (d,J = 8.1 Hz, 2 H), 7.35 (d, J = 2.7 Hz,2 H), 7.33 (d, J = 2.8 Hz,3 H), 7.19 (mult, 2 H), 6.92(mult, 1 H), 5.80 (s, 1 H),3.82 (s, 3 H), 3.26 (s, 3 H),2.94 (t, J = 7.7 Hz, 2 H),2.73 (t, J = 7.7 Hz, 2 H) | 366 | 31 |
| 68 | 2-amino-6-[2-(3′-methoxy-biphenyl-4-yl)ethyl]-3-methyl-pyrimidin-4(3H)-one | 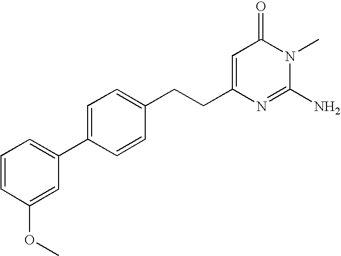 | ¹H NMR (300 MHz,DMSO) δ 8.24 (s, 2 H), 7.60(d, J = 8.1 Hz, 2 H),7.35(mult, 3 H), 7.19 (mult, 2 H),6.92 (mult, 1 H), 5.80 (s,1 H), 3.82 (s, 3 H), 2.94 (t,J = 7.7 Hz, 2 H), 2.73 (t, J = 7.7 Hz, 2 H) | 336 | 31 |
| 69 | 2-amino-3-methyl-6-{2-[4-(2-thienyl)phenyl]ethyl}pyrimidin-4(3H)-one | 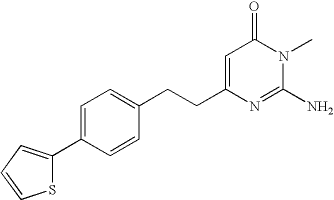 | ¹H NMR (300. MHz,DMSO) δ 7.58 (d,J = 8.1 Hz, 2 H), 7.51 (d, J = 5.1 Hz,2 H), 7.47 (d, J = 3.4 Hz,2 H), 7.29 (d, J = 8.1 Hz,2 H), 7.12 (t, J = 4.3 Hz,1 H), 5.78 (s, 1 H), 3.26 (s,3 H), 2.91 (t, J = 7.7 Hz,2 H), 2.71 (t, J = 7.7 Hz, 2 H) | 312 | 31 |
| 70 | 2-amino-6-[2-(1,1′-biphenyl-3-yl)ethyl]pyrimidin-4(3H)-one | 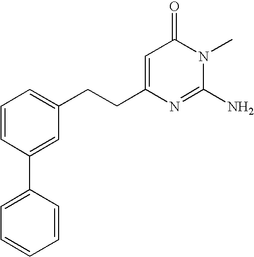 | ¹H NMR (300. MHz,DMSO) δ 7.63 (d,J = 7.3 Hz, 2 H), 7.46 (t,J = 7.6 Hz,4 H), 7.36 (t, J = 7.2 Hz,2 H), 7.21 (d, J = 7.1 Hz,1 H), 7.07 (s, 2 H), 5.54 (s,1 H), 3.22 (s, 3 H), 2.94 (t,J = 7.7 Hz, 2 H), 2.61 (t,J = 7.7 Hz, 2 H) | 292 | 29 |

### EXAMPLE 71

### N-{2-[2-Amino-4-{2-[3-(2-furyl)phenyl]ethyl}-6-oxopyrimidin-1(6H)-yl]ethyl}acetamide (Scheme 6, I)

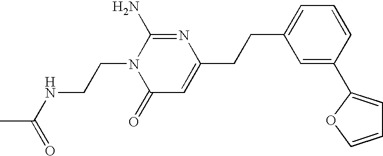

This material was prepared according to Scheme 6. To a stirred solution of 2-amino-3-(2-aminoethyl)-6-{2-[3-(2-furyl)phenyl]ethyl}pyrimidin-4(3H)-one (40 mg, 0.072 mmol as the bis-TFA salt) in CH₂Cl₂ (1 mL) and DMF (0.5 mL) was added triethylamine (33 μL, 0.24 mmol), then acetyl chloride (5.7 μL, 0.080 mmol). The reaction was allowed to stir for 15 min., then concentrated to remove the CH₂Cl₂. EtOH (0.5 mL) and H₂O (0.5 mL) were then added and the material purified by reverse phase HPLC (CH₃CN/H₂O/0.1% TFA) to afford the desired product as a white solid (20 mg, 77%). ¹H NMR (300 MHz, d₃-MeOD) δ 7.60 (s, 1H), 7.55 (mult, 2H), 7.33 (t, J=1.7 Hz, 1H), 7.17 (d, J=7.6 Hz, 1H), 6.75 (d, J=3.3 Hz, 1H), 6.50 (mult, 1H), 5.85 (s, 1H), 4.09 (t, J=6.6 Hz, 2H), 3.41 (t, J=6.6 Hz, 2H), 3.00 (t, J=7.8 Hz, 2H), 2.83 (t, J=7.8 Hz, 2H), 1.91 (s, 3H); m/z (APCI) 367 (MH⁺).

### 2-Amino-3-(2-amino-ethyl)-6-[2-(3-furan-2-yl-phenyl)-ethyl]-3H-pyrimidin-4-one (Scheme 6, H) was Prepared as Follows

### 3-(3-Bromo-phenyl)-propionic acid ethyl ester (Scheme 6, A)

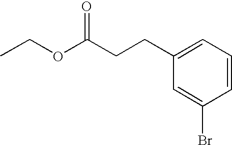

To a solution of 3-(3-bromophenyl)-propionic acid (25.0 g, 109 mmol) in DCM (300.0 mL) was added oxalyl chloride (11.9 mL, 136 mmol) and 2 drops of DMF. After stirring for 2 h the solution was concentrated under reduced pressure, dissolved in DCM (80 mL), and cooled to −10° C. To this solution, ethanol (80 mL) was added dropwise and stirred at room temperature for 4 h. The solution was concentrated under reduced pressure and dried under vacuum to afford the product in quantitative yield. ¹H NMR (300.132 MHz, DMSO) δ 7.45 (s, 1H), 7.38 (mult 1H), 7.24 (d, J=20.8 Hz, 2H), 4.04 (q, J=7.1 Hz, 2H), 2.84 (t, J=7.5 Hz, 2H), 2.62 (t, J=7.5 Hz, 2H), 1.15 (t, J=7.1 Hz, 3H); m/z (APCI) 258 (MH⁺).

### 3-(3-Furan-2-yl-phenyl)-propionic acid ethyl ester (Scheme 6, B)

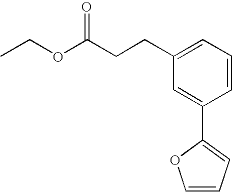

To a solution of 3-(3-bromo-phenyl)-propionic acid ethyl ester (13.0 g, 50.5 mmol) in dioxane (338 mL) was added 2-(tributylstannyl)furan (9.5 mL, 30.3 mmol, 0.6 eq.), and dichloro-bis-(triphenylphosphine) palladium (2.48 g, 3.53 mmol, 0.07 eq.). The mixture was headed at 100° C. for 20 min. then portions of 2-(tributylstannyl)furan (9.5 mL, 30.3 mmol, 0.6 eq.) were added at 20 min. intervals until the starting material was consumed. The solution was concentrated under reduced pressure, adsorbed onto silica gel and purified by flash chromatography (hexanes, hexanes:DCM; 9.5/0.5 DCM, hexanes:DCM; 4/1, hexanes:DCM; 1/1) to afford the desired product (11.16 g, 45.68 mmol, 90%) as a yellow/brown solid. ¹H NMR (300.132 MHz, DMSO) δ 7.73 (s, 1H), 7.54 (t, J=8.3 Hz, 2H), 7.33 (mult 1H), 7.14 (d, J=12.0 Hz, 1H), 6.91 (s, 1H), 6.58 (d, J=5.1 Hz, 1H), 4.05 (q, J=7.1 Hz, 2H), 2.89 (t, J=7.4 Hz, 2H), 2.65 (t, J=7.4 Hz, 2H), 1.15 (t, J=8.0 Hz, 3H); m/z (APCI) 245 (MH⁺).

### 3-(3-Furan-2-yl-phenyl)-propionic acid ester (Scheme 6, C)

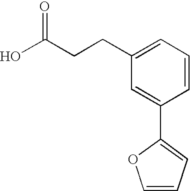

To a solution of 3-(3-furan-2-yl-phenyl)-propionic acid ethyl ester (23.23 g, 95.09 mmol) in THF (438 mL) and water (218 mL) was added a solution of LiOH (4.38 g, 104 mmol) in water (40 mL) by dropwise addition. After stirring overnight, the mixture was concentrated under reduced pressure to remove THF. The resulting solution was washed with diethyl ether and the aqueous phase was acidified by addition of HCl and washed with DCM. The DCM solution was dried Na₂SO₄), concentrated under reduced pressure, and dried under vacuum to afford the desired product (18.32 g, 84.72 mmol, 90%) as a yellow solid. ¹H NMR (300.132 MHz, DMSO) δ 12.09 (s, 1H), 7.73 (s, 1H), 7.54 (t, J=9.1 Hz, 2H), 7.33 (t, J=7.7 Hz, 1H), 7.16 (d, J=7.5 Hz, 1H), 6.92 (d, J=3.2 Hz, 1H), 6.58 (s, 1H), 2.87 (t, J=7.4 Hz, 2H), 2.57 (t, J=7.6 Hz, 2H); m/z (APCI) 217 (MH⁺)

### 5-(3-Furan-2-yl-phenyl)-3-oxo-pentanoic acid ethyl ester (Scheme 6, D)

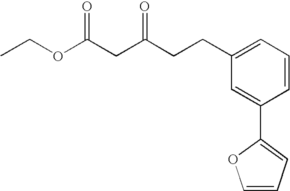

This material was prepared according to the procedure described for ethyl 5-(3-bromophenyl)-3-oxopentanoate, except 3-(3-furan-2-yl-phenyl)-propionic acid ester was used in place of 3-(3-bromophenyl)propionic acid to afford the desired product (11.69 g, 40.83 mmol, 48%). ¹H NMR (300. MHz, DMSO) δ 7.73 (s, 1H), 7.52 (t, J=7.1 Hz, 2H), 7.32 (t, J=7.6 Hz, 1H), 7.13 (d, J=7.5 Hz, 1H), 6.91 (d, J=3.3 Hz, 1H), 6.58 (mult, 1H), 4.08 (q, J=7.1 Hz, 2H), 3.61 (s, 2H), 2.87 (mult, 4H), 1.17 (t, J=7.1 Hz, 3H); m/z (APCI) 287 (MH⁺).

### 2-Amino-6-[2-(3-furan-2-yl-phenyl)-ethyl]-3H-pyrimidin-4-one (Scheme 6, E)

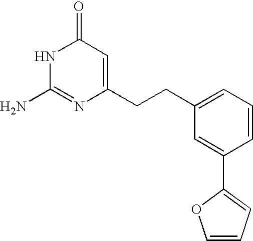

This material was prepared according to the procedure described for 2-amino-6-[2-(3-bromophenyl)ethyl]pyrimidin-4(3H)-one, except 5-(3-furan-2-yl-phenyl)-3-oxo-pentanoic acid ethyl ester was used in place of ethyl 5-(3-bromophenyl)-3-oxopentanoate to give the desired product as a light brown solid (8.86 g, 31.4 mmol, 77%). ¹H NMR (300. MHz, DMSO) δ 7.73 (s, 1H), 7.52 (t, J=7.8 Hz, 2H), 7.32 (t, J=7.6 Hz, 1H), 7.13 (d, J=7.5 Hz, 1H), 6.91 (d, J=3.3 Hz, 1H), 6.56 (mult, 4H), 5.40 (s, 1H), 2.90 (t, J=7.9 Hz, 2H), 2.58 (t, J=7.9 Hz, 2H); m/z (APCI) 282 (MH⁺).

### N′-(4-{2-[3-(2-Furyl)phenyl]ethyl}-6-oxo-1,6-dihydropyrimidin-2-yl)-N,N-dimethylimidoformamide (Scheme 6, F)

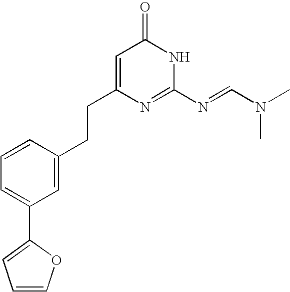

To a stirred solution of 2-amino-6-{2-[3-(2-furyl)phenyl]ethyl}pyrimidin-4(3H)-one (5.0 g, 17.8 mmol) in DMF (54 mL) under nitrogen was added DMF dimethyl acetal (3.5 mL, 26.7 mmol). The reaction was allowed to stir overnight, then H₂O (0.5 mL) was added and solution concentrated under reduced pressure. The resulting material was then dissolved in CH₃CN and again concentrated under reduced pressure to afford desired product as a gum (quantitative yield). ¹H NMR (300 MHz, d₃-MeOD) δ 8.58 (s, 1H), 7.52 (mult, 3H), 7.28 (t, J=7.7 Hz, 1H), 7.11 (d, J=7.6 Hz, 1H), 6.72 (d, J=2.8 Hz, 1H), 6.49 (mult, 1H), 5.78 (s, 1H), 3.17 (s, 3H), 3.11 (s, 3H), 3.00 (mult 2H), 2.78 (mult, 2H); m/z (APCI) 337 (MH⁺).

### Benzyl {2-[2-{[(1E)-(dimethylamino)methylene]amino}-4-{2-[3-(2-furyl)phenyl]ethyl}-6-oxopyrimidin-1(6H)-yl]ethyl}carbamate (Scheme 6, G)

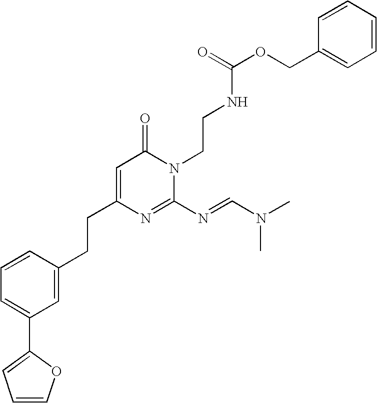

To a stirred solution of N′-(4-{2-[3-(2-furyl)phenyl]ethyl}-6-oxo-1,6-dihydropyrimidin-2-yl)-N,N-dimethylimidoformamide (4.0 g, 11.9 mmol) in THF (60 mL) under nitrogen atmosphere was added benzyl N-(2-hydroxyethyl)carbamate (4.6 g, 23.8 mmol) and triphenylphosphine (6.2 g, 23.8 mmol). The solution was allowed to stir for 10 min., then diethyl azodicarboxylate (DEAD) (3.7 mL, 238 mmol) was added and reaction allowed to stir for 0.5 h. A moderate exotherm occurred after the addition. This material was used without purification. m/z (APCI) 514 (MH⁺).

### 2-Amino-3-(2-aminoethyl)-6-{2-[3-(2-furyl)phenyl]ethyl}pyrimidin-4(3H)-one (Scheme 6, H)

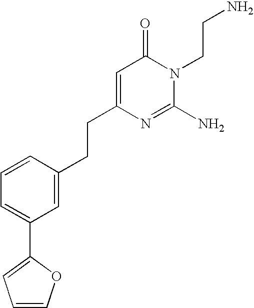

Benzyl {2-[2-{[(1E)-(dimethylamino)methylene]amino}-4-{2-[3-(2-furyl)phenyl]ethyl}-6-oxopyrimidin-1(6H)-yl]ethyl}carbamate (11.9 mmol) was dissolved in EtOH under nitrogen atmosphere. The solution was degassed with nitrogen, then Pd/C (10%, 4.0 g) was added and the mixture placed under 1 atmosphere of hydrogen. The reaction was stirred vigorously for 2.5 h, the catalyst removed by filtration, and the remaining solution concentrated under reduced pressure. The residue was then dissolved in CH₃CN (50 mL) and to this was added aqueous NH₄OH (50 mL) and the reaction heated in a sealed container at 70° C. for 6 h. It was then cooled, concentrated under reduced pressure, and partitioned between DCM and aqueous 1 N HCl. The layers were separated and the aqueous washed again with DCM. The aqueous was then basified with 50% aqueous sodium hydroxide solution and extracted with EtOAc (2×). The organic solution was then dried over Na₂SO₄, filtered, concentrated and purified by reverse phase HPLC (CH₃CN/H₂O with 0.1% TFA) to give the desired product as a white solid (2.7 g of the bis-TFA salt, 3 steps; 40%). ¹H NMR (300 MHz, d₃-MeOD) δ 7.61 (s, 1H), 7.54 (mult, 2H), 7.32 (t, J=7.7 Hz, 1H), 7.18 (d, J=7.7 Hz, 1H), 6.74 (d, J=3.4 Hz, 1H), 6.50 (mult, 1H), 5.89 (s, 1H), 4.29 (mult, 2H), 3.25 (mult, 2H), 3.01 (mult, 2H), 2.83 (mult, 2H); m/z (APCI) 325 (MH⁺).

The compounds below were prepared by reaction of 2-amino-3-(2-aminoethyl)-6-{2-[3-(2-furyl)phenyl]ethyl}pyrimidin-4(3H)-one (Scheme 6, H) with the appropriate acid chloride according to the procedure described for Example 71.

TABLE 3

| Ex. | Compound | Structure | ¹H NMR | m/z |
|---|---|---|---|---|
| 72 | N-{2-[2-amino-4-{2-[3-(2-furyl)phenyl]ethyl}-6-oxo-pyrimidin-1(6H)-yl]ethyl}butanamide | 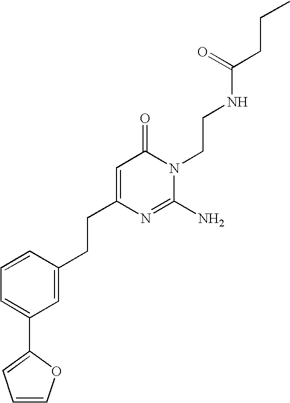 | ¹H NMR (300 MHz,d₃-MeOD) δ 7.50 (mult,3 H), 7.27 (t, J = 7.7 Hz,1 H), 7.11 (d, J = 7.5 Hz,1 H), 6.71 (d, J = 2.7 Hz,1 H), 6.48 (mult, 1 H),5.63 (s, 1 H), 4.04 (t,J = 7.1 Hz, 2 H), 3.38 (t,J = 7.0 Hz, 2 H), 2.96 (t,J = 7.8 Hz, 2 H), 2.67 (t,J = 7.8 Hz, 2 H), 2.16 (t,J = 7.4 Hz, 2 H), 1.62 (mult, 2 H),0.93 (t, J = 7.4 Hz, 3 H) | 395 |
| 73 | 1-{2-[2-amino-4-{2-[3-(2-furyl)phenyl]ethyl}-6-oxopyrimidin-1(6H)-yl]ethyl}pyrrolidine-2,5-dione | 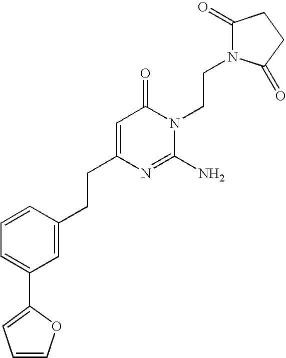 | ¹H NMR (300 MHz,d₃-MeOD) δ 7.51 (mult,3 H), 7.28 (q, J = 5.1 Hz,1 H), 7.10 (d, J = 7.6 Hz,1 H), 6.72 (d, J = 3.3 Hz,1 H), 6.48 (mult, 1 H),5.54 (s, 1 H), 4.18 (t,J = 5.4 Hz, 2 H), 3.80 (t,J = 5.4 Hz, 2 H), 2.95 (t,J = 7.7 Hz, 2 H), 2.65 (mult, 2 H),2.58 (s, 4 H) | 407 |
| 74 | methyl3-({2-[2-amino-4-{2-[3-(2-furyl)phenyl]ethyl}-6-oxo-pyrimidin-1(6H)-yl]ethyl}amino)-3-oxopropanoate | 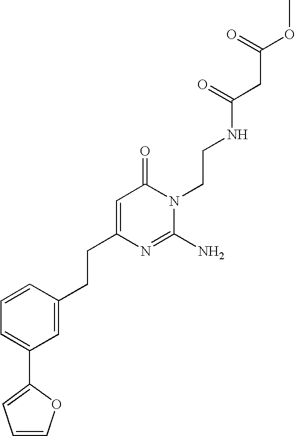 | ¹H NMR (300 MHz,d₃-MeOD) δ 7.50 (mult,3 H), 7.28 (t, J = 7.6 Hz,1 H), 7.11 (d, J = 7.3 Hz,1 H), 6.71 (d, J = 3.3 Hz,1 H), 6.49 (mult, 1 H),5.64 (s, 1 H), 4.07 (t,J = 6.9 Hz, 2 H), 3.71 (s, 3 H),3.43 (t, J = 6.9 Hz, 2 H),2.96 (t, J = 7.8 Hz, 2 H),2.67 (t, J = 7.8 Hz, 2 H) | 425 |
| 75 | methyl5-({2-[2-amino-4-{2-[3-(2-furyl)phenyl]ethyl}-6-oxo-pyrimidin-1(6H)-yl]ethyl}amino)-5-oxopentanoate | 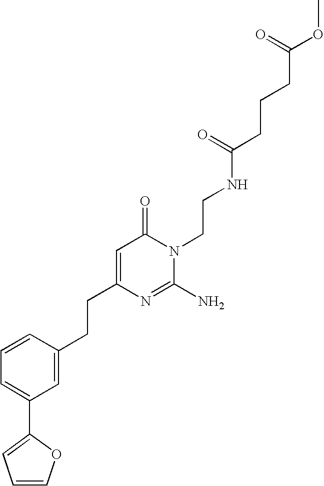 | ¹H NMR (300 MHz,d₃-MeOD) δ 7.50 (mult,3 H), 7.28 (t, J = 7.7 Hz,1 H), 7.11 (d, J = 7.6 Hz,1 H), 6.71 (d, J = 2.7 Hz,1 H), 6.48 (mult, 1 H),5.64 (s, 1 H), 4.05 (t,J = 7.0 Hz, 2 H), 3.65 (s, 3 H),3.38 (t, J = 6.9 Hz, 2 H),2.96 (t, J = 7.8 Hz, 2 H),2.67 (t, J = 7.8 Hz, 2 H),2.35 (t, J = 7.4 Hz, 2 H),2.23 (t, J = 7.4 Hz, 2 H),1.88 (mult, 2 H) | 453 |
| 76 | methyl6-({2-[2-amino-4-{2-[3-(2-furyl)phenyl]ethyl}-6-oxo-pyrimidin-1(6H)-yl]ethyl}amino)-6-oxohexanoate | 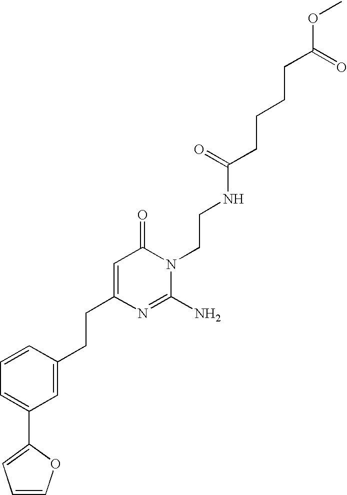 | ¹H NMR (300 MHz,d₃-MeOD) δ 7.50 (mult,3 H), 7.28 (t, J = 7.7 Hz,1 H), 7.11 (d, J = 7.9 Hz,1 H), 6.71 (d, J = 2.7 Hz,1 H), 6.48 (mult, 1 H),5.63 (s, 1 H), 4.05 (t,J = 7.0 Hz, 2 H), 3.65 (s, 3 H),3.38 (t, J = 7.0 Hz, 2 H),2.96 (t, J = 7.7 Hz, 2 H),2.67 (q, J = 5.2 Hz, 2 H),2.34 (mult, 2 H), 2.20(mult, 2 H), 1.61 (mult,4 H) | 467 |
| 77 | 6-({2-[2-amino-4-{2-[3-(2-furyl)phenyl]ethyl}-6-oxo-pyrimidin-1(6H)-yl]ethyl}amino)-6-oxohexanoicacid | 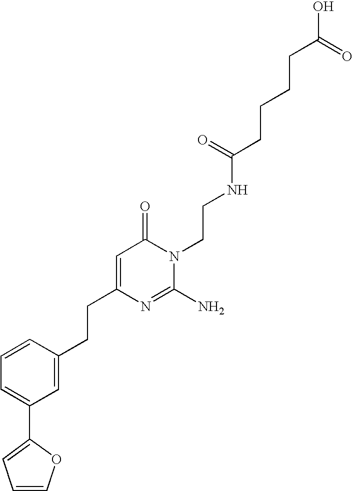 | ¹H NMR (300 MHz,d₃-MeOD) δ 7.50 (mult,3 H), 7.27 (t, J = 7.7 Hz,1 H), 7.11 (d, J = 7.6 Hz,1 H), 6.71 (d, J = 3.3 Hz,1 H), 6.48 (mult, 1 H),5.63 (s, 1 H), 4.04 (t,J = 7.0 Hz, 2 H), 3.38 (t,J = 7.0 Hz, 2 H), 2.96 (t,J = 7.8 Hz, 2 H), 2.67 (t,J = 7.8 Hz, 2 H), 2.30 (t,J = 6.9 Hz, 2 H), 2.21 (t,J = 7.0 Hz, 2 H), 1.62 (t,J = 3.5 Hz, 4 H) | 453 |
| 78 | N-{2-[2-amino-4-{2-[3-(2-furyl)phenyl]ethyl}-6-oxo-pyrimidin-1(6H)-yl]ethyl}-2-methoxyacetamide | 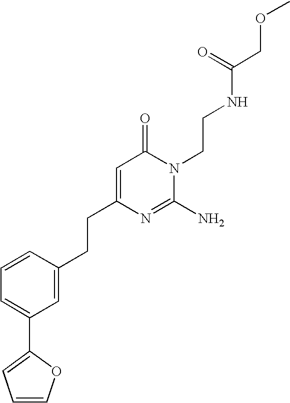 | ¹H NMR (300 MHz,d₃-MeOD) δ 7.56 (mult,3 H), 7.33 (t, J = 7.6 Hz,1 H), 7.16 (d, J = 7.6 Hz,1 H), 6.75 (d, J = 3.3 Hz,1 H), 6.50 (mult, 1 H),5.83 (s, 1 H), 4.13 (t,J = 6.3 Hz, 2 H), 3.83 (s, 2 H),3.50 (t, J = 6.3 Hz, 2 H),3.37 (s, 3 H), 3.00 (t,J = 7.7 Hz, 2 H), 2.82 (t,J = 7.7 Hz, 2 H) | 397 |

### EXAMPLE 79

### 2-Amino-3-[2-(benzylamino)ethyl]-6-{2-[3-(2-furyl)phenyl]ethyl}pyrimidin-4(3H)-one

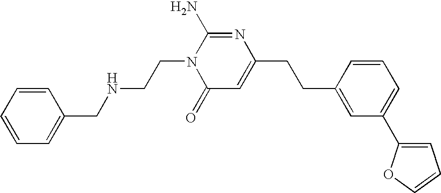

To a solution of 2-amino-3-(2-aminoethyl)-6-{2-[3-(2-furyl)phenyl]ethyl}pyrimidin-4(3H)-one (Scheme 6, H) (40 mg, 0.073 mmol) in MeOH (800 mL) was added benzaldehyde (7.7 mg, 0.073 mmol) and Et₃N (14.7 mg 0.145 mmol). After stirring for 30 min., a solution of NaBH₃CN (6.9 mg, 0.109 mmol) in MeOH (200 mL) was added and stirred for 30 min. The solution was concentrated under reduced pressure and purified by reverse phase HPLC using water/acetonitrile with 0.1% TFA to afford the desired product (6.0 mg, 0.015 mmol, 20%) as a white solid. ¹H NMR (300. MHz, DMSO) δ 7.73 (s, 1H), 7.50 (mult, 9H), 7.33 (t, J=7.6 Hz, 1H), 7.16 (d, J=7.8 Hz, 1H), 6.92 (d, J=3.3 Hz, 1H), 6.59 (mult, 1H), 5.69 (s, 1H), 4.18 (mult, 5H), 3.17 (mult, 2H), 2.92 (t, J=7.9 Hz, 2H), 2.65 (t, J=8.1 Hz, 2H); m/z (APCI) 415 (MH⁺).

TABLE 4

| Ex. | Compound | Structure | ¹H NMR | m/z |
|---|---|---|---|---|
| 80 | 2-amino-6-{2-[3-(2-furyl)phenyl]ethyl}-3-{2-[(3-methoxybenzyl)amino]ethyl}pyrimidin-4(3H)-one | 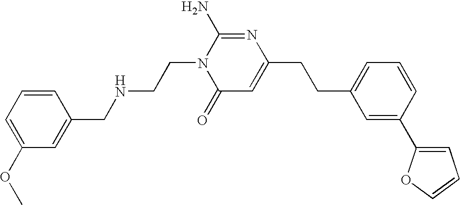 | ¹H NMR (300 MHz,DMSO) δ 8.71 (s, 1 H),7.73 (s, 1 H), 7.55 (mult,3 H), 7.35 (mult, 2 H),7.16 (d, J = 7.8 Hz,1 H), 7.03 (mult, 3 H),6.92 (d, J = 3.4 Hz,1 H), 6.59 (s, 1 H), 5.71(s, 1 H), 4.16 (s, 2 H),3.78 (s, 8 H), 2.92 (t,J = 8.1 Hz, 2 H), 2.65 (t,J = 7.8 Hz, 2 H) | 445 |

### EXAMPLE 81

### 2-({3′-[2-(2-Amino-1-methyl-6-oxo-1,6-dihydropyrimidin-4-yl)ethyl]-1,1′-biphenyl-3-yl}oxy)ethyl acetate

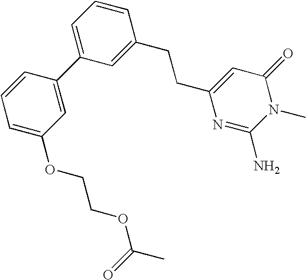

To a solution of 2-amino-6-[2-(3′-hydroxybiphenyl-3-yl)ethyl]-3-methylpyrimidin-4(3H)-one (Example 45) (80 mg, 0.249 mmol), in DMF (2.60 mL) was added K₂CO₃ (0.0344 g, 0.2489 mmol), 2-bromoethyl acetate (0.42 mg, 0.25 mmol) and stirred overnight. Additional K₂CO₃ and 2-bromoethyl acetate (one additional equivalent) were added every 6 h until the starting material was consumed. The mixture was purified by reverse phase HPLC using water/acetonitrile with 0.1% TFA to afford the desired product (24 mg, 0.059 mmol, 24%) as a white solid. ¹H NMR (300. MHz, DMSO) δ 7.48 (mult, 2H), 7.36 (mult, 2H), 7.20 (mult, 3H), 7.06 (s, 2H), 6.95 (mult, 1H), 5.54 (s, 1H), 4.36 (mult, 2H), 4.27 (mult, 2H), 3.23 (s, 3H), 2.94 (t, J=7.8 Hz, 2H), 2.61 (t, J=7.8 Hz, 2H), 2.05 (s, 3H); m/z (APCI) 408 (MH⁺).

### EXAMPLE 82

### 2-Amino-6-{2-[3′-(2-hydroxyethoxy)-1,1′-biphenyl-3-yl]ethyl}-3-methylpyrimidin-4(3H)-one

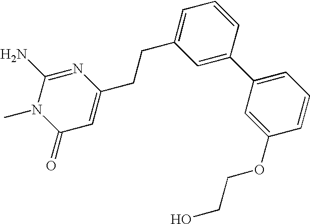

To a solution of 2-({3′-[2-(2-amino-1-methyl-6-oxo-1,6-dihydropyrimidin-4-yl)ethyl]-1,1′-biphenyl-3-yl}oxy)ethyl acetate (Example 81) (10 mg, 0.025 mmol), in CH₃CN (0.200 mL), H₂O (0.200 mL), was added 1N NaOH (0.0246 mL). After 4 h, additional 1N NaOH (0.0140 mL) was added. The mixture was neutralized by addition of 1N AcOH (0.0386 mL), and purified by reverse phase HPLC using water/acetonitrile with 0.1% TFA to afford the desired product (7 mg, 0.019 mmol, 78%) as a white solid. ¹H NMR (300. MHz, DMSO) δ 7.47 (d, J=11.7 Hz, 2H), 7.35 (t, J=7.7 Hz, 2H), 7.18 (mult, 3H), 7.05 (s, 2H), 6.93 (mult, 1H), 5.53 (s, 1H), 4.84 (s, 1H), 4.06 (t, J=5.0 Hz, 2H), 3.74 (s, 2H), 3.22 (s, 3H), 2.94 (t, J=7.8 Hz, 2H), 2.61 (t, J=7.8 Hz, 2H); m/z (APCI) 366 (MH⁺).

### EXAMPLE 83

### 2-Amino-6-{2-[3′-(2-methoxyethoxy)-1,1′-biphenyl-3-yl]ethyl}-3-methylpyrimidin-4(3H)-one

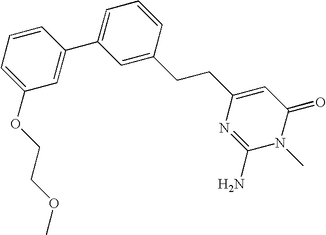

This compound was prepared according to the method described for 2-({3′-[2-(2-amino-1-methyl-6-oxo-1,6-dihydropyrimidin-4-yl)ethyl]-1,1′-biphenyl-3-yl}oxy)ethyl acetate (Example 81) except 2-bromoethyl methyl ether was used in place of 2-bromoethyl acetate to give the desired product (30 mg, 0.079 mmol, 32%) as a white solid. ¹H NMR (300. MHz, DMSO) δ 7.47 (mult, 2H), 7.35 (t, J=7.9 Hz, 2H), 7.18 (mult, 3H), 7.05 (s, 2H), 6.93 (mult, 1H), 5.54 (s, 1H), 4.17 (t, J=4.6 Hz, 2H), 3.69 (t, J=4.6 Hz, 2H), 3.33 (s, 3H), 3.33 (s, 3H), 2.94 (t, J=7.8 Hz, 2H), 2.61 (t, J=7.8 Hz, 2H); m/z (APCI) 380 (MH⁺).

### EXAMPLE 84

### 2-({3′-[2-(2-Amino-1-methyl-6-oxo-1,6-dihydropyrimidin-4-yl)ethyl]-1,1′-biphenyl-4-yl}oxy)ethyl acetate

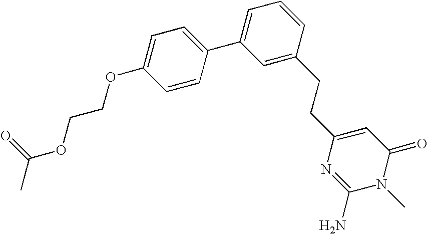

To a solution of 2-amino-6-[2-(4′-hydroxybiphenyl-3-yl)ethyl]-3-methylpyrimidin-4(3H)-one (Example 54) (0.80 g, 0.249 mmol), in DMF (2.60 mL) was added K₂CO₃ (0.0344 g, 0.2489 mmol), 2-bromoethylacetate (0.0416 g, 0.2489 mmol) and stirred overnight. More K₂CO₃ and 2-bromoethyl acetate (1 equiv. every 6 h) were added until the starting material was consumed. The mixture was purified by reverse phase HPLC using water/acetonitrile with 0.1% TFA to afford the desired product (34.0 mg, 0.084 mmol, 34%) as a white solid. ¹H NMR (300. MHz, DMSO) δ 7.57 (d, J=8.6 Hz, 2H), 7.42 (mult, 2H), 7.32 (t, J=7.5 Hz, 2H), 7.15 (d, J=7.4 Hz, 1H), 7.03 (d, J=8.7 Hz, 3H), 5.53 (s, 1H), 4.35 (mult, 2H), 4.23 (mult, 2H), 3.22 (s, 3H), 2.92 (t, J=7.8 Hz, 2H), 2.60 (t, J=7.8 Hz, 2H), 2.05 (s, 3H); m/z (APCI) 408 (MH⁺).

### EXAMPLE 85

### 2-Amino-6-{2-[4′-(2-methoxyethoxy)-1,1′-biphenyl-3-yl]ethyl}-3-methylpyrimidin-4(3H)-one

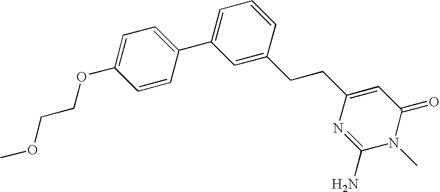

This compound was prepared according to the method described for 2-({3′-[2-(2-amino-1-methyl-6-oxo-1,6-dihydropyrimidin-4-yl)ethyl]-1,1′-biphenyl-4-yl}oxy)ethyl acetate (Example 84) except 2-bromoethyl methyl ether was used in place of 2-bromoethyl acetate to give the desired product (16.0 mg, 0.042 mmol, 17%) as a white solid. ¹H NMR (300. MHz, DMSO) δ 7.56 (d, J=8.6 Hz, 2H), 7.42 (mult, 2H), 7.32 (t, J=7.5 Hz, 2H), 7.14 (d, J=7.4 Hz, 1H), 7.03 (t, J=7.9 Hz, 3H), 5.53 (s, 1H), 4.13 (t, J=4.5 Hz, 2H), 3.68 (t, J=4.5 Hz, 2H), 3.32 (s, 3H), 3.22 (s, 3H), 2.92 (t, J=7.8 Hz, 2H), 2.60 (t, J=7.8 Hz, 2H); m/z (APCI) 380 (MH⁺).

### EXAMPLE 86

### 2-Amino-3-[2-(benzyloxy)ethyl]-6-{2-[3-(2-furyl)phenyl]ethyl}pyrimidin-4(3H)-one

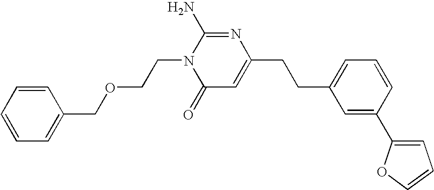

To a solution of 2-amino-6-{2-[3-(2-furyl)phenyl]ethyl}pyrimidin-4(3H)-one (Scheme 6, E) (60 mg, 0.21 mmol) in DMF (2.0 mL), was added K₂CO₃ (30 mg, 0.21 mmol), benzyl-2-bromoethyl ether (46 mg, 0.21 mmol) and the mixture was stirred at room temperature. Additional K₂CO₃ (30 mg, 0.21 mmol), benzyl-2-bromoethyl ether (46 mg, 0.21 mmol) were added and stirring was continued until the starting material was consumed. The mixture was purified by reverse phase HPLC using water/acetonitrile with 0.1% TFA to afford the desired product (14 mg, 0.034 mmol 16%) as a white solid. ¹H NMR (300.132 MHz, DMSO) δ 8.05 (s, 2H), 7.73 (s, 1H), 7.59 (s, 1H), 7.53 (d, J=7.9 Hz, 1H), 7.31 (mult, 6H), 7.17 (d, J=7.6 Hz, 1H), 6.91 (d, J=3.3 Hz, 1H), 6.59 (s, 1H), 5.76 (s, 1H), 4.48 (s, 2H), 4.15 (t, J=5.4 Hz, 2H), 3.62 (t, J=5.3 Hz, 2H), 2.93 (t, J=7.8 Hz, 2H), 2.71 (t, J=7.8 Hz, 2H); m/z (APCI) 416 (MH⁺), HRMS (ES) M⁺, found 416.1959; C₂₅H₂₅N₃O₃ requires 416.1974.

C(═O)N(C₅₋₆heterocyclyl)₂, SO₂C₅₋₆heterocyclyl, SO₂NHC₅₋₆heterocyclyl,
SO₂N(C₅₋₆heterocyclyl)₂, NH(C₅₋₆heterocyclyl), N(C₅₋₆heterocyclyl)₂, NC(═O)C₅₋₆heterocyclyl;

R² is H, C₁₋₆alkyl, C₁₋₆alkyl-C₅₋₁₀aryl;

t is 0 or 1;

R³ is independently selected from H, C₁₋₆alkyl, C₁₋₆alkylR^{c}R^{c}, C₁₋₆alkylNR^{c}R^{c}, C₁₋₆alkylOR^{c}R^{c}, C₁₋₆alkylNC₁₋₆alkylC₅₋₆arylR^{d}, C₁₋₆alkylOC₁₋₆alkylC₅₋₆arylR^{d}, C₁₋₆alkylC-heterocyclyl, C₁₋₆alkylNC₁₋₆alkylC₅₋₉heterocyclylR^{d}, C₁₋₆alkylOC₁₋₆alkylC₅₋₉heterocyclyl, C₁₋₆alkylNC₁₋₆alkylC₃₋₉cycloalkylR^{d}, C₁₋₆alkylOC₁₋₆alkylC₃₋₉cycloalkylR^{d};

R^{c} is independently selected from H, C(═O)C₁₋₄alkyl, C(═O)C₁₋₄alkylOC₁₋₄alkyl, C(═O)C₁₋₄alkylC(═O)OC₁₋₄alkyl, C(═O)C₁₋₄alkylC(═O)OH, C(═O)C₁₋₄alkylOC(═O)C₁₋₄alkyl, C₅₋₆arylR^{d}, C₅₋₉heterocyclylR^{d}, C₃₋₉cycloalkylR^{d}, C(═O)C₅₋₆arylR^{d}, C(═O)C₅₋₉heterocyclylR^{d}, C(═O)C₃₋₉cycloalkyl, C₁₋₄alkyl-C₅₋₆arylR^{d}, C₁₋₄alkyl-C₅₋₉heterocyclylR^{d}, C₁₋₄alkyl-C₃₋₉cycloalkylR^{d};

R^{d} is independently selected from H, C₁₋₃alkyl, NH₂, OH, COOH; OC₁₋₃alkyl, OC₁₋₃alkylOH.

One embodiment of the present invention provides compounds of formula IIa or formula Ib, or a pharmaceutically acceptable salt, tautomer or in vivo-hydrolysable precursors thereof wherein:

Q is C₆₋₁₀aryl or C₅₋₉heterocyclyl;

X is C₁₋₃alkyl;

q is 0 or 1;

m is 0 or 1 or 2;

R¹ is independently selected from H, halogen, optionally substituted C₅₋₁₀aryl, optionally substituted OC₅₋₁₀aryl or optionally substituted C₅₋₁₀heterocyclyl wherein such substituent(s) are independently selected from: halogen, OH, NH₂, CN, C(═O)NH₂, C₁₋₆alkyl, OC₁₋₆alkyl, C₁₋₄alkylOH, C₁₋₄alkylOC₁₋₃alkyl, CH₂OH, SO₂H, SO₂NHC(CH₃)₃, SO₂C₁₋₆alkyl, SO₂NHC₁₋₆alkyl, OC₁₋₃alkylOC₁₋₃alkyl, OC₁₋₃alkylOH, OC₁₋₃alkylOC(═O)C₁₋₃alkyl, C(═O)C₁₋₆alkyl, C(═O)OC₁₋₆alkyl, C(═O)NH₂, C₅₋₆heterocyclyl, OC₅₋₆aryl, OC₁₋₆alkyl-C₅₋₆aryl,

R² is H, C₁₋₆alkyl,

t is 0 or 1;

R³ is independently selected from H, C₁₋₃alkyl.

One embodiment of the present invention provides compounds of formula IIa or formula Ib, or a pharmaceutically acceptable salt, tautomer or in vivo-hydrolysable precursors wherein Q is C₅₋₆aryl and R¹, X, m, q, t, R², R³, R^{c} and R^{d} have any of the meanings defined hereinabove.

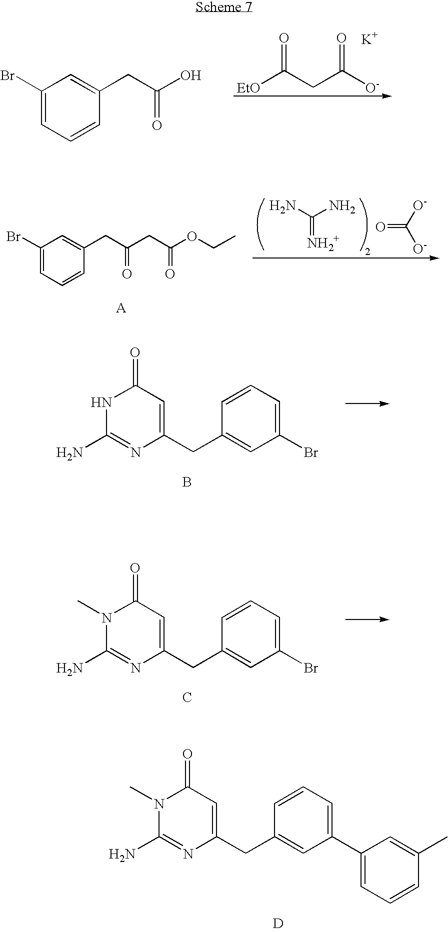

### EXAMPLE: 87

### 2-Amino-3-methyl-6-(3′-methyl-biphenyl-3-ylmethyl)-3H-pyrimidin-4-one (Scheme 7, D)

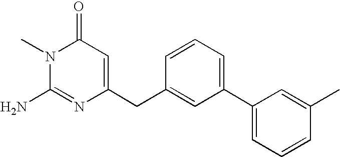

A thick-walled glass vial was charged with a stir bar, 2-Amino-6-(3-bromo-benzyl)-3-methyl-3H-pyrimidin-4-one (Scheme 7, C) (60 mg, 0.2 mmol), 3-methylphenylboronic acid (26 mg, 0.19 mmol), dichlorobis(triphenylphosphine)-palladium (II) (2.7 mg, 0.003 mmol), Cs₂CO₃ (123 mg, 0.38 mmol) and DME/H₂O/EtOH (7:3:2-ca. 5 mL). Crimp sealed and subject to microwave radiation for 5 min. at 150° C. The resultant black slurry was filtered through Celite and a 0.7 μm GMF filter, washing with MeOH (3×3 mL) then concentrated in vacuo. The resultant residue was subject to RP-HPLC purification (t_{R}=11.1 min). Appropriate fractions were concentrated via centrifugal evaporation to afford the white trifluoroacetic acid salt of the title compound (62 mg, 78%). ¹H NMR (300 MHz, MeOH-d₄) δ 2.40 (s, 3H), 3.39 (s, 3H), 3.91 (s, 2H), 5.83 (s, 1H), 7.17 (d, J=7.4 Hz, 1H), 7.26-7.33 (m, 2H), 7.38-7.46 (m, 3H), 7.52-7.57 (m, 2H); m/z (APCI+) M+1=306.2; LCMS t_{R}=1.81 min.

### 2-Amino-6-(3-bromo-benzyl)-3-methyl-3H-pyrimidin-4-one (Scheme 7, C) was Prepared as Follows

### 4-(3-Bromophenyl)-3-oxo-butyric acid ethyl ester (Scheme 7, A)

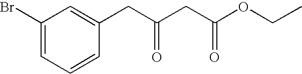

To a stirring suspension of potassium malonate (7.42 g, 43.6 mmol) in anhydrous acetonitrile (100 mL) at ambient temperature was added triethylamine (9.0 mL, 64.4 mmol) and magnesium chloride (4.94 g, 51.9 mmol) under an argon atmosphere. Stirring was continued for 3 hours before the rapid addition of the 2-(3-bromophenyl)ethanoic imidazolide in the same solvent (60 mL), prepared 20 min. prior by reaction between 3-bromophenylacetic acid (4.47 g, 20.8 mmol) and 1,1′-carbonyldiimidazole (4.04 g, 24.9 mmol) in dry acetonitrile (60 mL). The reaction mixture was allowed to stir for 17 hours at room temperature, followed by heating to reflux for 1.5 hrs before quenching by the slow addition of ca. 13% aqueous HCl (100 mL) at 5° C. The clear biphasic mixture was separated, wherein the organic layer was concentrated by rotary evaporation to a residue and treated with ethyl acetate (80 mL) while the aqueous remains were further extracted into ethyl acetate (2×50 mL). The combined organic extracts were washed with a saturated sodium carbonate aqueous solution (2×80 mL) and brine (1×50 mL), dried over MgSO₄, then concentrated in vacuo to afford the desired 4-(3-bromophenyl)-3-oxo-butyric acid ethyl ester (Scheme 7, A) as a clear yellow oil (5.93 g, quant). ¹H NMR (300 MHz, CDCl₃) δ 1.28 (t, J=7.1 Hz, 3H), 3.53 (s, 2H), 3.88 (s, 2H), 4.19 (q, J=7.1 Hz, 2H), 7.13-7.26 (m, 2H), 7.37-7.44 (m, 2H); m/z (ES+) M+1=285.0; LCMS t_{R}=2.52 min.

### 2-Amino-6-(3-bromo-benzyl)-3H-pyrimidin-4-one (Scheme 7, B)

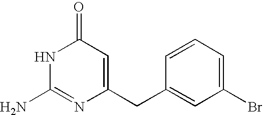

To a solution of 4-(3-bromophenyl)-3-oxo-butyric acid ethyl ester (Scheme 7, A) (5.93 g, 20.8 mmol) in ethanol (60 mL) was added guanidine carbonate (2.06 g, 11.4 mmol) and the reaction heated under reflux for 16 hours. Upon concentration by rotary evaporation to ca. ½ volume and cooling, the resulting solid was collected by filtration and washed with cold ethanol (3×10 mL). The precipitate was dried under high vacuum at 30° C. over night to afford the title compound as a white solid (4.8 g, 83%). ¹H NMR (300 MHz, DMSO-d₆) δ 3.63 (s, 2H), 5.49 (s, 1H), 6.47 (s, 2H), 7.25-7.29 (m, 2H), 7.40-7.43 (m, 1H), 7.45 (s, 1H), 10.61 (s, 1H); m/z (ES+) M+1=280.0 LCMS t_{R}=1.28 min.

### Amino-6-(3-bromo-benzyl)-3-methyl-3H-pyrimidin-4-one (Scheme 7, C)

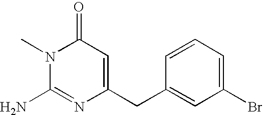

To a suspension of 2-Amino-6-(3-bromo-benzyl)-3H-pyrimidin-4-one (Scheme 7, B) (1.63 g, 5.83 mmol) in absolute ethanol (35 mL) was added solid potassium hydroxide (589 mg, 10.5 mmol), which was stirred until a homogeneous solution was achieved. Iodomethane (1.31 mL, 20.9 mmol) was added in one portion and the reaction heated in a sealed tube to 78° C. for 17 hours. Upon completion, was concentrated in vacuo to a pale yellow residue and subject to flash chromatography (SiO₂-40 g; gradient elution: 0.5% MeOH/CH₂Cl₂ for 3 min, then 0.5-5% MeOH/CH₂Cl₂ over 24 min at 60 mL/min) to provide the 2-Amino-6-(3-bromo-benzyl)-3-methyl-3H-pyrimidin-4-one (Scheme 7, C) as a white solid (1.3 g, 76%). ¹H NMR (300 MHz, DMSO-d₆) δ 3.20 (s, 3H), 3.58 (s, 2H), 5.52 (s, 1H), 7.07 (s, 2H), 7.26 (m, 2H), 7.41 (m, 1H), 7.46 (s, 1H); m/z (ES+) M+1=294.0 LCMS t_{R}=1.39 min.

The following compounds were synthesized using methods analogous to those previously described for Example 87 employing the appropriate boronic acid with the precursor aryl bromide; Scheme 7, C.

TABLE 5

| | | | | | LCMS |
|---|---|---|---|---|---|
| | Compound | | | m/z | t_{R} |
| Ex. | Namw | Structure | NMR | M + 1 | (min) |
| 88 | 2-Amino-6-(3′-methoxy-biphenyl-3-ylmethyl)-3-methyl-3H-pyrimidin-4-one | 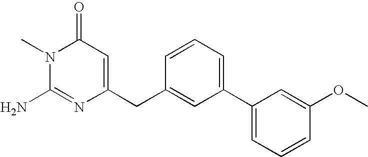 | 1H NMR (300 MHz,DMSO-d₆/TFA-d) δ 3.27 (s,3 H), 3.83 (s, 3 H), 3.93 (s,2 H), 5.98 (s, 1 H), 6.96 (d,J = 8.0 Hz, 1 H), 7.20-7.25(m, 2 H), 7.36-7.48 (m,3 H), 7.61 (d, J = 7.4 Hz,1 H), 7.69 (s, 1 H). | 322.1(ES+) | 1.73 |
| 89 | 2-Amino-6-(3′-ethoxy-biphenyl-3-ylmethyl)-3-methyl-3H-pyrimidin-4-one | 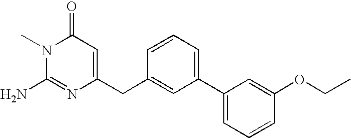 | 1H NMR (300. MHz,MeOH-d₄) δ 1.40 (t,J = 7.0 Hz, 4 H), 3.39 (s,5 H), 3.90 (s, 3 H), 4.07 (q,J = 7.0 Hz, 3 H), 5.83 (s,1 H), 6.97 (d, J = 8.7 Hz,3 H), 7.23 (d, J = 7.7 Hz,1 H), 7.40 (t, J = 7.7 Hz,2 H), 7.40 (t, J = 7.7 Hz,2 H), 7.52-7.67 (m, 7 H). | 336.1(ES+) | 1.80 |
| 90 | 3′-(2-Amino-1-methyl-6-oxo-1,6-dihydro-pyrimidin-4-ylmethyl)-biphenyl-3-carbonitrile | 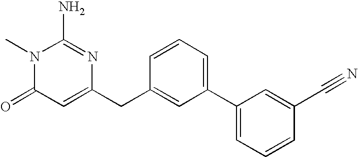 | 1H NMR (300 MHz,MeOH-d₄) δ 3.41 (s, 3 H),3.98 (s, 2 H), 5.86 (s, 1 H),7.40 (d, J = 7.6 Hz, 1 H),7.53 (t, J = 8.0 Hz, 1 H),7.63-7.75 (m, 4 H), 7.95-8.00 (m, 2 H). | 317.1(ES+) | 1.64 |
| 91 | 2-Amino-3-methyl-6-(3′-vinyl-biphenyl-3-ylmethyl)-3H-pyrimidin-4-one | 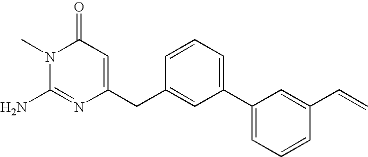 | 1H NMR (300 MHz,DMSO-d₆/TFA-d) δ 3.27 (s,3 H), 3.93 (s, 2 H), 5.33 (d,J = 11.0 Hz, 1 H), 5.93 (d,J = 17.7 Hz, 1 H), 5.98 (s,1 H), 6.83 (dd, J = 17.7,11.0 Hz, 1 H), 7.36-7.52 (m,4 H), 7.59 (d, J = 16.8 Hz,1 H), 7.62 (d, J = 17.2 Hz,1 H), 7.72-7.73 (m, 2 H). | 318.2(APCI+) | 2.17 |
| 92 | 2-Amino-6-(3′-isopropyl-biphenyl-3-ylmethyl)-3-methyl-3H-pyrimidin-4-one | 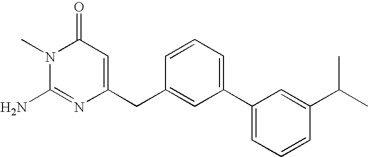 | 1H NMR (300 MHz,DMSO-d₆/TFA-d) δ 1.27 (d,J = 6.9 Hz, 6 H), 2.98(quintet, J = 6.9 Hz, 1 H),3.28 (s, 3 H), 3.93 (s, 2 H),5.98 (s, 1 H), 7.26 (d,J = 7.5 Hz, 1 H), 7.35-7.51(m, 3 H), 7.58-7.61 (m, 2 H),7.65 (d, J = 4.2 Hz, 1 H),7.68 (s, 1 H). | 334.1(APCI+) | 2.33 |
| 93 | 2-Amino-6-(4′-methoxy-biphenyl-3-ylmethyl)-3-methyl-3H-pyrimidin-4-one | 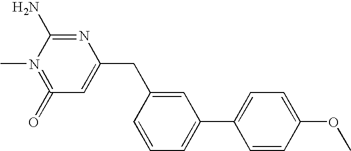 | 1H NMR (300 MHz,DMSO-d₆) δ 3.22 (s, 3 H),3.76 (s, 2 H), 3.79 (s, 3 H),5.75 (s, 1 H), 7.02 (d,J = 8.7 Hz, 2 H), 7.23 (d,J = 7.7 Hz, 1 H), 7.38 (t,J = 7.6 Hz, 1 H), 7.49 (d,J = 7.7 Hz, 1 H),7.55-7.60 (m, 3 H), 7.92 (s,2 H). | 322.1(ES+) | 1.71 |

### EXAMPLE 94

### 2-amino-6-[2-[3-(3-methoxyphenyl)phenyl]ethyl]-3-methyl-6-phenyl-5,6-dihydro-3H-pyrimidin-4-one

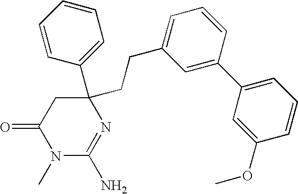

Prepared according to scheme 2 using the ketone prepared using standard Weinreb amide displacements with the appropriate organometallic according to Nahm, et al, Tet. Lett., 1981, 22, 3815-3818. ¹H NMR (300 MHz, DMSO-d6/TFA-d) δ 2.27 (m, 2H), 2.47 (m, 1H), 2.65 (m, 1H), 3.09 (s, 3H), 3.43 (dd, J=36.4, 16.3 Hz, 2H), 3.83 (s, 3H), 6.94 (dd, J=8.0, 2.2 Hz, 1H), 7.18 (m, 3H), 7.36 (m, 3H), 7.47 (m, 6H); m/z (APCI+) M+1 (414); LCMS t_{R}=2.37 min.

### EXAMPLE 95

### 2-amino-6-[3-(3-methoxyphenyl)phenyl]-3-methyl-6-(trifluoromethyl)-5,6-dihydro-3H-pyrimidinone

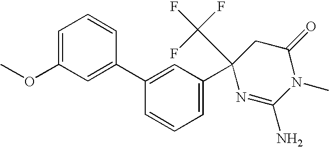

Prepared according to scheme 2 using the ketone prepared according to the method of Kogon et al, Leibigs Ann. Chem., 1992, 8, 879-882 using NBS as the brominating agent. ¹H NMR (300 MHz, DMSO-d6/TFA-d) δ 3.18 (s, 3H), 3.75 (d, J=16.6 Hz, 1H), 3.85 (s, 3H), 4.02 (d, J=16.6 Hz, 1H), 7.01 (dd, J=7.9, 2.1 Hz, 1H), 7.24-7.30 (m, 2H), 7.43 (t, J=7.9 Hz, 1H), 7.61-7.67 (m, 2H), 7.80-7.87 (m, 1H), 7.93 (s, 1H); m/z (APCI+) M+1 (378); LCMS t_{R}=2.05 min.

### EXAMPLE 96

### (R)-2-Amino-6-[2-(3′-methoxy-biphenyl-3-yl)-ethyl]-3,6-dimethyl-5,6-dihydro-3H-pyrimidin-4-one

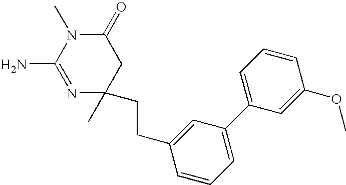

Racemic 2-Amino-6-[2-(3′-methoxy-biphenyl-3-yl)-ethyl]-3,6-dimethyl-5,6-dihydro-3H-pyrimidin-4-one, (the title compound for Example 25), 165 mg, prepared according to Scheme 2, was dissolved in methanol and enantiomers separated on a preparative supercritical fluid chromatography (prep SFC) system (Ret. time: 3.78 mins) using the following conditions: 21×250 mm ChiralPak AS-H 5 micron column, 50.0 mL/min, 15:85 (methanol containing 0.5% dimethylethylamine): supercritical carbon dioxide, UV-260 nm. The solvent was removed from the product fractions on a Genevac evaporator to give a waxy solid. This solid was then purified on an Agilent RP-HPLC (Ret. time: 15.58 mins). The combined purified fractions were lyophilized to give the title compound as a white powder (0.036 g, 22%). ¹H NMR (300 MHz, DMSO) δ 1.36 (s, 3H), 1.93 (t, J=8.5 Hz, 2H), 2.71 (t, J=14.7 Hz, 2H), 2.82 (d, J=16.4 Hz, 1H), 2.97 (d, J=16.4 Hz, 1H), 3.20 (s, 3H), 3.84 (s, 3H), 6.95 (dd, J=8.1, 2.0 Hz, 1H), 7.18 (d, J=2.2 Hz, 1H), 7.23 (t, J=6.9 Hz, 2H), 7.38 (t, J=7.9 Hz, 2H), 7.51 (t, J=7.7 Hz, 2H), m/z (APCI+) M+1 (352); LC RT 1.98 min, analytical chiral SCF RT 4.36 min >99% ee conditions: 4.6×250 mm ChiralPak AS-H 5 micron column, 2.20 mL/min, 15:85 (methanol containing 0.5% dimethylethylamine): supercritical carbon dioxide, UV-260 nm.

The following compounds were synthesized using the methods analogous to those previously described for Examples 94 and 95 employing the appropriate starting materials.

TABLE 6

| | | | | m/z | LC |
|---|---|---|---|---|---|
| | | | | M + 1 | t_{R} |
| Ex. | Compound | Structure | NMR | (Ionization) | (min) |
| 97 | 2-Amino-6-[2-(2′-fluoro-3′-methoxy-biphenyl-3-yl)-ethyl]-3,6-dimethyl-5,6-dihydro-3H-pyrimidin-4-one | 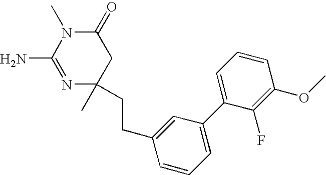 | ¹H NMR (300 MHz,DMSO-d6-/TFA-d): δ 1.35(s, 3 H), 1.92 (t,J = 8.5 Hz, 2 H), 2.65-2.74(m, 2 H), 2.81 (d,J = 16.4 Hz, 1 H),2.96 (d, J = 16.4 Hz, 1 H),3.20 (s, 3 H), 3.89 (s, 3 H),6.99-7.07 (m, 1 H), 7.13-7.31(m, 3 H), 7.33-7.44 (m, 3 H) | 370(APCI+) | 1.90 |
| 98 | 2-Amino-6-{2-[3-(5-chloro-thiophen-2-yl)-phenyl]-ethyl}-3,6-dimethyl-5,6-dihydro-3H-pyrimidin-4-one | 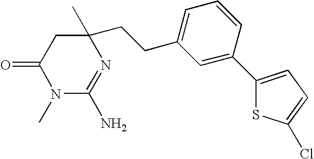 | ¹H NMR (300 MHz,DMSO-d6-/TFA-d): δ 1.36(s, 3 H), 1.92 (t, J = 14.9 Hz,2 H), 2.68 (t, J = 16.8 Hz,2 H), 2.82 (d, J = 16.4 Hz,1 H), 2.97 (d, J = 16.4 Hz,1 H), 3.21 (s, 3 H), 7.14 (d,J = 3.9 Hz, 1 H), 7.22 (d,J = 7.6 Hz, 1 H), 7.31-7.39(m, 2 H), 7.42-7.51 (m, 2 H) | 362(ES+) | 1.97 |
| 99 | 2-Amino-6-[2-(3-furan-2-yl-phenyl)-ethyl]-3,6-dimethyl-5,6-dihydro-3H-pyrimidin-4-one | 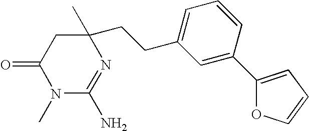 | ¹H NMR (300 MHz,DMSO-d6-/TFA-d): δ 1.36(s, 3 H), 1.92 (t, J = 14.4 Hz,2 H), 2.68 (t, J = 13.9 Hz,2 H), 2.82 (d, J = 16.4 Hz,1 H), 2.97 (d, J = 16.4 Hz,1 H), 3.21 (s, 3 H), 6.59 (dd,J = 3.3, 1.8 Hz, 1 H), 6.91(d, J = 3.3 Hz, 1 H), 7.17 (d,J = 7.7 Hz, 1 H), 7.35 (t,J = 7.7 Hz, 1 H), 7.55 (d,J = 7.9 Hz, 1 H), 7.60 (s,1 H), 7.72 (d, J = 1.1 Hz,1 H) | 312(ES+) | 1.71 |
| 100 | 2-Amino-6-[2-(3′,5′-dimethoxy-biphenyl-3-yl)-ethyl]-3,6-dimethyl-5,6-dihydro-3H-pyrimidin-4-one | 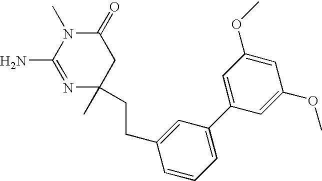 | 1H NMR (300 MHz,DMSO-d6-/TFA-d): δ 1.36(s, 3 H), 1.93 (t, J = 8.5 Hz,2 H), 2.66-2.75 (m, 2 H), 2.82(d, J = 16.4 Hz, 1 H), 2.97 (d,J = 16.4 Hz, 1 H), 3.21(s, 3 H), 3.82 (s, 6 H), 6.52(t, J = 2.1 Hz, 1 H), 6.78 (d,J = 2.2 Hz, 2 H), 7.24 (d,J = 7.6 Hz, 1 H), 7.37 (t,J = 7.6 Hz, 1 H), 7.45-7.55(m, 2 H) | 382(APCI+) | 1.94 |
| 101 | 2-Amino-6-[2-(4′-methoxy-biphenyl-3-yl)-ethyl]-3,6-dimethyl-5,6-dihydro-3H-pyrimidin-4-one | 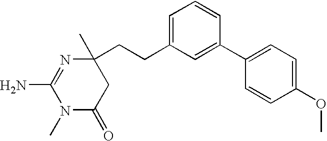 | ¹H NMR (300 MHz, DMSO-d6-/TFA-d): δ 1.36 (s, 3 H),1.93 (t, J = 8.5 Hz, 2 H), 2.69(m, 2 H), 2.82 (d,J = 16.4 Hz, 1 H), 2.97(d, J = 16.4 Hz, 1 H), 3.20 (s,3 H), 3.81 (s, 3 H), 7.03 (d,J = 8.8 Hz, 2 H), 7.18 (d,J = 7.6 Hz,1 H), 7.35 (t, J = 7.6 Hz,1 H), 7.46 (m, 2 H), 7.59 (d,J = 8.8 Hz,2 H) | 352(APCI+) | 1.97 |
| 102 | 2-Amino-6-(2-biphenyl-3-yl-ethyl)-3,6-dimethyl-5,6-dihydro-3H-pyrimidin-4-one | 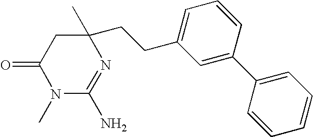 | 1H NMR (300 MHz, DMSO-d6-/TFA-d): δ 1.37 (s, 3 H),1.94 (t, J = 8.5 Hz, 2 H),2.72 (t, J = 13.9 Hz, 2 H),2.82 (d, J = 16.4 Hz,1 H), 2.98 (d, J = 16.4 Hz,1 H), 3.21 (s, 3 H), 7.25 (d,J = 7.5 Hz, 1 H), 7.33-7.52(m, 5 H), 7.55 (s, 1 H), 7.66(d, J = 21.2 Hz, 2 H) | 322(ES+) | 1.82 |
| 103 | 2-Amino-3-benzyl-6-[2-(3′-methoxy-biphenyl-3-yl)-ethyl]-6-methyl-5,6-dihydro-3H-pyrimidin-4-one | 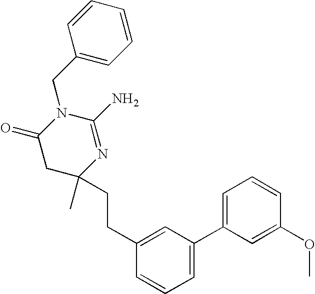 | ¹H NMR (300 MHz, DMSO-d6-/TFA-d): δ 1.37 (s, 3 H), 1.82-1.92 (m, 2 H), 2.56-2.77 (m,2 H), 3.02 (dd, J = 28.0, 16.2 Hz,2 H), 3.84 (s, 3 H), 5.09 (dd,J = 21.1, 16.6 Hz, 2 H), 6.96 (dd,J = 8.1, 2.0 Hz, 1 H), 7.10-7.33 (m,8 H), 7.34-7.43 (m, 2 H), 7.43-7.53 (m, 2 H) | 428(APCI+) | 2.34 |
| 104 | N-{3′-[2-(2-Amino-1,4-dimethyl-6-oxo-1,4,5,6-tetrahydro-pyrimidin-4-yl)-ethyl]-biphenyl-3-yl}-acetamide | 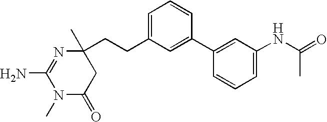 | ¹H NMR (300 MHz, DMSO-d6-/TFA-d): δ 1.36 (s, 3 H), 1.93 (t,J = 8.5 Hz, 1 H), 2.08 (s, 3 H),2.70 (m, 2 H), 2.82 (d,J = 16.4 Hz, 1 H), 2.97 (d, J = 16.4 Hz,1 H), 3.20 (s, 3 H), 7.21-7.50(m, 7 H), 7.55 (d, J = 7.9 Hz, 1 H) | 379(ES+) | 1.72 |
| 105 | 2-Amino-6-[2-(3′-hydroxymethyl-biphenyl-3-yl)-ethyl]-3,6-dimethyl-5,6-dihydro-3H-pyrimidin-4-one | 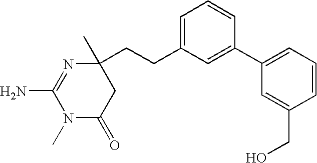 | ¹H NMR (300 MHz, DMSO-d6-/TFA-d): δ 1.37 (s, 3 H), 1.94 (t,J = 8.5 Hz, 2 H), 2.71 (t,J = 16.7 Hz, 2 H), 2.82 (d, J = 16.4 Hz,1 H), 2.98 (d, J = 16.4 Hz, 1 H),3.21 (s, 3 H), 4.59 (s, 2 H), 7.24(d, J = 7.5 Hz, 1 H), 7.30-7.58(m, 6 H), 7.61 (s, 1 H) | 352(ES+) | 1.50 |
| 106 | 2-Amino-6-methyl-6-naphthalen-2-yl-5,6-dihydro-3H-pyrimidin-4-one | 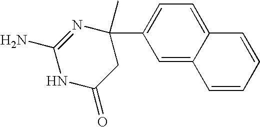 | ¹H NMR (300 MHz, DMSO-d6-/TFA-d): δ 1.76 (s, 3 H), 3.22 (d,J = 16.5 Hz, 1 H), 3.46 (d,J = 16.5 Hz, 1 H), 7.49-7.68 (m,3 H), 7.84-8.04 (m, 4 H) | 254(APCI+) | 1.48 |
| 107 | 2-Amino-3-benzyl-6-[2-(3-bromo-phenyl)-ethyl]-6-methyl-5,6-dihydro-3H-pyrimidin-4-one | 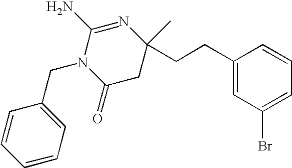 | ¹H NMR (300 MHz, DMSO-d6-/TFA-d): δ 1.34 (s, 3 H), 1.76-1.85 (m, 2 H), 2.55-2.68 (m,2 H), 2.99 (dd, J = 27.2, 16.2 Hz,2 H), 5.08 (s, 2 H), 7.14 (d,J = 7.9 Hz, 1 H), 7.19-7.45 (m, 8 H) | 400(APCI+) | 2.14 |
| 108 | 2-Amino-6-[2-(4′-methanesulfonyl-biphenyl-3-yl)-ethyl]-3,6-dimethyl-5,6-dihydro-3H-pyrimidin-4-one | 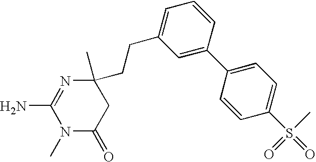 | ¹H NMR (300 MHz, DMSO-d6-/TFA-d): δ 1.36 (s, 3 H), 1.94 (t,J = 14.6 Hz, 2 H), 2.73 (m, 2 H),2.82 (d, J = 16.4 Hz, 1 H), 2.97(d, J = 16.4 Hz, 1 H), 3.20 (s,3 H), 3.25 (s, 3 H), 7.33 (d,J = 7.6 Hz, 1 H), 7.45 (t, J = 7.6 Hz,1 H), 7.59 (d, J = 7.8 Hz, 1 H),7.63 (s, 1 H), 7.93 (d, J = 8.5 Hz,2 H), 8.02 (d, J = 8.5 Hz, 2 H) | 400(APCI+) | 1.75 |
| 109 | 2-Amino-6-[2-(3′,4′-dimethoxy-biphenyl-3-yl)-ethyl]-3,6-dimethyl-5,6-dihydro-3H-pyrimidin-4-one | 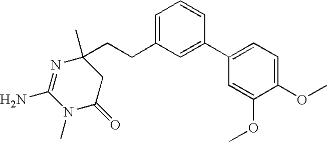 | ¹H NMR (300 MHz, DMSO-d6-/TFA-d): δ 1.36 (s, 3 H), 1.92 (t,J = 8.4 Hz, 2 H), 2.70 (m, 2 H),2.82 (d, J = 16.4 Hz, 1 H), 2.97(d, J = 16.4 Hz, 1 H), 3.20 (s,3 H), 3.81 (s, 3 H), 3.86 (s, 3 H),7.03 (d, J = 8.0 Hz, 1 H), 7.19 (d,J = 7.8 Hz, 3 H), 7.35 (t,J = 7.5 Hz, 1 H), 7.47 (d, J = 9.0 Hz,2 H), 7.47 (d, J = 9.0 Hz, 12 H) | 382(APCI+) | 1.87 |
| 110 | 3′-[2-(2-Amino-1,4-dimethyl-6-oxo-1,4,5,6-tetrahydro-pyrimidin-4-yl)-ethyl]-biphenyl-4-sulfonicacid dimethylamide | 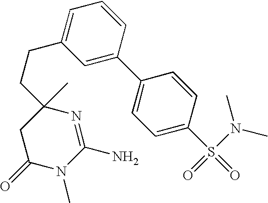 | ¹H NMR (300 MHz, DMSO-d6-/TFA-d): δ 1.36 (s, 3 H), 1.94 (t,J = 8.6 Hz, 2 H), 2.82 (d,J = 16.4 Hz, 1 H), 2.97 (d, J = 16.4 Hz,1 H), 3.20 (s, 3 H), 7.33 (d,J = 7.6 Hz, 1 H), 7.45 (t, J = 7.6 Hz,1 H), 7.61 (m, 2 H), 7.83 (d,J = 8.5 Hz, 2 H), 7.93 (d, J = 8.5 Hz,2 H), 2.63-2.77 (m, 8 H) | 429(APCI+) | 1.92 |
| 111 | 2-Amino-6-(2′-fluoro-3′-methoxy-biphenyl-3-yl)-3,6-dimethyl-5,6-dihydro-3H-pyrimidin-4-one | 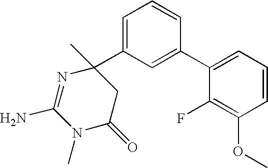 | ¹H NMR (300 MHz, DMSO-d6-TFA-d): δ 1.68 (s, 3 H), 3.11 (s,3 H), 3.21 (d, J = 16.4 Hz, 1 H),3.51 (d, J = 16.4 Hz, 1 H), 3.90(s, 3 H), 7.01-7.10 (m, 1 H),7.14-7.28 (m, 2 H), 7.43-7.59(m, 4 H) | 342(APCI+) | 1.88 |
| 112 | 2-Amino-6-(3′,5′-dimethoxy-biphenyl-3-yl)-3,6-dimethyl-5,6-dihydro-3H-pyrimidin-4-one | 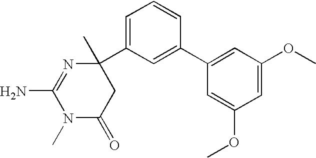 | ¹H NMR (300 MHz, DMSO-d6-/TFA-d): δ 1.69 (s, 3 H), 3.11 (s,3 H), 3.21 (d, J = 16.4 Hz, 1 H),3.58 (d, J = 16.4 Hz, 1 H), 3.83(s, 6 H), 6.56 (t, J = 2.1 Hz, 1 H),6.80 (d, J = 2.2 Hz, 2 H), 7.40 (d,J = 8.0 Hz, 1 H), 7.49 (t,J = 7.7 Hz, 1 H), 7.61-7.71 (m, 2 H) | 354(APCI+) | 1.90 |

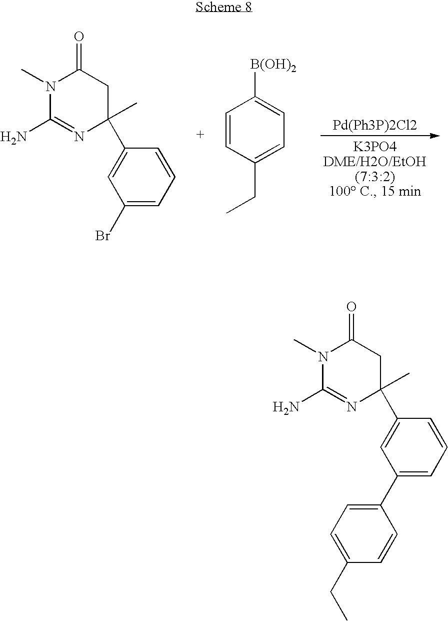

### EXAMPLE 113

### 2-Amino-6-(4′-ethylbiphenyl-3-yl)-3,6-dimethyl-5,6-dihydropyrimidin-4(3B)-one

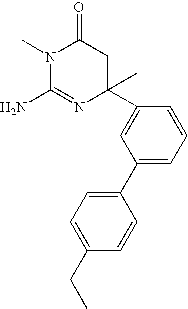

The title compound was prepared according to the method shown in Scheme 8. A solution of 2-amino-6-(3-bromophenyl)-3,6-dimethyl-5,6-dihydropyrimidin-4(3H)-one (50 mg, 0.169 mmol) in 2 mL of 1,2-dimethoxyethane/water/ethanol (7:3:2 v/v) was added to a mixture of 4-ethylphenylboronic acid (51 mg, 0.338 mmol), potassium phosphate (90 mg, 0.39 mmol) and bis(triphenylphosphine)palladium dichloride (12 mg, 0.017 mmol). The resulting mixture was heated for 15 minutes at 100° C. then allowed to cool to room temperature. Si-TAAcOH resin, (Silicycle, 100 mg) was added as a palladium scavenger and the resulting mixture stirred for 1 hour then filtered. The volatiles were evaporated on a Genevac HT-4 and the residue purified by mass-directed LCMS. [Waters Exterra column, 30×100 mm, 5μ, eluting with 12-88% acetonitrile/water buffered with 2.5 mM ammonium carbonate at 52 mL/min] to yield the title compound as a off-white solid. (23 mg, 43%). ¹H NMR (500.132 MHz, DMSO-d₆) δ 7.66 (s, 1H), 7.62-7.56 (m, 3H), 7.49 (t, J=7.7 Hz, 1H), 7.37-7.31 (m, 3H), 3.55 (d, J=16.3 Hz, 1H), 3.19 (d, J=16.3 Hz, 1H), 3.09 (s, 3H), 2.66 (q, J=7.6 Hz, 2H), 1.67 (s, 3H), 1.21 (t, J=7.6 Hz, 3H).

The following compounds in Table 7 were synthesized using the method analogous to that previously described for Example 113 employing the appropriate available boronic acid.

TABLE 7

| | | | | m/z | LC |
|---|---|---|---|---|---|
| | | | | M + 1 | t_{R} |
| Ex. | Compound | | NMR | (Ionization) | (min) |
| 114 | 2-amino-6-(2′-ethyl-biphenyl-3-yl)-3,6-dimethyl-5,6-dihydro-pyrimidin-4(3H)-one | 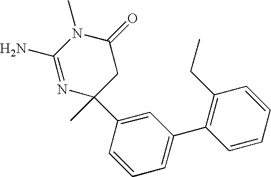 | ¹H NMR (500 MHz, DMSO-d6)δ 8.13 (s, 1 H), 7.49 (t,J = 8.2 Hz, 1 H), 7.40 (d,J = 7.7 Hz, 1 H), 7.36-7.32(m, 3 H), 7.29-7.24 (m, 2 H),7.15 (d, J = 7.5 Hz, 1 H), 6.51(s, 1 H), 3.49 (d, J = 17.2 Hz,1 H), 3.18 (d, J = 17.2 Hz,1 H), 3.07 (s, 3 H), 2.52-2.47(m, 2 H), 1.64 (s, 3 H), 0.99(t, J = 7.3 Hz, 3 H) | 321 | 6.04 |
| 115 | 2-amino-6-(3-isoquinolin-5-ylphenyl)-3,6-dimethyl-5,6-dihydropyrimidin-4(3H)-one | 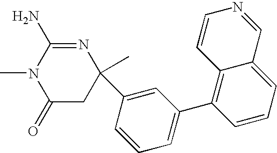 | ¹H NMR (500 MHz, DMSO-d6)δ 9.43 (s, 1 H), 8.51(d, J = 6.1 Hz, 1 H), 8.21(d, J = 7.9 Hz, 1 H), 8.13 (s,1 H), 7.80 (t, J = 7.6 Hz, 1 H),7.75 (d, J = 6.2 Hz, 1 H),7.64-7.59 (m, 1 H), 7.57-7.53(m, 3 H), 7.50 (d, J = 7.5 Hz,1 H), 3.32-3.27 (m, 2 H), 3.12(s, 3 H), 1.68 (s, 3 H) | 345 | 3.56 |
| 116 | 2-amino-3,6-dimethyl-6-[3′-(1H-pyrazol-1-yl)biphenyl-3-yl]-5,6-dihydropyrimidin-4(3H)-one | 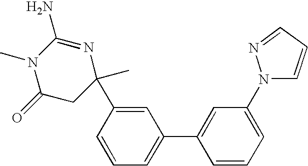 | ¹H NMR (500 MHz, DMSO-d6)δ 8.63 (d, J = 2.3 Hz,1 H), 8.09 (s, 1 H), 7.90-7.87(m, 1 H), 7.81-7.73 (m, 3 H),7.64-7.59 (m, 2 H), 7.56 (t,J = 7.6 Hz, 1 H), 7.44 (d,J = 7.9 Hz, 1 H), 6.61-6.59(m, 1 H), 3.59 (d,J = 16.4 Hz, 1 H), 3.32(s, 3 H), 3.21 (d, J = 16.2 Hz,1 H), 3.10 (s, 2 H), 1.70 (s,3 H) | 359 | 5.37 |
| 117 | 2-amino-6-(4′-isopropoxy-biphenyl-3-yl)-3,6-dimethyl-5,6-dihydropyrimidin-4(3H)-one | 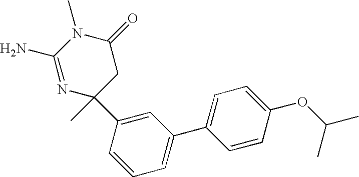 | ¹H NMR (500 MHz, DMSO-d6)δ 7.63 (s, 1 H), 7.58 (d,J = 8.2 Hz, 2 H), 7.56 (s,1 H), 7.46 (t, J = 7.7 Hz,1 H), 7.31 (d, J = 8.2 Hz,1 H), 7.02 (d, J = 8.7 Hz,2 H), 4.67 (septet,J = 5.1 Hz, 1 H), 3.55 (d,J = 15.7 Hz, 1 H), 3.18 (d,J = 16.5 Hz, 1 H), 3.08 (s,3 H), 1.66 (s, 3 H), 1.29 (d,J = 6.0 Hz, 6 H) | 351 | 6.1 |
| 118 | 2-amino-6-(3′-ethoxy-biphenyl-3-yl)-3,6-dimethyl-5,6-dihydro-pyrimidin-4(3H)-one | 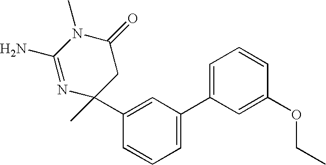 | ¹H NMR (500 MHz, DMSO-d6)δ 7.68 (s, 1 H), 7.63 (d,J = 6.6 Hz, 1 H), 7.49 (t,J = 7.7 Hz, 1 H), 7.42-7.35(m, 2 H), 7.21 (d, J = 8.7 Hz,1 H), 7.18 (s, 1 H), 6.96 (d,J = 8.7 Hz, 1 H), 4.10 (q,J = 6.4 Hz, 2 H), 3.56 (d,J = 16.6 Hz, 1 H), 3.18 (d,J = 16.6 Hz, 1 H), 3.08 (s,3 H), 3.08 (s, 2 H), 1.66 (s,3 H), 1.36 (t, J = 6.6 Hz, 3 H) | 337 | 5.82 |
| 119 | 2-amino-3,6-dimethyl-6-[2′-(trifluoro-methyl)biphenyl-3-yl]-5,6-dihydropyrimidin-4(3H)-one | 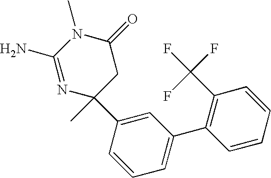 | ¹H NMR (500.132 MHz,DMSO)δ 7.85 (d, J = 7.6 Hz,1 H), 7.74 (t, J = 7.0 Hz,1 H), 7.64 (t, J = 7.6 Hz,1 H), 7.52-7.43 (m, 2 H),7.41 (d, J = 7.6 Hz, 1 H),7.33-7.24 (m, 2 H), 3.05 (s,3 H), 1.61 (s, 3 H) | 361 | 5.82 |
| 120 | 2-amino-6-(2′-chloro-biphenyl-3-yl)-3,6-dimethyl-5,6-dihydro-pyrimidin-4(3H)-one | 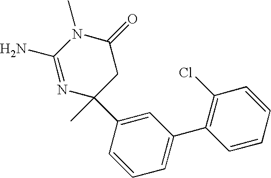 | ¹H NMR (500.132 MHz,DMSO)δ 7.66-7.38 (m, 8 H),3.08 (s, 3 H), 1.65 (s,3 H) | 327 | 5.67 |
| 121 | 2-amino-6-(2′-fluoro-biphenyl-3-yl)-3,6-dimethyl-5,6-dihydro-pyrimidin-4(3H)-one | 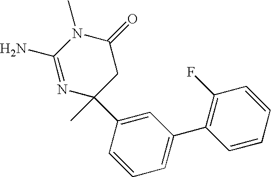 | ¹H NMR (500.132 MHz,DMSO)δ 7.58-7.49 (m, 4 H),7.48-7.43 (m, 2 H), 7.34 (m,2 H), 3.49 (d, J = 16.7 Hz,1 H), 3.18 (d, J = 16.7 Hz,1 H), 3.09 (s, 3 H), 1.66 (s,3 H) | 311 | 5.41 |
| 122 | 2-amino-6-(2′-methoxy-biphenyl-3-yl)-3,6-dimethyl-5,6-dihydro-pyrimidin-4(3H)-one | 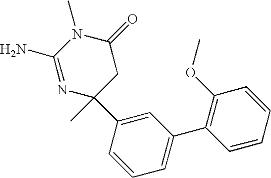 | ¹H NMR (500.132 MHz,DMSO)δ 7.50 (s, 1 H),7.46-7.41 (m, 2 H), 7.39-7.33(m, 2 H), 7.13 (d, J = 8.5 Hz,1 H), 7.29 (d, J = 7.2 Hz,1 H), 7.05 (t, J = 7.2 Hz, 1 H),3.76 (s, 3 H), 3.47 (d,J = 16.5 Hz, 1 H), 3.16 (d,J = 16.5 Hz, 1 H), 3.09 (s,3 H), 1.63 (s, 3 H) | 323 | 5.49 |
| 123 | 2-amino-6-(2′-ethoxy-biphenyl-3-yl)-3,6-dimethyl-5,6-dihydro-pyrimidin-4(3H)-one | 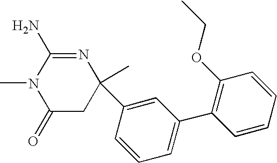 | ¹H NMR (500.132 MHz,DMSO)δ 7.58 (s, 1 H), 7.50-7.41 (m, 2 H), 7.38-7.27 (m,3 H), 7.12 (d, J = 9.0 Hz,1 H), 7.04 (t, J = 7.7 Hz, 1 H),4.05 (q, J = 6.8 Hz, 2 H),3.49 (d, J = 16.6 Hz, 1 H),3.19 (d, J = 16.6 Hz, 1 H),3.10 (s, 3 H), 1.65 (s, 3 H),1.28 (t, J = 7.0 Hz, 3 H) | 337 | 5.8 |
| 124 | 2-amino-6-(4′-ethoxy-biphenyl-3-yl)-3,6-dimethyl-5,6-dihydro-pyrimidin-4(3H)-one | 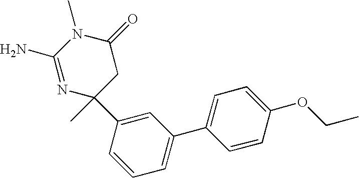 | ¹H NMR (500.132 MHz,DMSO)δ 7.64 (s, 1 H),7.60 (d, J = 8.4 Hz, 2 H),7.56 (d, J = 7.5 Hz, 1 H),7.45 (t, J = 8.0 Hz, 1 H),7.32 (d, J = 7.5 Hz, 1 H),7.03 (d, J = 8.4 Hz, 2 H),4.08 (q, J = 7.2 Hz, 2 H),3.07 (s, 3 H), 1.63 (s, 3 H),1.35 (t, J = 6.9 Hz, 3 H) | 337 | 5.81 |
| 125 | 2-amino-6-(2′,6′-dimethylbiphenyl-3-yl)-3,6-dimethyl-5,6-dihydropyrimidin-4(3H)-one | 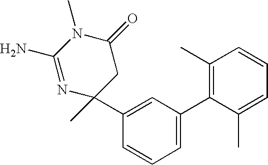 | ¹H NMR (500.132 MHz,DMSO)δ 7.50 (t, J = 7.6 Hz,1 H), 7.38 (d, J = 7.8 Hz,1 H), 7.20-7.08 (m, 5 H),3.48 (d, J = 16.8 Hz, 1 H),3.17 (d, J = 16.8 Hz, 1 H),3.05 (s, 3 H), 1.92 (s, 3 H),1.91 (s, 3 H), 1.64 (s, 3 H) | 321 | 6.04 |
| 126 | 2-amino-3,6-dimethyl-6-[3′-(trifluoromethyl)biphenyl-3-yl]-5,6-dihydropyrimidin-4(3H)-one | 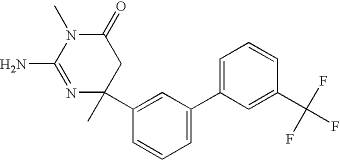 | ¹H NMR (500.132 MHz,DMSO)δ 8.02-7.98 (m, 2 H),7.80-7.77 (m, 2 H), 7.75 (d,J = 8.2 Hz, 1 H), 7.72 (d,J = 7.4 Hz, 1 H), 7.55 (t,J = 7.8 Hz, 1 H), 7.44 (d,J = 7.8 Hz, 1 H), 3.59 (d,J = 16.6 Hz, 1 H), 3.19 (d,J = 16.6 Hz, 1 H), 3.09 (s,3 H), 1.68 (s, 3 H) | 361 | 6.13 |
| 127 | 2-amino-3,6-dimethyl-6-[4′-(trifluoromethyl)biphenyl-3-yl]-5,6-dihydropyrimidin-4(3H)-one | 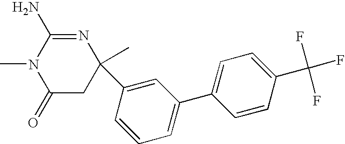 | ¹H NMR (500.132 MHz,DMSO)δ 7.91 (d, J = 7.3 Hz,2 H), 7.86 (d, J = 7.3 Hz,2 H), 7.78 (s, 1 H), 7.71 (d,J = 7.3 Hz, 1 H), 7.56 (t,J = 7.7 Hz, 1 H), 7.46 (d,J = 8.1 Hz, 1 H), 3.56 (d,J = 16.2 Hz, 1 H), 3.19 (d,J = 16.2 Hz, 1 H), 3.09 (s,3 H), 1.68 (s, 3 H) | 361 | 6.26 |
| 128 | 2-amino-6-[3′,5′-bis(trifluoromethyl)biphenyl-3-yl]-3,6-dimethyl-5,6-dihydro-pyrimidin-4(3H)-one | 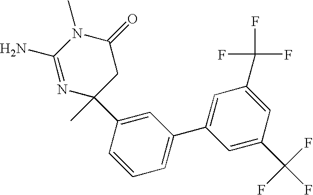 | ¹H NMR (500.132 MHz,DMSO)δ 8.36 (s, 2 H), 8.15(s, 1 H), 7.88 (s, 1 H), 7.85(d, J = 8.1 Hz, 1 H), 7.59(t, J = 8.0 Hz, 1 H), 7.49(d, J = 8.1 Hz, 1 H), 3.63(d, J = 16.7 Hz, 1 H), 3.22(d, J = 16.7 Hz, 1 H), 3.10 (s,3 H), 1.71 (s, 3 H) | 429 | 6.83 |
| 129 | 2-amino-6-(4′-tert-butyl-biphenyl-3-yl)-3,6-dimethyl-5,6-dihydro-pyrimidin-4(3H)-one | 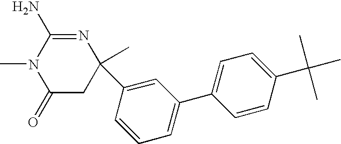 | ¹H NMR (500.132 MHz,DMSO)δ 7.65 (s, 1 H), 7.62-7.57 (m, 3 H), 7.53-7.48 (m,3 H), 7.37 (d, J = 7.4 Hz,1 H), 3.55 (d, J = 16.3 Hz,1 H), 3.19 (d, J = 16.3 Hz,1 H), 3.09 (s, 3 H), 1.67 (s,3 H), 1.33 (s, 9 H) | 349 | 6.84 |
| 130 | 2-amino-6-(3′,4′-dimethoxybiphenyl-3-yl)-3,6-dimethyl-5,6-dihydropyrimidin-4(3H)-one | 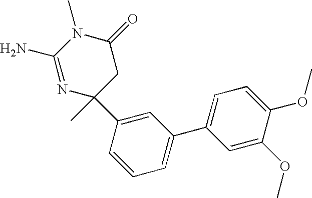 | ¹H NMR (500.132 MHz,DMSO)δ 7.64-7.58 (m, 3 H),7.47 (t, J = 7.7 Hz, 1 H), 7.32(d, J = 8.1 Hz, 1 H), 7.06 (d,J = 8.1 Hz, 1 H), 7.22-7.16(m, 3 H), 3.85 (s, 3 H), 3.80(s, 3 H), 3.55 (d, J = 16.7 Hz,1 H), 3.19 (d, J = 16.7 Hz,1 H), 3.09 (s, 3 H), 1.67 (s,3 H) | 353 | 5.05 |
| 131 | 2-amino-3,6-dimethyl-6-(4′-phenoxybiphenyl-3-yl)-5,6-dihydro-pyrimidin-4(3H)-one | 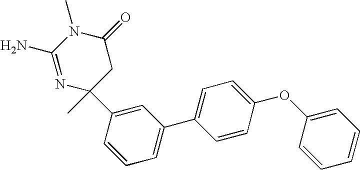 | ¹H NMR (500.132 MHz,DMSO)δ 7.71 (s, 1 H), 7.70-7.67 (m, 2 H), 7.62 (d,J = 8.4 Hz, 1 H), 7.50 (t,J = 7.7 Hz, 1 H), 7.43 (t,J = 8.0 Hz, 2 H), 7.36 (d,J = 8.0 Hz, 1 H), 7.19 (t,J = 7.7 Hz, 1 H), 7.13 (d,J = 8.9 Hz, 2 H), 7.07 (d,J = 8.0 Hz, 2 H), 3.56 (d,J = 16.4 Hz, 1 H), 3.19 (d,J = 16.4 Hz, 1 H), 3.09(s, 3 H), 1.68 (s, 3 H) | 385 | 6.66 |
| 132 | 2-amino-6-(2′,4′-dimethoxybiphenyl-3-yl)-3,6-dimethyl-5,6-dihydropyrimidin-4(3H)-one | 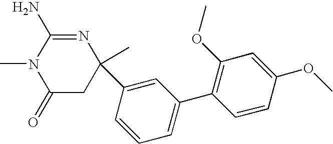 | ¹H NMR (500.132 MHz,DMSO)δ 7.45 (s, 1 H), 7.41-7.35 (m, 2 H), 7.31-7.27 (m,1 H), 7.21 (d, J = 8.7 Hz,1 H), 6.67 (s, 1 H), 6.63 (d,J = 8.7 Hz, 1 H), 3.81 (s,3 H), 3.76 (s, 3 H), 3.08 (s,3 H), 1.61 (s, 3 H) | 353 | 5.49 |
| 133 | 2-amino-6-[3-(1-benzo-furan-2-yl)phenyl]-3,6-dimethyl-5,6-dihydro-pyrimidin-4(3H)-one | 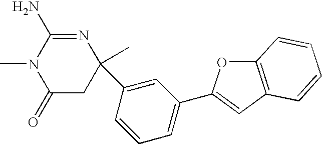 | ¹H NMR (500.132 MHz,DMSO)δ 7.96 (s, 1 H), 7.90(d, J = 7.9 Hz, 1 H), 7.70 (d,J = 7.9 Hz, 1 H), 7.63 (d,J = 7.9 Hz, 1 H), 7.56 (t,J = 7.5 Hz, 1 H),7.51 (s,1 H), 7.43 (d, J = 7.9 Hz,1 H), 7.36 (t, J = 7.5 Hz,1 H), 7.29 (t, J = 7.7 Hz,1 H), 3.56 (d, J = 16.7 Hz,1 H), 3.22 (d, J = 16.7 Hz,1 H), 3.10 (s, 3 H), 1.69(s, 3 H) | 333 | 5.93 |
| 134 | 2-amino-6-[3-(1,3-benzodioxol-5-yl)phenyl]-3,6-dimethyl-5,6-dihydropyrimidin-4(3H)-one | 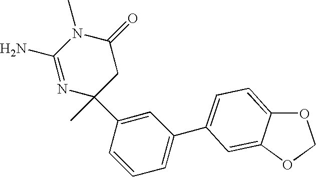 | ¹H NMR (500.132 MHz,DMSO)δ 7.66-7.60 (m, 2 H),7.58-7.54 (m, 2 H), 7.46 (t,J = 3.8 Hz, 1 H), 7.33 (d,J = 4.0 Hz, 1 H), 7.28 (s,1 H), 7.16 (d, J = 4.0 Hz,1 H), 7.03 (d, J = 4.5 Hz,1 H), 6.08 (s, 2 H), 3.09 (s,3 H), 1.65 (s, 3 H) | 337 | 5.34 |
| 135 | 2-amino-3,6-dimethyl-6-[3′-(trifluoromethoxy)biphenyl-3-yl]-5,6-dihydropyrimidin-4(3H)-one | 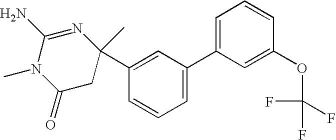 | ¹H NMR (500.132 MHz,DMSO)δ 7.77-7.71 (m, 2 H),7.68-7.61 (m, 3 H), 7.52 (t,J = 7.9 Hz, 1 H), 7.47-7.39(m, 2 H), 3.07 (s, 3 H), 1.62(s, 3 H) | 377 | 6.29 |
| 136 | 2-amino-6-[3′-(hydroxy-methyl)biphenyl-3-yl]-3,6-dimethyl-5,6-dihydropyrimidin-4(3H)-one | 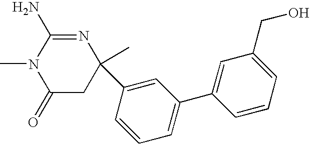 | ¹H NMR (500.132 MHz,DMSO)δ 7.68 (s, 1 H), 7.64-7.59 (m, 2 H), 7.55-7.49 (m,2 H), 7.45 (t, J = 7.6 Hz,1 H), 7.40-7.35 (m, 2 H),3.56 (d, J = 16.4 Hz, 1 H),3.20 (d, J = 16.4 Hz, 1 H),3.09 (s, 3 H), 1.68 (s, 3 H) | 323 | 4.48 |
| 137 | 2-amino-3,6-dimethyl-6-[3-(1-naphthyl)phenyl]-5,6-dihydropyrimidin-4(3H)-one | 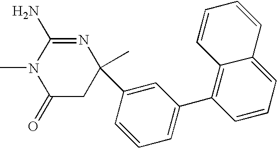 | ¹H NMR (500.132 MHz,DMSO)δ 8.03 (d, J = 8.5 Hz,1 H), 7.99 (d, J = 8.5 Hz,1 H), 7.70 (d, J = 8.5 Hz,1 H), 7.63-7.55 (m, 3 H),7.54-7.48 (m, 3 H), 7.47-7.42(m, 2 H), 3.11 (s, 3 H), 1.65(s, 3 H) | 343 | 6.09 |
| 138 | 2-amino-3,6-dimethyl-6-(1,1′:3′,1″-terphenyl-3-yl)-5,6-dihydro-pyrimidin-4(3H)-one | 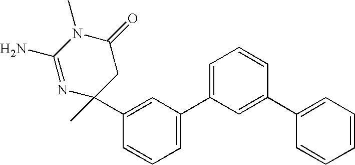 | ¹H NMR (500.132 MHz,DMSO)δ 7.88 (s, 1 H), 7.79-7.74 (m, 3 H), 7.72-7.63 (m,3 H), 7.59 (t, J = 7.6 Hz,1 H), 7.52-7.49 (m, 3 H),7.42 (d, J = 7.3 Hz, 2 H),3.07 (s, 3 H), 1.63 (s, 3 H) | 369 | 6.57 |
| 139 | 2-amino-6-[4′-(benzyloxy)biphenyl-3-yl]-3,6-dimethyl-5,6-dihydropyrimidin-4(3H)-one | 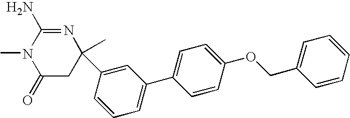 | ¹H NMR (500.132 MHz,DMSO)δ 7.65-7.57 (m, 4 H),7.50-7.44 (m, 3 H), 7.41 (t,J = 7.6 Hz, 2 H), 7.36 (d,J = 7.3 Hz, 1 H), 7.32 (d,J = 9.1 Hz, 1 H), 7.13(d, J = 9.1 Hz, 2 H), 5.18 (s,2 H), 3.56 (d, J = 16.4 Hz,1 H), 3.18 (d, J = 16.4 Hz,1 H), 3.09 (s, 3 H), 1.67 (s,3 H) | 399 | 6.66 |
| 140 | 2-amino-6-[3′-(benzyl-oxy)biphenyl-3-yl]-3,6-dimethyl-5,6-dihydro-pyrimidin-4(3H)-one | 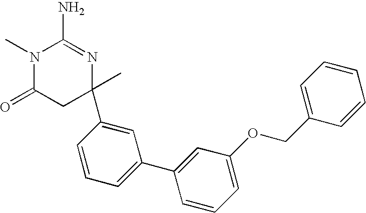 | ¹H NMR (500.132 MHz,DMSO)δ 7.69 (s, 1 H), 7.64(d, J = 7.6 Hz, 1 H), 7.53-7.47 (m, 3 H), 7.45-7.34 (m,5 H), 7.30 (s, 1 H), 7.26 (d,J = 7.6 Hz, 1 H), 7.07 (d,J = 7.6 Hz, 1 H), 5.20 (s,2 H), 3.57 (d, J = 16.4 Hz,1 H), 3.20 (d, J = 16.4 Hz,1 H), 3.09 (s, 3 H), 1.68(s, 3 H) | 399 | 6.64 |
| 141 | 2-amino-6-[4′-butyl-biphenyl-3-yl)-3,6-dimethyl-5,6-dihydro-pyrimidin-4(3H)-one | 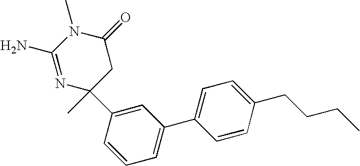 | ¹H NMR (500.132 MHz,DMSO)δ 7.67 (s, 1 H), 7.61(d, J = 7.8 Hz, 1 H), 7.58 (d,J = 8.2 Hz, 2 H), 7.49 (t,J = 7.6 Hz, 1 H), 7.36 (d,J = 8.2 Hz, 1 H), 7.31 (d,J = 8.2 Hz, 2 H), 3.56 (d,J = 16.4 Hz, 1 H), 3.19 (d,J = 16.4 Hz, 1 H), 3.09 (s,3 H), 2.66-2.61 (m, 2 H), 1.67(s, 3 H), 1.59 (quintet,J = 7.6 Hz, 2 H), 1.33(quintet, J = 7.6 Hz, 2 H),0.92 (t, J = 7.5 Hz, 3 H) | 349 | 6.97 |
| 142 | 2-amino-6-[3-(2,3-dihydro-1,4-benzodioxin-6-yl)phenyl]-3,6-dimethyl-5,6-dihydro-pyrimidin-4(3H)-one | 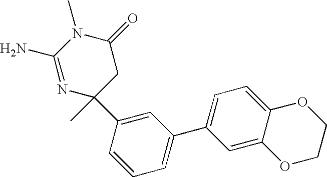 | ¹H NMR (500.132 MHz,DMSO)δ 7.62 (s, 1 H), 7.56(d, J = 7.8 Hz, 10 H), 7.46(t, J = 7.8 Hz, 1 H), 7.82 (d,J = 7.7 Hz, 1 H), 7.19 (s,1 H), 7.14 (d, J = 8.4 Hz,1 H), 6.97 (d, J = 8.4 Hz,1 H), 4.29 (s, 4 H), 3.56 (d,J = 16.4 Hz, 1 H), 3.17(d, J = 16.4 Hz, 1 H), 3.09(s, 3 H), 1.68 (s, 3 H) | 351 | 5.34 |
| 143 | 2-amino-6-[3-(2,3-dihydro-1-benxofuran-5-yl)phenyl]-3,6-dimethyl-5,6-dihydro-pyrimidin-4(3H)-one | 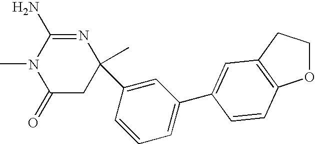 | ¹H NMR (500.132 MHz,DMSO)δ 7.62 (s, 1 H), 7.57-7.54 (m, 2 H), 7.45 (t,J = 7.9 Hz, 2 H), 7.40(d, J = 8.3 Hz, 1 H), 7.30(d, J = 8.3 Hz, 1 H), 6.87(d, J = 8.3 Hz, 1 H), 4.59 (t,J = 8.8 Hz, 2 H), 3.56 (d,J = 16.7 Hz, 1 H), 3.18 (d,J = 16.7 Hz, 1 H), 3.25 (t,J = 8.8 Hz, 3 H), 3.09 (s,3 H), 1.66 (s, 3 H) | 335 | 5.36 |
| 144 | 2-amino-3,6-dimethyl-6-(4′-propylbiphenyl-3-yl)-5,6-dihydro-pyrimidin-4(3H)-one | 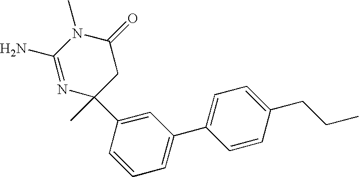 | ¹H NMR (500.132 MHz,DMSO)δ 7.67 (s, 1 H), 7.61(d, J = 7.8 Hz, 1 H), 7.58 (d,J = 7.9 Hz, 2 H), 7.49 (t,J = 7.8 Hz, 1 H), 7.35 (d,J = 7.9 Hz, 1 H), 7.31 (d,J = 7.9 Hz, 2 H), 3.66 (d,J = 16.3 Hz, 1 H), 3.19 (d,J = 16.3 Hz, 1 H), 3.09 (s,3 H), 2.61 (t, J = 7.6 Hz,2 H), 1.67 (s, 3 H), 1.64 (qt,J = 7.4 Hz, 2 H), 0.92 (t,J = 7.4 Hz, 3 H) | 335 | 6.61 |
| 145 | 2-amino-6-[2′,3′-dimethoxybiphenyl-3-yl)-3,6-dimethyl-5,6-dihydropyrimidin-4(3H)-one | 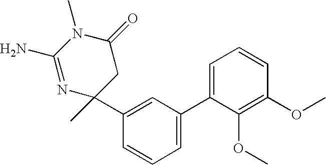 | ¹H NMR (500.132 MHz,DMSO)δ 7.51 (s, 1 H),7.45 (d, J = 7.9 Hz, 1 H),7.42-7.37 (m, 2 H), 7.15(t, J = 7.9 Hz, 1 H), 7.09(d, J = 8.3 Hz, 1 H), 6.90(d, J = 7.5 Hz, 1 H),3.85 (s, 3 H), 3.59-3.50 (m,4 H), 3.07 (s, 3 H), 1.60 (s,3 H) | 353 | 5.32 |
| 146 | 3′-(2-amino-1,4-dimethyl-6-oxo-1,4,5,6-tetrahydropyrimidin-4-yl)biphenyl-3-carboxylic acid | 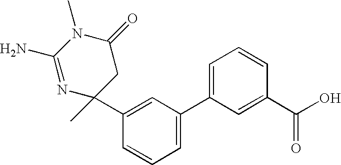 | ¹H NMR (500.132 MHz,DMSO)δ 8.19 (s, 1 H),7.97 (d, J = 8.2 Hz, 1 H),7.92 (d, J = 8.2 Hz, 1 H),7.67 (d, J = 8.2 Hz, 1 H),7.43 (d, J = 8.2 Hz, 1 H),7.74 (s, 1 H), 7.63 (t,J = 7.4 Hz, 1 H), 7.54 (t,J = 7.8 Hz, 1 H), 3.58 (d,J = 16.4 Hz, 1 H), 3.19(d, J = 16.4 Hz, 1 H), 3.09(s, 3 H), 1.68 (s, 3 H) | 337 | 4.7 |
| 147 | 2-amino-3,6-dimethyl-6-(3-thianthren-1-ylphenyl)-5,6-dihydro-pyrimidin-4(3H)-one | 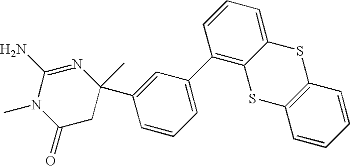 | ¹H NMR (500.132 MHz,DMSO)δ 7.66 (d, J = 7.8 Hz,1 H), 7.62 (d, J = 7.8 Hz,1 H), 7.57 (t, J = 7.7 Hz,1 H), 7.52 (d, J = 7.8 Hz,1 H), 7.46 (s, 1 H), 7.43 (t,J = 7.7 Hz, 2 H), 7.41-7.35 (m, 3 H), 7.31 (t,J = 7.5 Hz, 1 H), 3.56 (d,J = 16.7 Hz, 1 H), 3.23 (d,J = 16.7 Hz, 1 H), 3.14 (s,3 H), 1.69 (s, 3 H) | 431 | 6.79 |
| 148 | 2-amino-3,6-dimethyl-6-(1,1′:2′,1″-terphenyl-3-yl)-5,6-dihydro-pyrimidin-4(3H)-one | 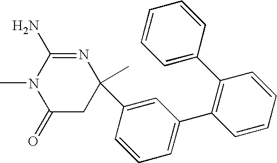 | ¹H NMR (500.132 MHz,DMSO)δ 7.51-7.47 (m, 2 H),7.46-7.40 (m, 2 H), 7.32 (t,J = 8.0 Hz, 1 H), 7.26-7.18(m, 4 H), 7.15 (d, J = 7.8 Hz,1 H), 7.09-7.04 (m, 3 H), 3.23(d, J = 16.5 Hz, 1 H), 3.03(s, 3 H), 1.69 (s, 3 H) | 369 | 6.37 |
| 149 | 2-amino-3,6-dimethyl-6-[3-(2-thienyl)phenyl]-5,6-dihydropyrimidin-4(3H)-one | 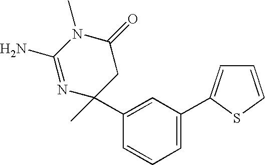 | ¹H NMR (500.132 MHz,DMSO)δ 7.67 (s, 1 H), 7.64-7.59 (m, 2 H), 7.56 (d,J = 4.4 Hz, 1 H), 7.47 (t,J = 7.8 Hz, 1 H), 7.33 (d,J = 8.3 Hz, 1 H), 7.18 (t,J = 4.4 Hz, 1 H), 3.52 (d,J = 16.5 Hz, 1 H), 3.19 (d,J = 16.5 Hz, 1 H), 3.09 (s,3 H), 1.67 (s, 3 H) | 299 | 5.21 |
| 150 | 2-amino-3,6-dimethyl-6-[3-(3-thienyl)phenyl]-5,6-dihydropyrimidin-4(3H)-one | 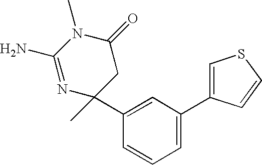 | ¹H NMR (500.132 MHz,DMSO)δ 7.89 (s, 1 H), 7.75(s, 1 H), 7.69-7.63 (m, 2 H),7.57 (d, J = 5.3 Hz, 1 H),7.42 (t, J = 7.8 Hz, 1 H),7.30 (d, J = 7.8 Hz, 1 H),3.06 (s, 3 H), 1.60 (s, 3 H) | 299 | 4.94 |
| 151 | 2-amino-6-(3′,5′-dimethylbiphenyl-3-yl)-3,6-dimethyl-5,6-dihydropyrimidin-4(3H)-one | 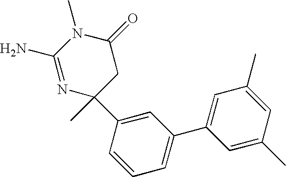 | ¹H NMR (500.132 MHz,DMSO)δ 7.65 (s, 1 H),7.59 (d, J = 7.7 Hz, 1 H),7.48 (t, J = 7.7 Hz, 1 H),7.35 (d, J = 7.7 Hz, 1 H),7.26 (s, 2 H), 7.03 (s, 1 H),3.55 (d, J = 16.7 Hz, 1 H),3.18 (d, J = 16.7 Hz, 1 H),3.09 (s, 3 H), 2.35 (s, 6 H),1.67 (s, 3 H) | 321 | 6.24 |
| 152 | 2-amino-3,6-dimethyl-6-[3-(2-naphthyl)phenyl]-5,6-dihydropyrimidin-4(3H)-one | 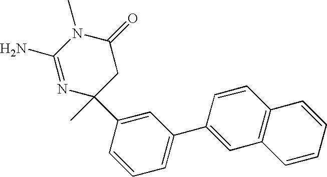 | ¹H NMR (500.132 MHz,DMSO)δ 8.21 (s, 1 H),8.05-7.94 (m, 3 H), 7.89-7.79(m, 2 H), 7.70 (t, J = 7.7 Hz,1 H), 7.59-7.42 (m, 4 H),3.05 (s, 3 H), 1.56 (s, 3 H) | 343 | 6.28 |
| 153 | 2-amino-6-[4′-(hydroxymethyl)biphenyl-3-yl]-3,6-dimethyl-5,6-dihydro-pyrimidin-4(3H)-one | 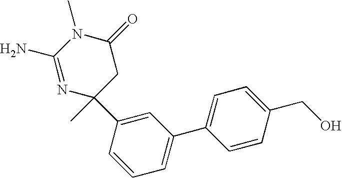 | ¹H NMR (500.132 MHz,DMSO)δ 7.70 (s, 1 H), 7.61(d, J = 7.7 Hz, 2 H), 7.55(d, J = 7.7 Hz, 1 H), 7.46-7.36 (m, 4 H), 4.54 (d,J = 5.6 Hz, 2 H), 3.03 (s,3 H), 1.54 (s, 3 H) | 323 | 4.4 |
| 154 | 2-amino-6-[3-(2-furyl)phenyl]-3,6-dimethyl-5,6-dihydropyrimidin-4(3H)-one | 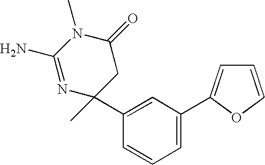 | ¹H NMR (500.132 MHz,DMSO)δ 7.77 (s, 1 H), 7.75(s, 1 H), 7.62 (d, J = 7.8 Hz,1 H), 7.42 (t, J = 7.4 Hz,1 H), 7.32 (d, J = 8.7 Hz,1 H), 6.98 (d, J = 3.5 Hz,1 H), 6.63-6.60 (m, 1 H),3.04 (s, 3 H), 1.56 (s, 3 H) | 283 | 4.95 |
| 155 | 2-amino-6-[4′-hydroxy-biphenyl-3-yl]-3,6-dimethyl-5,6-dihydro-pyrimidin-4(3H)-one | 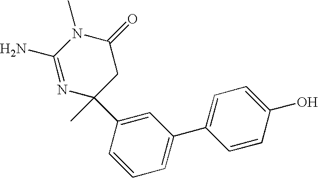 | ¹H NMR (500.132 MHz,DMSO)δ 7.61 (s, 1 H), 7.49-7.44 (m, 3 H), 7.38 (t,J = 7.8 Hz, 1 H), 7.30 (d,J = 7.8 Hz, 1 H), 6.85 (d,J = 7.8 Hz, 2 H), 3.03 (s,3 H), 1.53 (s, 3 H) | 309 | 4.53 |
| 156 | 6-(4′-acetylbiphenyl-3-yl)-2-amino-3,6-dimethyl-5,6-dihydro-pyrimidin-4(3H)-one | 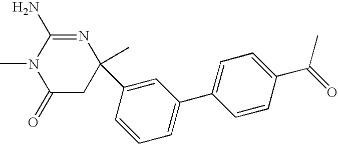 | ¹H NMR (500.132 MHz,DMSO)δ 8.05 (d, J = 8.0 Hz,2 H), 7.82 (d, J = 8.0 Hz,2 H), 7.79 (s, 1 H), 7.65 (d,J = 6.9 Hz, 1 H), 7.52-7.45(m, 2 H), 3.04 (s, 3 H), 2.62(s, 3 H), 1.55 (s, 3 H) | 335 | 5.03 |
| 157 | 6-(3′-acetylbiphenyl-3-yl)-2-amino-3,6-dimethyl-5,6-dihydro-pyrimidin-4(3H)-one | 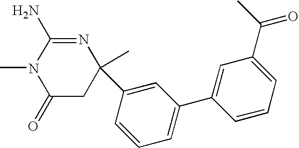 | ¹H NMR (500.132 MHz,DMSO)δ 8.14 (s, 1 H), 7.99(d, J = 8.0 Hz, 1 H), 7.93(d, J = 8.0 Hz, 1 H), 7.75 (s,1 H), 7.70-7.63 (m, 2 H), 7.53(t, J = 7.2 Hz, 1 H), 7.44(d, J = 8.0 Hz, 1 H), 3.07 (s,3 H), 2.67 (s, 3 H), 1.63 (s,3 H) | 335 | 5.08 |
| 158 | 2-amino-6-[3-(1-benzothien-2-yl)phenyl]-3,6-dimethyl-5,6-dihydropyrimidin-4(3H)-one | 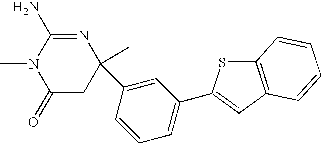 | ¹H NMR (500.132 MHz,DMSO)δ 8.01 (d, J = 7.4 Hz,1 H), 7.93 (s, 1 H), 7.88 (d,J = 7.4 Hz, 1 H), 7.83 (s,1 H), 7.74 (d, J = 7.4 Hz,1 H), 7.54 (t, J = 7.7 Hz,1 H), 7.45-7.37 (m, 3 H), 3.56(d, J = 16.5 Hz, 1 H), 3.22(d, J = 16.5 Hz, 1 H), 3.10(s, 3 H), 1.70 (s, 3 H) | 349 | 6.22 |
| 159 | 2-amino-3,6-dimethyl-6-(3′,4′,5′-trimethoxy-biphenyl-3-yl)-5,6-dihydropyrimidin-4(3H)-one | 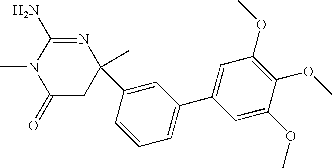 | ¹H NMR (500.132 MHz,DMSO)δ 7.64 (s, 1 H), 7.61(d, J = 8.0 Hz, 1 H), 7.38(d, J = 8.0 Hz, 1 H), 7.47 (t,J = 7.7 Hz, 1 H), 6.89 (s,2 H), 3.87 (s, 6 H), 3.70 (s,3 H), 3.13 (d, J = 16.5 Hz,1 H), 3.45 (d, J = 16.5 Hz,1 H), 3.07 (s, 3 H), 1.63 (s,3 H) | 383 | 5.14 |
| 160 | 6-[3-(5-acetyl-2-thienyl)phenyl]-2-amino-3,6-dimethyl-5,6-dihydro-pyrimidin-4(3H)-one | 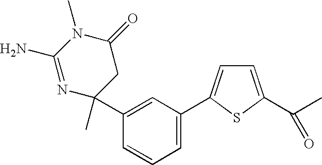 | ¹H NMR (500.132 MHz,DMSO)δ 7.99 (d, J = 3.7 Hz,1 H), 7.77 (s, 1 H), 7.75 (d,J = 8.0 Hz, 1 H), 7.69 (d,J = 3.7 Hz, 1 H), 7.51 (t,J = 7.7 Hz, 1 H), 7.43 (d,J = 8.0 Hz, 1 H), 3.51(d, J = 16.5 Hz, 1 H), 3.17(d, J = 16.5 Hz, 1 H), 3.08(s, 3 H), 2.56 (s, 3 H), 1.65(s, 3 H) | 341 | 4.9 |
| 161 | 2-amino-6-(3′-fluoro-1,1′:4′,1″-terphenyl-3-yl)-3,6-dimethyl-5,6-dihydropyrimidin-4(3H)-one | 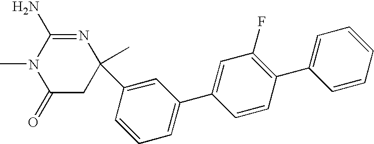 | ¹H NMR (500.13 MHz,DMSO)δ 7.80 (s, 1 H), 7.75(d, J = 7.4 Hz, 1 H), 7.69 (d,J = 12.7 Hz, 1 H), 7.67-7.64(m, 2 H), 7.63-7.59 (m, 2 H),7.57-7.49 (m, 3 H), 7.46-7.40(m, 2 H), 3.60 (d,J = 16.5 Hz, 1 H), 3.20 (d,J = 16.5 Hz, 1 H), 3.10 (s,3 H), 1.69 (s, 3 H) | 387 | 6.79 |
| 162 | 2-amino-3,6-dimethyl-6-(3-pyridin-3-ylphenyl)-5,6-dihydropyrimidin-4(3H)-one | 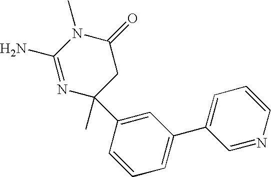 | ¹H NMR (500 MHz,DMSO-d6)δ 8.92 (s, 1 H),8.61 (d, J = 4.9 Hz, 1 H),8.14 (s, 1 H), 8.09 (d,J = 8.1 Hz, 1 H), 7.78 (s,1 H), 7.70 (d, J = 7.7 Hz,1 H), 7.59-7.50 (m, 2 H), 7.44(d, J = 7.9 Hz, 1 H), 3.56 (d,J = 18.8 Hz, 1 H), 3.18 (d,J = 16.7 Hz, 1 H), 3.09 (s,3 H), 1.67 (s, 3 H) | 294 | 2.63 |
| 163 | 2-amino-6-[3-(1-benzothien-3-yl)phenyl]-3,6-dimethyl-5,6-dihydropyrimidin-4(3H)-one | 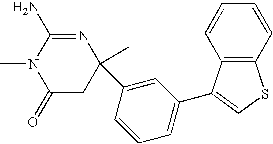 | ¹H NMR (500.132 MHz,DMSO)δ 8.12-8.07 (m, 1 H),7.84-7.79 (m, 2 H), 7.66 (s,1 H), 7.55-7.43 (m, 5 H), 3.08(s, 3 H), 1.59 (s, 3 H) | 349 | 6.11 |
| 164 | 3′-(2-amino-1,4-dimethyl-6-oxo-1,4,5,6-tetrahydro-pyrimidin-4-yl)biphenyl-3-carbonitrile | 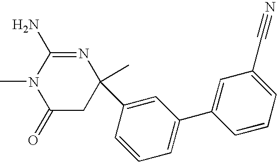 | ¹H NMR (500 MHz,DMSO-d6)δ 8.19 (s, 1 H),8.14 (s, 1 H), 8.03 (d,J = 8.1 Hz, 1 H), 7.86 (d,J = 7.5 Hz, 1 H), 7.81 (s,1 H), 7.73-7.66 (m, 2 H),7.52 (t, J = 3.8 Hz, 1 H),7.45 (d, J = 7.8 Hz, 1 H),3.33-3.27 (m, 2 H), 3.06 (s,3 H), 1.61 (s, 3 H) | 318 | 5.22 |
| 165 | 3′-(2-amino-1,4-dimethyl-6-oxo-1,4,5,6-tetrahydro-pyrimidin-4-yl)biphenyl-4-carbonitrile | 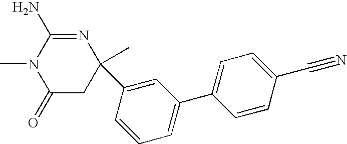 | ¹H NMR (500.132 MHz,DMSO)δ 7.98 (d, J = 8.0 Hz,2 H), 7.90 (d, J = 8.0 Hz,2 H), 7.79 (s, 1 H), 7.72 (d,J = 8.0 Hz, 1 H), 7.46 (d,J = 8.0 Hz, 1 H), 7.56 (t,J = 8.0 Hz, 1 H), 3.56 (d,J = 16.5 Hz, 1 H), 3.18(d, J = 16.5 Hz, 1 H), 3.08(s, 3 H), 1.66 (s, 3 H) | 318 | 5.19 |
| 166 | 6-(2′-acetylbiphenyl-3-yl)-2-amino-3,6-dimethyl-5,6-dihydropyrimidin-4(3H)-one | 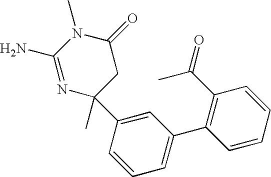 | ¹H NMR (500.132 MHz,DMSO)δ 7.64-7.57 (m, 2 H),7.50 (t, J = 7.4 Hz, 1 H),7.47-7.43 (m, 2 H), 7.40(d, J = 7.4 Hz, 1 H), 7.28-7.24 (m, 2 H), 3.05 (s, 3 H),2.07 (s, 3 H), 1.52 (s, 3 H) | 335 | 4.97 |
| 167 | 2-amino-3,6-dimethyl-6-[4′-(methylsulfonyl)biphenyl-3-yl]-5,6-dihydropyrimidin-4(3H)-one | 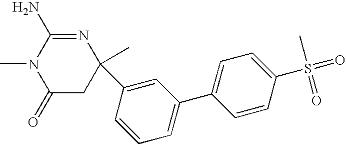 | ¹H NMR (500.132 MHz,DMSO)δ 8.04 (d, J = 8.0 Hz,2 H), 7.95 (d, J = 8.0 Hz,2 H), 7.78 (s, 1 H), 7.73 (d,J = 8.0 Hz, 1 H), 7.57 (t,J = 8.0 Hz, 1 H), 7.46 (d,J = 8.0 Hz, 1 H), 3.58 (d,J = 16.5 Hz, 1 H), 3.27(s, 3 H), 3.21 (d, J = 16.5 Hz,1 H), 3.09 (s, 3 H), 1.68(s, 3 H) | 371 | 4.99 |
| 168 | 2-amino-6-(4′-cyclo-hexylbiphenyl-3-yl)-3,6-dimethyl-5,6-dihydro-pyrimidin-4(3H)-one | 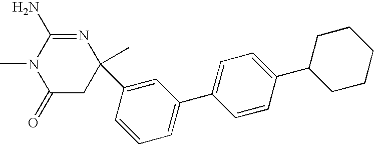 | ¹H NMR (500.132 MHz,DMSO) δ 7.65 (s, 1 H), 7.61-7.55 (m, 3 H), 7.48 (t,J = 7.7 Hz, 1 H), 7.37-7.31(m, 3 H), 3.55 (d,J = 16.5 Hz, 1 H), 3.18 (d,J = 16.5 Hz, 1 H), 3.08 (s,3 H), 1.84-1.78 (m, 4 H), 1.66(s, 3 H), 1.50-1.33 (m, 4 H),1.30-1.22 (m, 3 H) | 375 | 7.45 |
| 169 | 2-amino-3,6-dimethyl-6-(3′,4′,5′-trifluoro-biphenyl-3-yl)-5,6-dihydropyrimidin-4(3H)-one | 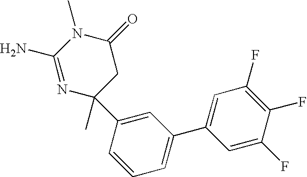 | ¹H NMR (500.132 MHz,DMSO) δ 7.78 (s, 1 H), 7.71-7.65 (m, 2 H), 7.58 (d,J = 8.6 Hz, 1 H), 7.49 (d,J = 8.6 Hz, 1 H), 7.42 (t,J = 7.9 Hz, 1 H), 3.31-3.28(m, 1 H), 3.00 (s, 3 H), 2.83(d, J = 16.5 Hz, 1 H), 1.43(s, 3 H) | 347 | 6 |
| 170 | 2-amino-6-[4′-(ethylsulfonyl)biphenyl-3-yl]-3,6-dimethyl-5,6-dihydropyrimidin-4(3H)-one | 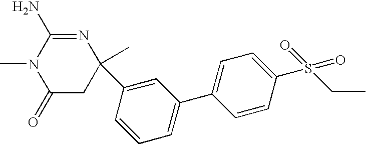 | ¹H NMR (500.131 MHz,DMSO) δ 7.81 (d, J = 8.6 Hz,2 H), 7.76 (d, J = 8.6 Hz,2 H), 7.66 (s, 1 H), 7.44 (d,J = 7.9 Hz, 1 H), 7.36 (d,J = 7.9 Hz, 1 H), 7.30 (t,J = 7.6 Hz, 1 H), 2.84 (s,3 H), 2.65 (d, J = 16.5 Hz,1 H), 1.28 (s, 3 H), 0.98(t, J = 7.2 Hz, 3 H) | 385 | 4.64 |
| 171 | 2-amino-6-(4′-hydroxy-3′-methoxybiphenyl-3-yl)-3,6-dimethyl-5,6-dihydro-pyrimidin-4(3H)-one | 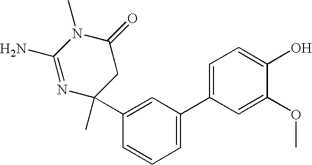 | ¹H NMR (500.132 MHz,DMSO) δ 7.64 (s, 1 H), 7.45(d, J = 7.2 Hz, 1 H), 7.38-7.32 (m, 2 H), 7.15 (s, 1 H),7.05 (d, J = 8.6 Hz, 1 H),6.86 (d, J = 8.6 Hz, 1 H),3.86 (s, 3 H), 3.33-3.28(m, 1 H), 3.00 (s, 3 H),2.81 (d, J = 16.5 Hz, 1 H),1.45 (s, 3 H) | 339 | 4.59 |
| 172 | 2-amino-6-(4′-hydroxy-3′,5′-dimethylbiphenyl-3-yl)-3,6-dimethyl-5,6-dihydropyrimidin-4(3H)-one | 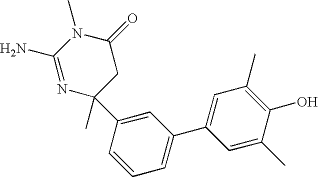 | ¹H NMR (500.131 MHz,DMSO) δ 7.58 (s, 1 H), 7.16(s, 2 H), 7.37-7.32 (m, 1 H),7.29-7.25 (m, 2 H), 2.95 (s,3 H), 2.19 (s, 3 H), 1.37 (s,3 H) | 337 | 5.19 |
| 173 | 2-amino-6-(2′-hydroxy-biphenyl-3-yl)-3,6-dimethyl-5,6-dihydro-pyrimidin-4(3H)-one | 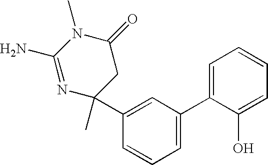 | ¹H NMR (500.131 MHz,DMSO) δ 7.35 (s, 1 H), 7.22-7.17 (m, 1 H), 7.14-7.10 (m,2 H), 7.00 (d, J = 7.9 Hz,1 H), 6.95 (t, J = 7.6 Hz,1 H), 6.72 (d, J = 7.9 Hz,1 H), 6.66 (t, J = 7.6 Hz,1 H), 2.80 (s, 3 H), 2.58 (d,J = 16.5 Hz, 1 H), 1.23 (s,3 H) | 309 | 4.88 |
| 174 | 2-amino-6-(3′-hydroxy-biphenyl-3-yl)-3,6-dimethyl-5,6-dihydro-pyrimidin-4(3H)-one | 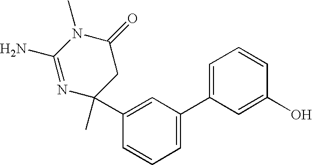 | ¹H NMR (500.132 MHz,DMSO) δ 7.66 (s, 1 H), 7.60-7.54 (m, 1 H), 7.45-7.37 (m,2 H), 7.26 (t, J = 8.2 Hz,1 H), 7.04 (d, J = 7.3 Hz,1 H), 6.77 (d, J = 7.3 Hz,1 H), 7.00 (s, 1 H), 3.00 (s,3 H), 2.79 (d, J = 16.3 Hz,1 H), 1.43 (s, 3 H) | 309 | 4.7 |
| 175 | 5-[3-(2-amino-1,4-dimethyl-6-oxo-1,4,5,6-tetrahydro-pyrimidin-4-yl)phenyl]thiophene-2-carbonitrile | 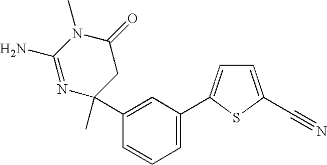 | ¹H NMR (500.131 MHz,DMSO) δ 8.03 (d, J = 4.2 Hz,1 H), 7.84 (s, 1 H), 7.70 (d,J = 4.2 Hz, 1 H), 7.63 (d,J = 7.7 Hz, 1 H), 7.55 (d,7.45 (t, J = 7.6 Hz, 1 H), 3.03(s, 3 H), 2.81 (d, J = 16.3 Hz,1 H), 1.42 (s, 3 H) | 324 | 5.16 |
| 176 | N-[3′-(2-amino-1,4-dimethyl-6-oxo-1,4,5,6-tetrahydro-pyrimidin-4-yl)biphenyl-4-yl]acetamide | 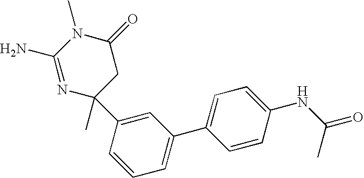 | ¹H NMR (500.132 MHz,DMSO) δ 7.72-7.65 (m, 4 H),7.58 (d, J = 8.1 Hz, 2 H),7.50-7.45 (m, 1 H), 7.40-7.36(m, 1 H), 3.00 (s, 3 H), 2.80(d, J = 16.2 Hz, 1 H), 2.07(s, 3 H), 1.44 (s, 3 H) | 350 | 4.44 |
| 177 | 2-amino-3,6-dimethyl-6-{3-[1-(phenylsulfonyl)-1H-indol-3-yl]phenyl}-5,6-dihydropyrimidin-4(3H)-one | 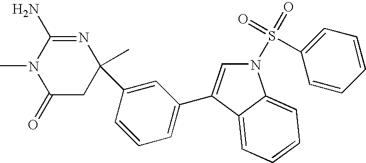 | ¹H NMR (500.132 MHz,DMSO) δ 8.22 (s, 1 H), 8.10(s, 1 H), 8.08 (d, J = 7.8 Hz,1 H), 8.04 (d, J = 7.8 Hz,1 H), 7.81 (d, J = 7.8 Hz,1 H), 7.78 (s, 1 H), 7.71(t, J = 7.4 Hz, 1 H), 7.61(t, J = 7.7 Hz, 1 H), 7.59-7.56(m, 2 H), 7.48-7.41 (m, 3 H),7.36 (t, J = 7.7 Hz, 1 H), 3.02(s, 3 H), 2.84 (d, J = 16.4 Hz,1 H), 1.46 (s, 3 H) | 472 | 6.66 |
| 178 | 2-amino-6-[3-(3,4-dihydro-2H-1,5-benzodioxepin-7-yl)phenyl]-3,6-dimethyl-5,6-dihydropyrimidin-4(3H)-one | 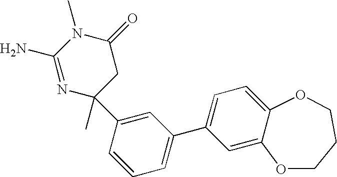 | ¹H NMR (500.131 MHz,DMSO) δ 7.67 (s, 1 H), 7.48-7.43 (m, 1 H), 7.40-7.35 (m,2 H), 7.25 (s, 1 H), 7.23 (d,J = 8.4 Hz, 1 H), 7.06(d, J = 8.4 Hz, 1 H), 4.22-4.15 (m, 4 H), 3.00 (s, 3 H),2.81 (d, J = 16.5 Hz, 1 H),2.18-2.11 (m, 2 H), 1.44 (s,3 H) | 365 | 5.52 |
| 179 | 2-amino-6-[3-(6-ethoxy-2-naphthyl)phenyl]-3,6-dimethyl-5,6-dihydro-pyrimidin-4(3H)-one | 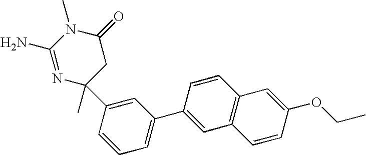 | ¹H NMR (500.132 MHz,DMSO) δ 8.11 (s, 1 H),7.90 (d, J = 8.9 Hz, 2 H),7.85 (s, 1 H), 7.77 (d,J = 8.9 Hz, 1 H), 7.65-7.61(m, 1 H), 7.45-7.42 (m, 2 H),7.35 (s, 1 H), 7.20 (d,J = 8.9 Hz, 1 H), 4.18 (q,J = 7.1 Hz, 2 H), 3.02 (s,3 H), 2.83 (d, J = 16.5 Hz,1 H), 1.47 (s, 3 H), 1.42 (t,J = 7.1 Hz, 3 H) | 387 | 6.68 |
| 180 | 2-amino-3,6-dimethyl-6-(3′-propoxybiphenyl-3-yl)-5,6-dihydro-pyrimidin-4(3H)-one | 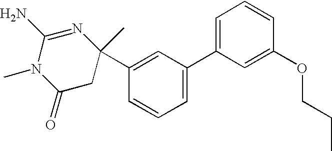 | ¹H NMR (500.131 MHz,DMSO) δ 7.85 (s, 1 H), 7.65(d, J = 7.3 Hz, 1 H), 7.59-7.49 (m, 3 H), 7.33 (d,J = 7.3 Hz, 2 H), 7.29 (s,1 H), 7.09 (d, J = 7.3 Hz,1 H), 4.15 (t, J = 6.5 Hz,1 H), 3.14 (s, 3 H), 2.95 (d,J = 16.1 Hz, 1 H), 1.91(sextet, J = 7.3 Hz, 2 H), 1.59(s, 3 H), 1.16 (t, J = 7.3 Hz,3 H) | 351 | 6.39 |
| 181 | methyl 3′-(2-amino-1,4-dimethyl-6-oxo-1,4,5,6-tetrahydro-pyrimidin-4-yl)biphenyl-4-carboxylate | 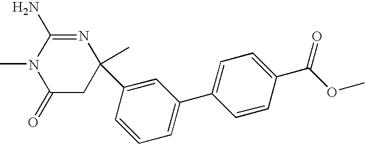 | #N/A | 351 | 5.47 |
| 182 | 2-amino-3,6-dimethyl-6-(2′-phenoxybiphenyl-3-yl)-5,6-dihydro-pyrimidin-4(3H)-one | 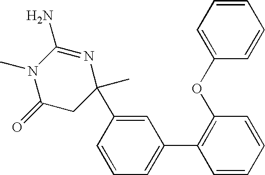 | ¹H NMR (500.132 MHz,DMSO) δ 7.59 (s, 1 H), 7.46(d, J = 8.1 Hz, 1 H), 7.40(t, J = 8.1 Hz, 1 H), 7.38-7.26(m, 6 H), 7.07-6.99 (m, 2 H),6.90 (d, J = 8.1 Hz, 2 H),2.97 (s, 3 H), 2.89 (d,J = 16.1 Hz, 1 H), 2.73(d, J = 16.1 Hz, 1 H), 1.35(s, 3 H) | 385 | 6.42 |
| 183 | 2-amino-6-(4′-isobutylbiphenyl-3-yl)-3,6-dimethyl-5,6-dihydropyrimidin-4(3H)-one | 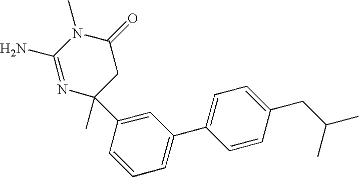 | ¹H NMR (500.132 MHz,DMSO) δ 7.61 (s, 1 H), 7.45(d, J = 8.2 Hz, 2 H), 7.16(d, J = 8.2 Hz, 2 H), 7.41-7.37 (m, 1 H), 7.29 (d,J = 4.6 Hz, 2 H), 2.72 (d,J = 16.1 Hz, 1 H), 2.94 (d,J = 16.1 Hz, 1 H), 2.90 (s,3 H), 1.78 (septet, J = 6.7 Hz,1 H), 1.35 (s, 3 H), 0.80 (d,J = 6.8 Hz, 6 H) | 349 | 7.01 |
| 184 | 2-amino-6-[2′-(benzyloxy)biphenyl-3-yl]-3,6-dimethyl-5,6-dihydropyrimidin-4(3H)-one | 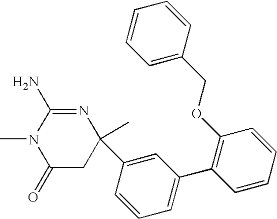 | ¹H NMR (500.132 MHz,DMSO) δ 7.63 (s, 1 H),7.44-7.26 (m, 10 H), 7.20 (d,J = 9.1 Hz, 1 H), 7.05 (t,J = 7.7 Hz, 1 H), 5.12 (s,2 H), 3.00 (s, 3 H), 2.91 (d,J = 16.4 Hz, 1 H), 2.77(d, J = 16.4 Hz, 1 H), 1.37(s, 3 H) | 399 | 6.59 |
| 185 | methyl 3′-(2-amino-1,4-dimethyl-6-oxo-1,4,5,6-tetrahydropyrimidin-4-yl)biphenyl-3-carboxylate | 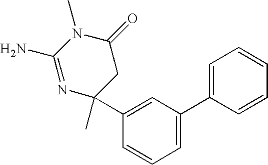 | ¹H NMR (500.131 MHz,DMSO) δ 7.96 (s, 1 H), 7.76(d, J = 8.3 Hz, 1 H), 7.72 (d,J = 8.3 Hz, 1 H), 7.57 (s,1 H), 7.44 (t, J = 7.8 Hz, 1 H),7.34 (d, J = 7.4 Hz, 1 H),7.30-7.21 (m, 2 H), 3.70 (s,3 H), 2.80 (s, 3 H), 2.61 (d,J = 15.7 Hz, 1 H), 1.24 (s,3 H) | 351 | 5.45 |
| 186 | benzyl 3′-(2-amino-1,4-dimethyl-6-oxo-1,4,5,6-tetrahydropyrimidin-4-yl)biphenyl-3-carboxylate | 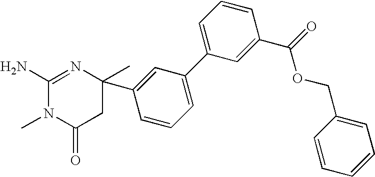 | ¹H NMR (500.132 MHz,DMSO) δ 8.20 (d, J = 9.2 Hz,2 H), 8.01 (d, J = 8.3 Hz,1 H), 7.93 (d, J = 8.3 Hz,1 H), 7.77 (s, 1 H), 7.65 (t,J = 8.3 Hz, 1 H), 7.56-7.34(m, 7 H), 5.41 (s, 2 H), 3.16(d, J = 15.7 Hz, 1 H), 3.00(s, 3 H), 2.82 (d, J = 15.7 Hz,1 H), 1.45 (s, 3 H) | 427 | 6.72 |
| 187 | 2-amino-6-(2′-methoxy-5′-methylbiphenyl-3-yl)-3,6-dimethyl-5,6-dihydropyrimidin-4(3H)-one | 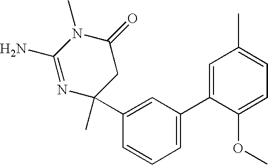 | ¹H NMR (500.131 MHz,DMSO) δ 3.04 (d,J = 16.4 Hz, 1 H), 3.01 (s,3 H), 2.83 (d, J = 16.4 Hz,1 H), 2.29 (s, 3 H), 1.45 (s,3 H), 3.72 (s, 3 H), 7.39-7.29 (m, 3 H), 7.52 (s, 1 H),7.08 (s, 1 H), 7.14 (d,J = 8.2 Hz, 1 H), 6.99 (d,J = 8.2 Hz, 1 H) | 337 | 5.89 |
| 188 | 2-amino-6-(4′-methoxy-3′,5′-dimethylbiphenyl-3-yl)-3,6-dimethyl-5,6-dihydropyrimidin-4(3H)-one | 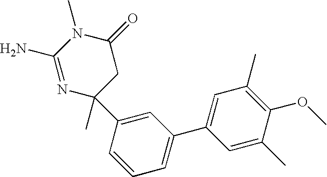 | ¹H NMR (500.132 MHz,DMSO) δ 7.66 (s, 1 H), 7.47-7.42 (m, 1 H), 7.40-7.34 (m,2 H), 7.29 (s, 2 H), 3.69 (s,3 H), 3.07-2.97 (m, 4 H),2.82 (d, J = 16.4 Hz, 1 H),2.30 (s, 6 H), 1.45 (s, 3 H) | 351 | 6 |
| 189 | benzyl [3′-(2-amino-1,4-dimethyl-6-oxo-1,4,5,6-tetrahydropyrimidin-4-yl)biphenyl-4-yl]carbamate | 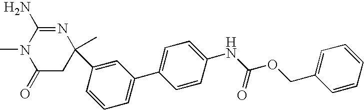 | ¹H NMR (500.131 MHz,DMSO) δ 7.69 (s, 1 H), 7.61-7.55 (m, 5 H), 7.50-7.34(m, 7 H), 5.18 (s, 2 H), 3.04(d, J = 16.4 Hz, 1 H), 3.00(s, 3 H), 2.83 (d, J = 16.4 Hz,1 H), 1.45 (s, 3 H) | 442 | 6.44 |
| 190 | ethyl 3′-(2-amino-1,4-dimethyl-6-oxo-1,4,5,6-tetrahydropyrimidin-4-yl)biphenyl-4-carboxylate | 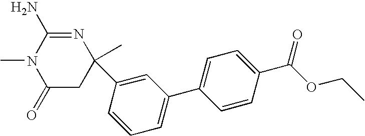 | ¹H NMR (500.132 MHz,DMSO) δ 8.05 (d, J = 8.9 Hz,2 H), 7.83-7.78 (m, 3 H), 7.59(d, J = 7.1 Hz, 1 H), 7.50(d, J = 7.1 Hz, 1 H), 7.46 (t,J = 7.6 Hz, 1 H), 4.35 (q,J = 7.1 Hz, 2 H), 3.07-2.97(m, 4 H), 2.84 (d,J = 16.4 Hz, 1 H), 1.46 (s,3 H), 1.35 (t, J = 7.1 Hz, 3 H) | 365 | 5.91 |
| 191 | benzyl 3′-(2-amino-1,4-dimethyl-6-oxo-1,4,5,6-tetrahydropyrimidin-4-yl)biphenyl-4-carboxylate | 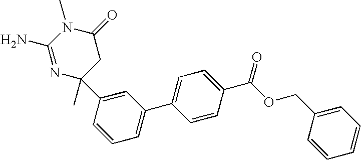 | ¹H NMR (500.131 MHz,DMSO) δ 7.95 (d, J = 7.9 Hz,2 H), 7.71-7.65 (m, 3 H), 7.45(d, J = 7.1 Hz, 1 H), 7.39-7.26 (m, 6 H), 7.24 (t,J = 7.3 Hz, 1 H), 5.26 (s,2 H), 2.92-2.84 (m, 4 H), 2.69(d, J = 16.1 Hz, 1 H), 1.31 (s,3 H) | 427 | 6.79 |
| 192 | N-[3′-(2-amino-1,4-dimethyl-6-oxo-1,4,5,6-tetrahydropyrimidin-4-yl)biphenyl-2-yl]methanesulfonamide | 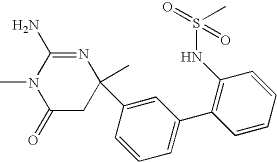 | ¹H NMR (500.132 MHz,DMSO) δ 7.52 (s, 1 H), 7.46-7.33 (m, 6 H), 7.31 (d,J = 7.6 Hz, 1 H), 3.04 (d,J = 16.5 Hz, 1 H), 3.01 (s,3 H), 2.86 (d, J = 16.5 Hz,1 H), 2.57 (s, 3 H), 1.46 (s,3 H) | 386 | 4.66 |
| 193 | 3′-(2-amino-1,4-dimethyl-6-oxo-1,4,5,6-tetrahydropyrimidin-4-yl)-N-(tert-butyl)biphenyl-2-sulfonamide | 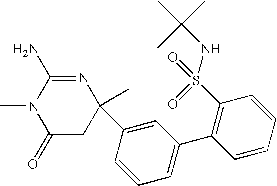 | ¹H NMR (500.131 MHz,DMSO) δ 7.99 (s, 1 H), 7.83(d, J = 7.9 Hz, 1 H), 7.44-7.36 (m, 2 H), 7.37-7.30 (m,2 H), 7.26 (s, 1 H), 7.21(d, J = 7.7 Hz, 1 H), 7.14 (t,J = 7.7 Hz, 1 H), 7.07 (t,J = 7.9 Hz, 2 H), 2.78 (s,3 H), 2.73 (d, J = 15.6 Hz,1 H), 2.56 (d, J = 15.6 Hz,1 H), 1.21 (s, 3 H), 0.72(s, 9 H) | 428 | 5.8 |
| 194 | 2-amino-6-[3-(3,5-dimethylisoxazol-4-yl)phenyl]-3,6-dimethyl-5,6-dihydropyrimidin-4(3H)-one | 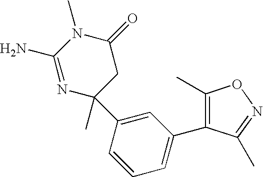 | ¹H NMR (500.132 MHz,DMSO) δ 7.22-7.14 (m, 3 H),7.00 (d, J = 7.0 Hz, 1 H),2.78-2.73 (m, 4 H), 2.55 (d,J = 16.4 Hz, 1 H), 2.15 (s,3 H), 1.98 (s, 3 H), 1.18 (s,3 H) | 312 | 4.53 |
| 195 | N-[3′-(2-amino-1,4-dimethyl-6-oxo-1,4,5,6-tetrahydropyrimidin-4-yl)biphenyl-3-yl]acetamide | 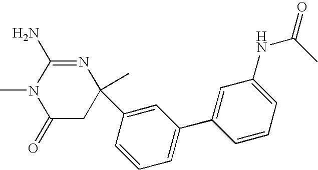 | ¹H NMR (500.131 MHz,DMSO) δ 10.08 (s, 1 H), 7.86(s, 1 H), 7.71 (s, 1 H), 7.64(d, J = 7.6 Hz, 1 H), 7.50-7.46 (m, 3 H), 7.43 (t,J = 8.0 Hz, 1 H), 7.32 (d,J = 7.6 Hz, 1 H), 3.08-3.00 (m, 4 H), 2.87 (d,J = 16.4 Hz, 1 H), 2.12 (s,3 H), 1.49 (s, 3 H) | 350 | 4.62 |
| 196 | 2-amino-6-(2′,6′-dichlorobiphenyl-3-yl)-3,6-dimethyl-5,6-dihydropyrimidin-4(3H)-one | 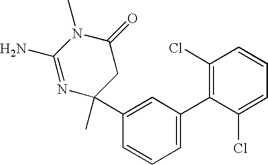 | ¹H NMR (500.132 MHz,DMSO) δ 7.43-7.38 (m, 2 H),7.33-7.29 (m, 1 H), 7.29-7.23 (m, 2 H),7.16-7.13 (m, 1 H), 6.94-6.91(m, 1 H), 2.92-2.76 (m, 4 H),1.23 (s, 3 H) | 361 | 5.95 |
| 197 | 2-amino-6-(3′-chloro-2′-fluorobiphenyl-3-yl)-3,6-dimethyl-5,6-dihydropyrimidin-4(3H)-one | 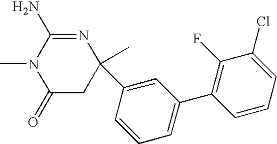 | ¹H NMR (500.131 MHz,DMSO)δ 7.66-7.59 (m, 2 H),7.53-7.44 (m, 3 H), 7.41 (d,J = 7.8 Hz, 1 H), 7.34 (t,J = 8.2 Hz, 1 H), 3.04-2.96(m, 4 H), 2.83 (d,J = 16.3 Hz, 1 H), 1.44 (s,3 H) | 345 | 5.98 |
| 198 | 2-amino-6-(4′-butoxy-3′-chlorobiphenyl-3-yl)-3,6-dimethyl-5,6-dihydropyrimidin-4(3H)-one | 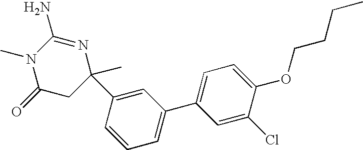 | ¹H NMR (500.132 MHz,DMSO) δ 7.83-7.77 (m, 2 H),7.67 (d, J = 8.3 Hz, 1 H),7.61-7.56 (m, 1 H), 7.52-7.44(m, 2 H), 7.34 (d, J = 9.1 Hz,1 H), 4.21 (t, J = 6.9 Hz,2 H), 3.13 (d, J = 16.4 Hz,1 H), 3.09 (s, 3 H), 2.91 (d,J = 16.4 Hz, 1 H), 1.85(quintet, J = 7.0 Hz, 2 H),1.59 (sextet, J = 7.4 Hz, 2 H),1.53 (s, 3 H), 1.06 (t,J = 7.4 Hz, 3 H) | 399 | 7.19 |
| 199 | [3′-(2-amino-1,4-dimethyl-6-oxo-1,4,5,6-tetrahydropyrimidin-4-yl)biphenyl-3-yl]formamide | 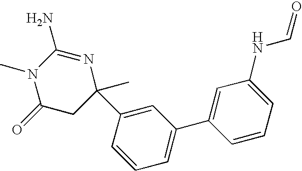 | — | 308 | |
| 200 | 2-amino-6-[3-(2-fluoro-pyridin-3-yl)phenyl]-3,6-dimethyl-5,6-dihydro-pyrimidin-4(3H)-one | 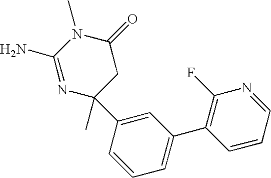 | ¹H NMR (500 MHz,DMSO-d6)δ 8.24 (dd, J = 27.2,3.9 Hz, 1 H), 8.08 (t,J = 4.0 Hz, 1 H), 7.68 (s,1 H), 7.54-7.43 (m, 4 H), 3.01(s, 3 H), 2.97 (d, J = 16.4 Hz,1 H), 2.81 (d, J = 16.4 Hz,1 H), 1.44 (s, 3 H) | 312 | 4.44 |
| 201 | 2-amino-6-(3′-bromo-2′-ethoxy-5′-methyl-biphenyl-3-yl)-3,6-dimethyl-5,6-dihydro-pyrimidin-4(3H)-one | 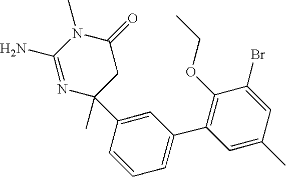 | ¹H NMR (500.132 MHz,DMSO) δ 7.67-7.51 (m, 1 H),7.49-7.28 (m, 4 H), 7.17-7.10(m, 1 H), 3.48 (q, J = 6.8 Hz,2 H), 2.98 (s, 3 H), 2.31 (s,3 H), 1.39 (s, 3 H), 0.99 (t,J = 7.0 Hz, 3 H) | 429 | 6.75 |
| 202 | 2-amino-6-[3-(2-ethoxy-1-naphthyl)phenyl]-3,6-dimethyl-5,6-dihydropyrimidin-4(3H)-one | 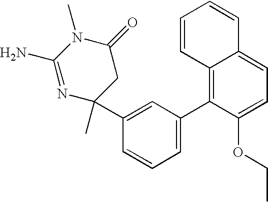 | ¹H NMR (500.131 MHz,DMSO) δ 7.97 (d, J = 8.6 Hz,1 H), 7.93-7.89 (m, 1 H),7.53-7.49 (m, 1 H), 7.48-7.43(m, 2 H), 7.40-7.31 (m, 4 H),7.19-7.13 (m, 1 H), 4.14-4.05 (m, 2 H), 3.07-2.95 (m,4 H), 2.84-2.75 (m, 1 H), 1.46(s, 3 H), 1.22-1.11 (m, 3 H) | 387 | 6.48 |
| 203 | 2-amino-6-[4′-(benzyloxy)-2′-fluorobiphenyl-3-yl]-3,6-dimethyl-5,6-dihydropyrimidin-4(3H)-one | 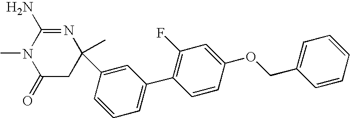 | ¹H NMR (500.131 MHz,DMSO) δ 7.78 (s, 1 H), 7.64(d, J = 12.5 Hz, 1 H), 7.61-7.55 (m, 3 H), 7.54-7.39 (m,7 H), 5.33 (s, 2 H), 3.16 (d,J = 16.3 Hz, 1 H), 3.08 (s,3 H), 2.93 (d, J = 16.3 Hz,1 H), 1.54 (s, 3 H) | 417 | 6.86 |
| 204 | 2-amino-3,6-dimethyl-6-[2′-(morpholin-4-ylmethyl)biphenyl-3-yl]-5,6-dihydro-pyrimidin-4(3H)-one | 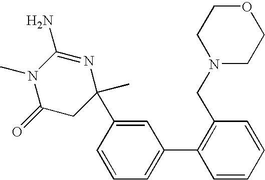 | ¹H NMR (500.131 MHz,DMSO) δ 8.32 (s, 1 H), 7.78(s, 1 H), 7.64 (d, J = 13.2 Hz,1 H), 7.61-7.54 (m, 1 H),7.54-7.39 (m, 6 H), 5.33 (s,2 H), 3.16 (d, J = 16.7 Hz,1 H), 3.08 (s, 3 H), 2.93 (d,J = 16.7 Hz, 1 H), 1.54 (s,3 H) | 392 | 3.12 |
| 205 | 3′-(2-amino-1,4-dimethyl-6-oxo-1,4,5,6-tetrahydropyrimidin-4-yl)-3-fluorobiphenyl-4-carbaldehyde | 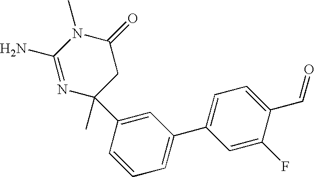 | ¹H NMR (500.132 MHz,DMSO) δ 10.27 (s, 1 H), 7.94(t, J = 8.2 Hz, 1 H), 7.88 (s,1 H), 7.78-7.71 (m, 2 H), 7.67(d, J = 7.2 Hz, 1 H), 7.55 (d,J = 7.2 Hz, 1 H), 7.48 (t,J = 7.9 Hz, 1 H), 3.05 (d,J = 16.1 Hz, 1 H), 3.01 (s,3 H), 2.86 (d, J = 16.1 Hz,1 H), 1.46 (s, 3 H) | 339 | 5.21 |
| 206 | 2-amino-3,6-dimethyl-6-[4′-(trimethylsilyl)biphenyl-3-yl]-5,6-dihydropyrimidin-4(3H)-one | 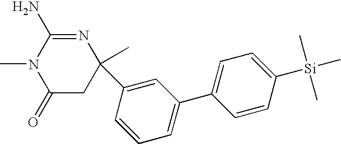 | ¹H NMR (500.131 MHz,DMSO) δ 7.54 (s, 1 H), 7.47-7.44 (m, 4 H), 7.35-7.32 (m,1 H), 7.29-7.22 (m, 2 H),2.87-2.80 (m, 4 H), 2.64 (d,J = 16.3 Hz, 1 H), 1.28 (s,3 H), 0.12 (s, 9 H) | 365 | 7.27 |
| 207 | 2-amino-6-(3′-butoxy-biphenyl-3-yl)-3,6-dimethyl-5,6-dihydro-pyrimidin-4(3H)-one | 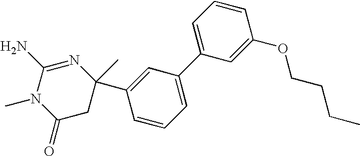 | ¹H NMR (500.132 MHz,DMSO) δ 7.62 (s, 1 H), 7.41(d, J = 7.8 Hz, 1 H), 7.37-7.24 (m, 3 H), 7.10 (d,J = 7.8 Hz, 1 H),7.06 (s, 1 H),6.85 (d, J = 7.8 Hz, 1 H),3.96 (t, J = 7.2 Hz, 2 H),2.95-2.86 (m, 4 H), 2.70 (d,J = 16.6 Hz, 1 H), 1.65(quintet, J = 7.2 Hz, 0 H),1.43-1.32 (m, 5 H), 0.87 (t,J = 7.4 Hz, 0 H) | 365 | 6.86 |
| 208 | 2-amino-3,6-dimethyl-6-(2′,4′,6′-trimethyl-biphenyl-3-yl)-5,6-dihydropyrimidin-4(3H)-one | 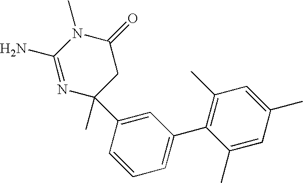 | ¹H NMR (500.132 MHz,DMSO)δ 7.41-7.33 (m, 2 H),7.12 (s, 1 H), 6.97-6.92 (m,1 H), 6.91 (s, 2 H), 2.94 (s,3 H), 2.73 (d, J = 16.6 Hz,1 H), 2.25 (s, 3 H), 1.89 (s,3 H), 1.89 (s, 3 H), 1.42 (s,3 H) | 335 | 6.51 |
| 209 | 2-amino-6-[3-(2-chloro-3-fluoropyridin-4-yl)phenyl]-3,6-dimethyl-5,6-dihydropyrimidin-4(3H)-one | 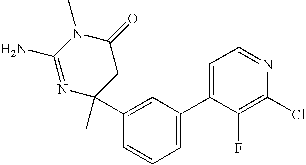 | NMR too dilute | 346 | 5.1 |
| 210 | 2-amino-6-(5′-chloro-2′-methylbiphenyl-3-yl)-3,6-dimethyl-5,6-dihydropyrimidin-4(3H)-one | 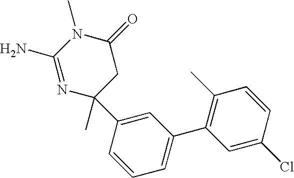 | ¹H NMR (500.132 MHz,DMSO)δ 7.60-7.50 (m, 3 H),7.48-7.44 (m, 2 H), 7.37-7.30(m, 2 H), 3.11 (s, 3 H), 2.90(d, J = 16.4 Hz, 1 H), 2.29(s, 3 H), 1.54 (s, 3 H) | 341 | 6.31 |
| 211 | 2-amino-6-(2′,5′-difluoro-biphenyl-3-yl)-3,6-dimethyl-5,6-dihydropyrimidin-4(3H)-one | 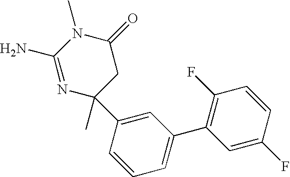 | ¹H NMR (500 MHz,DMSO-d6)δ 8.22 (s, 1 H),7.67-7.60 (m, 2 H), 7.59-7.53 (m, 1 H), 7.53-7.35 (m,3 H), 7.32-7.24 (m, 1 H), 3.01(s, 3 H), 2.82 (d, J = 16.0 Hz,1 H), 1.44 (s, 3 H) | 329 | 5.58 |
| 212 | 2-amino-3,6-dimethyl-6-(1, 1′:4′,1″-terphenyl-3-yl)-5,6-dihydro-pyrimidin-4(3H)-one | 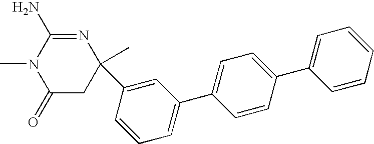 | ¹H NMR (500.132 MHz,DMSO) δ 7.82-7.71 (m,7 H), 7.61-7.55 (m, 1 H), 7.50(t, J = 7.8 Hz, 2 H), 7.46-7.43 (m, 2 H), 7.40 (t,J = 7.3 Hz, 1 H), 3.06 (d,J = 16.2 Hz, 1 H), 3.02(s, 3 H), 2.84 (d, J = 16.2 Hz,1 H), 1.47 (s, 3 H) | 369 | 6.73 |
| 213 | 2-amino-6-(3′-chloro-4′-fluorobiphenyl-3-yl)-3,6-dimethyl-5,6-dihydropyrimidin-4(3H)-one | 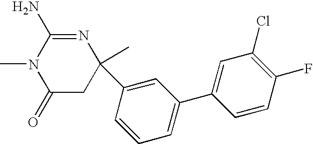 | ¹H NMR (500 MHz,DMSO-d6)δ 8.00 (s,1 H), 7.67 (d, J = 7.1 Hz,1 H), 7.54 (s, 1 H), 7.48-7.44(m, 1 H), 7.31 (dd, J = 8.9,9.3 Hz, 2 H), 7.25 (d,J = 7.4 Hz, 1 H), 7.20 (t,J = 7.7 Hz, 1 H), 2.79 (s,3 H), 2.60 (d, J = 16.0 Hz,1 H), 1.22 (s, 3 H) | 345 | 6.06 |
| 214 | 2-amino-6-(4′-fluoro-biphenyl-3-yl)-3,6-dimethyl-5,6-dihydro-pyrimidin-4(3H)-one | 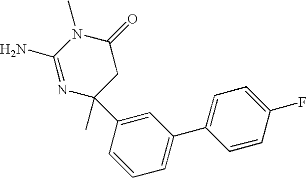 | | 311 | 5.56 |
| 215 | 2-amino-6-(3-dibenzo[b,d]thien-4-ylphenyl)-3,6-dimethyl-5,6-dihydro-pyrimidin-4(3H)-one | 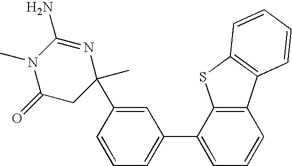 | | 399 | 6.72 |
| 216 | 2-amino-6-biphenyl-3-yl-3,6-dimethyl-5,6-dihydropyrimidin-4(3H)-one | 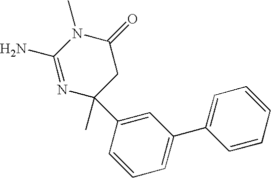 | ¹H NMR (500.132 MHz,DMSO) δ 7.51 (s, 1 H), 7.42(d, J = 7.9 Hz, 2 H), 7.32-7.24 (m, 3 H), 7.23-7.19 (m,2 H), 7.16 (t, J = 7.4 Hz,1 H), 2.83 (d, J = 16.4 Hz,1 H), 2.79 (s, 3 H), 2.61(d, J = 16.4 Hz, 1 H), 1.24(s, 3 H) | 293 | 5.2 |
| 217 | 2-amino-6-(2′,3′-dimethyl-biphenyl-3-yl)-3,6-dimethyl-5,6-dihydro-pyrimidin-4(3H)-one | 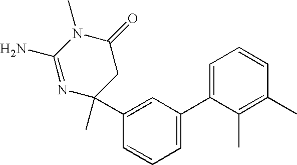 | ¹H NMR (500.132 MHz,DMSO) δ 7.24-7.18 (m,2 H), 7.16 (s, 1 H), 7.01-6.93(m, 3 H), 6.82 (d, J = 7.2 Hz,1 H), 2.87-2.78 (m, 4 H), 2.62(d, J = 16.1 Hz, 1 H), 2.11(s, 3 H), 1.88 (s, 3 H), 1.26(s, 3 H) | 321 | 6.06 |
| 218 | 2-amino-6-{3-[(E)-2-biphenyl-4-ylvinyl]phenyl}-3,6-dimethyl-5,6-dihydropyrimidin-4(3H)-one | 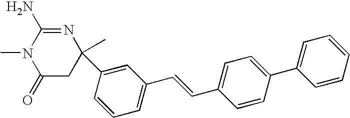 | | 395 | 7.21 |
| 219 | 2-amino-6-[4′-(benzyloxy)-3′-chlorobiphenyl-3-yl]-3,6-dimethyl-5,6-dihydro-pyrimidin-4(3H)-one | 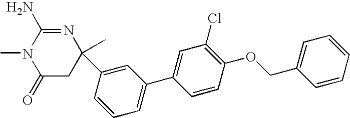 | ¹H NMR (500.132 MHz,DMSO) δ 7.65 (s, 1 H), 7.61(s, 1 H), 7.48 (d, J = 8.3 Hz,1 H), 7.43-7.37 (m, 3 H),7.36-7.22 (m, 6 H), 5.18 (s,2 H), 2.93-2.87 (m, 4 H),2.71 (d, J = 16.2 Hz, 1 H),1.34 (s, 3 H) | 433 | 7.05 |
| 220 | 2-amino-6-(4′-butoxy-2′-methylbiphenyl-3-yl)-3,6-dimethyl-5,6-dihydro-pyrimidin-4(3H)-one | 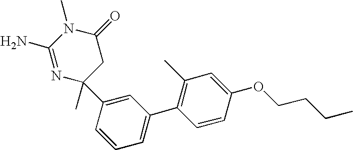 | ¹H NMR (500.132 MHz,DMSO)δ 7.39-7.33 (m, 3 H),7.18-7.11 (m, 1 H), 7.09 (d,J = 8.5 Hz, 1 H), 6.87 (d,J = 13.5 Hz, 1 H), 6.82(d, J = 8.5 Hz, 1 H), 3.99 (t,J = 6.4 Hz, 2 H), 3.07-2.95(m, 4 H), 2.80 (d,J = 16.4 Hz, 1 H), 2.18 (s,3 H), 1.71 (quintet,J = 7.0 Hz, 1 H), 1.50-1.41(m, 6 H), 0.95 (t, J = 7.4 Hz,3 H) | 379 | 7.05 |
| 221 | 3′-(2-amino-1,4-dimethyl-6-oxo-1,4,5,6-tetrahydropyrimidin-4-yl)biphenyl-2-carbaldehyde | 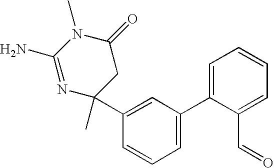 | ¹H NMR (500.132 MHz,DMSO) δ 9.58 (s, 1 H), 7.70(d, J = 8.7 Hz, 1 H), 7.53(t, J = 7.5 Hz, 1 H), 7.36(t, J = 7.5 Hz, 1 H), 7.31 (d,J = 7.6 Hz, 1 H), 7.29-7.21(m, 3 H), 7.09 (d, J = 8.7 Hz,1 H), 2.78 (s, 3 H), 2.76-2.70(m, 1 H), 2.57 (d,J = 15.8 Hz, 1 H), 1.20 (s,3 H) | 321 | 4.97 |
| 222 | 2-amino-6-(3′-bromo-2′-isopropoxy-5′-methylbiphenyl-3-yl)-3,6-dimethyl-5,6-dihydropyrimidin-4(3H)-one | 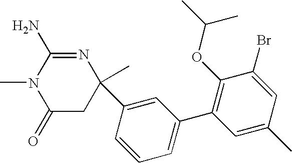 | ¹H NMR (500.132 MHz,DMSO) δ 7.57 (s, 1 H), 7.45(s, 1 H), 7.41 (t, J = 7.0 Hz,1 H), 7.37 (d, J = 8.3 Hz,1 H), 7.31 (d, J = 8.3 Hz,1 H), 7.12 (s, 1 H), 3.79(septet, J = 6.4 Hz, 1 H),2.97 (s, 3 H), 2.75 (d,J = 16.2 Hz, 1 H), 2.31(s, 3 H), 1.41 (s, 3 H), 0.90(d, J = 6.4 Hz, 3 H), 0.88 (d,J = 6.6 Hz, 3 H) | 443 | 6.92 |
| 223 | 3′-(2-amino-1,4-dimethyl-6-oxo-1,4,5,6-tetrahydropyrimidin-4-yl)biphenyl-4-carboxamide | 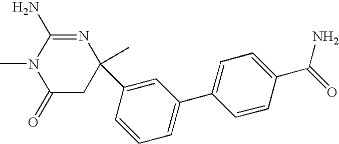 | ¹H NMR (500.132 MHz,DMSO) δ 8.34 (s, 1 H), 8.14(s, 1 H), 8.09 (d, J = 8.6 Hz,2 H), 7.90 (s, 1 H), 7.85 (d,J = 8.6 Hz, 2 H), 7.69 (d,J = 6.9 Hz, 1 H), 7.61-7.53(m, 2 H), 7.51 (s, 1 H), 3.20-3.09 (m, 4 H), 2.95 (d,J = 16.1 Hz, 1 H), 1.57(s, 3 H) | 336 | 3.93 |
| 224 | 2-amino-6-(3′,5′-difluorobiphenyl-3-yl)-3,6-dimethyl-5,6-dihydropyrimidin-4(3H)-one | 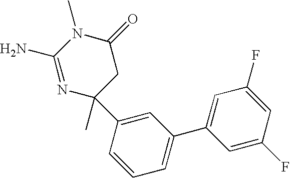 | ¹H NMR (500 MHz,DMSO-d6)δ 7.99 (s, 1 H),7.57 (s, 1 H), 7.43-7.30 (m,2 H), 7.27 (d, J = 7.9 Hz,1 H), 7.24-7.17 (m, 3 H),7.01 (t, J = 9.2 Hz, 1 H),2.84-2.72 (m, 4 H), 2.60 (d,J = 15.8 Hz, 1 H), 1.21 (s,3 H) | 329 | 5.76 |
| 225 | 2-amino-3,6-dimethyl-6-[3-(1H-pyrazol-3-yl)phenyl]-5,6-dihydro-pyrimidin-4(3H)-one | 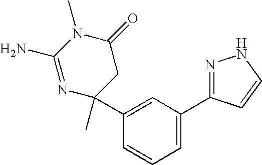 | ¹H NMR (300.132 MHz,DMSO) δ 8.26 (s, 2 H), 7.87(s, 1 H), 7.75-7.57 (m, 2 H),7.41-7.27 (m, 2 H), 6.67 (s,1 H), 3.07 (d, J = 14.6 Hz,1 H), 2.99 (s, 3 H), 2.87 (d,J = 17.5 Hz, 1 H), 1.46 (s,3 H) | 283 | 3.62 |
| 226 | 2-amino-3,6-dimethyl-6-(3-quinolin-5-ylphenyl)-5,6-dihydropyrimidin-4(3H)-one | 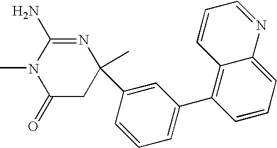 | ¹H NMR (300.132 MHz,DMSO) δ 8.94 (s, 1 H), 8.28-8.00 (m, 3 H), 7.83 (t,J = 6.8 Hz, 1 H), 7.62-7.32(m, 5 H), 3.14 (d,J = 17.5 Hz, 1 H), 3.04(s, 3 H), 2.91 (d, J = 16.5 Hz,1 H), 1.52 (s, 3 H) | 344 | 3.43 |
| 227 | 2-amino-6-[2′,5′-dimethoxybiphenyl-3-yl)-3,6-dimethyl-5,6-dihydropyrimidin-4(3H)-one | 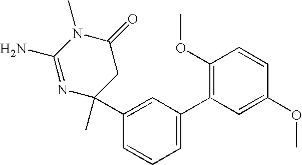 | ¹H NMR (600.134 MHz,CDCl3) δ 8.31 (s, 1 H), 7.54(s, 1 H), 7.38 (s, 2 H), 7.05(d, J = 9.6 Hz, 1 H), 6.91 (dd,J = 9.3, 2.6 Hz, 1 H), 6.85(d, J = 3.0 Hz, 1 H), 3.76 (s,3 H), 3.70 (s, 3 H), 3.21 (d,J = 16.4 Hz, 1 H), 3.02(s, 3 H), 2.96 (d, J = 16.4 Hz,1 H), 1.51 (s, 3 H) | 353 | 5.28 |
| 228 | 2-amino-6-(3′-chlorobiphenyl-3-yl)-3,6-dimethyl-5,6-dihydropyrimidin-4(3H)-one | 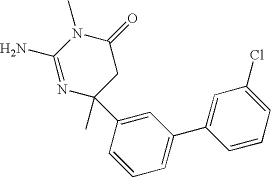 | ¹H NMR (300 MHz,DMSO-d6)δ 1.52 (s, 3 H),3.27 (d, J = 16.1 Hz, 1 H),3.01 (s, 3 H), 2.97 (d,J = 16.4 Hz, 1 H), 7.80-7.38(m, 8 H), 8.31 (s, 1 H) | 328 | 5.76 |
| 229 | 2-amino-6-(4′-chlorobiphenyl-3-yl)-3,6-dimethyl-5,6-dihydro-pyrimidin-4(3H)-one | 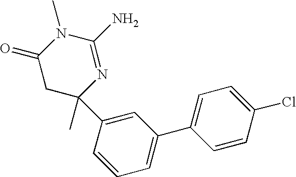 | ¹H NMR (300 MHz,DMSO-d6)δ 8.31 (s, 1 H),8.04 (s, 1 H), 7.98 (d,J = 8.3 Hz, 1 H), 7.83 (d,J = 9.0 Hz, 2 H), 7.68-7.60(m, 1 H), 7.48 (d, J = 4.9 Hz,2 H), 3.28 (d, J = 16.4 Hz,1 H), 3.05-2.93 (m, 4 H),1.53 (s, 3 H) | 328 | 6.42 |
| 230 | 2-amino-6-(3′-bromo-2′-butoxy-5′-methyl-biphenyl-3-yl)-3,6-dimethyl-5,6-dihydro-pyrimidin-4(3H)-one | 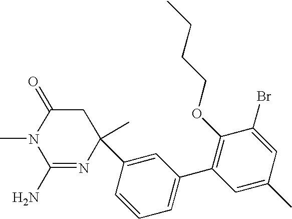 | ¹H NMR (300 MHz,DMSO-d6)δ 8.28 (s, 1 H),7.53 (s, 1 H), 7.48-7.31 (m,4 H), 7.11 (s, 1 H), 3.41 (t,J = 3.2 Hz, 21 H), 3.14 (d,J = 16.1 Hz, 1 H), 2.99 (s,3 H), 2.92 (d, J = 16.1 Hz,1 H), 2.30 (s, 3 H), 1.49 (s,3 H), 1.37 (quintet,J = 3.3 Hz, 2 H), 1.14(quintet, J = 3.8 Hz, 2 H),0.65 (t, J = 3.7 Hz, 3 H) | 458 | 7.17 |

The following compounds in Table were synthesized using methods analogous to those previously described for Example 36 employing the appropriate boronic acid with the precursor aryl bromide of the title compound in Example 29.

TABLE 8

| Ex. | name | Structure | m/z | NMR data |
|---|---|---|---|---|
| 231 | 2-amino-6-[2-[3-(2-aminophenyl)phenyl]ethyl]-3-methyl-3H-pyrimidin-4-one | 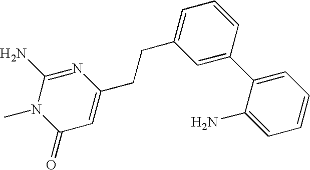 | 321 | (300 MHz, DMSO) δ 8.55 (s, 2H),7.40 (t, J = 7.6 Hz, 1H), 7.32 (s,1H), 7.28 (d, J = 6.0 Hz, 2H), 7.26(d, J = 6.4 Hz, 2H), 7.14 (t, J = 7.8Hz, 1H), 7.06 (d, J = 7.5 Hz, 1H),6.92 (d, J = 9.1 Hz, 1H), 6.82 (t,J = 7.4 Hz, 1H), 5.85 (s, 1H), 3.2 |
| 232 | 2-amino-3-methyl-6-[2-[3-(p-tolyl)phenyl]ethyl]-3H-pyrimidin-4-one | 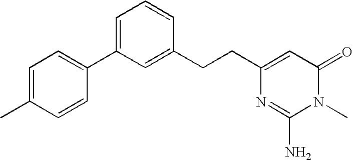 | 320 | (400 MHz, DMSO) δ 7.52 (d, J =8.0 Hz, 2H), 7.46 (s, 1H), 7.43 (d,J = 7.9 Hz, 1H), 7.34 (t, J = 7.6Hz, 1H), 7.26 (d, J = 7.9 Hz, 2H),7.17 (d, J = 7.6 Hz, 1H), 7.05 (s, 2H), 5.53 (s, 1H), 3.22 (s, 3H),2.93 (t, J = 7.9 Hz, 2H), 2.60 (t,J = 7 |
| 233 | 2-amino-3-methyl-6-[2-[3-(m-tolyl)phenyl]ethyl]-3H-pyrimidin-4-one | 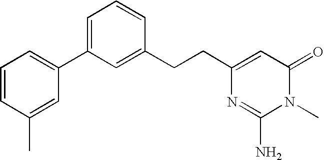 | 320 | (400 MHz, DMSO) δ 7.47-7.40m, 4H), 7.36 (d, J = 7.3 Hz, 1H),7.32 (d, J = 7.3 Hz, 1H), 7.18 (t,J = 8.8 Hz, 2H), 7.05 (s, 2H), 5.53(s, 1H), 3.22 (s, 3H), 2.94 (t, J =7.9 Hz, 2H), 2.60 (t, J = 7.9 Hz,2H), 2.37 (s, 3H) |
| 234 | 2-amino-3-methyl-6-[2-[3-(o-tolyl)phenyl]ethyl]-3H-pyrimidin-4-one | 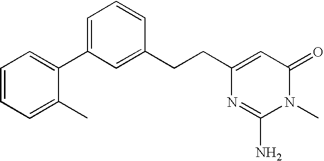 | 320 | (400 MHz, DMSO) δ 7.34 (t, J =7.8 Hz, 1H), 7.27-7.16 (m, 4H),7.13 (d, J = 1.8 Hz, 3H), 7.03 (s,2H), 5.49 (s, 1H), 3.21 (s, 3H),2.92(t, J = 7.8 Hz, 2H), 2.58 (t,J = 7.8 Hz, 2H), 2.18 (s, 3H) |
| 235 | 2-amino-6-[2-[3-[4-(hydroxymethyl)phenyl]phenyl]ethyl]-3-methyl-3H-pyrimidin-4-one | 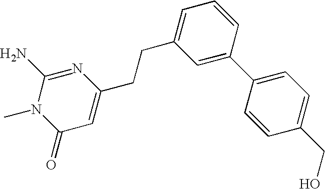 | 336 | (400 MHz, DMSO) δ 7.59 (d, J =7.5 Hz, 3H), 7.47 (d, J = 6.6 Hz,3H), 7.45 (d, J = 7.9 Hz, 4H), 7.39(d, J = 9.6 Hz, 5H), 7.35 (t, J = 7.2Hz, 4H), 7.19 (d, J = 8.8 Hz, 1H),7.05 (s, 2H), 5.53 (s, 1H), 4.54 (d,J = 5.7 Hz, 3H), 3.22 (s, 4H), 2.9 |
| 236 | 2-amino-6-[2-[3-(4-ethylsulfonylphenyl)phenyl]ethyl]-3-methyl-3H-pyrimidin-4-one | 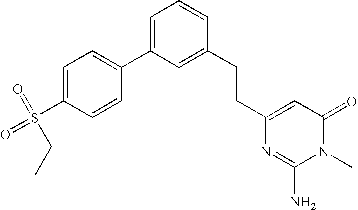 | 398 | (400 MHz, DMSO) δ 7.93 (d, J =2.8 Hz, 4H), 7.60 (s, 1H), 7.57 (d,J = 7.9 Hz, 1H), 7.42 (t, J = 7.6Hz, 1H), 7.30 (d, J = 7.5 Hz, 1H),7.05 (s, 2H), 5.53 (s, 1H), 3.31 (q,J = 10.5 Hz, 2H), 3.22 (s, 3H),2.96 (t, J = 7.8 Hz, 2H), 2.62 (t,J = |
| 237 | 2-amino-6-[2-[3-[2-(hydroxymethyl)phenyl]phenyl]ethyl]-3-methyl-3H-pyrimidin-4-one | 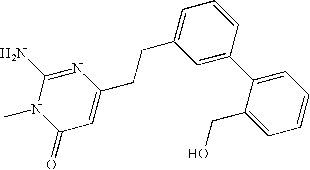 | 336 | (400 MHz, DMSO) δ 7.56 (d, J =8.4 Hz, 1H), 7.39-7.28 (m, 3H),7.20 (d, J = 12.7 Hz, 2H), 7.18(d, J = 10.1 Hz, 2H), 7.04 (s, 2H),5.52 (s, 1H), 4.37 (d, J = 3.1 Hz,2H), 3.22 (s, 3H), 3.20 (t, J = 5.5Hz, 1H), 2.91 (t, J = 8.6 Hz, 2H),2.58 |
| 238 | 2-amino-6-[2-[3-(4-hydroxy-2-methyl-phenyl)phenyl]ethyl]-3-methyl-3H-pyrimidin-4-one | 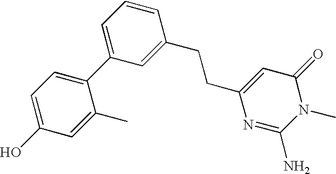 | 336 | (300 MHz, DMSO) δ 8.13 (s, 1H),7.32 (t, J = 7.7 Hz, 1H), 7.17 (d,J = 8.0 Hz, 3H), 7.13 (s, 1H), 7.10(d, J = 8.6 Hz, 1H), 6.97 (d, J =9.3 Hz, 1H), 6.67 (s, 1H), 6.64 (d,J = 8.6 Hz, 1H), 5.75 (s, 1H), 3.25(s, 3H), 2.92 (t, J = 7.8 Hz, 2H), |
| 239 | 2-amino-6-[2-[3-(4-methoxy-2-methyl-phenyl)pheny]ethyl]-3-methyl-3H-pyrimidin-4-one | 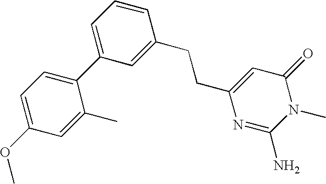 | 350 | (300 MHz, DMSO) δ 8.13 (s, 2H),7.34 (t, J = 8.2 Hz, 1H), 7.20 (d,J = 7.8 Hz, 1H), 7.14 (d, J = 5.8 Hz,1H), 7.10 (s, 1H), 7.07 (s, 1H),6.86 (d, J = 2.7 Hz, 1H), 6.81 (d,J = 8.5 Hz, 1H), 5.75 (s, 1H), 3.77(s, 3H), 3.25 (s, 3H), 2.93 (t, J |
| 240 | 2-amino-6-[2-[3-(5-chloro-2-thienyl)phenyl]ethyl]-3-methyl-3H-pyrimidin-4-one | 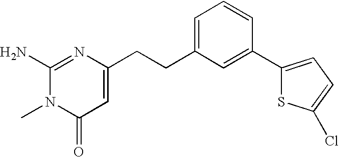 | 346 | (300 MHz, DMSO) δ 7.41 (d, J =6.4 Hz, 3H), 7.36 (t, J = 3.2 Hz,1H), 7.31 (d J = 7.1 Hz, 1H), 7.18(d, J = 7.7 Hz, 1H), 7.15 (d, J =4.0 Hz, 1H), 7.05 (s, 1H), 5.52 (s, 1H), 3.28 (s, 3H), 2.91 (t, J = 7.5Hz, 2H), 2.58 (t, J = 7.5 Hz, 2H) |
| 241 | 6-[2-[3-(5-acetyl-2-thienyl)phenyl]ethyl]-2-amino-3-methyl-3H-pyrimidin-4-one | 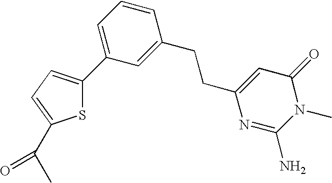 | 354 | (300 MHz, DMSO) δ 8.13 (s, 2H),7.93 (d, J = 2.8 Hz, 1H), 7.61 (d, J = 3.9 Hz, 1H), 7.58 (d, J = 6.3 Hz,1H), 7.37 (t, J = 7.3 Hz, 1H), 7.26(d, J = 7.3 Hz, 1H), 7.06 (s, 1H),5.52 (s, 1H), 3.29 (s, 3H), 2.93 (t,J = 7.2 Hz, 2H), 2.59 (t, J = 6 |
| 242 | 2-amino-6-[2-[3-[4-(methoxymethyl)phenyl]phenyl]ethyl]-3-methyl-3H-pyrimidin-4-one | 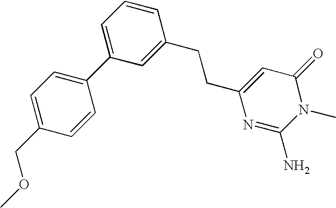 | 350 | (300 MHz, DMSO) δ7.61 (d, J =10.6 Hz, 2H), 7.47 (d, J = 12.8Hz, 2H), 7.39 (d, J = 8.5 Hz, 2H),7.36 (t, J = 8.0 Hz, 1H), 7.20 (d,J = 8.5 Hz, 1H), 7.06 (s, 2H), 5.53(s, 1H), 4.45 (s, 2H), 3.31 (s, 3H),3.22 (s, 3H), 2.94 (t, J = 7.8 Hz, 2 |
| 243 | 4-[3-[2-(2-amino-1-methyl-6-oxo-1H-pyrimidin-4-yl)ethyl]phenyl]benzamide | 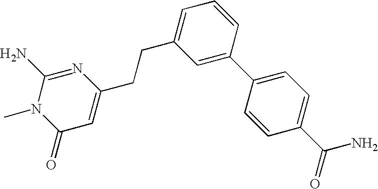 | 349 | (300 MHz, DMSO) δ 7.95 (d, J =7.9 Hz, 3H), 7.72 (d, J = 8.6 Hz,2H), 7.53 (d, J = 11.5 Hz, 2H),7.39 (t, J = 7.5 Hz, 1H), 7.34 (s,1H), 7.24 (d, J = 7.9 Hz, 1H), 7.06(s, 2H), 5.54 (s, 1H), 3.22 (s, 3H),2.95 (t, J = 7.8 Hz, 2H), 2.61 (t, J = |
| 244 | 2-amino-3-methyl-6-[2-[3-(2-methylsulfonylphenyl)phenyl]ethyl]-3H-pyrimidin-4-one | 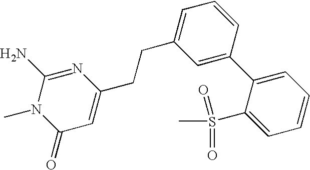 | 384 | (300 MHz, DMSO) δ 8.13 (s, 1H),8.01 (d, J = 10.9 Hz, 1H), 7.91 (d,J = 11.1 Hz, 1H), 7.74 (t, J = 8.8Hz, 1H), 7.59 (d, J = 9.0 Hz, 1H),7.58 (t, J = 6.1 Hz, 1H), 7.42 (t,J = 7.3 Hz, 1H), 7.29 (d, J = 10.1Hz, 1H), 7.05 (s, 2H), 5.53 (s, 1H), |
| 245 | 2-amino-3-methyl-6-[2-[3-(5-quinolyl)phenyl]ethyl]-3H-pyrimidin-4-one | 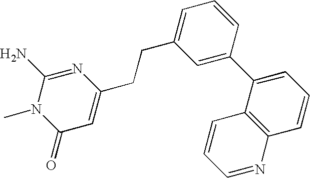 | 357 | (300 MHz, DMSO) δ 8.93 (d, J =4.6 Hz, 1H), 8.14 (d, J = 8.6 Hz,1H), 8.05 (d, J = 9.2 Hz, 1H), 7.81(t, J = 7.5 Hz, 1H), 7.54-7.43 (m,3H), 7.34 (d, J = 8.5 Hz, 1H), 7.30(d, J = 1.8 Hz, 2H), 7.04 (s, 2H),5.53 (s, 1H), 3.22 (s, 3H), 2.98 (t |

### EXAMPLE 246

### 2-Amino-3-(cyclohexylmethyl)-6-[2-[3-(2-furyl)phenyl]ethyl]-3H-pyrimidin-4-one

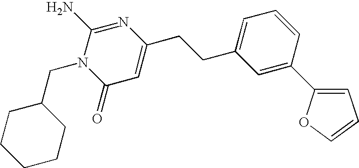

A solution of N′-(4-{2-[3-(2-furyl)phenyl]ethyl}-6-oxo-1,6-dihydropyrimidin-2-yl)-N,N-dimethylimidoformamide (Scheme 6, F) (40 mg) was dissolved in DMF (300 mL). The mixture was heated to 55° C. and to this was added potassium bicarbonate (100 mg) and cyclohexylmethyl bromide (100 μL) in portions over 24 h. To the cooled mixture was added acetonitrile (100 μL) and concentrated aqueous ammonium hydroxide (300 μL). This mixture was heated in a sealed tube at 80° C. The cooled solution was neutralized by addition of HCl and purified by preparative reverse phase HPLC to afford the desired product (6.7 mg) as the trifluoroacetate salt. ¹H NMR (300 MHz, DMSO) δ 8.23 (s, 4H), 7.73 (s, 5H), 7.58-7.52 (m, 12H), 7.33 (t, J=7.7 Hz, 7H), 7.16 (d, J=7.6 Hz, 6H), 6.90 (s, 6H), 6.60-6.58 (m, 6H), 5.73 (s, 5H), 3.74 (d, J=7.6 Hz, 23H), 2.94 (t, J=7.8 Hz, 14H), 2.71 (t, J=7.7 Hz, 13H), 1.65-1.51 (m, 36H), 1.11 (s, 16H), 1.01-0.93 (m, 16H); m/z 378.2.

### EXAMPLE 247

### 2-Amino-6-[2-[3-(2-furyl)phenyl]ethyl]-3-(tetrahydrofuran-2-ylmethyl)-3H-pyrimidin-4-one

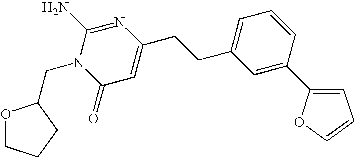

This material was prepared according to the procedure described for Example 246 with the exception that tetrahydrofuryl bromide was used in place of cyclohexylmethyl bromide. Following purification by preparative reverse phase chromatography, the desired product was contaminated with approximately 10% of the corresponding isocytosine 4-oxo ether. ¹H NMR (300 MHz, DMSO) δ 7.73 (s, 1H), 7.58-7.52 (m, 3H), 7.38-7.32 (m, 1H), 7.18-7.15 (m, 1H), 6.90 (s, 1H), 6.58 (s, 1H), 5.74 (s, 1H), 4.16-3.59 (m, 4H), 2.93 (t, J=8.0 Hz, 2H), 2.70 (t, J=7.7 Hz, 2H), 1.97-1.53 (m, 4H); m/z 366.1.

### EXAMPLE 248

### 2-Amino-6-[2-[3-(2-furyl)phenyl]ethyl]-3-(3-hydroxypropyl)-3H-pyrimidin-4-one

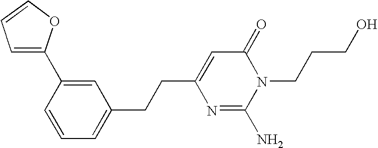

This material was prepared according to the procedure described for Example 246 with the exception that 1-bromo-3-(tetrahydropyranyloxy)propane was used in place of cyclohexylmethyl bromide. After deprotection of the DMF dimethyl acetal (with ammonium hydroxide, as described) the mixture was diluted with 6M HCl and incubated until the tetrahydropyran deprotection was complete. The mixture was neutralized and purified by preparative reverse phase HPLC to afford the desired product as the trifluoroacetate salt. ¹H NMR (300 MHz, DMSO) δ 8.02 (s, 1H), 7.74 (s, 1H), 7.58-7.53 (m, 2H), 7.34 (t, J=7.7 Hz, 1H), 7.17 (d, J=7.9 Hz, 1H), 6.91 (d, J=3.1 Hz, 1H), 6.60-6.58 (m, 1H), 5.75 (s, 1H), 3.90 (t, J=7.1 Hz, 2H), 3.44 (t, J=6.3 Hz, 2H), 2.93 (t, J=7.8 Hz, 2H), 2.70 (t, J=7.7 Hz, 2H), 1.74-1.65 (m, 2H); m/z 340.1.

### EXAMPLE 249

### 2-Amino-6-[2-[3-(2-furyl)phenyl]ethyl]-3-[(3-methoxyphenyl)methyl]-3H-pyrimidin-4-one

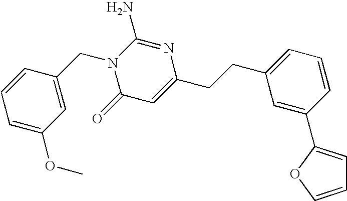

This material was prepared according to the procedure described for Example 246 with the exception that 3-methoxybenzyl bromide was used in place of cyclohexylmethyl bromide. ¹H NMR (300 MHz, DMSO) δ 7.87 (s, 1H), 7.73 (s, 1H), 7.59-7.53 (m, 2H), 7.34 (t, J=7.8 Hz, 1H), 7.23 (t, J=7.9 Hz, 1H), 7.16 (d, J=7.6 Hz, 1H), 6.91 (d, J=3.3 Hz, 1H), 6.86-6.81 (m, 2H), 6.72 (d, J=7.6 Hz, 1H), 6.60-6.58 (m, 1H), 5.77 (s, 1H), 5.11 (s, 2H), 3.73 (s, 3H), 2.95 (t, J=7.8 Hz, 2H), 2.72 (t, J=7.7 Hz, 2H); m/z 402.2.

### EXAMPLE 250

### 2-amino-6-[2-[3-(2-furyl)phenyl]ethyl]-3-[[3-(1H-tetrazol-5-yl)phenyl]methyl]-3H-pyrimidin-4-one

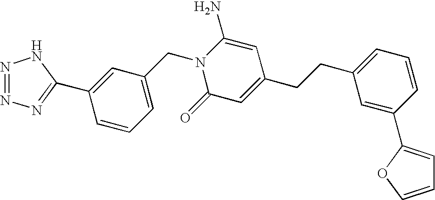

To a reaction tube was added 3-[[2-amino-4-[2-(3-furan-2-yl-phenyl)-ethyl]-6-oxo-6H-pyrimidin-1-ylmethyl]-benzonitrile (56.6 mg, 0.126 mmol), DMF (1.4 mL), NaN₃ (131 mg, 2.007 mmol), NH4Cl 128 mg, 2.383 mmol) and heated in a 1000 oil bath for 2 h. The cooled reaction was filtered and purified by preparative reverse phase chromatography to afford product as the trifluoroacetate salt (15 mg, 0.034 mmol, 27%). ¹H NMR (300 MHz, DMSO) δ 7.95-7.92 (m, 4H), 7.73 (s, 1H), 7.60 (s, 1H), 7.56 (d, J=4.7 Hz, 2H), 7.53 (d, J=6.1 Hz, 2H), 7.39 (d, J=8.1 Hz, 1H), 7.34 (t, J=7.9 Hz, 1H), 7.18 (d, J=7.2 Hz, 1H), 6.91 (d, J=2.9 Hz, 1H), 6.58 (s, 1H), 5.24 (s, 2H), 2.97 (t, J=7.7 Hz, 2H), 2.73 (t, J=8.0 Hz, 2H). m/z (APCI+) M+1 (440.0); t_{R} 2.17 min.

### 3-[[2-Amino-4-[2-(3-furan-2-yl-phenyl)-ethyl]-6-oxo-6H-pyrimidin-1-ylmethyl]-benzonitrile was Prepared as Follows

### EXAMPLE 251

### 3-[[2-amino-4-[2-(3-furan-2-yl-phenyl)-ethyl]-6-oxo-6H-pyrimidin-1-ylmethyl]-benzonitrile

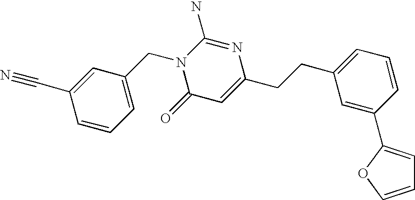

This material was prepared according to the procedure described for Example 252 with the exception that 3-bromomethyl-benzonitrile (58.2 mg, 0.297 mmol) was used in place of 3-bromomethyl-benzoic acid methyl ester. Purification by preparative reverse phase HPLC afforded product as the trifluoroacetate salt (67.6 mg, 0.171 mmol, 58%). ¹H NMR (300 MHz, DMSO) δ 7.77-7.73 (m, 3H), 7.68 (s, 2H), 7.59-7.48 (m, 4H), 7.34 (t, J=7.6 Hz, 1H), 7.16 (d, J=7.4 Hz, 1H), 6.91 (d, J=3.2 Hz, 1H), 6.59 (t, J=2.2 Hz, 1H), 5.75 (s, 1H), 5.17 (s, 2H), 2.96 (t, J=7.8 Hz, 2H), 2.70 (t, J=7.7 Hz, 2H).m/z (APCI) M (396.9); t_{R} 2.36 min.

### EXAMPLE 252

### 3-[[2-Amino-4-[2-[3-(2-furyl)phenyl]ethyl]-6-oxo-1H-pyrimidin-1-yl]methyl]benzoic acid

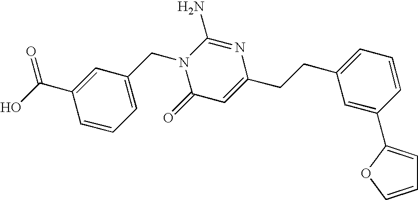

To a reaction tube was added 3-[[2-Amino-4-[2-[3-(2-furyl)phenyl]ethyl]-6-oxo-6H-pyrimidin-1-yl]methyl]benzoic acid methyl ester (43 mg, 0.100 mmol), THF (0.46 mL), water (0.23 mL), LiOH (4.6 mg, 0.110 mmol) and stirred at room temperature. After 1 h., additional LiOH (4.6 mg, 0.110 mmol) was added and reaction allowed to stir overnight Mixture was brought to pH 1-2 using 1N—HCl and purified by preparative reverse phase chromatography to afford product as the trifluoroacetate salt (7 mg, 0.017 mmol, 17%). ¹H NMR (300 MHz, DMSO) d 7.83 (s, 2H), 7.73 (d, J=1.3 Hz, 1H), 7.59 (s, 2H), 7.53 (d, J=7.7 Hz, 2H), 7.48-7.42 (m, 3H), 7.33 (t, J=7.7 Hz, 1H), 7.15 (d, J=7.5 Hz, 1H), 6.91 (d, J=3.2 Hz, 1H), 6.59 (d, J=5.2 Hz, 1H), 5.72 (s, 1H), 5.19 (s, 2H), 2.95 (t, J=8.0 Hz, 2H), 2.69 (t, J=7.6 Hz, 2H); m/z (APCI+) M+1(416.1); t_{R} 2.17 min.

### 3-[[2-Amino-4-[2-[3-(2-furyl)phenyl]ethyl]-6-oxo-6H-pyrimidin-1-yl]methyl]benzoic acid methyl ester was prepared as follows

### EXAMPLE 253

### 3-[[2-Amino-4-[2-[3-(2-furyl)phenyl]ethyl]-6-oxo-6H-pyrimidin-1-yl]methyl]benzoic acid methyl ester

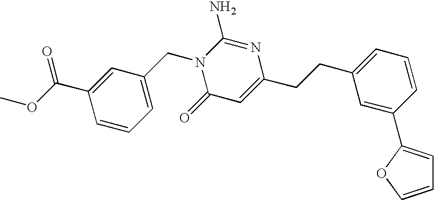

To a solution of N′-(4-{2-[3-(2-furyl)phenyl]ethyl}-6-oxo-1,6-dihydropyrimidin-2-yl)-N,Ndimethylimidoformamide (100 mg, 0.297 mmol) in DMF (2 mL) in a reaction tube was added potassium carbonate (41 mg, 0.297 mmol), 3-bromomethyl-benzoic acid methyl ester (68 mg, 0.297 mmol) and allowed to stir at room temperature overnight. To the reaction mixture was added acetonitrile (0.30 mL) and concentrated ammonium hydroxide (0.90 mL) and heated in a 80° oil bath for 4 h. The cooled solution was neutralized by addition of HCl and purified by preparative reverse phase HPLC to afford product as the trifluoroacetate salt (50 mg, 0.117 mmol, 39%). ¹H NMR (300 MHz, DMSO) δ 7.86 (s, 2H), 7.73 (d, J=1.1 Hz, 1H), 7.58-7.46 (m, 6H), 7.34 (t, J=9.7 Hz, 1H), 7.15 (d, J=7.3 Hz, 1H), 6.91 (d, J=3.0 Hz, 1H), 6.58 (d, J=2.9 Hz, 1H), 5.68 (s, 1H), 5.19 (s, 2H), 3.85 (s, 3H), 2.94 (t, J=8.0 Hz, 2H), 2.70 (t, J=10.1 Hz, 2H), m/z (APCI+) M+1 (430.0); t_{R} 2.44 min.

### EXAMPLE 254

### 3-[2-[2-Amino-4-[2-[3-(2-furyl)phenyl]ethyl]-6-oxo-1H-pyrimidin-1-yl]ethylamino]-3-oxo-propanoic acid

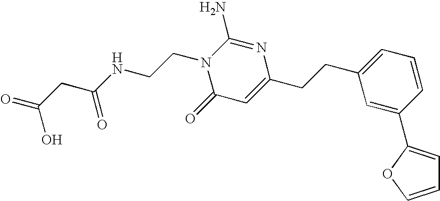

To a stirring solution of 2-amino-3-(2-aminoethyl)-6-{2-[3-(2-furyl)phenyl]ethyl}pyrimidin-4(3H)-one (30 mg of the bis TFA salt, 0.054 mmol) in DMF (0.5 mL) was added Et₃N (0.023 mL, 0.17 mmol), then methyl-3-chloro-3-oxopropionate (0.006 mL, 0.054 mmol). The reaction was stirred 30 min. during which more Et₃N and methyl-3-chloro-3-oxopropionate were added to drive the reaction to completion. Aqueous 1N NaOH (1 mL) was then added and reaction allowed to stir 1 hour after which it was neutralized with 1 N HCl and purified by reverse phase HPLC to give the desired product as a white solid (10 mg as the TFA salt, 35%). (300 MHz, MeOH) δ 7.59-7.53 (m, 3H), 7.33 (t, J=7.7 Hz, 1H), 7.16 (d, J=7.6 Hz, 1H), 6.75 (d, J=3.4 Hz, 1H), 6.51-6.49 (m, 1H), 5.84 (s, 1H), 4.12 (t, J=6.6 Hz, 2H), 3.48 (t, J=6.6 Hz, 2H), 3.25 (s, 2H), 3.03-2.98 (m, 2H), 2.84; m/z 411.2

### EXAMPLE 255

### 2-[2-[2-Amino-4-[2-[3-(2-furyl)phenyl]ethyl]-6-oxo-1H-pyrimidin-1-yl]ethylcarbamoyl]benzoic acid

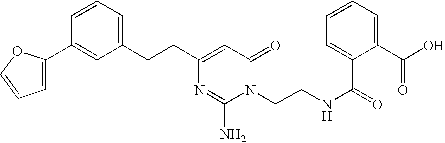

2-Amino-3-(2-aminoethyl)-6-{2-[3-(2-furyl)phenyl]ethyl}pyrimidin-4(3H)-one (30 mg of the bis TFA salt, 0.054 mmol), methyl hydrogen phthalate (11 mg, 0.060 mmol), and HOBt (8 mg, 0.060 mmol) were combined in a reaction tube and dissolved in DMF (1 mL). Triethylamine (0.024 mL, 0.18 mmol) was added next, then EDCI.HCl (12 mg, 0.065 mmol) and reaction allowed to stir overnight. Aqueous 1 N NaOH (0.5 mL) was added and reaction allowed to stir over the weekend. It was then neutralized to slightly basic using conc. HCl and purified by reverse phase HPLC to give the desired product as a solid (12 mg as the TFA salt, 38%). (300 MHz, MeOH) δ 7.99-7.96 (m, 1H), 7.64-7.52 (m, 5H), 7.44-7.41 (m, 1H), 7.30 (t, J=7.7 Hz, 1H), 7.15 (d, J=7.8 Hz, 1H), 6.75 (d, J=3.3 Hz, 1H), 6.51-6.49 (it, 1H), 5.88 (s, 1H), 4.25 (t, J=6.6 Hz, 2H), 3.60 (t, J=6.8 Hz, 2H); m/z 473.2.

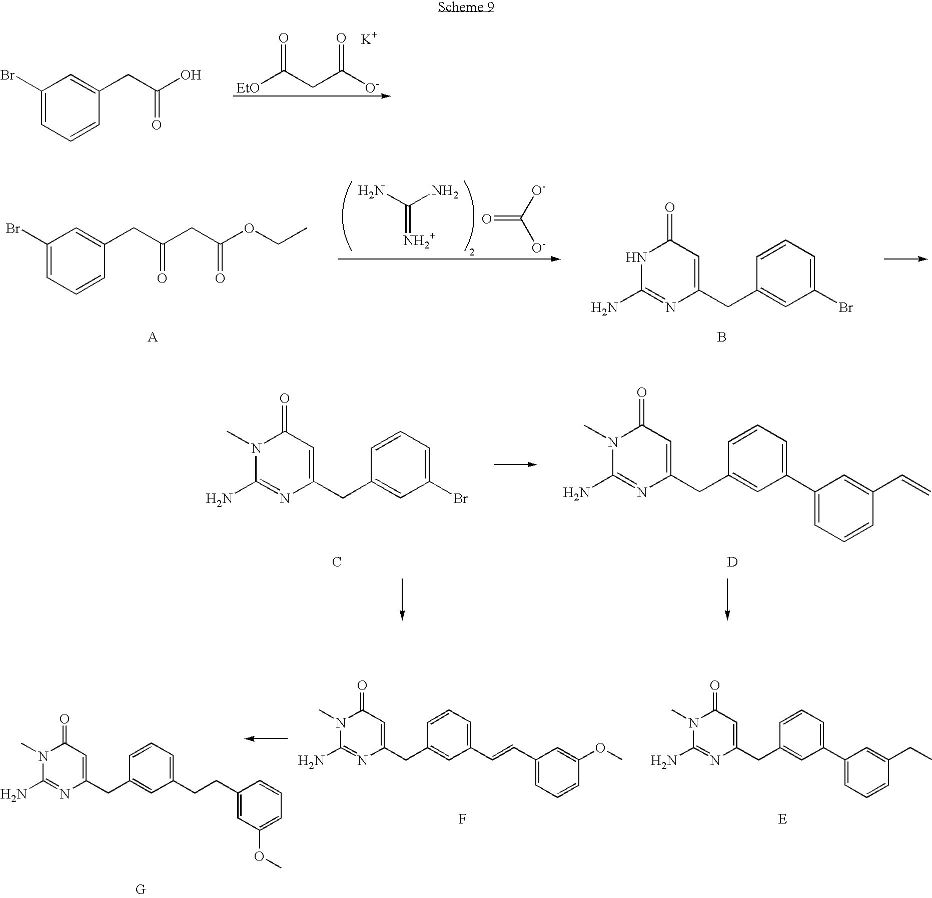

The precursor compounds in Scheme 9 were prepared as follows.

### 4-(3-Bromophenyl)-3-oxo-butyric acid ethyl ester (Scheme 9, A)

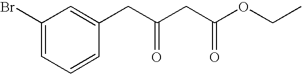

To a stirring suspension of potassium malonate (7.42 g, 43.6 mmol) in anhydrous acetonitrile (100 mL) at ambient temperature was added triethylamine (9.0 mL, 64.4 mmol) and magnesium chloride (4.94 g, 51.9 mmol) under an argon atmosphere. Stirring was continued for 3 h before the rapid addition of the 2-(3-bromophenyl)ethanoic imidazolide in the same solvent (60 mL), prepared 20 min prior by reaction between 3-bromophenylacetic acid (4.47 g, 20.8 mmol) and 1,1′-carbonyldiimidazole (4.04 g, 24.9 mmol) in dry acetonitrile (60 mL). The reaction mixture was allowed to stir for 17 h at room temperature, followed by heating to reflux for 1.5 h, before quenching by the slow addition of approximately 13% aqueous HCl (100 mL) at 5° C. The clear biphasic mixture was separated, wherein the organic layer was concentrated by rotary evaporation to a residue and treated with ethyl acetate (80 mL) while the aqueous remains were further extracted into ethyl acetate (2×50 mL). The combined organic extracts were washed with a saturated sodium carbonate aqueous solution (2×80 mL) and brine (1×50 mL), dried over MgSO₄ and concentrated in vacuo to afford the desired 4-(3-bromophenyl)-3-oxo-butyric acid ethyl ester (Scheme 100, A) as a clear yellow oil (5.93 g, quantitative). ¹H NMR (300 MHz, CDCl₃) δ 1.28 (t, J=7.1 Hz, 3H), 3.53 (s, 2H), 3.88 (s, 2H), 4.19 (q, J=7.1 Hz, 2H), 7.13-7.26 (m, 2H), 7.37-7.44 (m, 2H); m/z (ES+) M+1=285.0; t_{R}=2.52 min.

### 2-Amino-6-(3-bromo-benzyl)-3H-pyrimidin-4-one (Scheme 9, B)

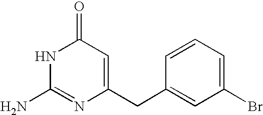

To a solution of 4-(3-bromophenyl)-3-oxo-butyric acid ethyl ester (Scheme 9, A) (5.93 g, 20.8 mmol) in ethanol (60 mL) was added guanidine carbonate (2.06 g, 11.4 mmol) and the reaction heated under reflux for 16 h. Upon concentration by rotary evaporation to about half the original volume, and cooling, the resulting solid was collected by filtration and washed with cold ethanol (3×10 mL). The precipitate was dried under high vacuum at 30° C. overnight to afford the title compound as a white solid (4.8 g, 83%). ¹H NMR (300 MHz, DMSO-d₆) δ 3.63 (s, 2H), 5.49 (s, 1H), 6.47 (s, 2H), 7.25-7.29 (m, 2H), 7.40-7.43 (m, 1H), 7.45 (s, 1H), 10.61 (s, 1H); m/z (ES+) M+=280.0; HPLC t_{R}=2.28 min.

### Amino-6-(3-bromo-benzyl)-3-methyl-3H-pyrimidin-4-one (Scheme 9, C)

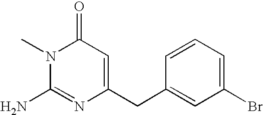

To a suspension of 2-amino-6-(3-bromo-benzyl)-3H-pyrimidin-4-one (Scheme 9, B) (1.63 g, 5.83 mmol) in absolute ethanol (35 mL) was added solid potassium hydroxide (589 mg, 10.5 mmol), which was stirred until a homogeneous solution achieved. Iodomethane (1.31 mL, 20.9 mmol) was added in one portion and the reaction heated in a sealed tube to 78° C. for 17 h. Upon completion, was concentrated in vacuo to a pale yellow residue and subject to flash chromatography eluting with. 0.5 to 5% MeOH in DCM to provide the 2-amino-6-(3-bromo-benzyl)-3-methyl-3H-pyrimidin-4-one (Scheme 100, C) as a white solid (1.3 g, 76%). ¹H NMR (300 MHz, DMSO-d₆) δ 3.20 (s, 3H), 3.58 (s, 2H), 5.52 (s, 1H), 7.07 (s, 2H), 7.26 (m, 2H), 7.41 (m, 1H), 7.46 (s, 1H); m/z (ES+) M+=294.0; HPLC t_{R}=1.39 min.

### 2-Amino-3-methyl-6-(3′-vinyl-biphenyl-3-ylmethyl)-3H-pyrimidin-4-one (Scheme 9, D)

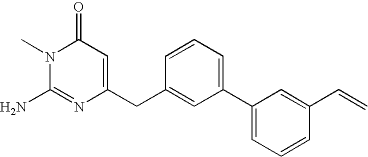

A thick-walled glass vial was charged with a stir bar, 2-amino-6-(3-bromo-benzyl)-3-methyl-3H-pyrimidin-4-one (Scheme 9, C) (120 mg, 0.2 mmol), 3-vinylphenylboronic acid (46 mg, 0.39 mmol), dichlorobis(triphenylphosphine)-palladium (II) (approximately 6 mg, 0.006 mmol), Cs₂CO₃ (246 mg, 0.76 mmol) and DME/H₂O/EtOH (7:3:2; 5 mL). The vial was crimp sealed and subjected to microwave radiation for 5 min at 150° C. The resultant black slurry was filtered, washed with methanol (3×3 mL) then concentrated in vacuo. The resultant residue was then purified by reverse phase HPLC. Appropriate fractions were concentrated via centrifugal evaporation to afford the white trifluoroacetic acid salt of the title compound (62 mg, 35%). ¹H NMR (300 MHz, DMSO-d₆/TFA-d) δ 3.27 (s, 3H), 3.93 (s, 2H), 5.33 (d, J=11.0 Hz, 1H), 5.93 (d, J=17.7 Hz, 1H), 5.98 (s, 1H), 6.83 (dd, J=17.7, 11.0 Hz, 1H), 7.36-7.52 (m, 4H), 7.59 (d, J=16.8 Hz, 1H), 7.62 (d, J=17.2 Hz, 1H), 7.72-7.73 (m, 2H); m/z (APCI+) M+1=318.2; t_{R}=2.17 min.

### EXAMPLE 257

### 2-Amino-6-(3′-ethyl-biphenyl-3-ylmethyl)-3-methyl-3H-pyrimidin-4-one (Scheme 9, E)

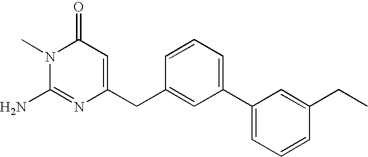

A glass reaction vessel was charged with palladium (10% on activated carbon, approximately 5 mg, ˜25% w/w) and 2-amino-3-methyl-6-(3′-vinyl-biphenyl-3-ylmethyl)-3H-pyridin-4-one (Scheme 9, D) (21 mg, 0.07 mmol) in ethanol (1 mL) then subjected to hydrogen (40 psi) for 5 min at 27° C. The resultant black slurry was filtered, washed with ethanol (3×3 mL) then concentrated in vacuo overnight to afford the title compound as a colorless film (18 mg, 82%). ¹H NMR (300 MHz, MeOH-d₄) δ 1.25 (t, J=7.6 Hz, 3H), 2.68 (q, J=7.6 Hz, 2H), 2.88 (s, 3H), 3.73 (s, 2H), 4.80 (s, 2H), 5.67 (s, 1H), 7.14-7.22 (m, 2H), 7.28-7.48 (m, 6H); m/z (ES+) M+1=320.2; t_{R}=1.88 min.

### EXAMPLE 258

### 2-Amino-6-{3-[(E)-2-(3-methoxy-phenyl)-vinyl]-benzyl}-3-methyl-3H-pyrimidin-4-one (Scheme 9, F)

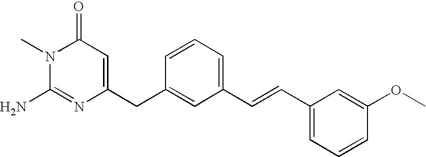

A thick-walled glass vial was charged with a stir bar, 2-amino-6-(3-bromo-benzyl)-3-methyl-3H-pyrimidin-4-one (Scheme 9, C) (130 mg, 0.44 mmol), 3-vinylanisole (89 mg, 0.66 mmol), tris(dibenzylideneacetone)dipalladium (0) (8 mg, 0.009 mmol), tri-t-butylphosphonium tetrafluoroborate (10 mg, 0.035 mmol), N,N-dicyclohexylmethylamine (104 mg, 0.53 mmol) and anhydrous 1,4-dioxane (2 mL). The reaction vial was sealed and subject to microwave radiation for 1 h at 150° C. The resultant slurry was filtered, washed with methanol (3×3 mL) then concentrated in vacuo. The resultant residue was subject to reverse phase purification (13-50% acetonitrile over 35 min). Appropriate fractions were concentrated via centrifugal evaporation to afford the white trifluoroacetic acid salt of the title compound (148 mg, 73%). ¹H NMR (300 MHz, DMSO-d₆/TFA-d) δ 3.29 (s, 3H), 3.81 (s, 3H), 3.89 (s, 2H), 5.95 (s, 1H), 6.87 (d, J=9.3 Hz, 1H), 7.16-7.19 (m, 2H), 7.24-7.30 (m, 3H), 7.36 (d, J=15.4 Hz, 1H), 7.38 (d, J=15.4 Hz, 1H), 7.54 (d, J=7.8 Hz, 1H), 7.61 (s, 1H); m/z (ES+) M+1=348.2; t_{R}=1.87 min.

### EXAMPLE 259

### 2-Amino-6-{3-[2-(3-methoxy-phenyl)-ethyl]-benzyl}-3-methyl-3H-pyrimidin-4-one (Scheme 9,G)

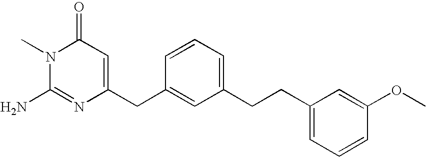

A glass Endeavor© reaction vessel was charged palladium (10% on activated carbon, approximately 9 mg, ˜10% w/w), and 2-amino-6-{3-[(E)-2-(3-methoxy-phenyl)-vinyl]-benzyl}-3-methyl-3H-pyrimidin-4-one (Example 258, Scheme 9, F) (98 mg, 0.28 mmol) in ethyl acetate/ethanol (1:4, 5 mL) then subjected to hydrogen (40 psi) for 20 min at 27° C. The resultant black slurry was filtered, washed with ethanol (3×3 mL) then concentrated in vacuo. The resultant residue was subjected to reverse phase HPLC purification (13-55% acetonitrile over 35 min). Appropriate fractions were concentrated via centrifugal evaporation giving rise to the white trifluoroacetic acid salt of the title compound (48 mg, 37%). ¹H NMR (300 MHz, DMSO-d₆/TFA-d) δ 2.52 (t, J=1.8 Hz, 2H), 2.87 (app s, 2H), 3.29 (s, 3H), 3.72 (s, 3H), 3.81 (s, 2H), 5.84 (s, 1H), 6.72-6.79 (m, 3H), 7.14-7.30 (m, 5H); m/z (ES+) M+1=350.1; t_{R} 2.18 min.

The following compounds in Table 10 were synthesized using methods analogous to those previously described for Example 258 (Scheme 9, F), employing the appropriate aryl olefin with the precursor aryl bromide of Scheme 9, C.

TABLE 10

| | | | | m/z |
|---|---|---|---|---|
| | | | | M + 1 |
| Ex. | Name | Structure | NMR | (Ioniz) |
| 266 | 2-Amino-3-methyl-6-[3-((E)-styryl)-benzyl]-3H-pyrimidin-4-one | 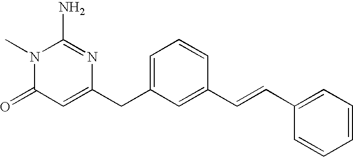 | 1H NMR (300 MHz, DMSO-d₆/TFA-d)3.29 (s, 3H), 3.89 (s, 2H), 5.95 (s, 1H),7.25-7.31 (m, 4H), 7.37-7.41 (m,3H), 7.55 (d, J = 7.6 Hz, 1H), 7.59-7.61 (m, 3H). | 318.2(ES+) |
| 267 | 2-Amino-6-{3-[(E)-2-(4-methoxy-phenyl)-vinyl]-benzyl}-3-methyl-3H-pyrimidin-4-one | 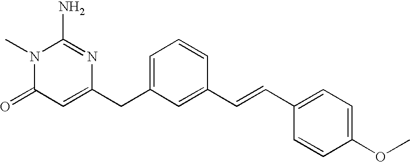 | 1H NMR (300 MHz, DMSO-d₆ /TFA-d)δ 3.28 (s, 3H), 3.79 (s, 3H), 3.87 (s,2H), 5.94 (s, 1H), 6.92-6.98 (m, 2H),7.07 (d, J = 16.4 Hz, 1H), 7.18-7.24(m, 2H), 7.33-7.38 (m, 2H), 7.49-7.57 (m, 3H). | 348.1(ES+) |
| 268 | 2-Amino-3-methyl-6-[3-((E)-3-methyl-pent-1-enyl)-benzyl]-3H-pyrimidin-4-one | 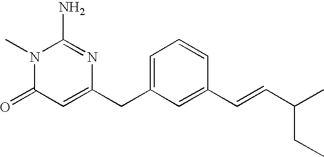 | 1H NMR (300 MHz, DMSO-d₆/TFA-d)δ 0.87 (t, J = 7.3 Hz, 3H), 1.05 (d, J =6.7 Hz, 3H), 1.38 (q, J = 7.3 Hz, 2H),2.16-2.25 (m, 1H), 3.27 (s, 3H), 3.83(s, 2H), 5.92 (s, 1H), 6.19 (dd, J = 16.1,7.7 Hz, 1H), 6.35 (d, J = 16.1 Hz, 1H),7.19 (d, J = 6.3 Hz, 1H), 7.27-7.31 (m,2H), 7.41 (s, 1H). | 298.1(ES+) |
| 269 | 2-Amino-3-methyl-6-[3-((E)-4-methyl-pent-1-enyl)-benzyl]-3H-pyrimidin-4-one | 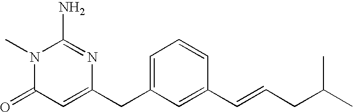 | 1H NMR (300 MHz, DMSO-d₆/TFA-d)δ 0.92 (d, J = 6.6 Hz, 6H), 1.71(quintet, J = 6.6 Hz, 1H), 2.08 (t, J =6.4 Hz, 2H), 3.27 (s, 3H), 3.83 (s, 2H),5.92 (s, 1H), 6.25-6.34 (m, 1H), 6.38(d, J = 16.0 Hz, 1H), 7.19 (d, J = 6.9Hz, 1H), 7.27-7.32 (m, 2H), 7.40 (s,1H). | 298.1(ES+) |

The following compounds in Table 11 were synthesized using methods analogous to those previously described for Example 259 (Scheme 9, G), employing the appropriate previously described benzyl styryl analogs as precursor (see e.g., Table 10).

TABLE 11

| | | | | m/z |
|---|---|---|---|---|
| | | | | M + 1 |
| Ex. | Compound | Structure | NMR | (Ioniz) |
| 270 | 2-Amino-3-methyl-6-(3-phenethyl-benzyl)-3H-pyrimidin-4-one | 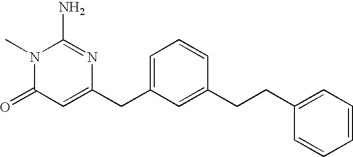 | 1H NMR (300 MHz,DMSO-d₆/TFA-d) δ 2.88 (app s, 4H),3.29 (s, 3H), 3.81 (s, 2H), 5.83 (s,1H), 7.16-7.26 (m, 9H). | 320.1 (ES+) |
| 271 | 2-Amino-3-methyl-6-[3-(2-pyridin-4-yl-ethyl)-benzyl]-3Hpyrimidin-4-one | 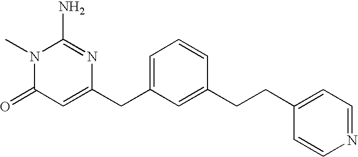 | 1H NMR (300 MHz,DMSO-d₆/TFA-d) δ 3.02 (t, J = 7.9Hz, 2H), 3.24 (t, J = 7.9 Hz, 2H),3.29 (s, 3H), 3.81 (s, 2H), 5.74 (s,1H), 7.19-7.32 (m, 4H), 7.97 (d, J =6.6 Hz, 2H), 8.83 (d, J = 6.6 Hz, 2H). | 321.2(ES+) |
| 272 | 2-Amino-3-methyl-6-[3-(3-methyl-pentyl)-benzyl]-3H-pyrimidin-4-one | 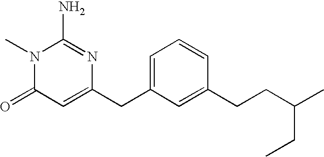 | 1H NMR (300 MHz,DMSO-d₆/TFA-d) δ 0.85 (t, J = 7.2Hz, 3H), 0.90 (d, J = 6.2 Hz, 3H),1.16 (app quintet, J = 3.4 Hz, 1H),1.30-1.41 (m, 3H), 1.57 (m, 1H),2.58 (m, 2H), 3.28 (s, 3H), 3.82 (s,2H), 5.87 (s, 1H), 7.11-7.29 (m, 4H). | 300.4(ES+) |
| 273 | 2-Amino-3-methyl-6-[3-(4-methyl-pentyl)-benzyl]-3H-pyrimidin-4-one | 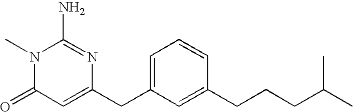 | 1H NMR (300 MHz,DMSO-d₆/TFA-d) δ 0.85 (d, J = 6.6Hz, 6H), 1.19 (dd, J = 14.7, 6.7 Hz,2H), 1.55 (app quintet, J = 6.7 Hz,2H), 2.55 (m, 3H), 3.28 (s, 3H), 3.82(s, 2H), 5.86 (s, 1H), 7.11-7.29 (m,4H). | 300.4(ES+) |

The following compounds in Table 12 were synthesized using methods analogous to those previously described for Examples 1 or 4 employing the appropriate commercially available boronic acid. The column “Method” contains three rows: the first is the scheme used; the second is the Suzuki method described in Example 2 (A) or Example 6 (B); and the third is the arylbromide used in the Suzuki. NA denotes that the Suzuki coupling with an arylbromide was not used.

TABLE 12

| | | | | | m/z | |
|---|---|---|---|---|---|---|
| | | | | | M + 1 | |
| | | | | | (Ioniz- | LC t_{R} |
| Ex. | Compound | Structure | Method | NMR | ation) | (min) |
| 274 | 2-amino-3,6-dimethyl-6-naphthalen-1-yl-5,6-dihydro-3H-pyrimidin-4-one | 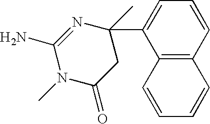 | Scheme-2NA | ¹H NMR (300 MHz,DMSO-d₆/TFA-d): δ 1.72 (s, 3H); 3.09 (s, 3H); 3.27 (d, 1H,J = 16.5 Hz); 3.57 (d, 1H, J =16.5 Hz); 7.59 (m, 3H); 7.97(m, 4H) | 268 (APCI+) | 1.56 |
| 276 | 2-amino-6-(4-chloro-3-naphthalen1-yl-phenyl)-3,6-dimethyl-5,6-dihydro-3H-pyrimidin-4-one | 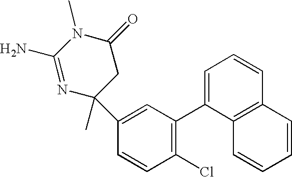 | Scheme-2Suzuki B10 | ¹H NMR (300 MHz,DMSO-d₆/TFA-d): δ 1.68 (s,3H); 3.12 (d, 3H, J = 6.6 Hz);3.19 (dd, 1H, J = 16.5 Hz);3.50 (dd, 1H, J = 16.5 Hz);7.27 (t, 1H, J = 7.8 Hz);7.41-7.72 (br m, 7H); 8.02 (d,1H, J = 6.6 Hz); 8.71 (d, 1H, J =6.6 Hz). | 378(APCI+) | 2.01 |
| 277 | 2-amino-6-(6,4′-dichloro-biphenyl-3-yl)-3,6-dimethyl-5,6-dihydro-3H-pyrimidin-4-one | 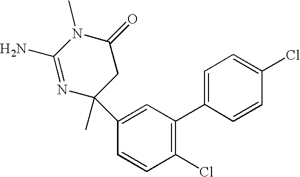 | Scheme-2Suzuki B10 | ¹H NMR (300 MHz,DMSO-d₆/TFA-d): δ 1.66 (s,3H); 3.11 (s, 3H); 3.19 (d, 1HJ = 16.5 Hz); 3.52 (d, 1H, J =16.5 Hz); 7.38-7.64 (br m, 7H). | 362(ES+) | 1.89 |
| 278 | 2-amino-6-(4-chloro-3-isoquinolin-5-yl-phenyl)-3,6-dimethyl-5,6-dihydro-3H-pyrimidin-4-one | 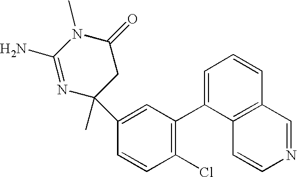 | Scheme-2Suzuki B10 | ¹H NMR (300 MHz,DMSO-d₆/TFA-d): δ 1.69 (s,3H); 3.13 (d, 3H, J = 6.6 Hz),3.22 (dd, 1H, J = 16.5 Hz),3.50 (dd, 1H, J = 16.5 Hz),7.62 (m, 2H); 7.78 (m, 2H),8.17 (m, 2H); 8.66 (m, 2H)10.66 (s, 1H). | 379(APCI+) | 1.35 |
| 279 | 2-amino-6-(6-chloro-4′-hyroxy-biphenyl-3-yl-phenyl)-3,6-dimethyl-5,6-dihydro-3H-pyrimidin-4-one | 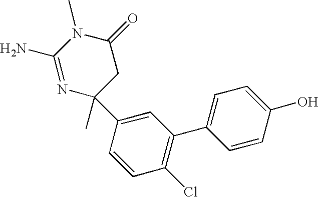 | Scheme-2Suzuki B10 | ¹H NMR (300 MHz,DMSO-d₆ /TFA-d): δ 1.65 (s,3H): 3.11 (s, 3H); 3.19 (d, 1H,J = 16.5 Hz); 3.52 (d, 1H, J =16.5 Hz); 6.87 (m, 2H),7.25-7.34 (br m, 3H), 7.42 (s,1H), 7.56 (d, 1H, J = 7.5 Hz). | 344(APCI+) | 1.66 |
| 280 | 2-amino-6-(4-chloro-3-dibenzofuran-4-yl-phenyl)-3,6-dimethyl-5,6-dihydro-3H-pyrimidin-4-one | 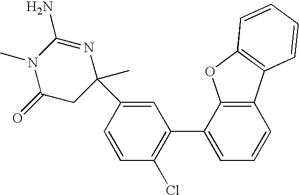 | Scheme-2Suzuki B10 | ¹H NMR (300 MHz,DMSO-d₆/TFA-d): δ 1.69 (s,3H); 3.16 (s, 3H); 3.21 (d, 1H,J = 16.5 Hz); 3.54 (d, 1H, J =16.5 Hz); 7.38-7.57 (br m,5H), 7.65-7.75 (br m, 4H),8.20 (m, 2H). | 418(APCI+) | 2.25 |
| 281 | 2-amino-6-[4-chloro-3-(2,3-dihydrobenzo[1,4]dioxin-6-yl-phenyl]-3,6-dimethyl-5,6-dihydro-3H-pyrimidin-4-one | 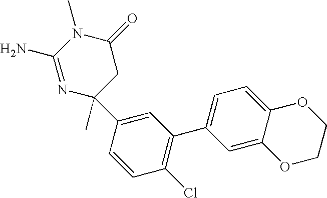 | Scheme-2Suzuki B10 | ¹H NMR (300 MHz,DMSO-d₆/TFA-d): δ 1.64 (s,3H); 3.11 (s, 3H); 3.17 (d, 1H,J = 16.5 Hz); 3.51 (d, 1H, J =16.5 Hz); 4.30 (s, 4H), 6.94m, 3H), 7.37 (m, 1H), 7.43 (s,1H); 7.56 (d, 1H, J = 8.4 Hz). | 3.86(APCI+) | 1.97 |
| 282 | 2-amino-3-benzyl-6-(3-bromo-phenyl)-6-methyl-5,6-dihydro-3H-pyrimidin-4-one | 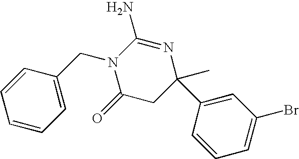 | Scheme-2NA | ¹H NMR (300 MHz,DMSO-d₆/TFA-d): δ 1.65 (s,3H); 3.35 (d, 1H, J = 16.5 Hz);3.68 (d, 1H, J = 16.5 Hz); 4.72(d, 1H, J = 16.8 Hz); 5.25 (d,1H, J = 16.8 Hz); 6.68 (m,2H); 7.15 (m, 3H); 7.35 (m,2H); 7.57 (m, 2H). | 372(APCI+) | 1.79 |
| 283 | 2-amino-6-[4-chloro-3-(1H-indol-6-yl)-phenyl]-3,6-dimethyl-5,6-dihydro-3H-pyrimidin-4-one | 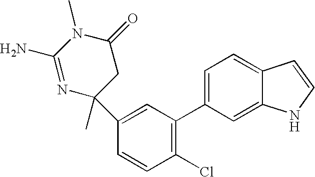 | Scheme -2Suzuki B10 | ¹H NMR (300 MHz,DMSO-d₆/TFA-d): δ 1.67 (s,3H); 3.12 (s, 3H); 3.19 (d, 1H,J = 16.5 Hz); 3.52 (d, 1H, J =16.5 Hz); 6.49 (s, 1H), 7.05(d,1H, J = 9.6 Hz), 7.43 (m, 4H),7.61 (m, 2H). | 367(APCI+) | 1.94 |
| 284 | 2-amino-3-benzyl-6-(3′-methoxy-biphenyl-3-yl)-6-methyl-5,6-dihydro-3H-pyrimidin-4-one | 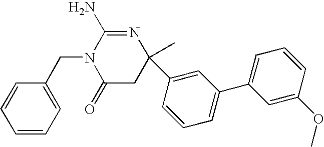 | Scheme-2Suzuki B282 | ¹H NMR (300 MHz,DMSO-d₆ /TFA-d): δ 1.68 (s,3H); 3.38 (d, 1H, J = 16.5 Hz);3.78 (d, 1H, J = 16.5 Hz); 4.70(d, 1H, J = 16.8 Hz); 5.24 (d,1H, J = 16.8 Hz); 6.60 (d, 2H,J = 7.2 Hz); 6.94-7.16 (m,4H); 7.37 (m, 2H); 7.49 (t, 1H,J = 7.2 Hz); 7.62 (s, 1H), 7.67(d, 1H, 7.2 Hz). | 400(APCI+) | 2.03 |
| 285 | 2-amino-3-benzyl-6-methyl-6-phenyl-5,6-dihydro-3H-pyrimidin-4-one | 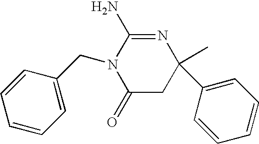 | Scheme-2NA | ¹H NMR (300 MHz,DMSO-d₆/TFA-d): δ 1.64 (s,3H); 3.35 (d, 1H, J = 16.5 Hz);3.64 (d, 1H, J = 16.5 Hz); 4.71(d, 1H, J = 16.8 Hz); 5.24 (d,1H, J = 16.8 Hz); 6.40 (d, 2H,J = 7.2 Hz); 7.01-7.20 (br m,3H); 7.35 (s, 5H). | 294(ES+) | 1.44 |
| 286 | 2-amino-6-(6-chloro-biphenyl-3-yl)-3,6-dimethyl-5,6-dihydro-3H-pyrimidin-4-one | 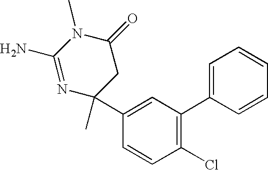 | Scheme-2Suzuki b10 | ¹H NMR (300 MHz,DMSO-d₆/TFA-d): δ 1.65 (s,3H); 3.11 (s, 3H; 3.18 (d, 1H,J = 16.5 Hz); 3.52(d, 1H, J =16.5 Hz); 7.45 (m, 7H); 7.61(d, 1H, J = 8.4 Hz). | 328(ES+) | 1.72 |
| 287 | 2-amino-3,6-dimethyl-6-phenyl-5,6-dihydro-3H-pyrimidin-4-one | 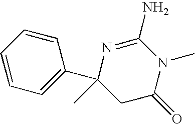 | Scheme-2NA | ¹H NMR (300 MHz,DMSO-d₆/TFA-d): δ 1.63 (s,3H); 3.08 (s, 3H); 3.18 (d, 1H,J = 16.5 Hz); 3.42 (d, 1H, J =16.5 Hz); 7.42 (m, 5H). | 218(APCI+) | 0.88 |
| 288 | 2-amino-6-(6-chloro-4′-methoxy-biphenyl-3-yl)-3,6-dimethyl-5,6-dihydro-3H-pyrimidin-4-one | 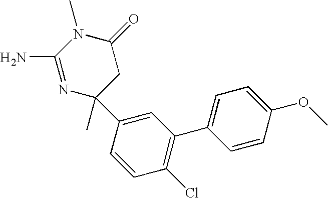 | Scheme-2suzuki B10 | ¹H NMR (300 MHz,DMSO-d₆ /TFA-d): δ 1.65 (s,3H); 3.11 (s, 3H); 3.19 (d, 1H,J = 16.5 Hz); 3.52 (d, 1H, J =16.5 Hz); 3.83 (s, 3H); 7.04 (d,2H, J = 8.4 Hz); 7.41 (m, 4H);7.58(d, 1H, J = 8.4 Hz). | 358(ES+) | 1.73 |
| 289 | 2-amino-6-(3-methoxy-phenyl)-3,6-dimethyl-5,6-dihydro-3H-pyrimidin-4-one | 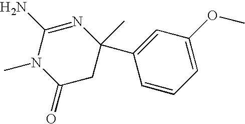 | Scheme-2NA | ¹H NMR (300 MHz,DMSO-d₆ /TFA-d): δ 1.62 (s,3H); 3.10 (s, 3H); 3.16 (d, 1H,J = 16.5 Hz); 3.49 (d, 1H, J =16.5 Hz); 3.77 (s, 3H); 6.95(m, 3H); 7.34 (t, 1H, J = 8.4 Hz). | 248(APCI+) | 1.13 |

The following compounds in Table 13 were also prepared and demonstrated a max affinity value of between 0.001 to 100 μM.

TABLE 13
IUPAC Name

2-amino-3-methyl-6-[2-[3-(2-thienyl)phenyl]ethyl]-3H-pyrimidin-4-one
2-amino-6-[2-[3-(3-methoxyphenyl)phenyl]ethyl]-3,6-dimethyl-5,6-dihydro-3H-pyrimidin-4-one
2-amino-3-methyl-6-phenyl-5,6-dihydro-3H-pyrimidin-4-one
3-[2-[2-amino-4-[2-[3-(2-furyl)phenyl]ethyl]-6-oxo-1H-pyrimidin-1-yl]ethylcarbamoyl]benzoic acid
2-amino-6-[2-[3-(2-furyl)phenyl]ethyl]-3-[2-(1H-imidazol-1-yl)ethyl]-3H-pyrimidin-4-one
2-amino-3-[2-[bis(3-furylmethyl)amino]ethyl]-6-[2-[3-(2-furyl)phenyl]ethyl]-3H-pyrimidin-4-one
N-[2-[2-amino-4-[2-[3-(2-furyl)phenyl]ethyl]-6-oxo-1H-pyrimidin-1-yl]ethyl]-2-(1H-tetrazol-5-yl)acetamide
5-[2-[2-amino-4-[2-[3-(2-furyl)phenyl]ethyl]-6-oxo-1H-pyrimidin-1-yl]ethylamino]-5-oxo-pentanoic
acid
acetic acid 2-[2-amino-4-[2-[3-(2-furyl)phenyl]ethyl]-6-oxo-1H-pyrimidin-1-yl]ethylcarbamoylmethyl
ester
N-[2-[2-amino-4-[2-[3-(2-furyl)phenyl]ethyl]-6-oxo-1H-pyrimidin-1-yl]ethyl]-3-hydroxy-benzamide
N-[2-[2-amino-4-[2-[3-(2-furyl)phenyl]ethyl]-6-oxo-1H-pyrimidin-1-yl]ethyl]-4-hydroxy-benzamide
4-amino-N-[2-[2-amino-4-[2-[3-(2-furyl)phenyl]ethyl]-6-oxo-1H-pyrimidin-1-yl]ethyl]benzamide
N-[2-[2-amino-4-[2-[3-(2-furyl)phenyl]ethyl]-6-oxo-1H-pyrimidin-1-yl]ethyl]piperidine-4-carboxamide
2-amino-6-[2-[3-(2-furyl)phenyl]ethyl]-3-(2-phenethylaminoethyl)-3H-pyrimidin-4-one
4-[2-[2-amino-4-[2-[3-(2-furyl)phenyl]ethyl]-6-oxo-1H-pyrimidin-1-yl]ethylaminomethyl]benzoic acid
2-amino-6-[2-[3-(2-furyl)phenyl]ethyl]-3-[2-[(3-hydroxyphenyl)methylamino]ethyl]-3H-pyrimidin-4-one
2-amino-6-[2-[3-(2-furyl)phenyl]ethyl]-3-[2-[[4-(2-hydroxyethoxy)phenyl]methylamino]ethyl]-3H-pyrimidin-
4-one
2-amino-6-[2-[3-(2-furyl)phenyl]ethyl]-3-(2-isobutylaminoethyl)-3H-pyrimidin-4-one
2-amino-6-[2-[3-(2-furyl)phenyl]ethyl]-3-[2-(1H-indol-5-ylmethylamino)ethyl]-3H-pyrimidin-4-one
2-amino-6-[2-[3-(2-furyl)phenyl]ethyl]-3-[2-[(4-hydroxyphenyl)methylamino]ethyl]-3H-pyrimidin-4-one
2-amino-6-[2-[3-(2-furyl)phenyl]ethyl]-3-[2-(3-pyridylmethylamino)ethyl]-3H-pyrimidin-4-one
2-amino-3-benzyl-6-[2-[3-(2-furyl)phenyl]ethyl]-3H-pyrimidin-4-one
2-amino-6-[2-[3-(2-furyl)phenyl]ethyl]-3-(2-hydroxyethyl)-3H-pyrimidin-4-one
2-amino-6-[2-[3-(2-furyl)phenyl]ethyl]-3-isopentyl-3H-pyrimidin-4-one
2-amino-6-[2-[3-(2-furyl)phenyl]ethyl]-3-[2-[(1-hydroxy-2,2,6,6-tetramethyl-4-piperidyl)amino]ethyl]-3H-
pyrimidin-4-one
2-amino-6-[2-[3-(2-furyl)phenyl]ethyl]-3-[2-[(1-hydroxy-2,2,6,6-tetramethyl-4-piperidyl)amino]ethyl]-3H-
pyrimidin-4-one
2-amino-3-[2-(1,3-dioxan-2-yl)ethyl]-6-[2-[3-(2-furyl)phenyl]ethyl]-3H-pyrimidin-4-one
4-[[2-amino-4-[2-[3-(2-furyl)phenyl]ethyl]-6-oxo-1H-pyrimidin-1-yl]methyl]benzonitrile
2-amino-6-[2-[3-(2-furyl)phenyl]ethyl]-3-[[4-(1H-tetrazol-5-yl)phenyl]methyl]-3H-pyrimidin-4-one
2-amino-6-[2-[3-(2-furyl)phenyl]ethyl]-3-(2-morpholinoethyl)-3H-pyrimidin-4-one
2-[[2-amino-4-[2-[3-(2-furyl)phenyl]ethyl]-6-oxo-1H-pyrimidin-1-yl]methyl]benzonitrile
2-amino-6-[2-[3-(2-furyl)phenyl]ethyl]-3-[2-(1-piperidyl)ethyl]-3H-pyrimidin-4-one

The following compounds in Table 14 were prepared and demonstrated a max affinity value of 100 μM or greater.

TABLE 14
IUPAC Name

2-amino-6-[3-(4-hydroxyphenyl)phenyl]-6-methyl-5,6-dihydro-3H-pyrimidin-4-one
2-amino-3-benzyl-6-phenyl-5,6-dihydro-3H-pyrimidin-4-one
2-amino-6-methyl-6-phenyl-5,6-dihydro-3H-pyrimidin-4-one
2-amino-6-(3,4-dichlorophenyl)-6-isopropyl-5,6-dihydro-3H-pyrimidin-4-one
2-amino-6-(3,4-dichlorophenyl)-3-methyl-5,6-dihydro-3H-pyrimidin-4-one
2-amino-6-(3-furyl)-6-phenyl-5,6-dihydro-3H-pyrimidin-4-one
2-amino-6-(3,4-dichlorophenyl)-5,6-dihydro-3H-pyrimidin-4-one
2-amino-6-methyl-6-(3-pyridyl)-5,6-dihydro-3H-pyrimidin-4-one
2-amino-6-benzyl-6-methyl-5,6-dihydro-3H-pyrimidin-4-one
2-amino-6-[3-(4-hydroxyphenyl)phenyl]-6-methyl-5,6-dihydro-3H-pyrimidin-4-one
2-amino-3-benzyl-6-phenyl-5,6-dihydro-3H-pyrimidin-4-one
2-amino-3-methyl-6-(o-tolyl)-5,6-dihydro-3H-pyrimidin-4-one
2-amino-6-(3-bromophenyl)-5,6-dihydro-3H-pyrimidin-4-one
2-amino-6-(o-tolyl)-5,6-dihydro-3H-pyrimidin-4-one
2-amino-6-[2-[3-(2-hydroxyphenyl)phenyl]ethyl]-3-methyl-3H-pyrimidin-4-one
2-amino-6-[[3-[2-(4-methoxyphenyl)ethyl]phenyl]methyl]-3-methyl-3H-pyrimidin-4-one
2-amino-3-methyl-6-[[3-[2-(4-methylthiazol-5-yl)ethyl]phenyl]methyl]-3H-pyrimidin-4-
one
2-amino-3-methyl-6-[[3-[2-(4-pyridyl)vinyl]phenyl]methyl]-3H-pyrimidin-4-one
2-amino-6-[2-(4-bromophenyl)ethyl]-3H-pyrimidin-4-one
2-amino-6-[2-[4-[2-(2-methoxyethoxy)ethoxy]phenyl]ethyl]-3-methyl-3H-pyrimidin-4-one
2-amino-3-methyl-6-[2-[3-(8-quinolyl)phenyl]ethyl]-3H-pyrimidin-4-one
2-amino-6-[2-[3-(3,4-dimethoxyphenyl)phenyl]ethyl]-3-methyl-3H-pyrimidin-4-one
2-amino-6-[2-[4-(2-methoxyethoxy)phenyl]ethyl]-3-methyl-3H-pyrimidin-4-one
2-amino-6-[2-[3-(2-furyl)phenyl]ethyl]-3-(2-phenoxyethyl)-3H-pyrimidin-4-one
2-amino-6-[2-[3-(2,6-dioxabicyclo[5.4.0]undeca-7,9,11-trien-9-yl)phenyl]ethyl]-3-methyl-
3H-pyrimidin-4-one
2-amino-3-methyl-6-phenethyl-3H-pyrimidin-4-one
2-amino-3-(2-aminoethyl)-6-[2-[3-(2-furyl)phenyl]ethyl]-3H-pyrimidin-4-one
2-amino-6-[2-(4-hydroxyphenyl)ethyl]-3-methyl-3H-pyrimidin-4-one
2-amino-6-[2-(3-bromophenyl)ethyl]-3H-pyrimidin-4-one
2-amino-6-[2-[3-(4-dimethylaminophenyl)phenyl]ethyl]-3-methyl-3H-pyrimidin-4-one
2-amino-3-[(3-aminophenyl)methyl]-6-[2-(3-tetrahydrofuran-2-ylphenyl)ethyl]-3H-pyrimidin-
4-one
2-amino-3-methyl-6-[[3-[2-(2-pyridyl)vinyl]phenyl]methyl]-3H-pyrimidin-4-one
2-amino-6-[2-[3-(3-pyridyl)phenyl]ethyl]-3H-pyrimidin-4-one
2-amino-6-(4-benzyloxyphenyl)-3H-pyrimidin-4-one
2-amino-6-phenethyl-3-(1H-tetrazol-5-ylmethyl)-3H-pyrimidin-4-one
2-amino-6-[[3-[3-(methoxymethyl)phenyl]phenyl]methyl]-3-methyl-3H-pyrimidin-4-one
2-amino-6-[3-(3-methoxyphenyl)phenyl]-3-methyl-3H-pyrimidin-4-one
2-amino-6-[2-(4-benzyloxyphenyl)ethyl]-3-methyl-3H-pyrimidin-4-one
2-amino-6-[2-(4-benzyloxyphenyl)ethyl]-3H-pyrimidin-4-one
2-amino-3-methyl-6-[2-[3-[3-(trifluoromethoxy)phenyl]phenyl]ethyl]-3H-pyrimidin-4-one
2-amino-6-[2-[3-(3-isopropylphenyl)phenyl]ethyl]-3-methyl-3H-pyrimidin-4-one
acetic acid 2-[2-amino-4-[2-(1H-indol-6-yl)ethyl]-6-oxo-1H-pyrimidin-1-yl]ethyl ester
2-amino-3-methyl-6-(3-phenylphenyl)-3H-pyrimidin-4-one
2-amino-3-[2-(3-furylmethylamino)ethyl]-6-[2-[3-(2-furyl)phenyl]ethyl]-3H-pyrimidin-4-
one
2-amino-6-[[5-(3-methoxyphenyl)-3-pyridyl]methyl]-3-methyl-3H-pyrimidin-4-one
2-amino-6-[2-[2-(3-methoxyphenyl)phenyl]ethyl]-3-methyl-3H-pyrimidin-4-one
2-amino-3-methyl-6-(1-methyl-2-phenyl-ethyl)-3H-pyrimidin-4-one
2-amino-6-[2-[3-[4-(aminomethyl)phenyl]phenyl]ethyl]-3-methyl-3H-pyrimidin-4-one
2-amino-6-[[3-[2-[4-(dimethylaminomethyl)phenyl]vinyl]phenyl]methyl]-3-methyl-3H-pyrimidin-
4-one
2-amino-6-[(3-bromophenyl)methyl]-3-methyl-3H-pyrimidin-4-one
2-[3-[2-(2-amino-1-methyl-6-oxo-1H-pyrimidin-4-yl)ethyl]phenyl]benzoic acid
2-amino-6-[(3-bromophenyl)methyl]-3H-pyrimidin-4-one
2-amino-6-[(5-bromo-3-pyridyl)methyl]-3H-pyrimidin-4-one
N-[2-[2-amino-4-[2-[3-(2-furyl)phenyl]ethyl]-6-oxo-1H-pyrimidin-1-yl]ethyl]-3-cyano-benzamide
N-[2-[2-amino-4-[2-[3-(2-furyl)phenyl]ethyl]-6-oxo-1H-pyrimidin-1-yl]ethyl]-3-phenyl-
propanamide
N-[2-[2-amino-4-[2-[3-(2-furyl)phenyl]ethyl]-6-oxo-1H-pyrimidin-1-yl]ethyl]benzamide
2-amino-3-(2-dibenzylaminoethyl)-6-[2-[3-(2-furyl)phenyl]ethyl]-3H-pyrimidin-4-one
2-amino-3-methyl-6-[[3-[2-(2-pyridyl)ethyl]phenyl]methyl]-3H-pyrimidin-4-one
2-amino-6-[[3-[3-(hydroxymethyl)phenyl]phenyl]methyl]-3-methyl-3H-pyrimidin-4-one
3-[2-[2-amino-4-[2-[3-(2-furyl)phenyl]ethyl]-6-oxo-1H-pyrimidin-1-yl]ethoxy]benzoic
acid
2-amino-3-methyl-6-[[3-[2-(4-methylthiazol-5-yl)vinyl]phenyl]methyl]-3H-pyrimidin-4-
one
2-amino-6-(3-bromophenyl)-3H-pyrimidin-4-one
2-amino-6-(3-bromophenyl)-3-methyl-3H-pyrimidin-4-one
2-amino-3-methyl-6-phenyl-3H-pyrimidin-4-one
N-[2-[2-amino-4-[2-[3-(2-furyl)phenyl]ethyl]-6-oxo-1H-pyrimidin-1-yl]ethyl]-3-methoxy-
benzamide
N-[2-[2-amino-4-[2-[3-(2-furyl)phenyl]ethyl]-6-oxo-1H-pyrimidin-1-yl]ethyl]-2-(4-methoxyphenyl)-
acetamide
2-amino-6-(2-bromophenyl)-3H-pyrimidin-4-one
2-amino-6-(2-bromophenyl)-3-methyl-3H-pyrimidin-4-one
2-amino-3-methyl-6-(2-phenylphenyl)-3H-pyrimidin-4-one
2-amino-6-[2-(3-methoxyphenyl)phenyl]-3-methyl-3H-pyrimidin-4-one
2-amino-6-[2-[3-(2-furyl)phenyl]ethyl]-3-[[2-(1H-tetrazol-5-yl)phenyl]methyl]-3H-pyrimidin-
4-one
2-amino-6-[2-[3-(2-furyl)phenyl]ethyl]-3-[(3-nitrophenyl)methyl]-3H-pyrimidin-4-one
3-[2-[2-amino-4-[2-[3-(2-furyl)phenyl]ethyl]-6-oxo-1H-pyrimidin-1-yl]ethylcarbamoyl]benzoic
acid methyl ester
2-amino-6-benzyl-3-methyl-3H-pyrimidin-4-one
2-amino-3-methyl-6-(2-phenylpropyl)-3H-pyrimidin-4-one
2-amino-6-[(5-bromo-3-pyridyl)methyl]-3-methyl-3H-pyrimidin-4-one
2-amino-6-[[5-(4-methoxyphenyl)-3-pyridyl]methyl]-3-methyl-3H-pyrimidin-4-one
2-amino-6-[4-[3-(4-methoxytetrahydropyran-4-yl)phenyl]sulfanylphenyl]-3H-pyrimidin-
4-one
2-amino-6-[(3,5-difluorophenyl)methyl]-3H-pyrimidin-4-one
4-[2-(2-amino-6-oxo-1H-pyrimidin-4-yl)ethyl]benzonitrile
2-(2-amino-6-oxo-4-phenethyl-1H-pyrimidin-1-yl)-N-benzyl-acetamide
2-amino-6-(3-benzyloxyphenyl)-3H-pyrimidin-4-one
2-amino-6-(2-benzyloxyphenyl)-3H-pyrimidin-4-one
2-amino-3-methyl-6-(1-methyl-1-phenyl-ethyl)-3H-pyrimidin-4-one
2-amino-6-(1-methyl-1-phenyl-ethyl)-3H-pyrimidin-4-one
2-amino-3-methyl-6-(1-phenylethyl)-3H-pyrimidin-4-one
2-amino-3-methyl-6-[2-[3-(4-phenylphenyl)phenyl]ethyl]-3H-pyrimidin-4-one
2-amino-3-methyl-6-(3-pyridylmethyl)-3H-pyrimidin-4-one
2-amino-6-methyl-3H-pyrimidin-4-one
2-amino-6-phenyl-3H-pyrimidin-4-one
2-amino-3-[2-(2-naphthyl)ethyl]-6-phenethyl-3H-pyrimidin-4-one
2-amino-6-[2-[4-(aminomethyl)phenyl]ethyl]-3H-pyrimidin-4-one
2-amino-6-[2-(2-naphthyl)ethyl]-3H-pyrimidin-4-one
2-amino-6-phenethyl-3H-pyrimidin-4-one
2-amino-3,6-dimethyl-6-[3-(2,3,4-trimethoxyphenyl)phenyl]-5,6-dihydro-3H-pyrimidin-
4-one
2-amino-6-[2-[3-(1H-indol-2-yl)phenyl]ethyl]-3-methyl-3H-pyrimidin-4-one

What is claimed is:

1-33. (canceled)

34. A compound of formula Ia or formula Ib: 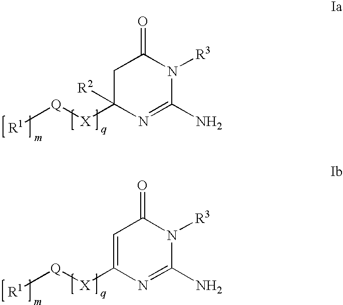
or a pharmaceutically acceptable salt, tautomer, or in vivo-hydrolysable precursor thereof, wherein:
Q is C₆₋₁₀aryl or C₅₋₁₀heterocyclyl;
X is C₁₋₃alkyl;
q is 0 or 1;
m is 0, 1 or 2;
each R¹ is, independently, selected from H, halogen, OH, C₁₋₆alkyl, C₂₋₆alkenyl, OC₁₋₄alkyl, OC₁₋₃alkylOC₁₋₃alkyl, OC₁₋₃alkylOC₁₋₃alkylOC₁₋₃alkyl, -L-R⁴, C₆₋₁₀aryl, OC₆₋₁₀aryl or C₅₋₁₅heterocyclyl, wherein said C₆₋₁₀aryl, OC₆₋₁₀aryl or C₅₋₁₅heterocyclyl is optionally substituted by 1, 2, or 3 substituents, independently, selected from: halogen, OH, NH₂, CN, C(═O)NH₂, C₁₋₆alkyl, C₁₋₆haloalkyl, OC₁₋₆alkyl, OC₁₋₆haloalkyl, C₁₋₄alkylOH, C₁₋₄alkylCN, C₁₋₄alkylN(C₁₋₆alkyl)₂, Si(C₁₋₄alkyl)₃, C₁₋₄alkylOC₁₋₃alkyl, CH₂OH, SO₂H, SO₂NHC(CH₃)₃, SO₂C₁₋₆alkyl, SO₂NHC₁₋₆alkyl, OC₁₋₃alkylOC₁₋₃alkyl, OC₁₋₃alkylOH, OC₁₋₃alkylOC(═O)C₁₋₃alkyl, C(═O)C₁₋₆alkyl, C(═O)OC₁₋₆alkyl, C(═O)NH₂, C₆₋₁₀aryl, SO₂C₆₋₁₀aryl, C₅₋₆heterocyclyl optionally substituted by OC₁₋₆alkyl, OC₅₋₆aryl, OC₁₋₆alkyl-C₆₋₁₀aryl, C(═O)H, COOH, C(═O)NHC₁₋₆alkyl, C(═O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(═O)H, NHC(═O)C₁₋₆alkyl, N(C₁₋₆alkyl)C(═O)C₁₋₆alkyl, NHC(═O)OC₁₋₆alkyl-C₆₋₁₀aryl, C₃₋₉cycloalkyl, C₅₋₆aryl, C(═O)C₅₋₆aryl, C(═O)OC₅₋₆aryl, C(═O)OC₁₋₆alkyl-C₆₋₁₀aryl, C(═O)NHC₅₋₆aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl), N(C₅₋₆aryl)₂, NHC(═O)C₅₋₆aryl, C₁₋₆alkyl-C₅₋₆heterocyclyl, OC₅₋₆heterocyclyl, C(═O)C₅₋₆heterocyclyl, C(═O)OC₅₋₆heterocyclyl, C(═O)NHC₅₋₆heterocyclyl, C(═O)N(C₅₋₆heterocyclyl)₂, SO₂C₅₋₆heterocyclyl, SO₂NHC₅₋₆heterocyclyl, SO₂N(C₅₋₆heterocyclyl)₂, NHSO₂C₁₋₆alkyl, NH(C₅₋₆heterocyclyl), N(C₅₋₆heterocyclyl)₂, or NHC(═O)C₅₋₆heterocyclyl;
R² is H, C₁₋₆alkyl, C₁₋₆haloalkyl, C₆₋₁₀aryl, or C₁₋₆alkyl-C₆₋₁₀aryl;
R³ is selected from H, C₁₋₆alkyl, C₁₋₆alkylR^{c}, C₁₋₆alkylNROR^{c}, C₁₋₆alkylOR^{c}, C₁₋₆alkylNHC₁₋₆alkylC₆₋₁₀arylR^{d}, C₁₋₆alkylNHC₆₋₁₀arylR^{d}, C₁₋₆alkylNHC(O)C₆₋₁₀arylR^{d}, C₁₋₆alkylOC₁₋₆alkylC₅₋₆arylR^{d}, C₁₋₆alkylC₅₋₆-arylR^{d}, C₁₋₆alkylC₅₋₉heterocyclylR^{d}, C₁₋₆alkylC₃₋₉cycloalkylR^{d}, C₁₋₆alkylNHC₁₋₆alkylC₅₋₉heterocyclylR^{d} C₁₋₆alkylNHC₅₋₉heterocyclyl(R^{d})ₙ, C₁₋₆alkylNHC(O)C₅₋₉heterocyclylR^{d}, C₁₋₆alkylOC₁₋₆alkylC₃₋₉heterocyclylR^{d}C₁₋₆alkylNHC₁₋₆alkylC₃₋₉cycloalkylR^{d}, or C₁₋₆alkylOC₁₋₆alkylC₃₋₉cycloalkylR^{d};
n is 0, 1, 2, 3, 4 or 5;
each L is, independently, absent, O, O—C₁₋₄alkylenyl, C₁₋₄alkylenyl, or C₂₋₄alkenylenyl;
each R⁴ is, independently, C₆₋₁₀aryl or C₅₋₁₅heterocyclyl, wherein each said aryl and heterocyclyl is optionally substituted by 1, 2, or 3 substituents, independently, selected from: halogen, OH, NH₂, CN, C(═O)NH₂, C₁₋₆alkyl, C₁₋₆haloalkyl, OC₁₋₆alkyl, OC₁₋₆haloalkyl, C₁₋₄alkylOH, C₁₋₄alkylCN, C₁₋₄alkylN(C₁₋₆alkyl)₂, Si(C₁₋₄alkyl)₃, C₁₋₄alkylOC₁₋₃alkyl, CH₂OH, SO₂H, SO₂NHC(CH₃)₃, SO₂C₁₋₆alkyl, SO₂NHC₁₋₆alkyl, OC₁₋₃alkylOC₁₋₃alkyl, OC₁₋₃alkylOH, OC₁₋₃alkylOC(═O)C₁₋₃alkyl, C(═O)C₁₋₆alkyl, C(═O)OC₁₋₆alkyl, C(═O)NH₂, C₆₋₁₀aryl, SO₂C₆₋₁₀aryl, C₅₋₆heterocyclyl optionally substituted by OC₁₋₆alkyl, OC₅₋₆aryl, OC₁₋₆alkyl-C₆₋₁₀aryl, C(═O)H, COOH, C(═O)NHC₁₋₆alkyl, C(═O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(═O)H, NHC(═O)C₁₋₆alkyl, N(C₁₋₆alkyl)C(═O)C₁₋₆alkyl, NHC(═O)OC₁₋₆alkyl-C₆₋₁₀aryl, C₃₋₉cycloalkyl, C₅₋₆aryl, C(═O)C₅₋₆aryl, C(═O)OC₅₋₆aryl, C(═O)OC₁₋₆alkyl-C₆₋₁₀aryl, C(═O)NHC₅₋₆aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl), N(C₅₋₆aryl)₂, NHC(═O)C₅₋₆aryl, C₁₋₆alkyl-C₅₋₆heterocyclyl, OC₅₋₆heterocyclyl, C(═O)C₅₋₆heterocyclyl, C(═O)OC₅₋₆heterocyclyl, C(═O)NHC₅₋₆heterocyclyl, C(═O)N(C₅₋₆heterocyclyl)₂, SO₂C₅₋₆heterocyclyl, SO₂NHC₅₋₆heterocyclyl, SO₂N(C₅₋₆heterocyclyl)₂, NHSO₂C₁₋₆alkyl, NH(C₅₋₆heterocyclyl), N(C₅₋₆heterocyclyl)₂, or NHC(═O)C₅₋₆heterocyclyl;
each R^{c} is, independently, selected from H, C₁₋₄alkyl, C(═O)C₁₋₄alkyl, C(═O)C₁₋₄alkylOC₁₋₄alkyl, C(═O)C₁₋₄alkylC(═O)OC₁₋₄alkyl, C(═O)C₁₋₄alkylC(═O)OH, C(═O)C₁₋₄alkylOC(═O)C₁₋₄alkyl, C₅₋₆arylR^{d}, C₅₋₉heterocyclylR^{d}, C₃₋₉cycloalkylR^{d}, C(═O)C₅₋₆arylR^{d}, C(═O)C₅₋₉heterocyclylR^{d}, C(═O)C₃₋₉cycloalkylR^{d}, C₁₋₄alkyl-C₅₋₆arylR^{d}, C₁₋₄alkyl-C₅₋₉heterocyclylR^{d}, or C₁₋₄alkyl-C₃₋₉cycloalkylR^{d}; and
R^{d} is selected from H, C₁₋₃alkyl, C₅₋₉heterocyclyl, NH₂, NO₂, OH, CN, COOH, C(═O)C₁₋₄alkyl, OC₁₋₃alkyl, or OC₁₋₃alkylOH;
provided that:
a) when the compound has formula Ia, R² is C₁₋₄alkyl, R³ is H, C₁₋₄alkyl, C₁₋₄alkylNR^{c}R^{c} or C₁₋₄alkylOR^{c}, and q is 0, then [R¹—]ₘ-Q is other than phenyl;
b) when the compound has formula Ia, R² is C₁₋₄alkyl, q is 0, Q is phenyl, R³ is H, C₁₋₄alkyl, C₁₋₄alkylNR^{c}R^{c} or C₁₋₄alkylOR^{c}, and m is 1, then R¹ is other than bromo, pyridyl, or methoxyphenyl;
c) when the compound has formula Ib, X is CH₂, R³ is H, C₁₋₄alkyl, C₁₋₄alkylNR^{c}R^{c} or C₁₋₄alkylOR^{c}, and q is 1, then [R¹—]ₘ-Q is other than phenyl; and
d) when the compound has formula Ib, X is CH₂ or CH(CH₃), q is 1, Q is phenyl, R³ is H, C₁₋₄alkyl, C₁₋₄alkylNR^{c}R^{c} or C₁₋₄alkylOR^{c}, and m is 2, then at least one of R¹ is other than fluoro.

35. A compound according to claim 1, or a pharmaceutically acceptable salt, tautomer or in vivo-hydrolysable precursor thereof, wherein said compound has the structure of said formula Ia.

36. A compound according to claim 1, or a pharmaceutically acceptable salt, tautomer or in vivo-hydrolysable precursor thereof, wherein said compound has the structure of said formula Ib.

37. A compound according to claim 1, or a pharmaceutically acceptable salt, tautomer or in vivo-hydrolysable precursor thereof, wherein R³ is selected from H, C₁₋₆alkyl, C₁₋₆alkylNR^{c}R^{c}, C₁₋₆alkylOR^{c}, C₁₋₆alkylNHC₁₋₆alkylC₆₋₁₀arylR^{d}, C₁₋₆alkylNHC(O)C₆ to arylR^{d}, C₁₋₆alkylOC₁₋₆alkylC₅₋₆arylR^{d}, C₁₋₆alkylC₅₋₉heterocyclylR^{d} or C₁₋₆alkylC₃₋₉cycloalkylR^{d}.

38. A compound according to claim 1, or a pharmaceutically acceptable salt, tautomer or in vivo-hydrolysable precursor thereof, wherein R³ is selected from H, C₁₋₆alkyl, C₁₋₆alkylNR^{c}R^{c}, or C₁₋₆alkyl-C₅₋₉heterocyclylR^{d}.

39. A compound according to claim 1, or a pharmaceutically acceptable salt, tautomer or in vivo-hydrolysable precursor thereof, wherein R³ is C₁₋₃alkyl.

40. A compound according to claim 1, or a pharmaceutically acceptable salt, tautomer or in vivo-hydrolysable precursor thereof, wherein Q is C₆₋₁₀aryl.

41. A compound according to claim 1, or a pharmaceutically acceptable salt, tautomer or in vivo-hydrolysable precursor thereof, wherein Q is C₆aryl.

42. A compound according to claim 1, or a pharmaceutically acceptable salt, tautomer or in vivo-hydrolysable precursor thereof, wherein X is methylenyl or ethylenyl, and q is 1.

43. A compound according to claim 1, or a pharmaceutically acceptable salt, tautomer or in vivo-hydrolysable precursor thereof, wherein q is 0.

44. A compound according to claim 1, or a pharmaceutically acceptable salt, tautomer or in vivo-hydrolysable precursor thereof, wherein m is 1 or 2, and each R¹ is, independently, selected from halogen, and -L-R⁴.

45. A compound according to claim 1, or a pharmaceutically acceptable salt, tautomer or in vivo-hydrolysable precursor thereof, wherein m is 1, R¹ is R⁴, and R⁴ is C₆₋₁₀aryl or C₅₋₁₅heterocyclyl, wherein each said aryl and heterocyclyl is optionally substituted by 1, or 2 substituents independently selected from: halogen, CN, C₁₋₄alkyl, C₁₋₄haloalkyl, OC₁₋₄alkyl, OC₁₋₄haloalkyl, —C(O)H, COOH, OC₁₋₄alkyl-C₆₋₁₀aryl, OH, and NHC(═O)C₁₋₄alkyl.

46. A compound according to claim 2 wherein R² is H or C₁₋₃alkyl.

47. A compound according to claim 2 wherein R² is C₁₋₃alkyl.

48. A compound according to claim 2 wherein:
each R¹ is, independently, selected from H, halogen, C₂₋₆alkenyl, -L-R⁴, C₆₋₁₀aryl, OC₆₋₁₀aryl or C₅₋₁₀heterocyclyl, wherein said C₆₋₁₀aryl, OC₆₋₁₀aryl or C₅₋₁₀heterocyclyl is optionally substituted by 1, 2, or 3 substituents, independently, selected from: halogen, OH, NH₂, CN, C(═O)NH₂, C₁₋₆alkyl, OC₁₋₆alkyl, C₁₋₄alkylOH, C₁₋₄alkylOC₁₋₃alkyl, CH₂OH, SO₂H, SO₂NHC(CH₃)₃, SO₂C₁₋₆alkyl, SO₂NHC₁₋₆alkyl, OC t₃alkylOC₁₋₃alkyl, OC₁₋₃alkylOH, OC₁₋₃alkylOC(═O)C₁₋₃alkyl, C(═O)C₁₋₆alkyl, C(═O)OC₁₋₆alkyl, C(═O)NH₂, C₅₋₆heterocyclyl, OC₅₋₆aryl, or OC₁₋₆alkyl-C₅₋₆aryl;
R² is H or C₁₋₆alkyl; and
R³ is H or C₁₋₃alkyl.

49. A compound according to claim 2 wherein:
Q is C₆aryl or C₉heterocyclyl;
R¹ is, independently, selected from H, halogen, -L-R⁴, C₆aryl, or C₅₋₆heterocyclyl wherein said C₆aryl, or C₅₋₆heterocyclyl is optionally substituted by 1, 2, or 3 substituents, independently, selected from: halogen, OH, NH₂, CN, C(═O)NH₂, C₁₋₆alkyl, OC₁₋₆alkyl, C₁₋₄alkylOH, C₁₋₄alkylOC₁₋₃alkyl, CH₂OH, SO₂H, SO₂NHC(CH₃)₃, SO₂C₁₋₆alkyl, SO₂NHC₁₋₆alkyl, OC₁₋₃alkylOC₁₋₃alkyl, OC₁₋₃alkylOH, OC₁₋₃alkylOC(═O)C₁₋₃alkyl, C(═O)C₁₋₆alkyl, C(═O)OC₁₋₆alkyl, C(═O)NH₂, C₅₋₆heterocyclyl, OC₅₋₆aryl, or OC₁₋₆alkyl-C₅₋₆aryl; and
R² is C₁₋₃alkyl.

50. A compound according to claim 2 wherein:
R² is methyl;
q is 0;
Q is phenyl;
m is 1;
R¹ is phenyl or heteroaryl, each optionally substituted by 1, 2 or 3 substituents independently selected from: methoxy, halogen, OCF₃, methyl, NH₂ and NHC(═O)CH₃; and
R³ is methyl.

51. A compound according to claim 2 wherein:
R² is methyl;
q is 1;
X is C₂ alkyl;
Q is phenyl;
m is 1;
R¹ is phenyl or heteroaryl, each optionally substituted by 1, 2 or 3 substituents independently selected from: methoxy, halogen, OCF₃, methyl, NH₂ and NHC(═O)CH₃; and
R³ is methyl.

52. A compound according to claim 3 wherein:
q is 1;
X is C₂ alkyl;
Q is phenyl;
m is 1; and
R¹ is phenyl or heteroaryl, each optionally substituted by 1, 2 or 3 substituents independently selected from: methoxy, halogen, OCF₃, methyl, NH₂ and NHC(═O)CH₃.

53. A compound according to claim 1 selected from:
2-amino-3,6-dimethyl-6-naphthalen-2-yl-5,6-dihydro-3H-pyrimidin-4-one;
2-Amino-6-[3-(5-chloro-thiophen-2-yl)-phenyl]-3,6-dimethyl-5,6-dihydro-3H-pyrimidin-4-one;
2-amino-6-(3,4-dichlorophenyl)-6-ethyl-5,6-dihydropyrimidin-4(3H)-one;
2-amino-6-(3,4-dichlorophenyl)-6-isobutyl-5,6-dihydropyrimidin-4(3H)-one;
2-amino-6-(3,4-dichlorophenyl)-3-methyl-6-(2-phenylethyl)-5,6-dihydropyrimidin-4(3H)-one;
2-amino-6-(3-bromo-4-chlorophenyl)-3,6-dimethyl-5,6-dihydropyrimidin-4(3H)-one;
2-amino-3,6-dimethyl-6-(2-phenylethyl)-5,6-dihydropyrimidin-4(3H)-one;
2-Amino-6-[2-(3-bromo-phenyl)-ethyl]-3,6-dimethyl-5,6-dihydro-3H-pyrimidin-4-one;
2-amino-6-[3-(benzyloxy)phenyl]-3,6-dimethyl-5,6-dihydropyrimidin-4(3H)-one;
2-amino-6-methyl-6-(3-phenoxyphenyl)-5,6-dihydropyrimidin-4(3H)-one;
2-amino-6-(3-bromo-4-chlorophenyl)-6-methyl-5,6-dihydropyrimidin-4(3H)-one;
2-amino-6-(3′-methoxy-1,1′-biphenyl-3-yl)-6-methyl-5,6-dihydropyrimidin-4(3H)-one;
2-amino-6-methyl-6-[3-(5-methylthien-2-yl)phenyl]-5,6-dihydropyrimidin-4(3H)-one;
2-amino-6-[3-(2-furyl)phenyl]-6-methyl-5,6-dihydropyrimidin-4(3H)-one;
2-amino-6-(3′-butoxy-1,1′-biphenyl-3-yl)-6-methyl-5,6-dihydropyrimidin-4(3H)-one;
2-amino-6-(6-chloro-3′-methoxy-1,1′-biphenyl-3-yl)-6-methyl-5,6-dihydropyrimidin-4(3H)-one;
2-amino-3,6-dimethyl-6-[3-(5-methylthien-2-yl)phenyl]-5,6-dihydropyrimidin-4(3H)-one;
2-amino-6-(3′-isopropoxy-1,1′-biphenyl-3-yl)-3,6-dimethyl-5,6-dihydropyrimidin-4(3H)-one;
2-amino-6-(6-chloro-3′-methoxy-1,1′-biphenyl-3-yl)-3,6-dimethyl-5,6-dihydropyrimidin-4(3H)-one;
2-amino-6-(4-chloro-3-quinolin-5-yl-phenyl)-3,6-dimethyl-5,6-dihydro-3H-pyrimidin-4-one;
2-Amino-6-[2-(3′-methoxy-biphenyl-3-yl)-ethyl]-3,6-dimethyl-5,6-dihydro-3H-pyrimidin-4-one;
2-Amino-6-methyl-6-quinolin-6-yl-5,6-dihydro-3H-pyrimidin-4-one;
2-Amino-6-(3,4-dichloro-phenyl)-3,6-dimethyl-5,6-dihydro-3H-pyrimidin-4-one;
2-Amino-6-(3,4-dichloro-phenyl)-6-methyl-5,6-dihydro-3H-pyrimidin-4-one;
2-Amino-6-[2-(3-bromophenyl)ethyl]-3-methylpyrimidin-4(3H)-one;
2-Amino-6-[2-(2-bromophenyl)ethyl]-3-methylpyrimidin-4(3H)-one;
2-Amino-6-[2-(4-bromophenyl)ethyl]-3-methylpyrimidin-4(3H)-one;
2-Amino-6-[2-(1H-indol-6-yl)ethyl]-3-methylpyrimidin-4(3H)-one;
2-Amino-6-[2-(1H-indol-6-yl)ethyl]pyrimidin-4(3H)-one;
2-Amino-3-methyl-6-[2-(2-naphthyl)ethyl]pyrimidin-4(3H)-one;
2-amino-3-(1,3-dioxolan-2-ylmethyl)-6-[2-(1H-indol-6-yl)ethyl]pyrimidin-4(3H)-one;
2-Amino-3-methyl-6-{2-[3-(2-thienyl)phenyl]ethyl}pyrimidin-4(3H)-one;
2-amino-3-methyl-6-{2-[4′-(methylsulfonyl)biphenyl-3-yl]ethyl}pyrimidin-4(3H)-one;
2-amino-6-{2-[3-(1H-indol-5-yl)phenyl]ethyl}-3-methylpyrimidin-4(3H)-one;
2-amino-6-{2-[3-(2,3-dihydro-1,4-benzodioxin-6-yl)phenyl]ethyl}-3-methylpyrimidin-4(3H)-one;
2-amino-3-methyl-6-{2-[3′-(1H-pyrazol-1-yl)biphenyl-3-yl]ethyl}pyrimidin-4(3H)-one
2-amino-6-{2-[3-(2,3-dihydro-1-benzofuran-5-yl)phenyl]ethyl}-3-methylpyrimidin-4(3H)-one;
2-amino-3-methyl-6-{2-[3-(1H-pyrrol-2-yl)phenyl]ethyl}pyrimidin-4(3H)-one;
methyl 3′-[2-(2-amino-1-methyl-6-oxo-1,6-dihydropyrimidin-4-yl)ethyl]biphenyl-4-carboxylate;
2-amino-6-{2-[3′-(hydroxymethyl)biphenyl-3-yl]ethyl}-3-methylpyrimidin-4(3H)-one;
2-amino-6-[2-(3′-hydroxybiphenyl-3-yl)ethyl]-3-methylpyrimidin-4(3H)-one;
6-[2-(3′-acetylbiphenyl-3-yl)ethyl]-2-amino-3-methylpyrimidin-4(3H)-one;
2-amino-3-methyl-6-[2-(3-pyridin-4-ylphenyl)ethyl]pyrimidin-4(3H)-one;
2-amino-6-{2-[3-(2-furyl)phenyl]ethyl}-3-methylpyrimidin-4(3H)-one;
2-amino-3-methyl-6-{2-[3-(5-methyl-2-thienyl)phenyl]ethyl}pyrimidin-4(3H)-one;
2-amino-6-[2-(3′-methoxybiphenyl-3-yl)ethyl]-3-methylpyrimidin-4(3H)-one;
2-amino-3-methyl-6-{2-[3-(4-methyl-2-thienyl)phenyl]ethyl}pyrimidin-4(3H)-one;
2-amino-6-(2-biphenyl-3-ylethyl)-3-methylpyrimidin-4(3H)-one;
2-amino-6-[2-(4′-chlorobiphenyl-3-yl)ethyl]-3-methylpyrimidin-4(3H)-one;
2-amino-6-[2-(4′-hydroxybiphenyl-3-yl)ethyl]-3-methylpyrimidin-4(3H)-one;
{3′-[2-(2-amino-1-methyl-6-oxo-1,6-dihydropyrimidin-4-yl)ethyl]biphenyl-4-yl}acetonitrile;
2-amino-3-methyl-6-{2-[3-(3-thienyl)phenyl]ethyl}pyrimidin-4(3H)-one;
2-amino-6-[2-(4′-methoxybiphenyl-3-yl)ethyl]-3-methylpyrimidin-4(3H)-one;
2-amino-6-(2-{3-[5-(hydroxymethyl)-2-thienyl]phenyl}ethyl) 3-methylpyrimidin-4(3H)-one;
2-amino-3-methyl-6-[2-(3-pyridin-3-ylphenyl)ethyl]pyrimidin-4(3H)-one;
2-amino-6-[2-(3′-ethoxybiphenyl-3-yl)ethyl]-3-methylpyrimidin-4(3H)-one;
2-amino-6-{2-[3-(3-furyl)phenyl]ethyl}-3-methylpyrimidin-4(3H)-one;
2-amino-6-[2-(2′-methoxybiphenyl-3-yl)ethyl]-3-methylpyrimidin-4(3H)-one;
3′-[2-(2-amino-1-methyl-6-oxo-1,6-dihydropyrimidin-4-yl)ethyl]-N-(tert-butyl) biphenyl-2-sulfonamide;
methyl 3′-[2-(2-amino-1-methyl-6-oxo-1,6-dihydropyrimidin-4-yl)ethyl]biphenyl-2-carboxylate;
2-amino-3-methyl-6-{2-[2-(2-thienyl)phenyl]ethyl}pyrimidin-4(3H)-one;
2-amino-6-{2-[3′-(hydroxymethyl)biphenyl-4-yl]ethyl}-3-methylpyrimidin-4(3H)-one;
2-amino-6-[2-(3′,4′-dimethoxybiphenyl-4-yl)ethyl]-3-methylpyrimidin-4(3H)-one;
2-amino-6-[2-(3′-methoxybiphenyl-4-yl)ethyl]-3-methylpyrimidin-4(3H)-one;
2-amino-3-methyl-6-{2-[4-(2-thienyl)phenyl]ethyl}pyrimidin-4(3H)-one;
2-amino-6-[2-(1,1′-biphenyl-3-yl)ethyl]pyrimidin-4(3H)-one;
N-{2-[2-Amino-4-{2-[3-(2-furyl)phenyl]ethyl}-6-oxopyrimidin-1(6H)-yl]ethyl}acetamide;
N-{2-[2-amino-4-{2-[3-(2-furyl)phenyl]ethyl}-6-oxopyrimidin-1(6H)-yl]ethyl}butanamide;
1-{2-[2-amino-4-{2-[3-(2-furyl)phenyl]ethyl}-6-oxopyrimidin-1(6H)-yl]ethyl}pyrrolidine-2,5-dione; methyl
3-({2-[2-amino-4-{2-[3-(2-furyl)phenyl]ethyl}-6-oxopyrimidin-1(6H)-yl]ethyl}amino)-3-oxopropanoate; methyl
5-({2-[2-amino-4-{2-[3-(2-furyl)phenyl]ethyl}-6-oxopyrimidin-1(6H)-yl]ethyl}amino)-5-oxopentanoate; methyl
6-({2-[2-amino-4-{2-[3-(2-furyl)phenyl]ethyl}-6-oxopyrimidin-1(6H)-yl]ethyl}amino)-6-oxohexanoate;
6-({2-[2-amino-4-{2-[3-(2-furyl)phenyl]ethyl}-6-oxopyrimidin-1(6H)-yl]ethyl}amino)-6-oxohexanoic acid;
N-{2-[2-amino-4-{2-[3-(2-furyl)phenyl]ethyl}-6-oxopyrimidin-1(6H)-yl]ethyl}-2-methoxyacetamide;
2-Amino-3-[2-(benzylamino)ethyl]-6-{2-[3-(2-furyl)phenyl]ethyl}pyrimidin-4(3H)-one;
2-amino-6-{2-[3-(2-furyl)phenyl]ethyl}-3-{2-[(3-methoxybenzyl)amino]ethyl}pyrimidin-4(3H)-one;
2-({3′-[2-(2-Amino-1-methyl-6-oxo-1,6-dihydropyrimidin-4-yl)ethyl]-1,1′-biphenyl-3-yl}oxy)ethyl acetate;
2-Amino-6-{2-[3′-(2-hydroxyethoxy)-1,1′-biphenyl-3-yl]ethyl}-3-methylpyrimidin-4(3H)-one;
2-Amino-6-{2-[3′-(2-methoxyethoxy)-1,1′-biphenyl-3-yl]ethyl}-3-methylpyrimidin-4(3H)-one;
2-({3′-[2-(2-Amino-1-methyl-6-oxo-1,6-dihydropyrimidin-4-yl)ethyl]-1,1′-biphenyl-4-yl}oxy)ethyl acetate;
2-Amino-6-{2-[4′-(2-methoxyethoxy)-1,1′-biphenyl-3-yl]ethyl}-3-methylpyrimidin-4(3H)-one;
2-Amino-3-[2-(benzyloxy)ethyl]-6-{2-[3-(2-furyl)phenyl]ethyl}pyrimidin-4(3H)-one;
2-Amino-3-methyl-6-(3′-methyl-biphenyl-3-ylmethyl)-3H-pyrimidin-4-one;
2-Amino-6-(3′-methoxy-biphenyl-3-ylmethyl)-3-methyl-3H-pyrimidin-4-one;
2-Amino-6-(3′-ethoxy-biphenyl-3-ylmethyl)-3-methyl-3H-pyrimidin-4-one;
3′-(2-Amino-1-methyl-6-oxo-1,6-dihydro-pyrimidin-4-ylmethyl)-biphenyl-3-carbonitrile;
2-Amino-3-methyl-6-(3′-vinyl-biphenyl-3-ylmethyl)-3H-pyrimidin-4-one;
2-Amino-6-(3′-isopropyl-biphenyl-3-ylmethyl)-3-methyl-3H-pyrimidin-4-one;
2-Amino-6-(4′-methoxy-biphenyl-3-ylmethyl)-3-methyl-3H-pyrimidin-4-one;
2-amino-6-[2-[3-(3-methoxyphenyl)phenyl]ethyl]-3-methyl-6-phenyl-5,6-dihydro-3H-pyrimidin-4-one;
2-amino-6-[3-(3-methoxyphenyl)phenyl]-3-methyl-6-(trifluoromethyl)-5,6-dihydro-3H-pyrimidin-4-one;
(R)-2-Amino-6-[2-(3′-methoxy-biphenyl-3-yl)-ethyl]-3,6-dimethyl-5,6-dihydro-3H-pyrimidin-4-one;
2-Amino-6-[2-(2′-fluoro-3′-methoxy-biphenyl-3-yl)-ethyl]-3,6-dimethyl-5,6-dihydro-3H-pyrimidin-4-one;
2-Amino-6-{2-[3-(5-chloro-thiophen-2-yl)-phenyl]-ethyl}-3,6-dimethyl-5,6-dihydro-3H-pyrimidin-4-one;
2-Amino-6-[2-(3-furan-2-yl-phenyl)-ethyl]-3,6-dimethyl-5,6-dihydro-3H-pyrimidin-4-one;
2-Amino-6-[2-(3′,5′-dimethoxy-biphenyl-3-yl)-ethyl]-3,6-dimethyl-5,6-dihydro-3H-pyrimidin-4-one;
2-Amino-6-[2-(4′-methoxy-biphenyl-3-yl)-ethyl]-3,6-dimethyl-5,6-dihydro-3H-pyrimidin-4-one;
2-Amino-6-(2-biphenyl-3-yl-ethyl)-3,6-dimethyl-5,6-dihydro-3H-pyrimidin-4-one;
2-Amino-3-benzyl-6-[2-(3′-methoxy-biphenyl-3-yl)-ethyl]-6-methyl-5,6-dihydro-3H-pyrimidin-4-one;
N-{3′-[2-(2-Amino-1,4-dimethyl-6-oxo-1,4,5,6-tetrahydro-pyrimidin-4-yl)-ethyl]-biphenyl-3-yl}-acetamide;
2-Amino-6-[2-(3′-hydroxymethyl-biphenyl-3-yl)-ethyl]-3,6-dimethyl-5,6-dihydro-3H-pyrimidin-4-one;
2-Amino-6-methyl-6-naphthalen-2-yl-5,6-dihydro-3H-pyrimidin-4-one;
2-Amino-3-benzyl-6-[2-(3-bromo-phenyl)-ethyl]-6-methyl-5,6-dihydro-3H-pyrimidin-4-one;
2-Amino-6-[2-(4′-methanesulfonyl-biphenyl-3-yl)-ethyl]-3,6-dimethyl-5,6-dihydro-3H-pyrimidin-4-one;
2-Amino-6-[2-(3′,4′-dimethoxy-biphenyl-3-yl)-ethyl]-3,6-dimethyl-5,6-dihydro-3H-pyrimidin-4-one;
3′-[2-(2-Amino-1,4-dimethyl-6-oxo-1,4,5,6-tetrahydro-pyrimidin-4-yl)-ethyl]-biphenyl-4-sulfonic acid dimethylamide;
2-Amino-6-(2′-fluoro-3′-methoxy-biphenyl-3-yl)-3,6-dimethyl-5,6-dihydro-3H-pyrimidin-4-one;
2-Amino-6-(3′,5′-dimethoxy-biphenyl-3-yl)-3,6-dimethyl-5,6-dihydro-3H-pyrimidin-4-one;
2-Amino-6-(4′-ethylbiphenyl-3-yl)-3,6-dimethyl-5,6-dihydropyrimidin-4(3H)-one;
2-amino-6-(2′-ethylbiphenyl-3-yl)-3,6-dimethyl-5,6-dihydropyrimidin-4(3H)-one;
2-amino-6-(3-isoquinolin-5-ylphenyl)-3,6-dimethyl-5,6-dihydropyrimidin-4(3H)-one;
2-amino-3,6-dimethyl-6-[3′-(1H-pyrazol-1-yl)biphenyl-3-yl]-5,6-dihydropyrimidin-4(3H)-one;
2-amino-6-(4′-isopropoxybiphenyl-3-yl)-3,6-dimethyl-5,6-dihydropyrimidin-4(3H)-one;
2-amino-6-(3′-ethoxybiphenyl-3-yl)-3,6-dimethyl-5,6-dihydropyrimidin-4(3H)-one;
2-amino-3,6-dimethyl-6-[2′-(trifluoromethyl)biphenyl-3-yl]-5,6-dihydropyrimidin-4(3H)-one;
2-amino-6-(2′-chlorobiphenyl-3-yl)-3,6-dimethyl-5,6-dihydropyrimidin-4(3H)-one;
2-amino-6-(2′-fluorobiphenyl-3-yl)-3,6-dimethyl-5,6-dihydropyrimidin-4(3H)-one;
2-amino-6-(2′-methoxybiphenyl-3-yl)-3,6-dimethyl-5,6-dihydropyrimidin-4(3H)-one;
2-amino-6-(2′-ethoxybiphenyl-3-yl)-3,6-dimethyl-5,6-dihydropyrimidin-4(3H)-one;
2-amino-6-(4′-ethoxybiphenyl-3-yl)-3,6-dimethyl-5,6-dihydropyrimidin-4(3H)-one;
2-amino-6-(2′,6′-dimethylbiphenyl-3-yl)-3,6-dimethyl-5,6-dihydropyrimidin-4(3H)-one;
2-amino-3,6-dimethyl-6-[3′-(trifluoromethyl)biphenyl-3-yl]-5,6-dihydropyrimidin-4(3H)-one;
2-amino-3,6-dimethyl-6-[4′-(trifluoromethyl)biphenyl-3-yl]-5,6-dihydropyrimidin-4(3H)-one;
2-amino-6-[3′,5′-bis(trifluoromethyl)biphenyl-3-yl]-3,6-dimethyl-5,6-dihydropyrimidin-4(3H)-one;
2-amino-6-(4′-tert-butylbiphenyl-3-yl)-3,6-dimethyl-5,6-dihydropyrimidin-4(3H)-one;
2-amino-6-(3′,4′-dimethoxybiphenyl-3-yl)-3,6-dimethyl-5,6-dihydropyrimidin-4(3H)-one;
2-amino-3,6-dimethyl-6-(4′-phenoxybiphenyl-3-yl)-5,6-dihydropyrimidin-4(3H)-one;
2-amino-6-(2′,4′-dimethoxybiphenyl-3-yl)-3,6-dimethyl-5,6-dihydropyrimidin-4(3H)-one;
2-amino-6-[3-(1-benzofuran-2-yl)phenyl]-3,6-dimethyl-5,6-dihydropyrimidin-4(3H)-one;
2-amino-6-[3-(1,3-benzodioxol-5-yl)phenyl]-3,6-dimethyl-5,6-dihydropyrimidin-4(3H)-one;
2-amino-3,6-dimethyl-6-[3′-(trifluoromethoxy)biphenyl-3-yl]-5,6-dihydropyrimidin-4(3H)-one;
2-amino-6-[3′-(hydroxymethyl)biphenyl-3-yl]-3,6-dimethyl-5,6-dihydropyrimidin-4(3H)-one;
2-amino-3,6-dimethyl-6-[3-(1-naphthyl)phenyl]-5,6-dihydropyrimidin-4(3H)-one;
2-amino-3,6-dimethyl-6-(1,1′:3′,1″-terphenyl-3-yl)-5,6-dihydropyrimidin-4(3H)-one;
2-amino-6-[4′-(benzyloxy)biphenyl-3-yl]-3,6-dimethyl-5,6-dihydropyrimidin-4(3H)-one;
2-amino-6-[3′-(benzyloxy)biphenyl-3-yl]-3,6-dimethyl-5,6-dihydropyrimidin-4(3H)-one;
2-amino-6-(4′-butylbiphenyl-3-yl)-3,6-dimethyl-5,6-dihydropyrimidin-4(3H)-one;
2-amino-6-[3-(2,3-dihydro-1,4-benzodioxin-6-yl)phenyl]-3,6-dimethyl-5,6-dihydropyrimidin-4(3H)-one;
2-amino-6-[3-(2,3-dihydro-1-benzofuran-5-yl)phenyl]-3,6-dimethyl-5,6-dihydropyrimidin-4(3H)-one;
2-amino-3,6-dimethyl-6-(4′-propylbiphenyl-3-yl)-5,6-dihydropyrimidin-4(3H)-one;
2-amino-6-(2′,3′-dimethoxybiphenyl-3-yl)-3,6-dimethyl-5,6-dihydropyrimidin-4(3H)-one;
3′-(2-amino-1,4-dimethyl-6-oxo-1,4,5,6-tetrahydropyrimidin-4-yl)biphenyl-3-carboxylic acid;
2-amino-3,6-dimethyl-6-(3-thianthren-1-ylphenyl)-5,6-dihydropyrimidin-4(3H)-one;
2-amino-3,6-dimethyl-6-(1,1′:2′,1″-terphenyl-3-yl)-5,6-dihydropyrimidin-4(3H)-one;
2-amino-3,6-dimethyl-6-[3-(2-thienyl)phenyl]-5,6-dihydropyrimidin-4(3H)-one;
2-amino-3,6-dimethyl-6-[3-(3-thienyl)phenyl]-5,6-dihydropyrimidin-4(3H)-one;
2-amino-6-(3′,5′-dimethylbiphenyl-3-yl)-3,6-dimethyl-5,6-dihydropyrimidin-4(3H)-one;
2-amino-3,6-dimethyl-6-[3-(2-naphthyl)phenyl]-5,6-dihydropyrimidin-4(3H)-one;
2-amino-6-[4′-(hydroxymethyl)biphenyl-3-yl]-3,6-dimethyl-5,6-dihydropyrimidin-4(3H)-one;
2-amino-6-[3-(2-furyl)phenyl]-3,6-dimethyl-5,6-dihydropyrimidin-4(3H)-one;
2-amino-6-(4′-hydroxybiphenyl-3-yl)-3,6-dimethyl-5,6-dihydropyrimidin-4(3H)-one;
6-(4′-acetylbiphenyl-3-yl)-2-amino-3,6-dimethyl-5,6-dihydropyrimidin-4(3H)-one;
6-(3′-acetylbiphenyl-3-yl)-2-amino-3,6-dimethyl-5,6-dihydropyrimidin-4(3H)-one;
2-amino-6-[3-(1-benzothien-2-yl)phenyl]-3,6-dimethyl-5,6-dihydropyrimidin-4(3H)-one;
2-amino-3,6-dimethyl-6-(3′,4′,5′-trimethoxybiphenyl-3-yl)-5,6-dihydropyrimidin-4(3H)-one;
6-[3-(5-acetyl-2-thienyl)phenyl]-2-amino-3,6-dimethyl-5,6-dihydropyrimidin-4(3H)-one;
2-amino-6-(3′-fluoro-1,1′:4′, 1″-terphenyl-3-yl)-3,6-dimethyl-5,6-dihydropyrimidin-4(3H)-one;
2-amino-3,6-dimethyl-6-(3-pyridin-3-ylphenyl)-5,6-dihydropyrimidin-4(3H)-one;
2-amino-6-[3-(1-benzothien-3-yl)phenyl]-3,6-dimethyl-5,6-dihydropyrimidin-4(3H)-one;
3′-(2-amino-1,4-dimethyl-6-oxo-1,4,5,6-tetrahydropyrimidin-4-yl)biphenyl-3-carbonitrile;
3′-(2-amino-1,4-dimethyl-6-oxo-1,4,5,6-tetrahydropyrimidin-4-yl)biphenyl-4-carbonitrile;
6-(2′-acetylbiphenyl-3-yl)-2-amino-3,6-dimethyl-5,6-dihydropyrimidin-4(3H)-one;
2-amino-3,6-dimethyl-6-[4′-(methylsulfonyl)biphenyl-3-yl]-5,6-dihydropyrimidin-4(3H)-one;
2-amino-6-(4′-cyclohexylbiphenyl-3-yl)-3,6-dimethyl-5,6-dihydropyrimidin-4(3H)-one
2-amino-3,6-dimethyl-6-(3′,4′,5′-trifluorobiphenyl-3-yl)-5,6-dihydropyrimidin-4(3H)-one;
2-amino-6-[4′-(ethylsulfonyl)biphenyl-3-yl]-3,6-dimethyl-5,6-dihydropyrimidin-4(3H)-one;
2-amino-6-(4′-hydroxy-3′-methoxybiphenyl-3-yl)-3,6-dimethyl-5,6-dihydropyrimidin-4(3H)-one;
2-amino-6-(4′-hydroxy-3′,5′-dimethylbiphenyl-3-yl)-3,6-dimethyl-5,6-dihydropyrimidin-4(3H)-one;
2-amino-6-(2′-hydroxybiphenyl-3-yl)-3,6-dimethyl-5,6-dihydropyrimidin-4(3H)-one;
2-amino-6-(3′-hydroxybiphenyl-3-yl)-3,6-dimethyl-5,6-dihydropyrimidin-4(3H)-one;
5-[3-(2-amino-1,4-dimethyl-6-oxo-1,4,5,6-tetrahydropyrimidin-4-yl)phenyl]thiophene-2-carbonitrile;
N-[3′-(2-amino-1,4-dimethyl-6-oxo-1,4,5,6-tetrahydropyrimidin-4-yl)biphenyl-4-yl]acetamide;
2-amino-3,6-dimethyl-6-{3-[1-(phenylsulfonyl)-1H-indol-3-yl]phenyl}-5,6-dihydropyrimidin-4(3H)-one;
2-amino-6-[3-(3,4-dihydro-2H-1,5-benzodioxepin-7-yl)phenyl]-3,6-dimethyl-5,6-dihydropyrimidin-4(3H)-one;
2-amino-6-[3-(6-ethoxy-2-naphthyl)phenyl]-3,6-dimethyl-5,6-dihydropyrimidin-4(3H)-one;
2-amino-3,6-dimethyl-6-(3′-propoxybiphenyl-3-yl)-5,6-dihydropyrimidin-4(3H)-one;
methyl 3′-(2-amino-1,4-dimethyl-6-oxo-1,4,5,6-tetrahydropyrimidin-4-yl) biphenyl-4-carboxylate;
2-amino-3,6-dimethyl-6-(2′-phenoxybiphenyl-3-yl)-5,6-dihydropyrimidin-4(3H)-one;
2-amino-6-(4′-isobutylbiphenyl-3-yl)-3,6-dimethyl-5,6-dihydropyrimidin-4(3H)-one;
2-amino-6-[2′-(benzyloxy)biphenyl-3-yl]-3,6-dimethyl-5,6-dihydropyrimidin-4(3H)-one;
methyl 3′-(2-amino-1,4-dimethyl-6-oxo-1,4,5,6-tetrahydropyrimidin-4-yl) biphenyl-3-carboxylate;
benzyl 3′-(2-amino-1,4-dimethyl-6-oxo-1,4,5,6-tetrahydropyrimidin-4-yl) biphenyl-3-carboxylate;
2-amino-6-(2′-methoxy-5′-methylbiphenyl-3-yl)-3,6-dimethyl-5,6-dihydropyrimidin-4(3H)-one;
2-amino-6-(4′-methoxy-3′,5′-dimethylbiphenyl-3-yl)-3,6-dimethyl-5,6-dihydropyrimidin-4(3H)-one;
benzyl [3′-(2-amino-1,4-dimethyl-6-oxo-1,4,5,6-tetrahydropyrimidin-4-yl) biphenyl-4-yl]carbamate;
ethyl 3′-(2-amino-1,4-dimethyl-6-oxo-1,4,5,6-tetrahydropyrimidin-4-yl) biphenyl-4-carboxylate;
benzyl 3′-(2-amino-1,4-dimethyl-6-oxo-1,4,5,6-tetrahydropyrimidin-4-yl) biphenyl-4-carboxylate;
N-[3′-(2-amino-1,4-dimethyl-6-oxo-1,4,5,6-tetrahydropyrimidin-4-yl) biphenyl-2-yl]methanesulfonamide;
3′-(2-amino-1,4-dimethyl-6-oxo-1,4,5,6-tetrahydropyrimidin-4-yl)-N-(tert-butyl)biphenyl-2-sulfonamide;
2-amino-6-[3-(3,5-dimethylisoxazol-4-yl)phenyl]-3,6-dimethyl-5,6-dihydropyrimidin-4(3H)-one;
N-[3′-(2-amino-1,4-dimethyl-6-oxo-1,4,5,6-tetrahydropyrimidin-4-yl)biphenyl-3-yl]acetamide;
2-amino-6-(2′,6′-dichlorobiphenyl-3-yl)-3,6-dimethyl-5,6-dihydropyrimidin-4(3H)-one
2-amino-6-(3′-chloro-2′-fluorobiphenyl-3-yl)-3,6-dimethyl-5,6-dihydropyrimidin-4(3H)-one;
2-amino-6-(4′-butoxy-3′-chlorobiphenyl-3-yl)-3,6-dimethyl-5,6-dihydropyrimidin-4(3H)-one;
[3′-(2-amino-1,4-dimethyl-6-oxo-1,4,5,6-tetrahydropyrimidin-4-yl)biphenyl-3-yl]formamide;
2-amino-6-[3-(2-fluoropyridin-3-yl)phenyl]-3,6-dimethyl-5,6-dihydropyrimidin-4(3H)-one;
2-amino-6-(3′-bromo-2′-ethoxy-5′-methylbiphenyl-3-yl)-3,6-dimethyl-5,6-dihydropyrimidin-4(3H)-one;
2-amino-6-[3-(2-ethoxy-1-naphthyl)phenyl]-3,6-dimethyl-5,6-dihydropyrimidin-4(3H)-one;
2-amino-6-[4′-(benzyloxy)-2′-fluorobiphenyl-3-yl]-3,6-dimethyl-5,6-dihydropyrimidin-4(3H)-one;
2-amino-3,6-dimethyl-6-[2′-(morpholin-4-ylmethyl)biphenyl-3-yl]-5,6-dihydropyrimidin-4(3H)-one;
3′-(2-amino-1,4-dimethyl-6-oxo-1,4,5,6-tetrahydropyrimidin-4-yl)-3-fluorobiphenyl-4-carbaldehyde;
2-amino-3,6-dimethyl-6-[4′-(trimethylsilyl)biphenyl-3-yl]-5,6-dihydropyrimidin-4(3H)-one;
2-amino-6-(3′-butoxybiphenyl-3-yl)-3,6-dimethyl-5,6-dihydropyrimidin-4(3H)-one;
2-amino-3,6-dimethyl-6-(2′,4′,6′-trimethylbiphenyl-3-yl)-5,6-dihydropyrimidin-4(3H)-one;
2-amino-6-[3-(2-chloro-3-fluoropyridin-4-yl)phenyl]-3,6-dimethyl-5,6-dihydropyrimidin-4(3H)-one;
2-amino-6-(5′-chloro-2′-methylbiphenyl-3-yl)-3,6-dimethyl-5,6-dihydropyrimidin-4(3H)-one;
2-amino-6-(2′,5′-difluorobiphenyl-3-yl)-3,6-dimethyl-5,6-dihydropyrimidin-4(3H)-one;
2-amino-3,6-dimethyl-6-(1,1′:4′, 1″-terphenyl-3-yl)-5,6-dihydropyrimidin-4(3H)-one;
2-amino-6-(3′-chloro-4′-fluorobiphenyl-3-yl)-3,6-dimethyl-5,6-dihydropyrimidin-4(3H)-one;
2-amino-6-(4′-fluorobiphenyl-3-yl)-3,6-dimethyl-5,6-dihydropyrimidin-4(3H)-one;
2-amino-6-(3-dibenzo[b,d]thien-4-ylphenyl)-3,6-dimethyl-5,6-dihydropyrimidin-4(3H)-one;
2-amino-6-biphenyl-3-yl-3,6-dimethyl-5,6-dihydropyrimidin-4(3H)-one;
2-amino-6-(2′,3′-dimethylbiphenyl-3-yl)-3,6-dimethyl-5,6-dihydropyrimidin-4(3H)-one;
2-amino-6-{3-[(E)-2-biphenyl-4-ylvinyl]phenyl}-3,6-dimethyl-5,6-dihydropyrimidin-4(3H)-one;
2-amino-6-[4′-(benzyloxy)-3′-chlorobiphenyl-3-yl]-3,6-dimethyl-5,6-dihydropyrimidin-4(3H)-one;
2-amino-6-(4′-butoxy-2′-methylbiphenyl-3-yl)-3,6-dimethyl-5,6-dihydropyrimidin-4(3H)-one;
3′-(2-amino-1,4-dimethyl-6-oxo-1,4,5,6-tetrahydropyrimidin-4-yl)biphenyl-2-carbaldehyde;
2-amino-6-(3′-bromo-2′-isopropoxy-5′-methylbiphenyl-3-yl)-3,6-dimethyl-5,6-dihydropyrimidin-4(3H)-one;
3′-(2-amino-1,4-dimethyl-6-oxo-1,4,5,6-tetrahydropyrimidin-4-yl)biphenyl-4-carboxamide;
2-amino-6-(3′,5′-difluorobiphenyl-3-yl)-3,6-dimethyl-5,6-dihydropyrimidin-4(3H)-one;
2-amino-3,6-dimethyl-6-[3-(1H-pyrazol-3-yl)phenyl]-5,6-dihydropyrimidin-4(3H)-one
2-amino-3,6-dimethyl-6-(3-quinolin-5-ylphenyl)-5,6-dihydropyrimidin-4(3H)-one;
2-amino-6-(2′,5′-dimethoxybiphenyl-3-yl)-3,6-dimethyl-5,6-dihydropyrimidin-4(3H)-one;
2-amino-6-(3′-chlorobiphenyl-3-yl)-3,6-dimethyl-5,6-dihydropyrimidin-4(3H)-one;
2-amino-6-(4′-chlorobiphenyl-3-yl)-3,6-dimethyl-5,6-dihydropyrimidin-4(3H)-one;
2-amino-6-(3′-bromo-2′-butoxy-5′-methylbiphenyl-3-yl)-3,6-dimethyl-5,6-dihydropyrimidin-4(3H)-one;
2-amino-6-[2-[3-(2-aminophenyl)phenyl]ethyl]-3-methyl-3H-pyrimidin-4-one;
2-amino-3-methyl-6-[2-[3-(p-tolyl)phenyl]ethyl]-3H-pyrimidin-4-one;
2-amino-3-methyl-6-[2-[3-(m-tolyl)phenyl]ethyl]-3H-pyrimidin-4-one;
2-amino-3-methyl-6-[2-[3-(o-tolyl)phenyl]ethyl]-3H-pyrimidin-4-one;
2-amino-6-[2-[3-[4-(hydroxymethyl)phenyl]phenyl]ethyl]-3-methyl-3H-pyrimidin-4-one;
2-amino-6-[2-[3-(4-ethylsulfonylphenyl)phenyl]ethyl]-3-methyl-3H-pyrimidin-4-one;
2-amino-6-[2-[3-[2-(hydroxymethyl)phenyl]phenyl]ethyl]-3-methyl-3H-pyrimidin-4-one;
2-amino-6-[2-[3-(4-hydroxy-2-methyl-phenyl)phenyl]ethyl]-3-methyl-3H-pyrimidin-4-one;
2-amino-6-[2-[3-(4-methoxy-2-methyl-phenyl)phenyl]ethyl]-3-methyl-3H-pyrimidin-4-one;
2-amino-6-[2-[3-(5-chloro-2-thienyl)phenyl]ethyl]-3-methyl-3H-pyrimidin-4-one;
6-[2-[3-(5-acetyl-2-thienyl)phenyl]ethyl]-2-amino-3-methyl-3H-pyrimidin-4-one;
2-amino-6-[2-[3-[4-(methoxymethyl)phenyl]phenyl]ethyl]-3-methyl-3H-pyrimidin-4-one;
4-[3-[2-(2-amino-1-methyl-6-oxo-1H-pyrimidin-4-yl)ethyl]phenyl]benzamide;
2-amino-3-methyl-6-[2-[3-(2-methylsulfonylphenyl)phenyl]ethyl]-3H-pyrimidin-4-one
2-amino-3-methyl-6-[2-[3-(5-quinolyl)phenyl]ethyl]-3H-pyrimidin-4-one;
2-Amino-3-(cyclohexylmethyl)-6-[2-[3-(2-furyl)phenyl]ethyl]-3H-pyrimidin-4-one;
2-Amino-6-[2-[3-(2-furyl)phenyl]ethyl]-3-(tetrahydrofuran-2-ylmethyl)-3H-pyrimidin-4-one;
2-Amino-6-[2-[3-(2-furyl)phenyl]ethyl]-3-(3-hydroxypropyl)-3H-pyrimidin-4-one;
2-Amino-6-[2-[3-(2-furyl)phenyl]ethyl]-3-[(3-methoxyphenyl)methyl]-3H-pyrimidin-4-one;
2-amino-6-[2-[3-(2-furyl)phenyl]ethyl]-3-[[3-(1H-tetrazol-5-yl)phenyl]methyl]-3H-pyrimidin-4-one;
3-[[2-amino-4-[2-(3-furan-2-yl-phenyl)-ethyl]-6-oxo-6H-pyrimidin-1-ylmethyl]-benzonitrile;
3-[[2-Amino-4-[2-[3-(2-furyl)phenyl]ethyl]-6-oxo-1H-pyrimidin-1-yl]methyl]benzoic acid;
3-[[2-Amino-4-[2-[3-(2-furyl)phenyl]ethyl]-6-oxo-6H-pyrimidin-1-yl]methyl]benzoic acid methyl ester;
3-[2-[2-Amino-4-[2-[3-(2-furyl)phenyl]ethyl]-6-oxo-1H-pyrimidin-1-yl]ethylamino]-3-oxo-propanoic acid;
2-[2-[2-Amino-4-[2-[3-(2-furyl)phenyl]ethyl]-6-oxo-1H-pyrimidin-1-yl]ethylcarbamoyl]benzoic acid;
2-Amino-6-(3′-ethyl-biphenyl-3-ylmethyl)-3-methyl-3H-pyrimidin-4-one;
2-Amino-6-{3-[(E)-2-(3-methoxy-phenyl)-vinyl]-benzyl}-3-methyl-3H-pyrimidin-4-one;
2-Amino-6-{3-[2-(3-methoxy-phenyl)-ethyl]-benzyl}-3-methyl-3H-pyrimidin-4-one;
2-Amino-3-methyl-6-[3-((E)-styryl)-benzyl]-3H-pyrimidin-4-one;
2-Amino-6-{3-[(E)-2-(4-methoxy-phenyl)-vinyl]-benzyl}-3-methyl-3H-pyrimidin-4-one;
2-Amino-3-methyl-6-[3-((E)-3-methyl-pent-1-enyl)-benzyl]-3H-pyrimidin-4-one;
2-Amino-3-methyl-6-[3-((E)-4-methyl-pent-1-enyl)-benzyl]-3H-pyrimidin-4-one;
2-Amino-3-methyl-6-(3-phenethyl-benzyl)-3H-pyrimidin-4-one;
2-Amino-3-methyl-6-[3-(2-pyridin-4-yl-ethyl)-benzyl]-3H-pyrimidin-4-one;
2-Amino-3-methyl-6-[3-(3-methyl-pentyl)-benzyl]-3H-pyrimidin-4-one;
2-Amino-3-methyl-6-[3-(4-methyl-pentyl)-benzyl]-3H-pyrimidin-4-one;
2-amino-3,6-dimethyl-6-naphthalen-1-yl-5,6-dihydro-3H-pyrimidin-4-one;
2-amino-6-(4-chloro-3-naphthalen1-yl-phenyl)-3,6-dimethyl-5,6-dihydro-3H-pyrimidin-4-one;
2-amino-6-(6,4′-dichloro-biphenyl-3-yl)-3,6-dimethyl-5,6-dihydro-3H-pyrimidin-4-one
2-amino-6-(4-chloro-3-isoquinolin-5-yl-phenyl)-3,6-dimethyl-5,6-dihydro-3H-pyrimidin-4-one;
2-amino-6-(6-chloro-4′-hydroxy-biphenyl-3-yl-phenyl)-3,6-dimethyl-5,6-dihydro-3H-pyrimidin-4-one;
2-amino-6-(4-chloro-3-dibenzofuran-4-yl-phenyl)-3,6-dimethyl-5,6-dihydro-3H-pyrimidin-4-one;
2-amino-6-[4-chloro-3-(2,3-dihydrobenzo[1,4]dioxin-6-yl-phenyl]-3,6-dimethyl-5,6-dihydro-3H-pyrimidin-4-one;
2-amino-3-benzyl-6-(3-bromo-phenyl)-6-methyl-5,6-dihydro-3H-pyrimidin-4-one;
2-amino-6-[4-chloro-3-(1H-indol-6-yl)-phenyl]-3,6-dimethyl-5,6-dihydro-3H-pyrimidin-4-one;
2-amino-3-benzyl-6-(3′-methoxy-biphenyl-3-yl)-6-methyl-5,6-dihydro-3H-pyrimidin-4-one;
2-amino-3-benzyl-6-methyl-6-phenyl-5,6-dihydro-3H-pyrimidin-4-one;
2-amino-6-(6-chloro-biphenyl-3-yl)-3,6-dimethyl-5,6-dihydro-3H-pyrimidin-4-one;
2-amino-6-(6-chloro-4′-methoxy-biphenyl-3-yl)-3,6-dimethyl-5,6-dihydro-3H-pyrimidin-4-one;
2-amino-6-(3-methoxy-phenyl)-3,6-dimethyl-5,6-dihydro-3H-pyrimidin-4-one;
2-amino-3-methyl-6-[2-[3-(2-thienyl)phenyl]ethyl]-3H-pyrimidin-4-one;
2-amino-6-[2-[3-(3-methoxyphenyl)phenyl]ethyl]-3,6-dimethyl-5,6-dihydro-3H-pyrimidin-4-one;
2-amino-3-methyl-6-phenyl-5,6-dihydro-3H-pyrimidin-4-one;
3-[2-[2-amino-4-[2-[3-(2-furyl)phenyl]ethyl]-6-oxo-1H-pyrimidin-1-yl]ethylcarbaamoyl]benzoic acid;
2-amino-6-[2-[3-(2-furyl)phenyl]ethyl]-3-[2-(1H-imidazol-1-yl)ethyl]-3H-pyrimidin-4-one;
2-amino-3-[2-[bis(3-furylmethyl)amino]ethyl]-6-[2-[3-(2-furyl)phenyl]ethyl]-3H-pyrimidin-4-one;
N-[2-[2-amino-4-[2-[3-(2-furyl)phenyl]ethyl]-6-oxo-1H-pyrimidin-1-yl]ethyl]-2-(1H-tetrazol-5-yl)acetamide;
5-[2-[2-amino-4-[2-[3-(2-furyl)phenyl]ethyl]-6-oxo-1H-pyrimidin-1-yl]ethylamino]-5-oxo-pentanoic acid;
acetic acid 2-[2-amino-4-[2-[3-(2-furyl)phenyl]ethyl]-6-oxo-1H-pyrimidin-1-yl]ethylcarbamoylmethyl ester;
N-[2-[2-amino-4-[2-[3-(2-furyl)phenyl]ethyl]-6-oxo-1H-pyrimidin-1-yl]ethyl]-3-hydroxy-benzamide;
N-[2-[2-amino-4-[2-[3-(2-furyl)phenyl]ethyl]-6-oxo-1H-pyrimidin-1-yl]ethyl]-4-hydroxy-benzamide;
4-amino-N-[2-[2-amino-4-[2-[3-(2-furyl)phenyl]ethyl]-6-oxo-1H-pyrimidin-1-yl]ethyl]benzamide;
N-[2-[2-amino-4-[2-[3-(2-furyl)phenyl]ethyl]-6-oxo-1H-pyrimidin-1-yl]ethyl]piperidine-4-carboxamide;
2-amino-6-[2-[3-(2-furyl)phenyl]ethyl]-3-(2-phenethylaminoethyl)-3H-pyrimidin-4-one;
4-[2-[2-amino-4-[2-[3-(2-furyl)phenyl]ethyl]-6-oxo-1H-pyrimidin-1-yl]ethylaminomethyl]benzoic acid;
2-amino-6-[2-[3-(2-furyl)phenyl]ethyl]-3-[2-[(3-hydroxyphenyl)methylamino]ethyl]-3H-pyrimidin-4-one;
2-amino-6-[2-[3-(2-furyl)phenyl]ethyl]-3-[2-[[4-(2-hydroxyethoxy)phenyl]methylamino]ethyl]-3H-pyrimidin-4-one;
2-amino-6-[2-[3-(2-furyl)phenyl]ethyl]-3-(2-isobutylaminoethyl)-3H-pyrimidin-4-one;
2-amino-6-[2-[3-(2-furyl)phenyl]ethyl]-3-[2-(1H-indol-5-ylmethylamino)ethyl]-3H-pyrimidin-4-one;
2-amino-6-[2-[3-(2-furyl)phenyl]ethyl]-3-[2-[(4-hydroxyphenyl)methylamino]ethyl]-3H-pyrimidin-4-one;
2-amino-6-[2-[3-(2-furyl)phenyl]ethyl]-3-[2-(3-pyridylmethylamino)ethyl]-3H-pyrimidin-4-one;
2-amino-3-benzyl-6-[2-[3-(2-furyl)phenyl]ethyl]-3H-pyrimidin-4-one;
2-amino-6-[2-[3-(2-furyl)phenyl]ethyl]-3-(2-hydroxyethyl)-3H-pyrimidin-4-one;
2-amino-6-[2-[3-(2-furyl)phenyl]ethyl]-3-isopentyl-3H-pyrimidin-4-one;
2-amino-6-[2-[3-(2-furyl)phenyl]ethyl]-3-[2-[(1-hydroxy-2,2,6,6-tetramethyl-4-piperidyl)amino]ethyl]-3H-pyrimidin-4-one;
2-amino-6-[2-[3-(2-furyl)phenyl]ethyl]-3-[2-[(1-hydroxy-2,2,6,6-tetramethyl-4-piperidyl)amino]ethyl]-3H-pyrimidin-4-one;
2-amino-3-[2-(1,3-dioxan-2-yl)ethyl]-6-[2-[3-(2-furyl)phenyl]ethyl]-3H-pyrimidin-4-one;
4-[[2-amino-4-[2-[3-(2-furyl)phenyl]ethyl]-6-oxo-1H-pyrimidin-1-yl]methyl]benzonitrile;
2-amino-6-[2-[3-(2-furyl)phenyl]ethyl]-3-[[4-(1H-tetrazol-5-yl)phenyl]methyl]-3H-pyrimidin-4-one;
2-amino-6-[2-[3-(2-furyl)phenyl]ethyl]-3-(2-morpholinoethyl)-3H-pyrimidin-4-one;
2-[[2-amino-4-[2-[3-(2-furyl)phenyl]ethyl]-6-oxo-1H-pyrimidin-1-yl]methyl]benzonitrile; and
2-amino-6-[2-[3-(2-furyl)phenyl]ethyl]-3-[2-(1-piperidyl)ethyl]-3H-pyrimidin-4-one,
or a pharmaceutically acceptable salt, tautomer, or in vivo-hydrolysable precursor thereof.

54. Use of a compound-according to claim 1, or a pharmaceutically acceptable salt, tautomer or in vivo-hydrolysable precursor thereof, as a medicament.

55. Use of a compound according to claim 1, or a pharmaceutically acceptable salt, tautomer or in vivo-hydrolysable precursor thereof, as a medicament for treating or preventing an Aβ-related pathology.

56. Use of a compound according to claim 1, or a pharmaceutically acceptable salt, tautomer or in vivo-hydrolysable precursor thereof, as a medicament for treating or preventing an Aβ-related pathology, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

57. Use of a compound according to claim 1, or a pharmaceutically acceptable salt, tautomer or in vivo-hydrolysable precursor thereof, in the manufacture of a medicament for treating or preventing an Aβ-related pathology.

58. Use of a compound according to claim 1, or a pharmaceutically acceptable salt, tautomer or in vivo-hydrolysable precursor thereof, in the manufacture of a medicament for treating or preventing an Aβ-related pathology, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

59. A method of inhibiting activity of BACE comprising contacting said BACE with a compound according to claim 1, or a pharmaceutically acceptable salt, tautomer or in vivo-hydrolysable precursor thereof.

60. A method of treating or preventing an Aβ-related pathology in a mammal, comprising administering to said patient a therapeutically effective amount of a compound according to claim 1, or a pharmaceutically acceptable salt, tautomer or in vivo-hydrolysable precursor thereof.

61. A method according to claim 27, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

62. The method according to claim 27, wherein said mammal is a human.

63. A method of treating or preventing an Aβ-related pathology in a mammal, comprising administering to said patient a therapeutically effective amount of a compound according to claim 1, or a pharmaceutically acceptable salt, tautomer or in vivo-hydrolysable precursor thereof, and at least one cognitive enhancing agent, memory enhancing agent, or choline esterase inhibitor.

64. A method according to claim 30, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

65. A method according to claim 30, wherein said mammal is a human.

66. A pharmaceutical composition comprising a compound according to claim 1, or a pharmaceutically acceptable salt, tautomer or in vivo-hydrolysable precursor thereof, and at least one pharmaceutically acceptable carrier, diluent or excipient.
